(19) United States
(12) Patent Application Publication
(10) Pub. No.: US 2011/0076271 A1
(43) Pub. Date: Mar. 31, 2011

(54) DIAGNOSTIC METHODS AND COMPOSITIONS FOR TREATMENT OF CANCER

(75) Inventors: Maike Schmidt, San Francisco, CA (US); Laura Sanders, El Granada, CA (US); Rajiv Raja, San Ramon, CA (US); Rajesh D. Patel, Fremont, CA (US)

(73) Assignee: Genentech, Inc., South San Francisco, CA (US)

(21) Appl. No.: 12/834,523

(22) Filed: Jul. 12, 2010

Related U.S. Application Data
(60) Provisional application No. 61/225,120, filed on Jul. 13, 2009.
(60) Provisional application No. 61/351,733, filed on Jun. 4, 2010.

(51) Int. Cl. A61K 39/395; C12Q 1/68; A61K 31/713; A61P 35/00

(52) U.S. Cl. 424/133.1; 435/6; 424/130.1; 424/172.1; 424/158.1; 514/44 A

(57) ABSTRACT

Disclosed herein are methods and compositions useful for the diagnosis and treatment of angiogenic disorders, including, e.g., cancer.

### RELATED APPLICATIONS

The present application claims the benefit of U.S. Provisional Patent Applications Nos. 61/225,120 filed Jul. 13, 2009 and 61/351,733 filed Jun. 4, 2010, the disclosures of which are hereby incorporated by reference in their entirety for all purposes.

### FIELD OF THE INVENTION

The present invention relates to diagnostic methods and compositions useful in the treatment of angiogenic disorders including, e.g., cancer.

### BACKGROUND OF THE INVENTION

Angiogenic disorders such as cancer are one of the most deadly threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Depending on the cancer type, patients typically have several treatment options available to them including chemotherapy, radiation and antibody-based drugs. Diagnostic methods useful for predicting clinical outcome from the different treatment regimens would greatly benefit clinical management of these patients. Several studies have explored the correlation of gene expression with the identification of specific cancer types, e.g., by mutation-specific assays, microarray analysis, qPCR, etc. Such methods may be useful for the identification and classification of cancer presented by a patient. However, much less is known about the predictive or prognostic value of gene expression with clinical outcome.

Thus, there is a need for objective, reproducible methods for the optimal treatment regimen for each patient.

### SUMMARY OF THE INVENTION

The methods of the present invention can be utilized in a variety of settings, including, for example, in selecting the optimal treatment course for a patient, in predicting the likelihood of success when treating an individual patient with a particular treatment regimen, in assessing disease progression, in monitoring treatment efficacy, in determining prognosis for individual patients and in assessing predisposition of an individual to benefit from a particular therapy, e.g., an anti-angiogenic therapy including, for example, an anti-cancer therapy).

The present invention is based, in part, on the use of biomarkers indicative for efficacy of therapy (e.g., anti-angiogenic therapy including, for example, an anti-cancer therapy). More particularly, the invention is based on measuring an increase or decrease in the expression level(s) of at least one gene selected from: 18S rRNA, ACTB, RPS13, VEGFA, VEGFC, VEGFD, Bv8, PlGF, VEGFR1/Flt1, VEGFR2, VEGFR3, NRP1, sNRP1, Podoplanin, Prox1, VE-Cadherin (CD144, CDH5), robo4, FGF2, IL8/CXCL8, HGF, THBS1/TSP1, Egfl7, NG3/Egfl8, ANG1, GM-CSF/CSF2, G-CSF/CSF3, FGF9, CXCL12/SDF1, TGFβ1, TNFα, Alk1, BMP9, BMP10, HSPG2/perlecan, ESM1, Sema3a, Sema3b, Sema3c, Sema3e, Sema3f, NG2, ITGa5, ICAM1, CXCR4, LGALS1/Galectin1, LGALS7B/Galectin7, Fibronectin, TMEM100, PECAM/CD31, PDGFβ, PDGFRβ, RGS5, CXCL1, CXCL2, robo4, LyPD6, VCAM1, collagen IV, Spred-1, Hhex, ITGa5, LGALS1/Galectin1, LGALS7/Galectin7, TMEM100, MFAP5, Fibronectin, fibulin2, fibulin4/Efemp2, HMBS, SDHA, UBC, NRP2, CD34, DLL4, CLECSF5/CLEC5a, CCL2/MCP1, CCL5, CXCL5/ENA-78, ANG2, FGF8, FGF8b, PDGFC, cMet, JAG1, CD105/Endoglin, Notch1, EphB4, EphA3, EFNB2, TIE2/TEK, LAMA4, NID2, Map4k4, Bcl2A1, IGFBP4, VIM/vimentin, FGFR4, FRAS1, ANTXR2, CLECSF5/CLEC5a, and Mincle/CLEC4E/CLECSF9 to predict the efficacy of therapy (e.g., anti-angiogenic therapy including, for example, an anti-cancer therapy).

One embodiment of the invention provides methods of identifying a patient who may benefit from treatment with an anti-cancer therapy other than or in addition to a VEGF antagonist. The methods comprise determining expression levels of at least one gene set forth in Table 1 in a sample obtained from the patient, wherein an increased expression level of the at least one gene in the sample as compared to a reference sample indicates that the patient may benefit from treatment with the anti-cancer therapy other than or in addition to a VEGF antagonist.

Another embodiment of the invention provides methods of identifying a patient who may benefit from treatment with an anti-cancer therapy other than or in addition to a VEGF antagonist. The methods comprise: determining expression levels of at least one gene set forth in Table 1 in a sample obtained from the patient, wherein a decreased expression level of the at least one gene in the sample as compared to a reference sample indicates that the patient may benefit from treatment with the anti-cancer therapy other than or in addition to a VEGF antagonist.

A further embodiment of the invention provides methods of predicting responsiveness of a patient suffering from cancer to treatment with an anti-cancer therapy other than or in addition to a VEGF antagonist. The methods comprise determining expression levels of at least one gene set forth in Table 1 in a sample obtained from the patient, wherein an increased expression level of the at least one gene in the sample as compared to a reference sample indicates that the patient is more likely to be responsive to treatment with the anti-cancer therapy other than or in addition to a VEGF antagonist.

Yet another embodiment of the invention provides methods of predicting responsiveness of a patient suffering from cancer to treatment with an anti-cancer therapy other than or in addition to a VEGF antagonist. The methods comprise: determining expression levels of at least one gene set forth in Table 1 in a sample obtained from the patient, wherein a decreased expression level of the at least one gene in the sample as compared to a reference sample indicates that the patient is more likely to be responsive to treatment with the anti-cancer therapy other than or in addition to a VEGF antagonist.

Even another embodiment of the invention provides methods for determining the likelihood that a patient with cancer will exhibit benefit from anti-cancer therapy other than or in addition to a VEGF antagonist. The methods comprise: determining expression levels of at least one gene set forth in Table 1 in a sample obtained from the patient, wherein an increased expression level of the at least one gene in the sample as compared to a reference sample indicates that the patient has increased likelihood of benefit from the anti-cancer therapy other than or in addition to a VEGF antagonist.

Another embodiment of the invention provides methods for determining the likelihood that a patient with cancer will exhibit benefit from anti-cancer therapy other than or in addition to a VEGF antagonist. The methods comprise: determining expression levels of at least one gene set forth in Table 1 in a sample obtained from the patient, wherein a decreased expression level of the at least one gene in the sample as compared to a reference sample indicates that the patient has increased likelihood of benefit from the anti-cancer therapy other than or in addition to a VEGF antagonist.

A further embodiment of the invention provides methods for treating cancer in a patient. The methods comprise: determining that a sample obtained from the patient has increased expression levels, as compared to a reference sample, of at least one gene set forth in Table 1, and administering an effective amount of an anti-cancer therapy other than or in addition to a VEGF antagonist to the patient, whereby the cancer is treated.

Another embodiment of the invention provides methods for treating cancer in a patient. The methods comprise determining that a sample obtained from the patient has decreased expression levels, as compared to a reference sample, of at least one gene set forth in Table 1, and administering an effective amount of an anti-cancer therapy other than or in addition to a VEGF antagonist to the patient, whereby the cancer is treated.

In some embodiments of the invention, the sample obtained from the patient is selected from: tissue, whole blood, blood-derived cells, plasma, serum, and combinations thereof. In some embodiments of the invention, the expression level is mRNA expression level. In some embodiments of the invention, the expression level is protein expression level.

In some embodiments of the invention, the methods further comprise detecting the expression of at least a second, third, fourth, fifth, sixth, seventh, eighth, ninth, tenth, eleventh, twelfth, thirteenth, fourteenth, fifteenth, sixteenth, seventeenth, eighteenth, nineteenth, or twentieth gene set forth in Table 1.

In some embodiments of the invention, the methods further comprising administering the anti-cancer therapy other than a VEGF antagonist to the patient. In some embodiments of the invention, the anti-cancer therapy is selected from: an antibody, a small molecule, and an siRNA. In some embodiments of the invention, the anti-cancer therapy is a member selected from: an EGFL7 antagonist, a NRP1 antagonist, and a VEGF-C antagonist. In some embodiments of the invention, the EGFL7 antagonist is an antibody. In some embodiments of the invention, the NRP1 antagonist is an antibody. In some embodiments of the invention, the VEGF-C antagonist is an antibody.

In some embodiments of the invention, the methods further comprise administering the VEGF antagonist to the patient. In some embodiments of the invention, the VEGF antagonist is an anti-VEGF antibody. In some embodiments of the invention, the anti-VEGF antibody is bevacizumab. In some embodiments of the invention, the anti-cancer therapy and the VEGF antagonist are administered concurrently. In some embodiments of the invention, the anti-cancer therapy and the VEGF antagonist are administered sequentially.

Even another embodiment of the invention provides kits for determining whether a patient may benefit from treatment with an anti-cancer therapy other than or in addition to a VEGF antagonist. The kits comprise an array comprising polynucleotides capable of specifically hybridizing to at least one gene set forth in Table 1 and instructions for using said array to determine the expression levels of the at least one gene to predict responsiveness of a patient to treatment with an anti-cancer therapy in addition to a VEGF antagonist, wherein an increase in the expression level of the at least one gene as compared to the expression level of the at least one gene in a reference sample indicates that the patient may benefit from treatment with the anti-cancer therapy in addition to a VEGF antagonist.

A further embodiment of the invention provides kits for determining whether a patient may benefit from treatment with an anti-cancer therapy other than or in addition to a VEGF antagonist. The kits comprise an array comprising polynucleotides capable of specifically hybridizing to at least one gene set forth in Table 1 and instructions for using said array to determine the expression levels of the at least one gene to predict responsiveness of a patient to treatment with an anti-cancer therapy in addition to a VEGF antagonist, wherein a decrease in the expression level of the at least one gene as compared to the expression level of the at least one gene in a reference sample indicates that the patient may benefit from treatment with an anti-cancer therapy in addition to a VEGF antagonist.

Another embodiment of the invention provides sets of compounds for detecting expression levels of at least one gene set forth in Table 1 to determine the expression levels of the at least one gene in a sample obtained from a cancer patient. The sets comprise at least one compound capable of specifically hybridizing to at least one gene set forth in Table 1, wherein an increase in the expression level of the at least one gene as compared to the expression level of the at least one gene in a reference sample indicates that the patient may benefit from treatment with an anti-cancer therapy in addition to a VEGF antagonist. In some embodiments of the invention, the compounds are polynucleotides. In some embodiments of the invention, the polynucleotides comprise three sequences set forth in Table 2. In some embodiments of the invention, the compounds are proteins, such as, for example, antibodies.

Yet another embodiment of the invention provides sets of compounds for detecting expression levels of at least one gene set forth in Table 1 to determine the expression levels of the at least one gene in a sample obtained from a cancer patient. The sets comprise at least one compound capable of specifically hybridizing to at least one gene set forth in Table 1, wherein a decrease in the expression level of the at least one gene as compared to the expression level of the at least one gene in a reference sample indicates that the patient may benefit from treatment with an anti-cancer therapy in addition to a VEGF antagonist. In some embodiments of the invention, the compounds are polynucleotides. In some embodiments of the invention, the polynucleotides comprise three sequences set forth in Table 2. In some embodiments of the invention, the compounds are proteins, such as, for example, antibodies.

One embodiment of the invention provides methods of identifying a patient suffering from cancer who may benefit from treatment with a neuropilin-1 (NRP1) antagonist. The methods comprise determining expression levels of at least one gene selected from: TGFβ1, Bv8, Sema3A, PlGF, LGALS1, ITGa5, CSF2, Vimentin, CXCL5, CCL2, CXCL2, Alk1, and FGF8 in a sample obtained from the patient, wherein increased expression levels of the at least one gene in the sample as compared to a reference sample indicates that the patient may benefit from treatment with the NRP1 antagonist.

Another embodiment of the invention provides methods of identifying a patient suffering from cancer who may benefit from treatment with a neuropilin-1 (NRP1) antagonist. The methods comprise determining expression levels of at least one gene selected from: Prox1, RGS5, HGF, Sema3B, Sema3F, LGALS7, FGRF4, PLC, IGFB4, and TSP1 in a sample obtained from the patient, wherein decreased expression levels of the at least one gene in the sample as compared to a reference sample indicates that the patient may benefit from treatment with the NRP1 antagonist.

A further embodiment of the invention provides methods of predicting responsiveness of a patient suffering from cancer to treatment with a NRP1 antagonist. The methods comprise determining expression levels of at least one gene selected from: TGFβ1, Bv8, Sema3A, PlGF, LGALS1, ITGa5, CSF2, Vimentin, CXCL5, CCL2, CXCL2, Alk1, and FGF8 in a sample obtained from the patient, wherein increased expression levels of the at least one gene in the sample as compared to a reference sample indicates that the patient is more likely to be responsive to treatment with the NRP1 antagonist.

Even another further embodiment of the invention provides methods of predicting responsiveness of a patient suffering from cancer to treatment with a NRP1 antagonist. The methods comprise determining expression levels of at least one gene selected from: Prox1, RGS5, HGF, Sema3B, Sema3F, LGALS7, FGRF4, PLC, IGFB4, and TSP1 in a sample obtained from the patient, wherein decreased expression levels of the at least one gene in the sample as compared to a reference sample indicates that the patient is more likely to be responsive to treatment with the NRP1 antagonist.

Yet another embodiment of the invention provides methods of determining the likelihood that a patient will exhibit a benefit from treatment with a NRP1 antagonist. The methods comprise determining expression levels of at least one gene selected from: TGFβ1, Bv8, Sema3A, PlGF, LGALS1, ITGa5, CSF2, Vimentin, CXCL5, CCL2, CXCL2, Alk1, and FGF8 in a sample obtained from the patient, wherein increased expression levels of the at least one gene in the sample as compared to a reference sample indicates that the patient has increased likelihood of benefit from treatment with the NRP1 antagonist.

Another embodiment of the invention provide methods of determining the likelihood that a patient will exhibit a benefit from treatment with a NRP1 antagonist. The methods comprise determining expression levels of at least one gene selected from: Prox1, RGS5, HGF, Sema3B, Sema3F, LGALS7, FGRF4, PLC, IGFB4, and TSP1 in a sample obtained from the patient, wherein decreased expression levels of the at least one gene in the sample as compared to a reference sample indicates that the patient has increased likelihood of benefit of treatment with the NRP1 antagonist.

Yet another embodiment of the invention provides methods of optimizing therapeutic efficacy of a NRP1 antagonist. The methods comprise determining expression levels of at least one gene selected from: TGFβ1, Bv8, Sema3A, PlGF, LGALS1, ITGa5, CSF2, Vimentin, CXCL5, CCL2, CXCL2, Alk1, and FGF8 in a sample obtained from the patient, wherein increased expression levels of the at least one gene in the sample as compared to a reference sample indicates that the patient has increased likelihood of benefit from treatment with the NRP1 antagonist.

Another embodiment of the invention provide methods of optimizing therapeutic efficacy of a NRP1 antagonist. The methods comprise determining expression levels of at least one gene selected from: Prox1, RGS5, HGF, Sema3B, Sema3F, LGALS7, FGRF4, PLC, IGFB4, and TSP1 in a sample obtained from the patient, wherein decreased expression levels of the at least one gene in the sample as compared to a reference sample indicates that the patient has increased likelihood of benefit of treatment with the NRP1 antagonist.

A further embodiment of the invention provides methods for treating a cell proliferative disorder in a patient. The methods comprise determining that a sample obtained from the patient has increased expression levels, as compared to a reference sample, of at least one gene selected from: TGFβ1, Bv8, Sema3A, PlGF, LGALS1, ITGa5, CSF2, Vimentin, CXCL5, CCL2, CXCL2, Alk1, and FGF8, and administering to the patient an effective amount of a NRP1 antagonist, whereby the cell proliferative disorder is treated.

Yet another embodiment of the invention provides methods for treating a cell proliferative disorder in a patient. The methods comprise determining that a sample obtained from the patient has decreased expression levels, as compared to a reference sample, of at least one gene selected from: Prox1, RGS5, HGF, Sema3B, Sema3F, LGALS7, FGRF4, PLC, IGFB4, and TSP1, and administering to the patient an effective amount of a NRP1 antagonist, whereby the cell proliferative disorder is treated.

In some embodiments of the invention, the sample obtained from the patient is a member selected from: tissue, whole blood, blood-derived cells, plasma, serum, and combinations thereof. In some embodiments of the invention, the expression level is mRNA expression level. In some embodiments of the invention, the expression level is protein expression level. In some embodiments of the invention, the NRP1 antagonist is an anti-NRP1 antibody.

In some embodiments of the invention, the methods further comprise administering a VEGF antagonist to the patient. In some embodiments of the invention, the VEGF antagonist and the NRP1 antagonist are administered concurrently. In some embodiments of the invention, the VEGF antagonist and the NRP1 antagonist are administered sequentially. In some embodiments of the invention, the VEGF antagonist is an anti-VEGF antibody. In some embodiments of the invention, the anti-VEGF antibody is bevacizumab.

Another embodiment of the invention provides methods of identifying a patient suffering from cancer who may benefit from treatment with a NRP1 antagonist. The methods comprise determining expression levels of PlGF in a sample obtained from the patient, wherein increased expression levels of PlGF in the sample as compared to a reference sample indicates that the patient may benefit from treatment with the NRP1 antagonist.

Even another embodiment of the invention provides methods of predicting responsiveness of a patient suffering from cancer to treatment with a NRP1 antagonist. The methods comprise determining expression levels of PlGF in a sample obtained from the patient, wherein increased expression levels of PlGF in the sample as compared to a reference sample indicates that the patient is more likely to be responsive to treatment with the NRP1 antagonist.

Yet another embodiment of the invention provides methods of determining the likelihood that a patient will exhibit a benefit from treatment with a NRP1 antagonist. The methods comprise determining expression levels of PlGF in a sample obtained from the patient, wherein increased expression levels of PlGF in the sample as compared to a reference sample indicates that the patient has increased likelihood of benefit from treatment with the NRP1 antagonist.

Even another embodiment of the invention provides methods of optimizing therapeutic efficacy of a NRP1 antagonist. The methods comprise determining expression levels of PlGF in a sample obtained from the patient, wherein increased expression levels of PlGF in the sample as compared to a reference sample indicates that the patient has increased likelihood of benefit from treatment with the NRP1 antagonist.

A further embodiment of the invention provides methods for treating a cell proliferative disorder in a patient. The methods comprise determining that a sample obtained from the patient has increased expression levels of PlGF as compared to a reference sample, and administering to the patient an effective amount of a NRP1 antagonist, whereby the cell proliferative disorder is treated.

Even a further embodiment of the invention provides methods of identifying a patient suffering from cancer who may benefit from treatment with a neuropilin-1 (NRP1) antagonist. The methods comprise determining expression levels of Sema3A in a sample obtained from the patient, wherein increased expression levels of Sema3A in the sample as compared to a reference sample indicates that the patient may benefit from treatment with the NRP1 antagonist.

Yet a further embodiment of the invention provides methods of predicting responsiveness of a patient suffering from cancer to treatment with a NRP1 antagonist. The methods comprise determining expression levels of Sema3A in a sample obtained from the patient, wherein increased expression levels of Sema3A in the sample as compared to a reference sample indicates that the patient is more likely to be responsive to treatment with the NRP1 antagonist.

Another embodiment of the invention provides methods of determining the likelihood that a patient will exhibit a benefit from treatment with a NRP1 antagonist. The methods comprise determining expression levels of Sema3A in a sample obtained from the patient, wherein increased expression levels of Sema3A in the sample as compared to a reference sample indicates that the patient has increased likelihood of benefit from treatment with the NRP1 antagonist.

Another embodiment of the invention provides methods of optimizing therapeutic efficacy of a NRP1 antagonist. The methods comprise determining expression levels of Sema3A in a sample obtained from the patient, wherein increased expression levels of Sema3A in the sample as compared to a reference sample indicates that the patient has increased likelihood of benefit from treatment with the NRP1 antagonist.

Even another embodiment of the invention provides methods for treating a cell proliferative disorder in a patient. The methods comprise determining that a sample obtained from the patient has increased expression levels of Sema3A as compared to a reference sample, and administering to the patient an effective amount of a NRP1 antagonist, whereby the cell proliferative disorder is treated

Yet another embodiment of the invention provides methods of identifying a patient suffering from cancer who may benefit from treatment with a neuropilin-1 (NRP1) antagonist. The methods comprise determining expression levels of TGFβ1 in a sample obtained from the patient, wherein increased expression levels of TGFβ1 in the sample as compared to a reference sample indicates that the patient may benefit from treatment with the NRP1 antagonist.

A further embodiment of the invention provides methods of predicting responsiveness of a patient suffering from cancer to treatment with a NRP1 antagonist. The methods comprise determining expression levels of TGFβ1 in a sample obtained from the patient, wherein increased expression levels of TGFβ1 in the sample as compared to a reference sample indicates that the patient is more likely to be responsive to treatment with the NRP1 antagonist. In some embodiments of the invention, the methods further comprise administering an effective amount of a NRP1 antagonist to the patient.

Even a further embodiment of the invention provides methods of determining the likelihood that a patient will exhibit a benefit from treatment with a NRP1 antagonist. The methods comprise determining expression levels of TGFβ1 in a sample obtained from the patient, wherein increased expression levels of TGFβ1 in the sample as compared to a reference sample indicates that the patient has increased likelihood of benefit from treatment with the NRP1 antagonist.

Even a further embodiment of the invention provides methods of optimizing therapeutic efficacy of a NRP1 antagonist. The methods comprise determining expression levels of TGFβ1 in a sample obtained from the patient, wherein increased expression levels of TGFβ1 in the sample as compared to a reference sample indicates that the patient has increased likelihood of benefit from treatment with the NRP1 antagonist.

Yet a further embodiment of the invention provides methods for treating a cell proliferative disorder in a patient. The methods comprise determining that a sample obtained from the patient has increased expression levels of TGFβ1 as compared to a reference sample, and administering to the patient an effective amount of a NRP1 antagonist, whereby the cell proliferative disorder is treated

In some embodiments of the invention, the NRP1 antagonist is an anti-NRP1 antibody. In some embodiments of the invention, the methods further comprises administering a VEGF-A antagonist to the patient. In some embodiments of the invention, the VEGF-A antagonist and the NRP1 antagonist are administered concurrently. In some embodiments of the invention, the VEGF-A antagonist and the NRP1 antagonist are administered sequentially. In some embodiments of the invention, the VEGF-A antagonist is an anti-VEGF-A antibody. In some embodiments of the invention, the anti-VEGF-A antibody is bevacizumab.

Another embodiment of the invention provides kits for determining the expression levels of at least one gene selected from: TGFβ1, Bv8, Sema3A, PlGF, LGALS1, ITGa5, CSF2, Vimentin, CXCL5, CCL2, CXCL2, Alk1, and FGF8. The kits comprise an array comprising polynucleotides capable of specifically hybridizing to at least one gene selected from: TGFβ1, Bv8, Sema3A, PlGF, LGALS1, ITGa5, CSF2, Vimentin, CXCL5, CCL2, CXCL2, Alk1, and FGF8 and instructions for using the array to determine the expression levels of the at least one gene to predict responsiveness of a patient to treatment with a NRP1 antagonist, wherein an increase in the expression level of the at least one gene as compared to the expression level of the at least one gene in a reference sample indicates that the patient may benefit from treatment with the NRP1 antagonist.

Even another embodiment of the invention provides kits for determining the expression levels of at least one gene selected from: Prox1, RGS5, HGF, Sema3B, Sema3F, LGALS7, FGRF4, PLC, IGFB4, and TSP1. The kits comprise an array comprising polynucleotides capable of specifically hybridizing to at least one gene selected from: Prox1, RGS5, HGF, Sema3B, Sema3F, LGALS7, FGRF4, PLC, IGFB4, and TSP1 and instructions for using the array to determine the expression levels of the at least one gene to predict responsiveness of a patient to treatment with a NRP1 antagonist, wherein a decrease in the expression level of the at least one gene as compared to the expression level of the at least one gene in a reference sample indicates that the patient may benefit from treatment with the NRP1 antagonist.

Yet another embodiment of the invention provides sets of compounds capable of detecting expression levels of at least one gene selected from: TGFβ1, Bv8, Sema3A, PlGF, LGALS1, ITGa5, CSF2, Vimentin, CXCL5, CCL2, CXCL2, Alk1, and FGF8 to determine the expression levels of the at least one gene in a sample obtained from a cancer patient. The sets comprise at least one compound capable of specifically hybridizing to at least one gene selected from: TGFβ1, Bv8, Sema3A, PlGF, LGALS1, ITGa5, CSF2, Vimentin, CXCL5, CCL2, CXCL2, Alk1, and FGF8, wherein an increase in the expression level of the at least one gene as compared to the expression level of the at least one gene in a reference sample indicates that the patient may benefit from treatment with a NRP1 antagonist. In some embodiments of the invention, the compounds are polynucleotides. In some embodiments of the invention, the polynucleotides comprise three sequences set forth in Table 2. In some embodiments of the invention, the compounds are proteins, including, for example, antibodies.

A further embodiment of the invention provides sets of compounds capable of detecting expression levels of at least one gene selected from: Prox1, RGS5, HGF, Sema3B, Sema3F, LGALS7, FGRF4, PLC, IGFB4, and TSP1 to determine the expression levels of the at least one gene in a sample obtained from a cancer patient. The sets comprise at least one compound capable of specifically hybridizing to at least one gene selected from: Prox1, RGS5, HGF, Sema3B, Sema3F, LGALS7, FGRF4, PLC, IGFB4, and TSP1, wherein a decrease in the expression level of said at least gene as compared to the expression level of the at least one gene in a reference sample indicates that the patient may benefit from treatment with a NRP1 antagonist. In some embodiments of the invention, the compounds are polynucleotides. In some embodiments of the invention, the polynucleotides comprise three sequences set forth in Table 2. In some embodiments of the invention, the compounds are proteins, including, for example, antibodies.

Another embodiment of the invention provides methods of identifying a patient suffering from cancer who may benefit from treatment with a Vascular Endothelial Growth Factor C (VEGF-C) antagonist. The methods comprise determining expression levels of at least one gene selected from: VEGF-C, VEGF-D, VEGFR3, FGF2, RGS5/CDH5, IL-8, CXCL1, and CXCL2 in a sample obtained from the patient, wherein increased expression levels of the at least one gene in the sample as compared to a reference sample indicates that the patient may benefit from treatment with the VEGF-C antagonist.

Even another embodiment of the invention provides methods of identifying a patient suffering from cancer who may benefit from treatment with a VEGF-C antagonist. The methods comprise determining expression levels of at least one gene selected from: VEGF-A, CSF2, Prox1, ICAM1, ESM1, PlGF, ITGa5, TGFβ, Hhex, Col4a1, Col4a2, and Alk1 in a sample obtained from the patient, wherein decreased expression levels of the at least one gene in the sample as compared to a reference sample indicates that the patient may benefit from treatment with the VEGF-C antagonist.

Yet another embodiment of the invention provides methods of predicting responsiveness of a patient suffering from cancer to treatment with a VEGF-C antagonist. The methods comprise determining expression levels of at least one gene selected from: VEGF-C, VEGF-D, VEGFR3, FGF2, RGS5/CDH5, IL-8, CXCL1, and CXCL2 in a sample obtained from the patient, wherein increased expression levels of the at least one gene in the sample as compared to a reference sample indicates that the patient is more likely to be responsive to treatment with the VEGF-C antagonist.

A further embodiment of the invention provides methods of predicting responsiveness of a patient suffering from cancer to treatment with a VEGF-C antagonist. The methods comprise determining expression levels of at least one gene selected from: VEGF-A, CSF2, Prox1, ICAM1, ESM1, PlGF, ITGa5, TGFβ, Hhex, Col4a1, Col4a2, and Alk1 in a sample obtained from the patient, wherein decreased expression levels of the at least one gene in the sample as compared to a reference sample indicates that the patient is more likely to be responsive to treatment with the VEGF-C antagonist.

Even a further embodiment of the invention provides methods of determining the likelihood that a patient will exhibit a benefit from treatment with a VEGF-C antagonist. The methods comprise determining expression levels of at least one gene selected from: VEGF-C, VEGF-D, VEGFR3, FGF2, RGS5/CDH5, IL-8, CXCL1, and CXCL2 in a sample obtained from the patient, wherein increased expression levels of the at least one gene in the sample as compared to a reference sample indicates that the patient has increased likelihood of benefit from treatment with the VEGF-C antagonist.

Yet a further embodiment of the invention provides methods of determining the likelihood that a patient will exhibit a benefit from treatment with a VEGF-C antagonist. The methods comprise determining expression levels of at least one gene selected from: VEGF-A, CSF2, Prox1, ICAM1, ESM1, PlGF, ITGa5, TGFβ, Hhex, Col4a1, Col4a2, and Alk1 in a sample obtained from the patient, wherein decreased expression levels of the at least one gene in the sample as compared to a reference sample indicates that the patient has increased likelihood of benefit of treatment with the VEGF-C antagonist.

Even a further embodiment of the invention provides methods of optimizing therapeutic efficacy of a VEGF-C antagonist. The methods comprise determining expression levels of at least one gene selected from: VEGF-C, VEGF-D, VEGFR3, FGF2, RGS5/CDH5, IL-8, CXCL1, and CXCL2 in a sample obtained from the patient, wherein increased expression levels of the at least one gene in the sample as compared to a reference sample indicates that the patient has increased likelihood of benefit from treatment with the VEGF-C antagonist.

Yet a further embodiment of the invention provides methods of optimizing therapeutic efficacy of a VEGF-C antagonist. The methods comprise determining expression levels of at least one gene selected from: VEGF-A, CSF2, Prox1, ICAM1, ESM1, PlGF, ITGa5, TGFβ, Hhex, Col4a1, Col4a2, and Alk1 in a sample obtained from the patient, wherein decreased expression levels of the at least one gene in the sample as compared to a reference sample indicates that the patient has increased likelihood of benefit of treatment with the VEGF-C antagonist.

Another embodiment of the invention provides methods for treating a cell proliferative disorder in a patient. The methods comprise determining that a sample obtained from the patient has increased expression levels, as compared to a reference sample, of at least one gene selected from: VEGF-C, VEGF-D, VEGFR3, FGF2, RGS5/CDH5, IL-8, CXCL1, and CXCL2, and administering to the patient an effective amount of a VEGF-C antagonist, whereby the cell proliferative disorder is treated.

Even another embodiment of the invention provides methods for treating a cell proliferative disorder in a patient. The methods comprise determining that a sample obtained from the patient has decreased expression levels, as compared to a reference sample, of at least one gene selected from: VEGF-A, CSF2, Prox1, ICAM1, ESM1, PlGF, ITGa5, TGFβ, Hhex, Col4a1, Col4a2, and Alk1, and administering to the patient an effective amount of a VEGF-C antagonist, whereby the cell proliferative disorder is treated.

In some embodiments of the invention, the sample obtained from the patient is selected from: tissue, whole blood, blood-derived cells, plasma, serum, and combinations thereof. In some embodiments of the invention, the expression level is mRNA expression level. In some embodiments of the invention, the expression level is protein expression level. In some embodiments of the invention, the VEGF-C antagonist is an anti-VEGF-C antibody.

In some embodiments of the invention, the methods further comprise administering a VEGF-A antagonist to the patient. In some embodiments of the invention, the VEGF-A antagonist and the VEGF-C antagonist are administered concurrently. In some embodiments of the invention, the VEGF-A antagonist and the VEGF-C antagonist are administered sequentially. In some embodiments of the invention, the VEGF-A antagonist is an anti-VEGF-A antibody. In some embodiments of the invention, the anti-VEGF-A antibody is bevacizumab.

Another embodiment of the invention provides methods of identifying a patient suffering from cancer who may benefit from treatment with a VEGF-C antagonist. The methods comprise determining expression levels of VEGF-C in a sample obtained from the patient, wherein increased expression levels of VEGF-C in the sample as compared to a reference sample indicates that the patient may benefit from treatment with the VEGF-C antagonist.

Even another embodiment of the invention provides methods of predicting responsiveness of a patient suffering from cancer to treatment with a VEGF-C antagonist. The methods comprise determining expression levels of VEGF-C in a sample obtained from the patient, wherein increased expression levels of VEGF-C in the sample as compared to a reference sample indicates that the patient is more likely to be responsive to treatment with the VEGF-C antagonist.

Yet another embodiment of the invention provides methods of determining the likelihood that a patient will exhibit a benefit from treatment with a VEGF-C antagonist. The methods comprise determining expression levels of VEGF-C in a sample obtained from the patient, wherein increased expression levels of VEGF-C in the sample as compared to a reference sample indicates that the patient has an increased likelihood of benefit from treatment with the VEGF-C antagonist.

Yet another embodiment of the invention provides methods of optimizing therapeutic efficacy of a VEGF-C antagonist. The methods comprise determining expression levels of VEGF-C in a sample obtained from the patient, wherein increased expression levels of VEGF-C in the sample as compared to a reference sample indicates that the patient has an increased likelihood of benefit from treatment with the VEGF-C antagonist.

A further embodiment of the invention provides methods for treating a cell proliferative disorder in a patient. The methods comprise determining that a sample obtained from the patient has increased expression levels of VEGF-C as compared to a reference sample, and administering to the patient an effective amount of a VEGF-C antagonist, whereby the cell proliferative disorder is treated.

Even a further embodiment of the invention provides methods of identifying a patient suffering from cancer who may benefit from treatment with a VEGF-C antagonist. The methods comprise determining expression levels of VEGF-D in a sample obtained from the patient, wherein increased expression levels of VEGF-D in the sample as compared to a reference sample indicates that the patient may benefit from treatment with the VEGF-C antagonist.

Yet a further embodiment of the invention provides methods of predicting responsiveness of a patient suffering from cancer to treatment with a VEGF-C antagonist. The methods comprise determining expression levels of VEGF-D in a sample obtained from the patient, wherein increased expression levels of VEGF-D in the sample as compared to a reference sample indicates that the patient is more likely to be responsive to treatment with the VEGF-C antagonist.

Another embodiment of the invention provides methods of determining the likelihood that a patient will exhibit a benefit from treatment with a VEGF-C antagonist. The methods comprise determining expression levels of VEGF-D in a sample obtained from the patient, wherein increased expression levels of VEGF-D in the sample as compared to a reference sample indicates that the patient has an increased likelihood of benefit from treatment with the VEGF-C antagonist.

Another embodiment of the invention provides methods of optimizing therapeutic efficacy of a VEGF-C antagonist. The methods comprise determining expression levels of VEGF-D in a sample obtained from the patient, wherein increased expression levels of VEGF-D in the sample as compared to a reference sample indicates that the patient has an increased likelihood of benefit from treatment with the VEGF-C antagonist.

Even another embodiment of the invention provides methods for treating a cell proliferative disorder in a patient. The methods comprise determining that a sample obtained from the patient has increased expression levels of VEGF-D as compared to a reference sample, and administering to the patient an effective amount of a VEGF-C antagonist, whereby the cell proliferative disorder is treated

Yet another embodiment of the invention provides methods of identifying a patient suffering from cancer who may benefit from treatment with a VEGF-C antagonist. The methods comprise determining expression levels of VEGFR3 in a sample obtained from the patient, wherein increased expression levels of VEGFR3 in the sample as compared to a reference sample indicates that the patient may benefit from treatment with the VEGF-C antagonist.

A further embodiment of the invention provides methods of predicting responsiveness of a patient suffering from cancer to treatment with a VEGF-C antagonist. The methods comprise determining expression levels of VEGFR3 in a sample obtained from the patient, wherein increased expression levels of VEGFR3 in the sample as compared to a reference sample indicates that the patient is more likely to be responsive to treatment with the VEGF-C antagonist.

Even a further embodiment of the invention provides methods of determining the likelihood that a patient will exhibit a benefit from treatment with a VEGF-C antagonist. The methods comprise determining expression levels of VEGFR3 in a sample obtained from the patient, wherein increased expression levels of VEGFR3 in the sample as compared to a reference sample indicates that the patient has an increased likelihood of benefit from treatment with the VEGF-C antagonist.

A further embodiment of the invention provides methods of optimizing therapeutic efficacy of a VEGF-C antagonist. The methods comprise determining expression levels of VEGFR3 in a sample obtained from the patient, wherein increased expression levels of VEGFR3 in the sample as compared to a reference sample indicates that the patient has an increased likelihood of benefit from treatment with the VEGF-C antagonist.

Yet a further embodiment of the invention provides methods for treating a cell proliferative disorder in a patient. The methods comprise determining that a sample obtained from the patient has increased expression levels of VEGFR3 as compared to a reference sample, and administering to the patient an effective amount of a VEGF-C antagonist, whereby the cell proliferative disorder is treated

Another embodiment of the invention provides methods of identifying a patient suffering from cancer who may benefit from treatment with a VEGF-C antagonist. The methods comprise determining expression levels of FGF2 in a sample obtained from the patient, wherein increased expression levels of FGF2 in the sample as compared to a reference sample indicates that the patient may benefit from treatment with the VEGF-C antagonist.

Even another embodiment of the invention provides methods of predicting responsiveness of a patient suffering from cancer to treatment with a VEGF-C antagonist. The methods comprise determining expression levels of FGF2 in a sample obtained from the patient, wherein increased expression levels of FGF2 in the sample as compared to a reference sample indicates that the patient is more likely to be responsive to treatment with the VEGF-C antagonist.

Yet another embodiment of the invention provides methods of determining the likelihood that a patient will exhibit a benefit from treatment with a VEGF-C antagonist. The methods comprise determining expression levels of FGF2 in a sample obtained from the patient, wherein increased expression levels of FGF2 in the sample as compared to a reference sample indicates that the patient has an increased likelihood of benefit from treatment with the VEGF-C antagonist.

Even another embodiment of the invention provides methods of optimizing therapeutic efficacy of a VEGF-C antagonist. The methods comprise determining expression levels of FGF2 in a sample obtained from the patient, wherein increased expression levels of FGF2 in the sample as compared to a reference sample indicates that the patient has an increased likelihood of benefit from treatment with the VEGF-C antagonist.

A further embodiment of the invention provides methods for treating a cell proliferative disorder in a patient. The methods comprise determining that a sample obtained from the patient has increased expression levels of FGF2 as compared to a reference sample, and administering to the patient an effective amount of a VEGF-C antagonist, whereby the cell proliferative disorder is treated

Even a further embodiment of the invention provides methods of identifying a patient suffering from cancer who may benefit from treatment with a VEGF-C antagonist. The methods comprise determining expression levels of VEGF-A in a sample obtained from the patient, wherein decreased expression levels of VEGF-A in the sample as compared to a reference sample indicates that the patient may benefit from treatment with the VEGF-C antagonist.

Yet a further embodiment of the invention provides methods of predicting responsiveness of a patient suffering from cancer to treatment with a VEGF-C antagonist. The methods comprise determining expression levels of VEGF-A in a sample obtained from the patient, wherein decreased expression levels of VEGF-A in the sample as compared to a reference sample indicates that the patient is more likely to be responsive to treatment with the VEGF-C antagonist.

Another embodiment of the invention provides methods of determining the likelihood that a patient will exhibit a benefit from treatment with a VEGF-C antagonist. The methods comprise determining expression levels of VEGF-A in a sample obtained from the patient, wherein decreased expression levels of VEGF-A in the sample as compared to a reference sample indicates that the patient has an increased likelihood of benefit from treatment with the VEGF-C antagonist.

Another embodiment of the invention provides methods of optimizing therapeutic efficacy of a VEGF-C antagonist. The methods comprise determining expression levels of VEGF-A in a sample obtained from the patient, wherein decreased expression levels of VEGF-A in the sample as compared to a reference sample indicates that the patient has an increased likelihood of benefit from treatment with the VEGF-C antagonist.

Even another embodiment of the invention provides methods for treating a cell proliferative disorder in a patient. The methods comprise determining that a sample obtained from the patient has decreased expression levels of VEGF-A as compared to a reference sample, and administering to the patient an effective amount of a VEGF-C antagonist, whereby the cell proliferative disorder is treated.

Yet another embodiment of the invention provides methods of identifying a patient suffering from cancer who may benefit from treatment with a VEGF-C antagonist. The methods comprise determining expression levels of PlGF in a sample obtained from the patient, wherein decreased expression levels of PlGF in the sample as compared to a reference sample indicates that the patient may benefit from treatment with the VEGF-C antagonist.

A further embodiment of the invention provides methods of predicting responsiveness of a patient suffering from cancer to treatment with a VEGF-C antagonist. The methods comprise determining expression levels of PlGF in a sample obtained from the patient, wherein decreased expression levels of PlGF in the sample as compared to a reference sample indicates that the patient is more likely to be responsive to treatment with the VEGF-C antagonist.

Even a further embodiment of the invention provides methods of determining the likelihood that a patient will exhibit a benefit from treatment with a VEGF-C antagonist. The methods comprise determining expression levels of PlGF in a sample obtained from the patient, wherein decreased expression levels of PlGF in the sample as compared to a reference sample indicates that the patient has an increased likelihood of benefit from treatment with the VEGF-C antagonist.

Even a further embodiment of the invention provides methods of optimizing therapeutic efficacy of a VEGF-C antagonist. The methods comprise determining expression levels of PlGF in a sample obtained from the patient, wherein decreased expression levels of PlGF in the sample as compared to a reference sample indicates that the patient has an increased likelihood of benefit from treatment with the VEGF-C antagonist.

Yet a further embodiment of the invention provides methods for treating a cell proliferative disorder in a patient. The methods comprise determining that a sample obtained from the patient has decreased expression levels of PlGF as compared to a reference sample, and administering to the patient an effective amount of a VEGF-C antagonist, whereby the cell proliferative disorder is treated.

In some embodiments of the invention, the VEGF-C antagonist is an anti-VEGF-C antibody. In some embodiments of the invention, the methods further comprise administering a VEGF-A antagonist to the patient. In some embodiments of the invention, the VEGF-A antagonist and the VEGF-C antagonist are administered concurrently. In some embodiments of the invention, the VEGF-A antagonist and the VEGF-C antagonist are administered sequentially. In some embodiments of the invention, the VEGF-A antagonist is an anti-VEGF-A antibody. In some embodiments of the invention, the anti-VEGF-A antibody is bevacizumab.

Another embodiment of the invention provides kits for determining the expression levels of at least one gene selected from: VEGF-C, VEGF-D, VEGFR3, FGF2, RGS5/CDH5, IL-8, CXCL1, and CXCL2. The kits comprise an array comprising polynucleotides capable of specifically hybridizing to at least one gene selected from: VEGF-C, VEGF-D, VEGFR3, FGF2, RGS5/CDH5, IL-8, CXCL1, and CXCL2, and instructions for using the array to determine the expression levels of the at least one gene to predict responsiveness of a patient to treatment with a VEGF-C antagonist, wherein an increase in the expression level of the at least one gene as compared to the expression level of the at least one gene in a reference sample indicates that the patient may benefit from treatment with the VEGF-C antagonist.

Another embodiment of the invention provides kits for determining the expression levels of at least one gene selected from: VEGF-A, CSF2, Prox1, ICAM1, ESM1, PlGF, ITGa5, TGFβ, Hhex, Col4a1, Col4a2, and Alk1. The kits comprise an array comprising polynucleotides capable of specifically hybridizing to at least one gene selected from: VEGF-A, CSF2, Prox1, ICAM1, ESM1, PlGF, ITGa5, TGFβ, Hhex, Col4a1, Col4a2, and Alk1 and instructions for using the array to determine the expression levels of the at least one gene to predict responsiveness of a patient to treatment with a VEGF-C antagonist, wherein a decrease in the expression level of the at least one gene as compared to the expression level of the at least one gene in a reference sample indicates that the patient may benefit from treatment with the VEGF-C antagonist.

A further embodiment of the invention provides sets of compounds capable of detecting expression levels of at least one gene selected from: VEGF-C, VEGF-D, VEGFR3, FGF2, RGS5/CDH5, IL-8, CXCL1, and CXCL2 to determine the expression levels of the at least one gene in a sample obtained from a cancer patient. The sets comprise at least one compound capable of specifically hybridizing to at least one gene selected from: VEGF-C, VEGF-D, VEGFR3, FGF2, RGS5/CDH5, IL-8, CXCL1, and CXCL2, wherein an increase in the expression level of the at least one gene as compared to the expression level of the at least one gene in a reference sample indicates that the patient may benefit from treatment with a VEGF-C antagonist. In some embodiments of the invention, the compounds are polynucleotides. In some embodiments of the invention, the compounds are proteins, such as, for example, antibodies.

Even another embodiment of the invention provides sets of compounds capable of detecting expression levels of at least one gene selected from: VEGF-A, CSF2, Prox1, ICAM1, ESM1, PlGF, ITGa5, TGFβ, Hhex, Col4a1, Col4a2, and Alk1 to determine the expression levels of the at least one gene in a sample obtained from a cancer patient. The sets comprise at least one compound capable of specifically hybridizing to at least one gene selected from: VEGF-A, CSF2, Prox1, ICAM1, ESM1, PlGF, ITGa5, TGFβ, Hhex, Col4a1, Col4a2, and Alk1, wherein a decrease in the expression level of the at least gene as compared to the expression level of the at least one gene in a reference sample indicates that the patient may benefit from treatment with a VEGF-C antagonist. In some embodiments of the invention, the compounds are polynucleotides. In some embodiments of the invention, the compounds are proteins, such as, for example, antibodies.

One embodiment of the invention provides methods of identifying a patient suffering from cancer who may benefit from treatment with an EGF-like-domain, multiple 7 (EGFL7) antagonist. The methods comprise determining expression levels of at least one gene selected from: VEGF-C, BV8, CSF2, TNFα, CXCL2, PDGF-C, and Mincle in a sample obtained from the patient, wherein increased expression levels of the at least one gene in the sample as compared to a reference sample indicates that the patient may benefit from treatment with the EGFL7 antagonist.

Another embodiment of the invention provides methods of identifying a patient suffering from cancer who may benefit from treatment with an EGFL7 antagonist. The methods comprise determining expression levels of at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1 in a sample obtained from the patient, wherein decreased expression levels of the at least one gene in the sample as compared to a reference sample indicates that the patient may benefit from treatment with the EGFL7 antagonist.

A further embodiment of the invention provides methods of predicting responsiveness of a patient suffering from cancer to treatment with an EGFL7 antagonist. The methods comprise determining expression levels of at least one gene selected from: VEGF-C, BV8, CSF2, TNFα, CXCL2, PDGF-C, and Mincle in a sample obtained from the patient, wherein increased expression levels of the at least one gene in the sample as compared to a reference sample indicates that the patient is more likely to be responsive to treatment with the EGFL7 antagonist.

Yet another embodiment of the invention provides methods of predicting responsiveness of a patient suffering from cancer to treatment with an EGFL7 antagonist. The methods comprise determining expression levels of at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1 in a sample obtained from the patient, wherein decreased expression levels of the at least one gene in the sample as compared to a reference sample indicates that the patient is more likely to be responsive to treatment with the EGFL7 antagonist.

Even another embodiment of the invention provides methods of determining the likelihood that a patient will exhibit a benefit from treatment with an EGFL7 antagonist. The methods comprise determining expression levels of at least one gene selected from: VEGF-C, BV8, CSF2, TNFα, CXCL2, PDGF-C, and Mincle in a sample obtained from the patient, wherein increased expression levels of the at least one gene in the sample as compared to a reference sample indicates that the patient has increased likelihood of benefit from treatment with the EGFL7 antagonist.

Yet a further embodiment of the invention provides methods of determining the likelihood that a patient will exhibit a benefit from treatment with an EGFL7 antagonist. The methods comprise determining expression levels of at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1 in a sample obtained from the patient, wherein decreased expression levels of the at least one gene in the sample as compared to a reference sample indicates that the patient has increased likelihood of benefit of treatment with the EGFL7 antagonist.

Even another embodiment of the invention provides methods of optimizing therapeutic efficacy of an EGFL7 antagonist. The methods comprise determining expression levels of at least one gene selected from: VEGF-C, BV8, CSF2, TNFα, CXCL2, PDGF-C, and Mincle in a sample obtained from the patient, wherein increased expression levels of the at least one gene in the sample as compared to a reference sample indicates that the patient has increased likelihood of benefit from treatment with the EGFL7 antagonist.

Yet a further embodiment of the invention provides methods of optimizing therapeutic efficacy of an EGFL7 antagonist. The methods comprise determining expression levels of at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1 in a sample obtained from the patient, wherein decreased expression levels of the at least one gene in the sample as compared to a reference sample indicates that the patient has increased likelihood of benefit of treatment with the EGFL7 antagonist.

Another embodiment of the invention provides methods for treating a cell proliferative disorder in a patient. The methods comprise determining that a sample obtained from the patient has increased expression levels, as compared to a reference sample, of at least one gene selected from: VEGF-C, BV8, CSF2, TNFα, CXCL2, PDGF-C, and Mincle, and administering to the patient an effective amount of an EGFL7 antagonist, whereby the cell proliferative disorder is treated.

A further embodiment of the invention provides methods for treating a cell proliferative disorder in a patient. The methods comprise determining that a sample obtained from the patient has decreased expression levels, as compared to a reference sample, of at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1, and administering to the patient an effective amount of an EGFL7 antagonist, whereby the cell proliferative disorder is treated.

In some embodiments of the invention, the sample obtained from the patient is selected from: tissue, whole blood, blood-derived cells, plasma, serum, and combinations thereof. In some embodiments of the invention, the expression level is mRNA expression level. In some embodiments of the invention, the expression level is protein expression level. In some embodiments of the invention, the EGFL7 antagonist is an anti-EGFL7 antibody.

In some embodiments of the invention, the methods further comprises administering a VEGF-A antagonist to the patient. In some embodiments of the invention, the VEGF-A antagonist and the EGFL7 antagonist are administered concurrently. In some embodiments of the invention, the VEGF-A antagonist and the EGFL7 antagonist are administered sequentially. In some embodiments of the invention, the VEGF-A antagonist is an anti-VEGF-A antibody. In some embodiments of the invention, the anti-VEGF-A antibody is bevacizumab.

A further embodiment of the invention provides methods of identifying a patient suffering from cancer who may benefit from treatment with an EGFL7 antagonist. The methods comprise determining expression levels of VEGF-C in a sample obtained from the patient, wherein increased expression levels of VEGF-C in the sample as compared to a reference sample indicates that the patient may benefit from treatment with the EGFL7 antagonist.

Another embodiment of the invention provides methods of predicting responsiveness of a patient suffering from cancer to treatment with an EGFL7 antagonist. The methods comprise determining expression levels of VEGF-C in a sample obtained from the patient, wherein increased expression levels of VEGF-C in the sample as compared to a reference sample indicates that the patient is more likely to be responsive to treatment with the EGFL7 antagonist.

Even another embodiment of the invention provides methods of determining the likelihood that a patient will exhibit a benefit from treatment with an EGFL7 antagonist. The methods comprise determining expression levels of VEGF-C in a sample obtained from the patient, wherein increased expression levels of VEGF-C in the sample as compared to a reference sample indicates that the patient has an increased likelihood of benefit from treatment with the EGFL7 antagonist.

Even another embodiment of the invention provides methods of optimizing therapeutic efficacy of an EGFL7 antagonist. The methods comprise determining expression levels of VEGF-C in a sample obtained from the patient, wherein increased expression levels of VEGF-C in the sample as compared to a reference sample indicates that the patient has an increased likelihood of benefit from treatment with the EGFL7 antagonist.

A further embodiment of the invention provides methods for treating a cell proliferative disorder in a patient. The methods comprise determining that a sample obtained from the patient has increased expression levels of VEGF-C as compared to a reference sample, and administering to the patient an effective amount of an EGFL7 antagonist, whereby the cell proliferative disorder is treated.

Yet another embodiment of the invention provides methods of identifying a patient suffering from cancer who may benefit from treatment with an EGFL7 antagonist. The methods comprise determining expression levels of BV8 in a sample obtained from the patient, wherein increased expression levels of BV8 in the sample as compared to a reference sample indicates that the patient may benefit from treatment with the EGFL7 antagonist.

Another embodiment of the invention provides methods of predicting responsiveness of a patient suffering from cancer to treatment with an EGFL7 antagonist. The methods comprise determining expression levels of BV8 in a sample obtained from the patient, wherein increased expression levels of BV8 in the sample as compared to a reference sample indicates that the patient is more likely to be responsive to treatment with the EGFL7 antagonist.

Even another embodiment of the invention provides methods of determining the likelihood that a patient will exhibit a benefit from treatment with an EGFL7 antagonist. The methods comprise determining expression levels of BV8 in a sample obtained from the patient, wherein increased expression levels of BV8 in the sample as compared to a reference sample indicates that the patient has an increased likelihood of benefit from treatment with the EGFL7 antagonist.

Even another embodiment of the invention provides methods of optimizing therapeutic efficacy of an EGFL7 antagonist. The methods comprise determining expression levels of BV8 in a sample obtained from the patient, wherein increased expression levels of BV8 in the sample as compared to a reference sample indicates that the patient has an increased likelihood of benefit from treatment with the EGFL7 antagonist.

Yet a further embodiment of the invention provides methods for treating a cell proliferative disorder in a patient. The methods comprise determining that a sample obtained from the patient has increased expression levels of BV8 as compared to a reference sample, and administering to the patient an effective amount of an EGFL7 antagonist, whereby the cell proliferative disorder is treated

Another embodiment of the invention provides methods of identifying a patient suffering from cancer who may benefit from treatment with an EGFL7 antagonist. The methods comprise determining expression levels of CSF2 in a sample obtained from the patient, wherein increased expression levels of CSF2 in the sample as compared to a reference sample indicates that the patient may benefit from treatment with the EGFL7 antagonist.

Even another embodiment of the invention provides methods of predicting responsiveness of a patient suffering from cancer to treatment with an EGFL7 antagonist. The methods comprise determining expression levels of CSF2 in a sample obtained from the patient, wherein increased expression levels of CSF2 in the sample as compared to a reference sample indicates that the patient is more likely to be responsive to treatment with the EGFL7 antagonist.

A further embodiment of the invention provides methods of determining the likelihood that a patient will exhibit a benefit from treatment with an EGFL7 antagonist. The methods comprise determining expression levels of CSF2 in a sample obtained from the patient, wherein increased expression levels of CSF2 in the sample as compared to a reference sample indicates that the patient has an increased likelihood of benefit from treatment with the EGFL7 antagonist.

A further embodiment of the invention provides methods of optimizing therapeutic efficacy of an EGFL7 antagonist. The methods comprise determining expression levels of CSF2 in a sample obtained from the patient, wherein increased expression levels of CSF2 in the sample as compared to a reference sample indicates that the patient has an increased likelihood of benefit from treatment with the EGFL7 antagonist.

A yet further embodiment provides methods for treating a cell proliferative disorder in a patient. The methods comprise determining that a sample obtained from the patient has increased expression levels of CSF2 as compared to a reference sample, and administering to the patient an effective amount of an EGFL7 antagonist, whereby the cell proliferative disorder is treated

Another embodiment of the invention provides methods of identifying a patient suffering from cancer who may benefit from treatment with an EGFL7 antagonist. The methods comprise determining expression levels of TNFα in a sample obtained from the patient, wherein increased expression levels of TNFα in the sample as compared to a reference sample indicates that the patient may benefit from treatment with the EGFL7 antagonist.

Even another embodiment of the invention provides methods of predicting responsiveness of a patient suffering from cancer to treatment with an EGFL7 antagonist. The methods comprise determining expression levels of TNFα in a sample obtained from the patient, wherein increased expression levels of TNFα in the sample as compared to a reference sample indicates that the patient is more likely to be responsive to treatment with the EGFL7 antagonist.

Yet another embodiment of the invention provides methods of determining the likelihood that a patient will exhibit a benefit from treatment with an EGFL7 antagonist. The methods comprise determining expression levels of TNFα in a sample obtained from the patient, wherein increased expression levels of TNFα in the sample as compared to a reference sample indicates that the patient has an increased likelihood of benefit from treatment with the EGFL7 antagonist.

Yet another embodiment of the invention provides methods of optimizing therapeutic efficacy of an EGFL7 antagonist. The methods comprise determining expression levels of TNFα in a sample obtained from the patient, wherein increased expression levels of TNFα in the sample as compared to a reference sample indicates that the patient has an increased likelihood of benefit from treatment with the EGFL7 antagonist.

A further embodiment of the invention provides methods for treating a cell proliferative disorder in a patient. The methods comprise determining that a sample obtained from the patient has increased expression levels of TNFα as compared to a reference sample, and administering to the patient an effective amount of an EGFL7 antagonist, whereby the cell proliferative disorder is treated.

Even a further embodiment of the invention provides methods of identifying a patient suffering from cancer who may benefit from treatment with an EGFL7 antagonist. The methods comprise determining expression levels of Sema3B in a sample obtained from the patient, wherein decreased expression levels of Sema3B in the sample as compared to a reference sample indicates that the patient may benefit from treatment with the EGFL7 antagonist.

Yet a further embodiment of the invention provides methods of predicting responsiveness of a patient suffering from cancer to treatment with an EGFL7 antagonist. The methods comprise determining expression levels of Sema3B in a sample obtained from the patient, wherein decreased expression levels of Sema3B in the sample as compared to a reference sample indicates that the patient is more likely to be responsive to treatment with the EGFL7 antagonist.

Another embodiment of the invention provides methods of determining the likelihood that a patient will exhibit a benefit from treatment with an EGFL7 antagonist. The methods comprise determining expression levels of Sema3B in a sample obtained from the patient, wherein decreased expression levels of Sema3B in the sample as compared to a reference sample indicates that the patient has an increased likelihood of benefit from treatment with the EGFL7 antagonist.

Another embodiment of the invention provides methods of optimizing therapeutic efficacy of an EGFL7 antagonist. The methods comprise determining expression levels of Sema3B in a sample obtained from the patient, wherein decreased expression levels of Sema3B in the sample as compared to a reference sample indicates that the patient has an increased likelihood of benefit from treatment with the EGFL7 antagonist.

Even another embodiment of the invention provides methods for treating a cell proliferative disorder in a patient. The methods comprise determining that a sample obtained from the patient has decreased expression levels of Sema3B as compared to a reference sample, and administering to the patient an effective amount of an EGFL7 antagonist, whereby the cell proliferative disorder is treated.

Yet another embodiment of the invention provides methods of identifying a patient suffering from cancer who may benefit from treatment with an EGFL7 antagonist. The methods comprise determining expression levels of FGF9 in a sample obtained from the patient, wherein decreased expression levels of FGF9 in the sample as compared to a reference sample indicates that the patient may benefit from treatment with the EGFL7 antagonist.

A further embodiment of the invention provides methods of predicting responsiveness of a patient suffering from cancer to treatment with an EGFL7 antagonist. The methods comprise determining expression levels of FGF9 in a sample obtained from the patient, wherein decreased expression levels of FGF9 in the sample as compared to a reference sample indicates that the patient is more likely to be responsive to treatment with the EGFL7 antagonist.

Even a further embodiment of the invention provides methods of determining the likelihood that a patient will exhibit a benefit from treatment with an EGFL7 antagonist. The methods comprise determining expression levels of FGF9 in a sample obtained from the patient, wherein decreased expression levels of FGF9 in the sample as compared to a reference sample indicates that the patient has an increased likelihood of benefit from treatment with the EGFL7 antagonist.

Even a further embodiment of the invention provides methods of optimizing therapeutic efficacy of an EGFL7 antagonist. The methods comprise determining expression levels of FGF9 in a sample obtained from the patient, wherein decreased expression levels of FGF9 in the sample as compared to a reference sample indicates that the patient has an increased likelihood of benefit from treatment with the EGFL7 antagonist.

Another embodiment of the invention provides methods for treating a cell proliferative disorder in a patient. The methods comprise determining that a sample obtained from the patient has decreased expression levels of FGF9 as compared to a reference sample, and administering to the patient an effective amount of an EGFL7 antagonist, whereby the cell proliferative disorder is treated.

Even another embodiment of the invention provides methods of identifying a patient suffering from cancer who may benefit from treatment with an EGFL7 antagonist. The methods comprise determining expression levels of HGF in a sample obtained from the patient, wherein decreased expression levels of HGF in the sample as compared to a reference sample indicates that the patient may benefit from treatment with the EGFL7 antagonist.

Yet another embodiment of the invention provides methods of predicting responsiveness of a patient suffering from cancer to treatment with an EGFL7 antagonist. The methods comprise determining expression levels of HGF in a sample obtained from the patient, wherein decreased expression levels of HGF in the sample as compared to a reference sample indicates that the patient is more likely to be responsive to treatment with the EGFL7 antagonist.

A further embodiment of the invention provides methods of determining the likelihood that a patient will exhibit a benefit from treatment with an EGFL7 antagonist. The methods comprise determining expression levels of HGF in a sample obtained from the patient, wherein decreased expression levels of HGF in the sample as compared to a reference sample indicates that the patient has an increased likelihood of benefit from treatment with the EGFL7 antagonist.

A further embodiment of the invention provides methods of optimizing therapeutic efficacy of an EGFL7 antagonist. The methods comprise determining expression levels of HGF in a sample obtained from the patient, wherein decreased expression levels of HGF in the sample as compared to a reference sample indicates that the patient has an increased likelihood of benefit from treatment with the EGFL7 antagonist.

Even a further embodiment of the invention provides methods for treating a cell proliferative disorder in a patient. The methods comprise determining that a sample obtained from the patient has decreased expression levels of HGF as compared to a reference sample, and administering to the patient an effective amount of an EGFL7 antagonist, whereby the cell proliferative disorder is treated.

Another embodiment of the invention provides methods of identifying a patient suffering from cancer who may benefit from treatment with an EGFL7 antagonist. The methods comprise determining expression levels of RGS5 in a sample obtained from the patient, wherein decreased expression levels of RGS5 in the sample as compared to a reference sample indicates that the patient may benefit from treatment with the EGFL7 antagonist.

Yet another embodiment of the invention provides methods of predicting responsiveness of a patient suffering from cancer to treatment with an EGFL7 antagonist. The methods comprise determining expression levels of RGS5 in a sample obtained from the patient, wherein decreased expression levels of RGS5 in the sample as compared to a reference sample indicates that the patient is more likely to be responsive to treatment with the EGFL7 antagonist.

A further embodiment of the invention provides methods of determining the likelihood that a patient will exhibit a benefit from treatment with an EGFL7 antagonist. The methods comprise determining expression levels of RGS5 in a sample obtained from the patient, wherein decreased expression levels of RGS5 in the sample as compared to a reference sample indicates that the patient has an increased likelihood of benefit from treatment with the EGFL7 antagonist.

A further embodiment of the invention provides methods of optimizing therapeutic efficacy of an EGFL7 antagonist. The methods comprise determining expression levels of RGS5 in a sample obtained from the patient, wherein decreased expression levels of RGS5 in the sample as compared to a reference sample indicates that the patient has an increased likelihood of benefit from treatment with the EGFL7 antagonist.

Yet a further embodiment of the invention provides methods for treating a cell proliferative disorder in a patient. The methods comprise determining that a sample obtained from the patient has decreased expression levels of RGS5 as compared to a reference sample, and administering to the patient an effective amount of an EGFL7 antagonist, whereby the cell proliferative disorder is treated.

Even a further embodiment of the invention provides methods of identifying a patient suffering from cancer who may benefit from treatment with an EGFL7 antagonist. The methods comprise determining expression levels of NRP1 in a sample obtained from the patient, wherein decreased expression levels of NRP1 in the sample as compared to a reference sample indicates that the patient may benefit from treatment with the EGFL7 antagonist.

Another embodiment of the invention provides methods of predicting responsiveness of a patient suffering from cancer to treatment with an EGFL7 antagonist. The methods comprise determining expression levels of NRP1 in a sample obtained from the patient, wherein decreased expression levels of NRP1 in the sample as compared to a reference sample indicates that the patient is more likely to be responsive to treatment with the EGFL7 antagonist.

Yet another embodiment of the invention provides methods of determining the likelihood that a patient will exhibit a benefit from treatment with an EGFL7 antagonist. The methods comprise determining expression levels of NRP1 in a sample obtained from the patient, wherein decreased expression levels of NRP1 in the sample as compared to a reference sample indicates that the patient has an increased likelihood of benefit from treatment with the EGFL7 antagonist.

Yet another embodiment of the invention provides methods of optimizing therapeutic efficacy of an EGFL7 antagonist. The methods comprise determining expression levels of NRP1 in a sample obtained from the patient, wherein decreased expression levels of NRP1 in the sample as compared to a reference sample indicates that the patient has an increased likelihood of benefit from treatment with the EGFL7 antagonist.

Even another embodiment of the invention provides methods for treating a cell proliferative disorder in a patient. The methods comprise determining that a sample obtained from the patient has decreased expression levels of NRP1 as compared to a reference sample, and administering to the patient an effective amount of an EGFL7 antagonist, whereby the cell proliferative disorder is treated.

Even another embodiment of the invention provides methods of identifying a patient suffering from cancer who may benefit from treatment with an EGFL7 antagonist. The methods comprise determining expression levels of FGF2 in a sample obtained from the patient, wherein decreased expression levels of FGF2 in the sample as compared to a reference sample indicates that the patient may benefit from treatment with the EGFL7 antagonist.

Yet another embodiment of the invention provides methods of predicting responsiveness of a patient suffering from cancer to treatment with an EGFL7 antagonist. The methods comprise determining expression levels of FGF2 in a sample obtained from the patient, wherein decreased expression levels of FGF2 in the sample as compared to a reference sample indicates that the patient is more likely to be responsive to treatment with the EGFL7 antagonist.

A further embodiment of the invention provides methods of determining the likelihood that a patient will exhibit a benefit from treatment with an EGFL7 antagonist. The methods comprise determining expression levels of FGF2 in a sample obtained from the patient, wherein decreased expression levels of FGF2 in the sample as compared to a reference sample indicates that the patient has an increased likelihood of benefit from treatment with the EGFL7 antagonist.

A further embodiment of the invention provides methods of optimizing therapeutic efficacy of an EGFL7 antagonist. The methods comprise determining expression levels of FGF2 in a sample obtained from the patient, wherein decreased expression levels of FGF2 in the sample as compared to a reference sample indicates that the patient has an increased likelihood of benefit from treatment with the EGFL7 antagonist.

Yet a further embodiment of the invention provides methods for treating a cell proliferative disorder in a patient. The methods comprise determining that a sample obtained from the patient has decreased expression levels of FGF2 as compared to a reference sample, and administering to the patient an effective amount of an EGFL7 antagonist, whereby the cell proliferative disorder is treated.

Even a further embodiment of the invention provides methods of identifying a patient suffering from cancer who may benefit from treatment with an EGFL7 antagonist. The methods comprise determining expression levels of CXCR4 in a sample obtained from the patient, wherein decreased expression levels of CXCR4 in the sample as compared to a reference sample indicates that the patient may benefit from treatment with the EGFL7 antagonist.

Another embodiment of the invention provides methods of predicting responsiveness of a patient suffering from cancer to treatment with an EGFL7 antagonist. The methods comprise determining expression levels of CXCR4 in a sample obtained from the patient, wherein decreased expression levels of CXCR4 in the sample as compared to a reference sample indicates that the patient is more likely to be responsive to treatment with the EGFL7 antagonist.

Even another embodiment of the invention provides methods of determining the likelihood that a patient will exhibit a benefit from treatment with an EGFL7 antagonist. The methods comprise determining expression levels of CXCR4 in a sample obtained from the patient, wherein decreased expression levels of CXCR4 in the sample as compared to a reference sample indicates that the patient has an increased likelihood of benefit from treatment with the EGFL7 antagonist.

Even another embodiment of the invention provides methods of optimizing therapeutic efficacy of an EGFL7 antagonist. The methods comprise determining expression levels of CXCR4 in a sample obtained from the patient, wherein decreased expression levels of CXCR4 in the sample as compared to a reference sample indicates that the patient has an increased likelihood of benefit from treatment with the EGFL7 antagonist.

Yet another embodiment of the invention provides methods for treating a cell proliferative disorder in a patient. The methods comprise determining that a sample obtained from the patient has decreased expression levels of CXCR4 as compared to a reference sample, and administering to the patient an effective amount of an EGFL7 antagonist, whereby the cell proliferative disorder is treated.

Another embodiment of the invention provides methods of identifying a patient suffering from cancer who may benefit from treatment with an EGFL7 antagonist. The methods comprise determining expression levels of cMet in a sample obtained from the patient, wherein decreased expression levels of cMet in the sample as compared to a reference sample indicates that the patient may benefit from treatment with the EGFL7 antagonist.

Yet another embodiment of the invention provides methods of predicting responsiveness of a patient suffering from cancer to treatment with an EGFL7 antagonist. The methods comprise determining expression levels of cMet in a sample obtained from the patient, wherein decreased expression levels of cMet in the sample as compared to a reference sample indicates that the patient is more likely to be responsive to treatment with the EGFL7 antagonist.

Even another embodiment of the invention provides methods of determining the likelihood that a patient will exhibit a benefit from treatment with an EGFL7 antagonist. The methods comprise determining expression levels of cMet in a sample obtained from the patient, wherein decreased expression levels of cMet in the sample as compared to a reference sample indicates that the patient has an increased likelihood of benefit from treatment with the EGFL7 antagonist.

Even another embodiment of the invention provides methods of optimizing therapeutic efficacy of an EGFL7 antagonist. The methods comprise determining expression levels of cMet in a sample obtained from the patient, wherein decreased expression levels of cMet in the sample as compared to a reference sample indicates that the patient has an increased likelihood of benefit from treatment with the EGFL7 antagonist.

A further embodiment of the invention provides methods for treating a cell proliferative disorder in a patient. The methods comprise determining that a sample obtained from the patient has decreased expression levels of cMet as compared to a reference sample, and administering to the patient an effective amount of an EGFL7 antagonist, whereby the cell proliferative disorder is treated.

Yet a further embodiment of the invention provides methods of identifying a patient suffering from cancer who may benefit from treatment with an EGFL7 antagonist. The methods comprise determining expression levels of FN1 in a sample obtained from the patient, wherein decreased expression levels of FN1 in the sample as compared to a reference sample indicates that the patient may benefit from treatment with the EGFL7 antagonist.

Even a further embodiment of the invention provides methods of predicting responsiveness of a patient suffering from cancer to treatment with an EGFL7 antagonist. The methods comprise determining expression levels of FN1 in a sample obtained from the patient, wherein decreased expression levels of FN1 in the sample as compared to a reference sample indicates that the patient is more likely to be responsive to treatment with the EGFL7 antagonist.

Another embodiment of the invention provides methods of determining the likelihood that a patient will exhibit a benefit from treatment with an EGFL7 antagonist. The methods comprise determining expression levels of FN1 in a sample obtained from the patient, wherein decreased expression levels of FN1 in the sample as compared to a reference sample indicates that the patient has an increased likelihood of benefit from treatment with the EGFL7 antagonist.

Another embodiment of the invention provides methods of optimizing therapeutic efficacy of an EGFL7 antagonist. The methods comprise determining expression levels of FN1 in a sample obtained from the patient, wherein decreased expression levels of FN1 in the sample as compared to a reference sample indicates that the patient has an increased likelihood of benefit from treatment with the EGFL7 antagonist.

Even another embodiment of the invention provides methods for treating a cell proliferative disorder in a patient. The methods comprise determining that a sample obtained from the patient has decreased expression levels of FN1 as compared to a reference sample, and administering to the patient an effective amount of an EGFL7 antagonist, whereby the cell proliferative disorder is treated.

Yet another embodiment of the invention provides methods of identifying a patient suffering from cancer who may benefit from treatment with an EGFL7 antagonist. The methods comprise determining expression levels of Fibulin 2 in a sample obtained from the patient, wherein decreased expression levels of Fibulin 2 in the sample as compared to a reference sample indicates that the patient may benefit from treatment with the EGFL7 antagonist.

A further embodiment of the invention provides methods of predicting responsiveness of a patient suffering from cancer to treatment with an EGFL7 antagonist. The methods comprise determining expression levels of Fibulin 2 in a sample obtained from the patient, wherein decreased expression levels of Fibulin 2 in the sample as compared to a reference sample indicates that the patient is more likely to be responsive to treatment with the EGFL7 antagonist.

Even a further embodiment of the invention provides methods of determining the likelihood that a patient will exhibit a benefit from treatment with an EGFL7 antagonist. The methods comprise determining expression levels of Fibulin 2 in a sample obtained from the patient, wherein decreased expression levels of Fibulin 2 in the sample as compared to a reference sample indicates that the patient has an increased likelihood of benefit from treatment with the EGFL7 antagonist.

Even a further embodiment of the invention provides methods of optimizing therapeutic efficacy of an EGFL7 antagonist. The methods comprise determining expression levels of Fibulin 2 in a sample obtained from the patient, wherein decreased expression levels of Fibulin 2 in the sample as compared to a reference sample indicates that the patient has an increased likelihood of benefit from treatment with the EGFL7 antagonist.

Yet a further embodiment of the invention provides methods for treating a cell proliferative disorder in a patient. The methods comprise determining that a sample obtained from the patient has decreased expression levels of Fibulin 2 as compared to a reference sample, and administering to the patient an effective amount of an EGFL7 antagonist, whereby the cell proliferative disorder is treated.

Another embodiment of the invention provides methods of identifying a patient suffering from cancer who may benefit from treatment with an EGFL7 antagonist. The methods comprise determining expression levels of Fibulin4 in a sample obtained from the patient, wherein decreased expression levels of Fibulin4 in the sample as compared to a reference sample indicates that the patient may benefit from treatment with the EGFL7 antagonist.

Even another embodiment of the invention provides methods of predicting responsiveness of a patient suffering from cancer to treatment with an EGFL7 antagonist. The methods comprise determining expression levels of Fibulin4 in a sample obtained from the patient, wherein decreased expression levels of Fibulin4 in the sample as compared to a reference sample indicates that the patient is more likely to be responsive to treatment with the EGFL7 antagonist.

A further embodiment of the invention provides methods of determining the likelihood that a patient will exhibit a benefit from treatment with an EGFL7 antagonist. The methods comprise determining expression levels of Fibulin4 in a sample obtained from the patient, wherein decreased expression levels of Fibulin4 in the sample as compared to a reference sample indicates that the patient has an increased likelihood of benefit from treatment with the EGFL7 antagonist.

A further embodiment of the invention provides methods of optimizing therapeutic efficacy of an EGFL7 antagonist. The methods comprise determining expression levels of Fibulin4 in a sample obtained from the patient, wherein decreased expression levels of Fibulin4 in the sample as compared to a reference sample indicates that the patient has an increased likelihood of benefit from treatment with the EGFL7 antagonist.

Even a further embodiment of the invention provides methods for treating a cell proliferative disorder in a patient. The methods comprise determining that a sample obtained from the patient has decreased expression levels of Fibulin4 as compared to a reference sample, and administering to the patient an effective amount of an EGFL7 antagonist, whereby the cell proliferative disorder is treated.

Yet a further embodiment of the invention provides methods of identifying a patient suffering from cancer who may benefit from treatment with an EGFL7 antagonist. The methods comprise determining expression levels of MFAP5 in a sample obtained from the patient, wherein decreased expression levels of MFAP5 in the sample as compared to a reference sample indicates that the patient may benefit from treatment with the EGFL7 antagonist.

Another embodiment of the invention provides methods of predicting responsiveness of a patient suffering from cancer to treatment with an EGFL7 antagonist. The methods comprise determining expression levels of MFAP5 in a sample obtained from the patient, wherein decreased expression levels of MFAP5 in the sample as compared to a reference sample indicates that the patient is more likely to be responsive to treatment with the EGFL7 antagonist.

Even another embodiment of the invention provides methods of determining the likelihood that a patient will exhibit a benefit from treatment with an EGFL7 antagonist. The methods comprise determining expression levels of MFAP5 in a sample obtained from the patient, wherein decreased expression levels of MFAP5 in the sample as compared to a reference sample indicates that the patient has an increased likelihood of benefit from treatment with the EGFL7 antagonist.

Even another embodiment of the invention provides methods of optimizing therapeutic efficacy of an EGFL7 antagonist. The methods comprise determining expression levels of MFAP5 in a sample obtained from the patient, wherein decreased expression levels of MFAP5 in the sample as compared to a reference sample indicates that the patient has an increased likelihood of benefit from treatment with the EGFL7 antagonist.

Yet another embodiment of the invention provides methods for treating a cell proliferative disorder in a patient. The methods comprise determining that a sample obtained from the patient has decreased expression levels of MFAP5 as compared to a reference sample, and administering to the patient an effective amount of an EGFL7 antagonist, whereby the cell proliferative disorder is treated.

A further embodiment of the invention provides methods of identifying a patient suffering from cancer who may benefit from treatment with an EGFL7 antagonist. The methods comprise determining expression levels of PDGF-C in a sample obtained from the patient, wherein decreased expression levels of PDGF-C in the sample as compared to a reference sample indicates that the patient may benefit from treatment with the EGFL7 antagonist.

Even a further embodiment of the invention provides methods of predicting responsiveness of a patient suffering from cancer to treatment with an EGFL7 antagonist. The methods comprise determining expression levels of PDGF-C in a sample obtained from the patient, wherein decreased expression levels of PDGF-C in the sample as compared to a reference sample indicates that the patient is more likely to be responsive to treatment with the EGFL7 antagonist.

Yet a further embodiment of the invention provides methods of determining the likelihood that a patient will exhibit a benefit from treatment with an EGFL7 antagonist. The methods comprise determining expression levels of PDGF-C in a sample obtained from the patient, wherein decreased expression levels of PDGF-C in the sample as compared to a reference sample indicates that the patient has an increased likelihood of benefit from treatment with the EGFL7 antagonist.

Yet a further embodiment of the invention provides methods of optimizing therapeutic efficacy of an EGFL7 antagonist. The methods comprise determining expression levels of PDGF-C in a sample obtained from the patient, wherein decreased expression levels of PDGF-C in the sample as compared to a reference sample indicates that the patient has an increased likelihood of benefit from treatment with the EGFL7 antagonist.

Another embodiment of the invention provides methods for treating a cell proliferative disorder in a patient. The methods comprise determining that a sample obtained from the patient has decreased expression levels of PDGF-C as compared to a reference sample, and administering to the patient an effective amount of an EGFL7 antagonist, whereby the cell proliferative disorder is treated.

Even another embodiment of the invention provides methods of identifying a patient suffering from cancer who may benefit from treatment with an EGFL7 antagonist. The methods comprise determining expression levels of Sema3F in a sample obtained from the patient, wherein decreased expression levels of Sema3F in the sample as compared to a reference sample indicates that the patient may benefit from treatment with the EGFL7 antagonist.

Yet another embodiment of the invention provides methods of predicting responsiveness of a patient suffering from cancer to treatment with an EGFL7 antagonist. The methods comprise determining expression levels of Sema3F in a sample obtained from the patient, wherein decreased expression levels of Sema3F in the sample as compared to a reference sample indicates that the patient is more likely to be responsive to treatment with the EGFL7 antagonist.

A further embodiment of the invention provides methods of determining the likelihood that a patient will exhibit a benefit from treatment with an EGFL7 antagonist. The methods comprise determining expression levels of Sema3F in a sample obtained from the patient, wherein decreased expression levels of Sema3F in the sample as compared to a reference sample indicates that the patient has an increased likelihood of benefit from treatment with the EGFL7 antagonist.

A further embodiment of the invention provides methods of optimizing therapeutic efficacy of an EGFL7 antagonist. The methods comprise determining expression levels of Sema3F in a sample obtained from the patient, wherein decreased expression levels of Sema3F in the sample as compared to a reference sample indicates that the patient has an increased likelihood of benefit from treatment with the EGFL7 antagonist.

Even a further embodiment of the invention provides methods for treating a cell proliferative disorder in a patient. The methods comprise determining that a sample obtained from the patient has decreased expression levels of Sema3F as compared to a reference sample, and administering to the patient an effective amount of an EGFL7 antagonist, whereby the cell proliferative disorder is treated.

In some embodiments of the invention, the EGFL7 antagonist is an anti-EGFL7 antibody. In some embodiments of the invention, the methods further comprises administering a VEGF-A antagonist to the patient. In some embodiments of the invention, the VEGF-A antagonist and the EGFL7 antagonist are administered concurrently. In some embodiments of the invention, the VEGF-A antagonist and the EGFL7 antagonist are administered sequentially. In some embodiments of the invention, the VEGF-A antagonist is an anti-VEGF-A antibody. In some embodiments of the invention, the anti-VEGF-A antibody is bevacizumab.

Another embodiment of the invention provides kits for determining the expression levels of at least one gene selected from: VEGF-C, BV8, CSF2, TNFα, CXCL2, PDGF-C, and Mincle. The kits comprise an array comprising polynucleotides capable of specifically hybridizing to at least one gene selected from: VEGF-C, BV8, CSF2, TNFα, CXCL2, PDGF-C, and Mincle, and instructions for using the array to determine the expression levels of the at least one gene to predict responsiveness of a patient to treatment with an EGFL7 antagonist, wherein an increase in the expression level of the at least one gene as compared to the expression level of the at least one gene in a reference sample indicates that the patient may benefit from treatment with the EGFL7 antagonist.

Even another embodiment of the invention provides kits for determining the expression levels of at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1. The kits comprise an array comprising polynucleotides capable of specifically hybridizing to at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1 and instructions for using the array to determine the expression levels of the at least one gene to predict responsiveness of a patient to treatment with an EGFL7 antagonist, wherein a decrease in the expression level of the at least one gene as compared to the expression level of the at least one gene in a reference sample indicates that the patient may benefit from treatment with the EGFL7 antagonist.

Yet another embodiment of the invention provides sets of compounds capable of detecting expression levels of at least one gene selected from: VEGF-C, BV8, CSF2, TNFα, CXCL2, PDGF-C, and Mincle. The sets comprise at least one compound capable of specifically hybridizing to at least one gene selected from: VEGF-C, BV8, CSF2, TNFα, CXCL2, PDGF-C, and Mincle, wherein an increase in the expression level of the at least one gene as compared to the expression level of the at least one gene in a reference sample indicates that the patient may benefit from treatment with an EGFL7 antagonist. In some embodiments of the invention, the compounds are polynucleotides. In some embodiments of the invention, the polynucleotides comprise three sequences set forth in Table 2. In some embodiments of the invention, the compounds are proteins, such as, for example, antibodies.

A further embodiment of the invention provides sets of compounds capable of detecting expression levels of at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1. The sets comprise at least one compound that specifically hybridizes to at least one gene selected from: Sema3B, FGF9, HGF, RGS5, NRP1, FGF2, CXCR4, cMet, FN1, Fibulin 2, Fibulin4/EFEMP2, MFAP5, PDGF-C, Sema3F, and FN1, wherein a decrease in the expression level of the at least gene as compared to the expression level of the at least one gene in a reference sample indicates that the patient may benefit from treatment with an EGFL7 antagonist. In some embodiments of the invention, the compounds are polynucleotides. In some embodiments of the invention, the polynucleotides comprise three sequences set forth in Table 2. In some embodiments of the invention, the compounds are proteins, such as, for example, antibodies.

These and other embodiments of the invention are further described in the detailed description that follows.

### BRIEF DESCRIPTION OF THE DRAWINGS

FIG. 1 is a table showing the efficacy of combination treatment with anti-VEGF antibody and anti-NRP1 antibody in inhibiting tumor growth in various tumor xenograft models.

FIG. 2 is a table showing p- and r-values for the correlation of marker RNA expression (qPCR) and efficacy of combination treatment with anti-VEGF antibody and anti-NRP1 antibody.

FIG. 3 is a graph showing improved efficacy of combination treatment with anti-VEGF antibody and anti-NRP1 antibody versus relative expression of TGFβ1 (transforming growth factor (β1).

FIG. 4 is a graph showing improved efficacy of combination treatment with anti-VEGF antibody and anti-NRP1 antibody versus relative expression of Bv8/Prokineticin 2.

FIG. 5 is a graph showing improved efficacy of combination treatment with anti-VEGF antibody and anti-NRP1 antibody versus relative expression of Sema3A (semaphorin3A).

FIG. 6 is a graph showing improved efficacy of combination treatment with anti-VEGF antibody and anti-NRP1 antibody versus relative expression of PlGF (placental growth factor).

FIG. 7 is a graph showing improved efficacy of combination treatment with anti-VEGF antibody and anti-NRP1 antibody versus relative expression of LGALS1 (Galectin-1).

FIG. 8 is a graph showing improved efficacy of combination treatment with anti-VEGF antibody and anti-NRP1 antibody versus relative expression of ITGa5 (integrin alpha 5).

FIG. 9 is a graph showing improved efficacy of combination treatment with anti-VEGF antibody and anti-NRP1 antibody versus relative expression of CSF2/GM-CSF (colony stimulating factor 2/granulocyte macrophage colony-stimulating factor).

FIG. 10 is a graph showing improved efficacy of combination treatment with anti-VEGF antibody and anti-NRP1 antibody versus relative expression of Prox1(prospero-related homeobox 1).

FIG. 11 is a graph showing improved efficacy of combination treatment with anti-VEGF antibody and anti-NRP1 antibody versus relative expression of RGS5 (regulator of G-protein signaling 5).

FIG. 12 is a graph showing improved efficacy of combination treatment with anti-VEGF antibody and anti-NRP1 antibody versus relative expression of HGF (hepatocyte growth factor).

FIG. 13 is a graph showing improved efficacy of combination treatment with anti-VEGF antibody and anti-NRP1 antibody versus relative expression of Sema3B (semaphorin 3B).

FIG. 14 is a graph showing improved efficacy of combination treatment with anti-VEGF antibody and anti-NRP1 antibody versus relative expression of Sema3F (semaphorin 3F).

FIG. 15 is a graph showing improved efficacy of combination treatment with anti-VEGF antibody and anti-NRP1 antibody versus relative expression of LGALS7 (Galectin-7).

FIG. 16 is a table showing the efficacy of combination treatment with anti-VEGF-A antibody and anti-VEGF-C antibody in inhibiting tumor growth in various tumor xenograft models.

FIG. 17 is a table showing p- and r-values for the correlation of marker RNA expression (qPCR) and efficacy of combination treatment with anti-VEGF-A antibody and anti-VEGF-C antibody.

FIG. 18 is a graph showing improved efficacy of the combination treatment with anti-VEGF-A antibody and anti-VEGF-C antibody versus relative expression of VEGF-A.

FIG. 19 is a graph showing improved efficacy of the combination treatment with anti-VEGF-A antibody and anti-VEGF-C antibody versus relative expression of VEGF-C.

FIG. 20 is a graph showing improved efficacy of the combination treatment with anti-VEGF-A antibody and anti-VEGF-C antibody versus relative expression of VEGF-D.

FIG. 21 is a graph showing improved efficacy of the combination treatment with anti-VEGF-A antibody and anti-VEGF-C antibody versus relative expression of VEGFR3.

FIG. 22 is a graph showing improved efficacy of the combination treatment with anti-VEGF-A antibody and anti-VEGF-C antibody versus relative expression of FGF2.

FIG. 23 is a graph showing improved efficacy of the combination treatment with anti-VEGF-A antibody and anti-VEGF-C antibody versus relative expression of CSF2 (colony stimulating factor 2).

FIG. 24 is a graph showing improved efficacy of the combination treatment with anti-VEGF-A antibody and anti-VEGF-C antibody versus relative expression of ICAM1.

FIG. 25 is a graph showing improved efficacy of the combination treatment with anti-VEGF-A antibody and anti-VEGF-C antibody versus relative expression of RGS5 (regulator of G-protein signaling 5).

FIG. 26 is a graph showing improved efficacy of the combination treatment with anti-VEGF-A antibody and anti-VEGF-C antibody versus relative expression of ESM1.

FIG. 27 is a graph showing improved efficacy of the combination treatment with anti-VEGF-A antibody and anti-VEGF-C antibody with anti-VEGF-A antibody and anti-VEGF-C antibody versus relative expression of Prox1 (prospero-related homeobox 1).

FIG. 28 is a graph showing improved efficacy of the combination treatment with anti-VEGF-A antibody and anti-VEGF-C antibody versus relative expression of PlGF.

FIG. 29 is a graph showing improved efficacy of the combination treatment with anti-VEGF-A antibody and anti-VEGF-C antibody versus relative expression of ITGa5.

FIG. 30 is a graph showing improved efficacy of the combination treatment with anti-VEGF-A antibody and anti-VEGF-C antibody versus relative expression of TGF-β.

FIG. 31 is a table showing the efficacy of combination treatment with anti-VEGF-A antibody and anti-EGFL7 antibody in inhibiting tumor growth in various tumor xenograft models.

FIG. 32 is a table showing p- and r-values for the correlation of marker RNA expression (qPCR) and efficacy of combination treatment with anti-VEGF-A antibody and anti-EGFL7 antibody.

FIG. 33 is a graph showing improved efficacy of the combination treatment with anti-VEGF-A antibody and anti-EGFL7 antibody versus relative expression of Sema3B.

FIG. 34 is a graph showing improved efficacy of the combination treatment with anti-VEGF-A antibody and anti-EGFL7 antibody versus relative expression of FGF9.

FIG. 35 is a graph showing improved efficacy of the combination treatment with anti-VEGF-A antibody and anti-EGFL7 antibody versus relative expression of HGF.

FIG. 36 is a graph showing improved efficacy of the combination treatment with anti-VEGF-A antibody and anti-EGFL7 antibody versus relative expression of VEGF-C.

FIG. 37 is a graph showing improved efficacy of the combination treatment with anti-VEGF-A antibody and anti-EGFL7 antibody versus relative expression of RGS5.

FIG. 38 is a graph showing improved efficacy of the combination treatment with anti-VEGF-A antibody and anti-EGFL7 antibody versus relative expression of NRP1.

FIG. 39 is a graph showing improved efficacy of the combination treatment with anti-VEGF-A antibody and anti-EGFL7 antibody versus relative expression of FGF2.

FIG. 40 is a graph showing improved efficacy of the combination treatment with anti-VEGF-A antibody and anti-EGFL7 antibody versus relative expression of CSF2.

FIG. 41 is a graph showing improved efficacy of the combination treatment with anti-VEGF-A antibody and anti-EGFL7 antibody versus relative expression of Bv8.

FIG. 42 is a graph showing improved efficacy of the combination treatment with anti-VEGF-A antibody and anti-EGFL7 antibody versus relative expression of CXCR4.

FIG. 43 is a graph showing improved efficacy of the combination treatment with anti-VEGF-A antibody and anti-EGFL7 antibody versus relative expression of TNFa.

FIG. 44 is a graph showing improved efficacy of the combination treatment with anti-VEGF-A antibody and anti-EGFL7 antibody versus relative expression of cMet.

FIG. 45 is a graph showing improved efficacy of the combination treatment with anti-VEGF-A antibody and anti-EGFL7 antibody versus relative expression of FN1.

FIG. 46 is a graph showing improved efficacy of the combination treatment with anti-VEGF-A antibody and anti-EGFL7 antibody versus relative expression of Fibulin2.

FIG. 47 is a graph showing improved efficacy of the combination treatment with anti-VEGF-A antibody and anti-EGFL7 antibody versus relative expression of Fibulin4.

FIG. 48 is a graph showing improved efficacy of the combination treatment with anti-VEGF-A antibody and anti-EGFL7 antibody versus relative expression of MFAP5.

FIG. 49 is a graph showing improved efficacy of the combination treatment with anti-VEGF-A antibody and anti-EGFL7 antibody versus relative expression of PDGF-C.

FIG. 50 is a table showing the efficacy of combination treatment with anti-VEGF antibody and anti-NRP1 antibody in inhibiting tumor growth in various tumor xenograft models.

FIG. 51 is a table showing p- and r-values for the correlation of marker RNA expression (qPCR) and efficacy of combination treatment with anti-VEGF antibody and anti-NRP1 antibody.

FIG. 52 is a graph showing improved efficacy of combination treatment with anti-VEGF antibody and anti-NRP1 antibody versus relative expression of Sema3B.

FIG. 53 is a graph showing improved efficacy of combination treatment with anti-VEGF antibody and anti-NRP1 antibody versus relative expression of TGFβ.

FIG. 54 is a graph showing improved efficacy of combination treatment with anti-VEGF antibody and anti-NRP1 antibody versus relative expression of FGFR4.

FIG. 55 is a graph showing improved efficacy of combination treatment with anti-VEGF antibody and anti-NRP1 antibody versus relative expression of Vimectin.

FIG. 56 is a graph showing improved efficacy of combination treatment with anti-VEGF antibody and anti-NRP1 antibody versus relative expression of Sema3A.

FIG. 57 is a graph showing improved efficacy of combination treatment with anti-VEGF antibody and anti-NRP1 antibody versus relative expression of PLC.

FIG. 58 is a graph showing improved efficacy of combination treatment with anti-VEGF antibody and anti-NRP1 antibody versus relative expression of CXCL5.

FIG. 59 is a graph showing improved efficacy of combination treatment with anti-VEGF antibody and anti-NRP1 antibody versus relative expression of ITGa5.

FIG. 60 is a graph showing improved efficacy of combination treatment with anti-VEGF antibody and anti-NRP1 antibody versus relative expression of PlGF.

FIG. 61 is a graph showing improved efficacy of combination treatment with anti-VEGF antibody and anti-NRP1 antibody versus relative expression of CCL2.

FIG. 62 is a graph showing improved efficacy of combination treatment with anti-VEGF antibody and anti-NRP1 antibody versus relative expression of IGFB4.

FIG. 63 is a graph showing improved efficacy of combination treatment with anti-VEGF antibody and anti-NRP1 antibody versus relative expression of LGALS1.

FIG. 64 is a graph showing improved efficacy of combination treatment with anti-VEGF antibody and anti-NRP1 antibody versus relative expression of HGF.

FIG. 65 is a graph showing improved efficacy of combination treatment with anti-VEGF antibody and anti-NRP1 antibody versus relative expression of TSP1.

FIG. 66 is a graph showing improved efficacy of combination treatment with anti-VEGF antibody and anti-NRP1 antibody versus relative expression of CXCL1.

FIG. 67 is a graph showing improved efficacy of combination treatment with anti-VEGF antibody and anti-NRP1 antibody versus relative expression of CXCL2.

FIG. 68 is a graph showing improved efficacy of combination treatment with anti-VEGF antibody and anti-NRP1 antibody versus relative expression of Alk1.

FIG. 69 is a graph showing improved efficacy of combination treatment with anti-VEGF antibody and anti-NRP1 antibody versus relative expression of FGF8.

FIG. 70 is a table showing the efficacy of combination treatment with anti-VEGF-A antibody and anti-VEGF-C antibody in inhibiting tumor growth in various tumor xenograft models.

FIG. 71 is a table showing values for the correlation of marker RNA expression (qPCR) and efficacy of combination treatment with anti-VEGF-A antibody and anti-VEGF-C antibody.

FIG. 72 is a graph showing improved efficacy of the combination treatment with anti-VEGF-A antibody and anti-VEGF-C antibody versus relative expression of VEGF-A.

FIG. 73 is a graph showing improved efficacy of the combination treatment with anti-VEGF-A antibody and anti-VEGF-C antibody versus relative expression of VEGF-C.

FIG. 74 is a graph showing improved efficacy of the combination treatment with anti-VEGF-A antibody and anti-VEGF-C antibody versus relative expression of VEGF-C.

FIG. 75 is a graph showing improved efficacy of the combination treatment with anti-VEGF-A antibody and anti-VEGF-C antibody versus relative expression of VEGF-D.

FIG. 76 is a graph showing improved efficacy of the combination treatment with anti-VEGF-A antibody and anti-VEGF-C antibody versus relative expression of VEGFR3.

FIG. 77 is a graph showing improved efficacy of the combination treatment with anti-VEGF-A antibody and anti-VEGF-C antibody versus relative expression of ESM1.

FIG. 78 is a graph showing improved efficacy of the combination treatment with anti-VEGF-A antibody and anti-VEGF-C antibody versus relative expression of ESM1.

FIG. 79 is a graph showing improved efficacy of the combination treatment with anti-VEGF-A antibody and anti-VEGF-C antibody versus relative expression of PlGF.

FIG. 80 is a graph showing improved efficacy of the combination treatment with anti-VEGF-A antibody and anti-VEGF-C antibody versus relative expression of IL-8.

FIG. 81 is a graph showing improved efficacy of the combination treatment with anti-VEGF-A antibody and anti-VEGF-C antibody versus relative expression of IL-8.

FIG. 82 is a graph showing improved efficacy of the combination treatment with anti-VEGF-A antibody and anti-VEGF-C antibody versus relative expression of CXCL1.

FIG. 83 is a graph showing improved efficacy of the combination treatment with anti-VEGF-A antibody and anti-VEGF-C antibody versus relative expression of CXCL1.

FIG. 84 is a graph showing improved efficacy of the combination treatment with anti-VEGF-A antibody and anti-VEGF-C antibody versus relative expression of CXCL2.

FIG. 85 is a graph showing improved efficacy of the combination treatment with anti-VEGF-A antibody and anti-VEGF-C antibody versus relative expression of CXCL2.

FIG. 86 is a graph showing improved efficacy of the combination treatment with anti-VEGF-A antibody and anti-VEGF-C antibody versus relative expression of Hhex.

FIG. 87 is a graph showing improved efficacy of the combination treatment with anti-VEGF-A antibody and anti-VEGF-C antibody versus relative expression of Hhex.

FIG. 88 is a graph showing improved efficacy of the combination treatment with anti-VEGF-A antibody and anti-VEGF-C antibody versus relative expression of Col4a1 and Col4a2.

FIG. 89 is a graph showing improved efficacy of the combination treatment with anti-VEGF-A antibody and anti-VEGF-C antibody versus relative expression of Col4a1 and Col4a2.

FIG. 90 is a graph showing improved efficacy of the combination treatment with anti-VEGF-A antibody and anti-VEGF-C antibody versus relative expression of Alk1.

FIG. 91 is a graph showing improved efficacy of the combination treatment with anti-VEGF-A antibody and anti-VEGF-C antibody versus relative expression of Alk1.

FIG. 92 is a graph showing improved efficacy of the combination treatment with anti-VEGF-A antibody and anti-VEGF-C antibody versus relative expression of Mincle.

FIG. 93 is a table showing the efficacy of combination treatment with anti-VEGF-A antibody and anti-EGFL7 antibody in inhibiting tumor growth in various tumor xenograft models.

FIG. 94 is a table showing p- and r-values for the correlation of marker RNA expression (qPCR) and efficacy of combination treatment with anti-VEGF-A antibody and anti-EGFL7 antibody.

FIG. 95 is a graph showing improved efficacy of the combination treatment with anti-VEGF-A antibody and anti-EGFL7 antibody versus relative expression of Sema3B.

FIG. 96 is a graph showing improved efficacy of the combination treatment with anti-VEGF-A antibody and anti-EGFL7 antibody versus relative expression of FGF9.

FIG. 97 is a graph showing improved efficacy of the combination treatment with anti-VEGF-A antibody and anti-EGFL7 antibody versus relative expression of HGF.

FIG. 98 is a graph showing improved efficacy of the combination treatment with anti-VEGF-A antibody and anti-EGFL7 antibody versus relative expression of VEGF-C.

FIG. 99 is a graph showing improved efficacy of the combination treatment with anti-VEGF-A antibody and anti-EGFL7 antibody versus relative expression of FGF2.

FIG. 100 is a graph showing improved efficacy of the combination treatment with anti-VEGF-A antibody and anti-EGFL7 antibody versus relative expression of Bv8.

FIG. 101 is a graph showing improved efficacy of the combination treatment with anti-VEGF-A antibody and anti-EGFL7 antibody versus relative expression of TNFa.

FIG. 102 is a graph showing improved efficacy of the combination treatment with anti-VEGF-A antibody and anti-EGFL7 antibody versus relative expression of cMet.

FIG. 103 is a graph showing improved efficacy of the combination treatment with anti-VEGF-A antibody and anti-EGFL7 antibody versus relative expression of FN1.

FIG. 104 is a graph showing improved efficacy of the combination treatment with anti-VEGF-A antibody and anti-EGFL7 antibody versus relative expression of Fibulin 2.

FIG. 105 is a graph showing improved efficacy of the combination treatment with anti-VEGF-A antibody and anti-EGFL7 antibody versus relative expression of EFEMP2/fibulin 4.

FIG. 106 is a graph showing improved efficacy of the combination treatment with anti-VEGF-A antibody and anti-EGFL7 antibody versus relative expression of MFAP5.

FIG. 107 is a graph showing improved efficacy of the combination treatment with anti-VEGF-A antibody and anti-EGFL7 antibody versus relative expression of PDGF-C.

FIG. 108 is a graph showing improved efficacy of the combination treatment with anti-VEGF-A antibody and anti-EGFL7 antibody versus relative expression of Fras1.

FIG. 109 is a graph showing improved efficacy of the combination treatment with anti-VEGF-A antibody and anti-EGFL7 antibody versus relative expression of CXCL2.

FIG. 110 is a graph showing improved efficacy of the combination treatment with anti-VEGF-A antibody and anti-EGFL7 antibody versus relative expression of Mincle.

### DETAILED DESCRIPTION OF THE INVENTION

### I. Introduction

The present invention provides methods and compositions for identifying patients who may benefit from treatment with an anti-angiogenic therapy including, for example, anti-cancer therapy, other than or in addition to a VEGF antagonist. The invention is based on the discovery that measuring an increase or decrease in expression of at least one gene selected from 18S rRNA, ACTB, RPS13, VEGFA, VEGFC, VEGFD, Bv8, PlGF, VEGFR1/Flt1, VEGFR2, VEGFR3, NRP1, sNRP1, Podoplanin, Prox1, VE-Cadherin (CD144, CDH5), robo4, FGF2, IL8/CXCL8, HGF, THBS1/TSP1, Egf17, NG3/Egfl8, ANG1, GM-CSF/CSF2, G-CSF/CSF3, FGF9, CXCL12/SDF1, TGFβ1, TNFα, Alk1, BMP9, BMP10, HSPG2/perlecan, ESM1, Sema3a, Sema3b, Sema3c, Sema3e, Sema3f, NG2, ITGa5, ICAM1, CXCR4, LGALS1/Galectin1, LGALS7B/Galectin7, Fibronectin, TMEM100, PECAM/CD31, PDGFβ, PDGFRβ, RGS5, CXCL1, CXCL2, robo4, LyPD6, VCAM1, collagen IV (a1), collagen IV (a2), collagen IV (a3), Spred-1, Hhex, ITGa5, LGALS1/Galectin1, LGALS7/Galectin7, TMEM100, MFAP5, Fibronectin, fibulin2, and fibulin4/Efemp2 is useful for monitoring a patient's responsiveness or sensitivity to treatment with an anti-angiogenic therapy other than or in addition to a VEGF antagonist or for determining the likelihood that a patient will benefit or exhibit benefit from treatment with an anti-angiogenic therapy other than or in addition to a VEGF antagonist. Suitable anti-angiogenic therapies include treatment with, e.g., a NRP1 antagonist, a VEGF-C antagonist, or an EGFL7 antagonist.

### II. Definitions

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. All references cited herein, including patent applications, patent publications, and Genbank Accession numbers are herein incorporated by reference, as if each individual reference were specifically and individually indicated to be incorporated by reference.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

An “individual,” “subject,” or “patient” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, a mammal is a human.

The term “sample,” or “test sample” as used herein, refers to a composition that is obtained or derived from a subject of interest that contains a cellular and/or other molecular entity that is to be characterized and/or identified, for example based on physical, biochemical, chemical and/or physiological characteristics. In one embodiment, the definition encompasses blood and other liquid samples of biological origin and tissue samples such as a biopsy specimen or tissue cultures or cells derived therefrom. The source of the tissue sample may be solid tissue as from a fresh, frozen and/or preserved organ or tissue sample or biopsy or aspirate; blood or any blood constituents; bodily fluids; and cells from any time in gestation or development of the subject or plasma.

The term “sample,” or “test sample” includes biological samples that have been manipulated in any way after their procurement, such as by treatment with reagents, solubilization, or enrichment for certain components, such as proteins or polynucleotides, or embedding in a semi-solid or solid matrix for sectioning purposes. For the purposes herein a “section” of a tissue sample is meant a single part or piece of a tissue sample, e.g. a thin slice of tissue or cells cut from a tissue sample.

Samples include, but not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

In one embodiment, the sample is a clinical sample. In another embodiment, the sample is used in a diagnostic assay. In some embodiments, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood.

In one embodiment, a sample is obtained from a subject or patient prior to anti-angiogenic therapy. In another embodiment, a sample is obtained from a subject or patient prior to VEGF antagonist therapy. In yet another embodiment, a sample is obtained from a subject or patient prior to anti-VEGF antibody therapy. In even another embodiment, a sample is obtained from a subject or patient following at least one treatment with VEGF antagonist therapy.

In one embodiment, a sample is obtained from a subject or patient after at least one treatment with an anti-angiogenic therapy. In yet another embodiment, a sample is obtained from a subject or patient following at least one treatment with an anti-VEGF antibody. In some embodiments, a sample is obtained from a patient before cancer has metastasized. In certain embodiments, a sample is obtained from a patient after cancer has metastasized.

A “reference sample,” as used herein, refers to any sample, standard, or level that is used for comparison purposes. In one embodiment, a reference sample is obtained from a healthy and/or non-diseased part of the body (e.g., tissue or cells) of the same subject or patient. In another embodiment, a reference sample is obtained from an untreated tissue and/or cell of the body of the same subject or patient. In yet another embodiment, a reference sample is obtained from a healthy and/or non-diseased part of the body (e.g., tissues or cells) of an individual who is not the subject or patient. In even another embodiment, a reference sample is obtained from an untreated tissue and/or cell part of the body of an individual who is not the subject or patient.

In certain embodiments, a reference sample is a single sample or combined multiple samples from the same subject or patient that are obtained at one or more different time points than when the test sample is obtained. For example, a reference sample is obtained at an earlier time point from the same subject or patient than when the test sample is obtained. Such reference sample may be useful if the reference sample is obtained during initial diagnosis of cancer and the test sample is later obtained when the cancer becomes metastatic.

In certain embodiments, a reference sample includes all types of biological samples as defined above under the term “sample” that is obtained from one or more individuals who is not the subject or patient. In certain embodiments, a reference sample is obtained from one or more individuals with an angiogenic disorder (e.g., cancer) who is not the subject or patient.

In certain embodiments, a reference sample is a combined multiple samples from one or more healthy individuals who are not the subject or patient. In certain embodiments, a reference sample is a combined multiple samples from one or more individuals with a disease or disorder (e.g., an angiogenic disorder such as, for example, cancer) who are not the subject or patient. In certain embodiments, a reference sample is pooled RNA samples from normal tissues or pooled plasma or serum samples from one or more individuals who are not the subject or patient. In certain embodiments, a reference sample is pooled RNA samples from tumor tissues or pooled plasma or serum samples from one or more individuals with a disease or disorder (e.g., an angiogenic disorder such as, for example, cancer) who are not the subject or patient.

Expression levels/amount of a gene or biomarker can be determined qualitatively and/or quantitatively based on any suitable criterion known in the art, including but not limited to mRNA, cDNA, proteins, protein fragments and/or gene copy number. In certain embodiments, expression/amount of a gene or biomarker in a first sample is increased as compared to expression/amount in a second sample. In certain embodiments, expression/amount of a gene or biomarker in a first sample is decreased as compared to expression/amount in a second sample. In certain embodiments, the second sample is reference sample. Additional disclosures for determining expression level/amount of a gene are described hereinbelow under Methods of the Invention and in Examples 1 and 2.

In certain embodiments, the term “increase” refers to an overall increase of 5%, 10%, 20%, 25%, 30%, 40%, 50%, 60%, 70%, 80%, 85%, 90%, 95%, 96%, 97%, 98%, 99% or greater, in the level of protein or nucleic acid, detected by standard art known methods such as those described herein, as compared to a reference sample. In certain embodiments, the term increase refers to the increase in expression level/amount of a gene or biomarker in the sample wherein the increase is at least about 1.5×, 1.75×, 2×, 3×, 4×, 5×, 6×, 7×, 8×, 9×, 10×, 25×, 50×, 75×, or 100× the expression level/amount of the respective gene or biomarker in the reference sample.

In certain embodiments, the term “decrease” herein refers to an overall reduction of 5%, 10%, 20%, 25%, 30%, 40%, 50%, 60%, 70%, 80%, 85%, 90%, 95%, 96%, 97%, 98%, 99% or greater, in the level of protein or nucleic acid, detected by standard art known methods such as those described herein, as compared to a reference sample. In certain embodiments, the term decrease refers to the decrease in expression level/amount of a gene or biomarker in the sample wherein the decrease is at least about 0.9×, 0.8×, 0.7×, 0.6×, 0.5×, 0.4×, 0.3×, 0.2×, 0.1×, 0.05×, or 0.01× the expression level/amount of the respective gene or biomarker in the reference sample.

“Detection” includes any means of detecting, including direct and indirect detection.

In certain embodiments, by “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

“Neuropilin” or “NRP” refers collectively to neuropilin-1 (NRP1), neuropilin-2 (NRP2) and their isoforms and variants, as described in Rossignol et al. (2000) Genomics 70:211-222. Neuropilins are 120 to 130 kDa non-tyrosine kinase receptors. There are multiple NRP-1 and NRP-2 splice variants and soluble isoforms. The basic structure of neuropilins comprises five domains: three extracellular domains (a1a2, b1b2 and c), a transmembrane domain, and a cytoplasmic domain. The a1a2 domain is homologous to complement components C1r and C1s (CUB), which generally contains four cysteine residues that form two disculfid bridges. The b1b2 domain is homologous to coagulation factors V and VIII. The central portion of the c domain is designated as MAM due to its homology to meprin, AS and receptor tyrosine phosphotase μ proteins. The a1a2 and b1b2 domains are responsible for ligand binding, whereas the c domain is critical for homodimerization or heterodimerization. Gu et al. (2002) J. Biol. Chem. 277:18069-76; He and Tessier-Lavigne (1997) Cell 90:739-51.

“Neuropilin mediated biological activity” or “NRP mediated biological activity” refers in general to physiological or pathological events in which neuropilin-1 and/or neuropilin-2 plays a substantial role. Non-limiting examples of such activities are axon guidance during embryonic nervous system development or neuron-regeneration, angiogenesis (including vascular modeling), tumorgenesis and tumor metastasis.

A “NRP1 antagonist” or “NRP1-specific antagonist” refers to a molecule capable of neutralizing, blocking, inhibiting, abrogating, reducing or interfering with NRP mediated biological activities including, but not limited to, its binding to one or more NRP ligands, e.g., VEGF, PlGF, VEGF-B, VEGF-C, VEGF-D, Sema3A, Sema3B, Sema3C, HGF, FGF1, FGF2, Galectin-1. NRP1 antagonists include, without limitation, anti-NRP1 antibodies and antigen-binding fragments thereof and small molecule inhibitors of NRP1. The term “NRP1 antagonist,” as used herein, specifically includes molecules, including antibodies, antibody fragments, other binding polypeptides, peptides, and non-peptide small molecules, that bind to NRP1 and are capable of neutralizing, blocking, inhibiting, abrogating, reducing or interfering with NRP1 activities. Thus, the term “NRP1 activities” specifically includes NRP1 mediated biological activities of NRP1. In certain embodiments, the NRP1 antagonist reduces or inhibits, by at least 10%, 20%, 30%, 40%, 50%, 60%, 70%, 80%, 90% or more, the expression level or biological activity of NRP1.

An “anti-NRP1 antibody” is an antibody that binds to NRP1 with sufficient affinity and specificity. An “anti-NRP1^{B} antibody” is an antibody that binds to the coagulation factor V/VIII domains (b1b2) of NRP1. In certain embodiments, the antibody selected will normally have a sufficiently binding affinity for NRP1, for example, the antibody may bind human NRP1 with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiment, the anti-NRP1 antibody can be used as a therapeutic agent in targeting and interfering with diseases or conditions wherein the NRP1 activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-NRP1 antibody will usually not bind to other neuropilins such as NRP2. In one embodiment the anti-NRP1^{B} antibody of the invention preferably comprises a light chain variable domain comprising the following CDR amino acid sequences: CDRL1 (RASQYFSSYLA), CDRL2 (GASSRAS) and CDRL3 (QQYLGSPPT). For example, the anti-NRP1^{B} antibody comprises a light chain variable domain sequence of SEQ ID NO:5 of PCT publication No. WO2007/056470. The anti-NRP1^{B} antibody of the invention preferably comprises a heavy chain variable domain comprising the following CDR amino acid sequences: CDRH1 (GFTFSSYAMS), CDRH2 (SQISPAGGYTNYADSVKG) and CDRH3 (ELPYYRMSKVMDV). For example, the anti-NRP1^{B} antibody comprises a heavy chain variable domain sequence of SEQ ID NO:6 of PCT publication No. WO2007/056470. In another embodiment the anti-NRP1^{B} antibody is generated according to PCT publication No. WO2007/056470 or US publication No. US2008/213268.

The terms “EGFL7” or “EGF-like-domain, multiple 7” are used interchangeably herein to refers to any native or variant (whether native or synthetic) EGFL7 polypeptide. The term “native sequence” specifically encompasses naturally occurring truncated or secreted forms (e.g., an extracellular domain sequence), naturally occurring variant forms (e.g., alternatively spliced forms) and naturally-occurring allelic variants. The term “wild type EGFL7” generally refers to a polypeptide comprising the amino acid sequence of a naturally occurring EGFL7 protein. The term “wild type EGFL7 sequence” generally refers to an amino acid sequence found in a naturally occurring EGFL7.

An “EGFL7 antagonist” or “EGFL7-specific antagonist” refers to a molecule capable of neutralizing, blocking, inhibiting, abrogating, reducing or interfering with EGFL7-mediated biological activities including, but not limited to, EGFL7-mediated HUVEC cell adhesion or HUVEC cell migration. EGFL7 antagonists include, without limitation, anti-EGFL7 antibodies and antigen-binding fragments thereof and small molecule inhibitors of EGFL7. The term “EGFL7 antagonist,” as used herein, specifically includes molecules, including antibodies, antibody fragments, other binding polypeptides, peptides, and non-peptide small molecules, that bind to EGFL7 and are capable of neutralizing, blocking, inhibiting, abrogating, reducing or interfering with EGFL7 activities. Thus, the term “EGFL7 activities” specifically includes EGFL7-mediated biological activities of EGFL7. In certain embodiments, the EGFL7 antagonist reduces or inhibits, by at least 10%, 20%, 30%, 40%, 50%, 60%, 70%, 80%, 90% or more, the expression level or biological activity of EGFL7.

An “anti-EGFL7 antibody” is an antibody that binds to EGFL7 with sufficient affinity and specificity. In certain embodiments, the antibody selected will normally have a sufficiently binding affinity for EGFL7, for example, the antibody may bind human EGFL7 with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiment, the anti-EGFL7 antibody can be used as a therapeutic agent in targeting and interfering with diseases or conditions wherein the EGFL7 activity is involved. Also, the antibody may be subjected to other biological activity assays, e.g., in order to evaluate its effectiveness as a therapeutic. Such assays are known in the art and depend on the target antigen and intended use for the antibody. Examples include inhibition of HUVEC cell adhesion and/or migration; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). In some embodiments, the anti-EGFL7 antibody of the invention comprises a light chain variable domain comprising the following CDR amino acid sequences: CDRL1 (KASQSVDYSGDSYMS), CDRL2 (GASYRES) and CDRL3 (QQNNEEPYT). In some embodiments, the anti-EGFL7 antibody of the invention comprises a light chain variable domain comprising the following CDR amino acid sequences: CDRL1 (RTSQSLVHINAITYLH), CDRL2 (RVSNRFS) and CDRL3 (GQSTHVPLT). In some embodiments, the anti-EGFL7 antibody of the invention preferably comprises a heavy chain variable domain comprising the following CDR amino acid sequences: CDRH1 (GHTFTTYGMS), CDRH2 (GWINTHSGVPTYADDFKG) and CDRH3 (LGSYAVDY). In some embodiments, the anti-EGFL7 antibody of the invention preferably comprises a heavy chain variable domain comprising the following CDR amino acid sequences: CDRH1 (GYTFIDYYMN), CDRH2 (GDINLDNSGTHYNQKFKG) and CDRH3 (AREGVYHDYDDYAMDY).

The terms “vascular endothelial growth factor-C”, “VEGF-C”, “VEGFC”, “VEGF-related protein”, “VRP”, “VEGF2” and “VEGF-2” are used interchangeably, and refer to a member of the VEGF family, is known to bind at least two cell surface receptor families, the tyrosine kinase VEGF receptors and the neuropilin (Nrp) receptors. Of the three VEGF receptors, VEGF-C can bind VEGFR2 (KDR receptor) and VEGFR3 (Flt-4 receptor) leading to receptor dimerization (Shinkai et al., J Biol Chem 273, 31283-31288 (1998)), kinase activation and autophosphorylation (Heldin, Cell 80, 213-223 (1995); Waltenberger et al., J. Biol Chem 269, 26988-26995 (1994)). The phosphorylated receptor induces the activation of multiple substrates leading to angiogenesis and lymphangiogenesis (Ferrara et al., Nat Med 9, 669-676 (2003)). Overexpression of VEGF-C in tumor cells was shown to promote tumor-associated lymphangiogenesis, resulting in enhanced metastasis to regional lymph nodes (Karpanen et al., Faseb J 20, 1462-1472 (2001); Mandriota et al., EMBO J 20, 672-682 (2001); Skobe et al., Nat Med 7, 192-198 (2001); Stacker et al., Nat Rev Cancer 2, 573-583 (2002); Stacker et al., Faseb J 16, 922-934 (2002)). VEGF-C expression has also been correlated with tumor-associated lymphangiogenesis and lymph node metastasis for a number of human cancers (reviewed in Achen et al., 2006, supra. In addition, blockade of VEGF-C-mediated signaling has been shown to suppress tumor lymphangiogenesis and lymph node metastases in mice (Chen et al., Cancer Res 65, 9004-9011 (2005); He et al., J. Natl Cancer Inst 94, 8190825 (2002); Krishnan et al., Cancer Res 63, 713-722 (2003); Lin et al., Cancer Res 65, 6901-6909 (2005)).

“Vascular endothelial growth factor-C”, “VEGF-C”, “VEGFC”, “VEGF-related protein”, “VRP”, “VEGF2” and “VEGF-2” refer to the full-length polypeptide and/or the active fragments of the full-length polypeptide. In one embodiment, active fragments include any portions of the full-length amino acid sequence which have less than the full 419 amino acids of the full-length amino acid sequence as shown in SEQ ID NO:3 of U.S. Pat. No. 6,451,764, the entire disclosure of which is expressly incorporated herein by reference. Such active fragments contain VEGF-C biological activity and include, but not limited to, mature VEGF-C. In one embodiment, the full-length VEGF-C polypeptide is proteolytically processed produce a mature form of VEGF-C polypeptide, also referred to as mature VEGF-C. Such processing includes cleavage of a signal peptide and cleavage of an amino-terminal peptide and cleavage of a carboxyl-terminal peptide to produce a fully-processed mature form. Experimental evidence demonstrates that the full-length VEGF-C, partially-processed forms of VEGF-C and fully processed mature forms of VEGF-C are able to bind VEGFR3 (Flt-4 receptor). However, high affinity binding to VEGFR2 occurs only with the fully processed mature forms of VEGF-C.

The term “biological activity” and “biologically active” with regard to a VEGF-C polypeptide refer to physical/chemical properties and biological functions associated with full-length and/or mature VEGF-C. In some embodiments, VEGF-C “biological activity” means having the ability to bind to, and stimulate the phosphorylation of, the Flt-4 receptor (VEGFR3). Generally, VEGF-C will bind to the extracellular domain of the Flt-4 receptor and thereby activate or inhibit the intracellular tyrosine kinase domain thereof. Consequently, binding of VEGF-C to the receptor may result in enhancement or inhibition of proliferation and/or differentiation and/or activation of cells having the Flt-4 receptor for the VEGF-C in vivo or in vitro. Binding of VEGF-C to the Flt-4 receptor can be determined using conventional techniques, including competitive binding methods, such as RIAs, ELISAs, and other competitive binding assays. Ligand/receptor complexes can be identified using such separation methods as filtration, centrifugation, flow cytometry (see, e.g., Lyman et al., Cell, 75:1157-1167 [1993]; Urdal et al., J. Biol. Chem., 263:2870-2877 [1988]; and Gearing et al., EMBO J., 8:3667-3676 [1989]), and the like. Results from binding studies can be analyzed using any conventional graphical representation of the binding data, such as Scatchard analysis (Scatchard, Ann. NY Acad. Sci., 51:660-672 [1949]; Goodwin et al., Cell, 73:447-456 [1993]), and the like. Since VEGF-C induces phosphorylation of the Flt-4 receptor, conventional tyrosine phosphorylation assays can also be used as an indication of the formation of a Flt-4 receptor/VEGF-C complex. In another embodiment, VEGF-C “biological activity” means having the ability to bind to KDR receptor (VEGFR2). vascular permeability, as well as the migration and proliferation of endothelial cells. In certain embodiments, binding of VEGF-C to the KDR receptor may result in enhancement or inhibition of vascular permeability as well as migration and/or proliferation and/or differentiation and/or activation of endothelial cells having the KDR receptor for the VEGF-C in vivo or in vitro.

The term “VEGF-C antagonist” is used herein to refer to a molecule capable of neutralizing, blocking, inhibiting, abrogating, reducing or interfering with VEGF-C activities. In certain embodiments, VEGF-C antagonist refers to a molecule capable of neutralizing, blocking, inhibiting, abrogating, reducing or interfering with the ability of VEGF-C to modulate angiogenesis, lymphatic endothelial cell (EC) migration, proliferation or adult lymphangiogenesis, especially tumoral lymphangiogenesis and tumor metastasis. VEGF-C antagonists include, without limitation, anti-VEGF-C antibodies and antigen-binding fragments thereof, receptor molecules and derivatives which bind specifically to VEGF-C thereby sequestering its binding to one or more receptors, anti-VEGF-C receptor antibodies and VEGF-C receptor antagonists such as small molecule inhibitors of the VEGFR2 and VEGFR3. The term “VEGF-C antagonist,” as used herein, specifically includes molecules, including antibodies, antibody fragments, other binding polypeptides, peptides, and non-peptide small molecules, that bind to VEGF-C and are capable of neutralizing, blocking, inhibiting, abrogating, reducing or interfering with VEGF-C activities. Thus, the term “VEGF-C activities” specifically includes VEGF-C mediated biological activities (as hereinabove defined) of VEGF-C.

The term “anti-VEGF-C antibody” or “an antibody that binds to VEGF-C” refers to an antibody that is capable of binding VEGF-C with sufficient affinity such that the antibody is useful as a diagnostic and/or therapeutic agent in targeting VEGF-C. Anti-VEGF-C antibodies are described, for example, in Attorney Docket PR4291, the entire content of the patent application is expressly incorporated herein by reference. In one embodiment, the extent of binding of an anti-VEGF-C antibody to an unrelated, non-VEGF-C protein is less than about 10% of the binding of the antibody to VEGF-C as measured, e.g., by a radioimmunoassay (RIA). In certain embodiments, an antibody that binds to VEGF-C has a dissociation constant (Kd) of ≦1 μm, ≦100 nM, ≦10 nM, ≦1 nM, or ≦0.1 nM. In certain embodiments, an anti-VEGF-C antibody binds to an epitope of VEGF-C that is conserved among VEGF-C from different species.

The term “VEGF” or “VEGF-A” as used herein refers to the 165-amino acid human vascular endothelial cell growth factor and related 121-, 189-, and 206-amino acid human vascular endothelial cell growth factors, as described by Leung et al. (1989) Science 246:1306, and Houck et al. (1991) Mol. Endocrin, 5:1806, together with the naturally occurring allelic and processed forms thereof. The term “VEGF” also refers to VEGFs from non-human species such as mouse, rat or primate. Sometimes the VEGF from a specific species are indicated by terms such as hVEGF for human VEGF, mVEGF for murine VEGF, and etc. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

“VEGF biological activity” includes binding to any VEGF receptor or any VEGF signaling activity such as regulation of both normal and abnormal angiogenesis and vasculogenesis (Ferrara and Davis-Smyth (1997) Endocrine Rev. 18:4-25; Ferrara (1999) J. Mol. Med. 77:527-543); promoting embryonic vasculogenesis and angiogenesis (Carmeliet et al. (1996) Nature 380:435-439; Ferrara et al. (1996) Nature 380:439-442); and modulating the cyclical blood vessel proliferation in the female reproductive tract and for bone growth and cartilage formation (Ferrara et al. (1998) Nature Med. 4:336-340; Gerber et al. (1999) Nature Med. 5:623-628). In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx (Ferrara and Davis-Smyth (1997), supra and Cebe-Suarez et al. Cell. Mol. Life. Sci. 63:601-615 (2006)). Moreover, recent studies have reported mitogenic effects of VEGF on a few non-endothelial cell types, such as retinal pigment epithelial cells, pancreatic duct cells, and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors and VEGF mediated angiogenesis and endothelial cell survival or proliferation. Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers directed to VEGF, small RNA molecules directed to VEGF, RNA aptamers, peptibodies, and ribozymes against VEGF. VEGF-specific antagonists also include nonpeptide small molecules that bind to VEGF and are capable of blocking, inhibiting, abrogating, reducing, or interfering with VEGF biological activities. Thus, the term “VEGF activities” specifically includes VEGF mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF.

An “anti-VEGF antibody” is an antibody that binds to VEGF with sufficient affinity and specificity. In certain embodiments, the antibody selected will normally have a sufficiently binding affinity for VEGF, for example, the antibody may bind hVEGF with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example.

In certain embodiment, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PlGF, PDGF or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (1997) Cancer Res. 57:4593-4599, including but not limited to the antibody known as bevacizumab (BV; AVASTIN®).

The anti-VEGF antibody “Bevacizumab (BV),” also known as “rhuMAb VEGF” or “AVASTIN®,” is a recombinant humanized anti-VEGF monoclonal antibody generated according to Presta et al. (1997) Cancer Res. 57:4593-4599. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference.

The two best characterized VEGF receptors are VEGFR1 (also known as Flt-1) and VEGFR2 (also known as KDR and FLK-1 for the murine homolog). The specificity of each receptor for each VEGF family member varies but VEGF-A binds to both Flt-1 and KDR. The full length Flt-1 receptor includes an extracellular domain that has seven Ig domains, a transmembrane domain, and an intracellular domain with tyrosine kinase activity. The extracellular domain is involved in the binding of VEGF and the intracellular domain is involved in signal transduction.

VEGF receptor molecules, or fragments thereof, that specifically bind to VEGF can be used as VEGF inhibitors that bind to and sequester the VEGF protein, thereby preventing it from signaling. In certain embodiments, the VEGF receptor molecule, or VEGF binding fragment thereof, is a soluble form, such as sFlt-1. A soluble form of the receptor exerts an inhibitory effect on the biological activity of the VEGF protein by binding to VEGF, thereby preventing it from binding to its natural receptors present on the surface of target cells. Also included are VEGF receptor fusion proteins, examples of which are described below.

A chimeric VEGF receptor protein is a receptor molecule having amino acid sequences derived from at least two different proteins, at least one of which is a VEGF receptor protein (e.g., the flt-1 or KDR receptor), that is capable of binding to and inhibiting the biological activity of VEGF. In certain embodiments, the chimeric VEGF receptor proteins of the present invention consist of amino acid sequences derived from only two different VEGF receptor molecules; however, amino acid sequences comprising one, two, three, four, five, six, or all seven Ig-like domains from the extracellular ligand-binding region of the flt-1 and/or KDR receptor can be linked to amino acid sequences from other unrelated proteins, for example, immunoglobulin sequences. Other amino acid sequences to which Ig-like domains are combined will be readily apparent to those of ordinary skill in the art. Examples of chimeric VEGF receptor proteins include, but not limited to, soluble Flt-1/Fc, KDR/Fc, or Flt-1/KDR/Fc (also known as VEGF Trap). (See for example PCT Application Publication No. WO97/44453).

A soluble VEGF receptor protein or chimeric VEGF receptor proteins includes VEGF receptor proteins which are not fixed to the surface of cells via a transmembrane domain. As such, soluble forms of the VEGF receptor, including chimeric receptor proteins, while capable of binding to and inactivating VEGF, do not comprise a transmembrane domain and thus generally do not become associated with the cell membrane of cells in which the molecule is expressed.

Additional VEGF inhibitors are described in, for example in WO 99/24440, PCT International Application PCT/IB99/00797, in WO 95/21613, WO 99/61422, U.S. Pat. No. 6,534,524, U.S. Pat. No. 5,834,504, WO 98/50356, U.S. Pat. No. 5,883,113, U.S. Pat. No. 5,886,020, U.S. Pat. No. 5,792,783, U.S. Pat. No. 6,653,308, WO 99/10349, WO 97/32856, WO 97/22596, WO 98/54093, WO 98/02438, WO 99/16755, and WO 98/02437, all of which are herein incorporated by reference in their entirety.

The term “B20 series polypeptide” as used herein refers to a polypeptide, including an antibody that binds to VEGF. B20 series polypeptides includes, but not limited to, antibodies derived from a sequence of the B20 antibody or a B20-derived antibody described in US Publication No. 20060280747, US Publication No. 20070141065 and/or US Publication No. 20070020267, the content of these patent applications are expressly incorporated herein by reference. In one embodiment, B20 series polypeptide is B20-4.1 as described in US Publication No. 20060280747, US Publication No. 20070141065 and/or US Publication No. 20070020267. In another embodiment, B20 series polypeptide is B20-4.1.1 described in U.S. Patent Application 60/991,302, the entire disclosure of which is expressly incorporated herein by reference.

The term “G6 series polypeptide” as used herein refers to a polypeptide, including an antibody that binds to VEGF. G6 series polypeptides includes, but not limited to, antibodies derived from a sequence of the G6 antibody or a G6-derived antibody described in US Publication No. 20060280747, US Publication No. 20070141065 and/or US Publication No. 20070020267. G6 series polypeptides, as described in US Publication No. 20060280747, US Publication No. 20070141065 and/or US Publication No. 20070020267 include, but not limited to, G6-8, G6-23 and G6-31.

For additional antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). In certain embodiments, other antibodies include those that bind to a functional epitope on human VEGF comprising of residues F17, M18, D19, Y21, Y25, Q89, I91, K101, E103, and C104 or, alternatively, comprising residues F17, Y21, Q22, Y25, D63, I83 and Q89.

Other anti-VEGF antibodies and anti-NRP1 antibodies are also known, and described, for example, in Liang et al., J Mol Biol 366, 815-829 (2007) and Liang et al., J Biol Chem 281, 951-961 (2006), PCT publication number WO2007/056470 and PCT Application No. PCT/US2007/069179, the content of these patent applications is expressly incorporated herein by reference.

The word “label” when used herein refers to a compound or composition which is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable.

A “small molecule” is defined herein to have a molecular weight below about 500 Daltons.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs.

“Oligonucleotide,” as used herein, generally refers to short, generally single-stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

In certain embodiments, polynucleotides are capable of specifically hybridizing to a gene under various stringency conditions. “Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

Stringent conditions or high stringency conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

An “isolated” nucleic acid molecule is a nucleic acid molecule that is identified and separated from at least one contaminant nucleic acid molecule with which it is ordinarily associated in the natural source of the polypeptide nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

A “primer” is generally a short single stranded polynucleotide, generally with a free 3′-OH group, that binds to a target potentially present in a sample of interest by hybridizing with a target sequence, and thereafter promotes polymerization of a polynucleotide complementary to the target.

The term “housekeeping gene” refers to a group of genes that codes for proteins whose activities are essential for the maintenance of cell function. These genes are typically similarly expressed in all cell types.

The term “biomarker” as used herein refers generally to a molecule, including a gene, protein, carbohydrate structure, or glycolipid, the expression of which in or on a mammalian tissue or cell can be detected by standard methods (or methods disclosed herein) and is predictive, diagnostic and/or prognostic for a mammalian cell's or tissue's sensitivity to treatment regimes based on inhibition of angiogenesis e.g. an anti-angiogenic agent such as a VEGF-specific inhibitor. In certain embodiments, the expression of such a biomarker is determined to be higher or lower than that observed for a reference sample. Expression of such biomarkers can be determined using a high-throughput multiplexed immunoassay such as those commercially available from Rules Based Medicine, Inc. or Meso Scale Discovery. Expression of the biomarkers may also be determined using, e.g., PCR or FACS assay, an immunohistochemical assay or a gene chip-based assay.

The term “array” or “microarray,” as used herein refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane. The nucleotide sequences can be DNA, RNA, or any permutations thereof.

A “gene,” “target gene,” “target biomarker,” “target sequence,” “target nucleic acid” or “target protein,” as used herein, is a polynucleotide or protein of interest, the detection of which is desired. Generally, a “template,” as used herein, is a polynucleotide that contains the target nucleotide sequence. In some instances, the terms “target sequence,” “template DNA,” “template polynucleotide,” “target nucleic acid,” “target polynucleotide,” and variations thereof, are used interchangeably.

“Amplification,” as used herein, generally refers to the process of producing multiple copies of a desired sequence. “Multiple copies” mean at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

A “native sequence” polypeptide comprises a polypeptide having the same amino acid sequence as a polypeptide derived from nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

An “isolated” polypeptide or “isolated” antibody is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In certain embodiments, the polypeptide will be purified (1) to greater than 95% by weight of polypeptide as determined by the Lowry method, or more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or silver stain. Isolated polypeptide includes the polypeptide in situ within recombinant cells since at least one component of the polypeptide's natural environment will not be present. Ordinarily, however, isolated polypeptide will be prepared by at least one purification step.

A polypeptide “variant” means a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

Unless indicated otherwise, the expression “multivalent antibody” denotes an antibody comprising three or more antigen binding sites. In certain embodiment, the multivalent antibody is engineered to have the three or more antigen binding sites and is generally not a native sequence IgM or IgA antibody.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

“Framework” or “FR” residues are those variable domain residues other than the HVR residues as herein defined.

An “affinity matured” antibody is one with one or more alterations in one or more HVRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies may be produced using certain procedures known in the art. For example, Marks et al. Bio/Technology 10:779-783 (1992) describes affinity maturation by VH and VL domain shuffling. Random mutagenesis of HVR and/or framework residues is described by, for example, Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

The term “Fc region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, a composition comprising an antibody having an Fc region according to this invention can comprise an antibody with K447, with all K447 removed, or a mixture of antibodies with and without the K447 residue.

A “blocking” antibody or an “antagonist” antibody is one which inhibits or reduces biological activity of the antigen it binds. For example, a VEGF-specific antagonist antibody binds VEGF and inhibits the ability of VEGF to induce vascular endothelial cell proliferation or vascular permeability. Certain blocking antibodies or antagonist antibodies substantially or completely inhibit the biological activity of the antigen.

As used herein, “treatment” (and variations such as “treat” or “treating”) refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods and compositions of the invention are used to delay development of a disease or disorder or to slow the progression of a disease or disorder.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A therapeutically effective amount also encompasses an amount sufficient to confer benefit, e.g., clinical benefit.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount. A prophylactically effective amount encompasses an amount sufficient to confer benefit, e.g., clinical benefit.

In the case of pre-cancerous, benign, early or late-stage tumors, the therapeutically effective amount of the angiogenic inhibitor may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit or delay, to some extent, tumor growth or tumor progression; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

To “reduce” or “inhibit” is to decrease or reduce an activity, function, and/or amount as compared to a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater. Reduce or inhibit can refer to the symptoms of the disorder being treated, the presence or size of metastases, the size of the primary tumor, or the size or number of the blood vessels in angiogenic disorders.

A “disorder” is any condition that would benefit from treatment including, but not limited to, chronic and acute disorders or diseases including those pathological conditions which predispose the mammal to the disorder in question. Disorders include angiogenic disorders. “Angiogenic disorder” as used herein refers to any condition involving abnormal angiogenesis or abnormal vascular permeability or leakage. Non-limiting examples of angiogenic disorders to be treated herein include malignant and benign tumors; non-leukemias and lymphoid malignancies; and, in particular, tumor (cancer) metastasis.

“Abnormal angiogenesis” occurs when new blood vessels grow either excessively or otherwise inappropriately (e.g., the location, timing, degree, or onset of the angiogenesis being undesired from a medical standpoint) in a diseased state or such that it causes a diseased state. In some cases, excessive, uncontrolled, or otherwise inappropriate angiogenesis occurs when there is new blood vessel growth that contributes to the worsening of the diseased state or cause of a diseased state. The new blood vessels can feed the diseased tissues, destroy normal tissues, and in the case of cancer, the new vessels can allow tumor cells to escape into the circulation and lodge in other organs (tumor metastases). Examples of disorders involving abnormal angiogenesis include, but are not limited to cancer, especially vascularized solid tumors and metastatic tumors (including colon, lung cancer (especially small-cell lung cancer), or prostate cancer), diseases caused by ocular neovascularisation, especially diabetic blindness, retinopathies, primarily diabetic retinopathy or age-related macular degeneration, choroidal neovascularization (CNV), diabetic macular edema, pathological myopia, von Hippel-Lindau disease, histoplasmosis of the eye, Central Retinal Vein Occlusion (CRVO), corneal neovascularization, retinal neovascularization and rubeosis; psoriasis, psoriatic arthritis, haemangioblastoma such as haemangioma; inflammatory renal diseases, such as glomerulonephritis, especially mesangioproliferative glomerulonephritis, haemolytic uremic syndrome, diabetic nephropathy or hypertensive nephrosclerosis; various imflammatory diseases, such as arthritis, especially rheumatoid arthritis, inflammatory bowel disease, psorsasis, sarcoidosis, arterial arteriosclerosis and diseases occurring after transplants, endometriosis or chronic asthma and other conditions.

“Abnormal vascular permeability” occurs when the flow of fluids, molecules (e.g., ions and nutrients) and cells (e.g., lymphocytes) between the vascular and extravascular compartments is excessive or otherwise inappropriate (e.g., the location, timing, degree, or onset of the vascular permeability being undesired from a medical standpoint) in a diseased state or such that it causes a diseased state. Abnormal vascular permeability may lead to excessive or otherwise inappropriate “leakage” of ions, water, nutrients, or cells through the vasculature. In some cases, excessive, uncontrolled, or otherwise inappropriate vascular permeability or vascular leakage exacerbates or induces disease states including, e.g., edema associated with tumors including, e.g., brain tumors; ascites associated with malignancies; Meigs' syndrome; lung inflammation; nephrotic syndrome; pericardial effusion; pleural effusion; permeability associated with cardiovascular diseases such as the condition following myocardial infarctions and strokes and the like. The present invention contemplates treating those patients that have developed or are at risk of developing the diseases and disorders associated with abnormal vascular permeability or leakage.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, and multiple myeloma. In some embodiments, the cancer is selected from: small cell lung cancer, gliblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenic agents, apoptotic agents, anti-tubulin agents, and other agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, B1yS, APRIL, BCMA or VEGF receptor(s), TRAIL/Apo2, and other bioactive and organic chemical agents, etc. Combinations thereof are also included in the invention.

An “angiogenic factor or agent” is a growth factor or its receptor which is involved in stimulating the development of blood vessels, e.g., promote angiogenesis, endothelial cell growth, stability of blood vessels, and/or vasculogenesis, etc. For example, angiogenic factors, include, but are not limited to, e.g., VEGF and members of the VEGF family and their receptors (VEGF-B, VEGF-C, VEGF-D, VEGFR1, VEGFR2 and VEGFR3), PlGF, PDGF family, fibroblast growth factor family (FGFs), TIE ligands (Angiopoietins, ANGPT1, ANGPT2), TIE1, TIE2, ephrins, Bv8, Delta-like ligand 4 (DLL4), Del-1, fibroblast growth factors: acidic (aFGF) and basic (bFGF), FGF4, FGF9, BMP9, BMP10, Follistatin, Granulocyte colony-stimulating factor (G-CSF), GM-CSF, Hepatocyte growth factor (HGF)/scatter factor (SF), Interleukin-8 (IL-8), CXCL12, Leptin, Midkine, neuropilins, NRP1, NRP2, Placental growth factor, Platelet-derived endothelial cell growth factor (PD-ECGF), Platelet-derived growth factor, especially PDGF-BB, PDGFR-alpha, or PDGFR-beta, Pleiotrophin (PTN), Progranulin, Proliferin, Transforming growth factor-alpha (TGF-alpha), Transforming growth factor-beta (TGF-beta), Tumor necrosis factor-alpha (TNF-alpha), Alk1, CXCR4, Notch1, Notch4, Sema3A, Sema3C, Sema3F, Robo4, etc. It would further include factors that promote angiogenesis, such as ESM1 and Perlecan. It would also include factors that accelerate wound healing, such as growth hormone, insulin-like growth factor-I (IGF-I), VIGF, epidermal growth factor (EGF), EGF-like domain, multiple 7 (EGFL7), CTGF and members of its family, and TGF-alpha and TGF-beta. See, e.g., Klagsbrun and D'Amore (1991) Annu. Rev. Physiol. 53:217-39; Streit and Detmar (2003) Oncogene 22:3172-3179; Ferrara & Alitalo (1999) Nature Medicine 5(12):1359-1364; Tonini et al. (2003) Oncogene 22:6549-6556 (e.g., Table 1 listing known angiogenic factors); and, Sato (2003) Int. J. Clin. Oncol. 8:200-206.

An “anti-angiogenic agent” or “angiogenic inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenic agent includes those agents that bind and block the angiogenic activity of the angiogenic factor or its receptor. For example, an anti-angiogenic agent is an antibody or other antagonist to an angiogenic agent as defined above, e.g., antibodies to VEGF-A or to the VEGF-A receptor (e.g., KDR receptor or Flt-1 receptor), anti-PDGFR inhibitors, small molecules that block VEGF receptor signaling (e.g., PTK787/ZK2284, SU6668, SUTENT®/SU11248 (sunitinib malate), AMG706, or those described in, e.g., international patent application WO 2004/113304). Anti-angiogenic agents include, but are not limited to, the following agents: VEGF inhibitors such as a VEGF-specific antagonist, EGF inhibitor, EGFR inhibitors, Erbitux® (cetuximab, ImClone Systems, Inc., Branchburg, N.J.), Vectibix® (panitumumab, Amgen, Thousand Oaks, Calif.), TIE2 inhibitors, IGF1R inhibitors, COX-II (cyclooxygenase II) inhibitors, MMP-2 (matrix-metalloproteinase 2) inhibitors, and MMP-9 (matrix-metalloproteinase 9) inhibitors, CP-547,632 (Pfizer Inc., NY, USA), Axitinib (Pfizer Inc.; AG-013736), ZD-6474 (AstraZeneca), AEE788 (Novartis), AZD-2171), VEGF Trap (Regeneron/Aventis), Vatalanib (also known as PTK-787, ZK-222584: Novartis & Schering A G), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. Other angiogenesis inhibitors include thrombospondin1, thrombospondin2, collagen IV and collagen XVIII. VEGF inhibitors are disclosed in U.S. Pat. Nos. 6,534,524 and 6,235,764, both of which are incorporated in their entirety for all purposes. Anti-angiogenic agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known antiangiogenic factors); and, Sato (2003) Int. J. Clin. Oncol. 8:200-206 (e.g., Table 1 listing anti-angiogenic agents used in clinical trials).

The term “anti-angiogenic therapy” refers to a therapy useful for inhibiting angiogenesis which comprises the administration of an anti-angiogenic agent.

The term “cytotoxic agent” as used herein refers to a substance that inhibits or prevents a cellular function and/or causes cell death or destruction. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu), chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “toxin” is any substance capable of having a detrimental effect on the growth or proliferation of a cell.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhôme-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bc1-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above; as well as combinations of two or more of the above.

A “growth inhibitory agent” when used herein refers to a compound or composition which inhibits growth of a cell either in vitro or in vivo. In one embodiment, growth inhibitory agent is growth inhibitory antibody that prevents or reduces proliferation of a cell expressing an antigen to which the antibody binds. In another embodiment, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

The term “pharmaceutical formulation” refers to a preparation which is in such form as to permit the biological activity of the active ingredient to be effective, and which contains no additional components which are unacceptably toxic to a subject to which the formulation would be administered. Such formulations may be sterile.

A “sterile” formulation is aseptic or free from all living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “liposome” is a small vesicle composed of various types of lipids, phospholipids and/or surfactant which is useful for delivery of a drug (such as a anti-VEGF antibody or anti-NRP1 antibody) to a mammal. The components of the liposome are commonly arranged in a bilayer formation, similar to the lipid arrangement of biological membranes.

The term “diagnosis” is used herein to refer to the identification of a molecular or pathological state, disease or condition, such as the identification of cancer or to refer to identification of a cancer patient who may benefit from a particular treatment regimen.

The term “prognosis” is used herein to refer to the prediction of the likelihood of benefit from anti-cancer therapy.

The term “prediction” or “predicting” is used herein to refer to the likelihood that a patient will respond either favorably or unfavorably to a particular anti-cancer therapy. In one embodiment, prediction or predicting relates to the extent of those responses. In one embodiment, the prediction or predicting relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc., or whether long-term survival of the patient, following a therapeutic regimen is likely.

Responsiveness of a patient can be assessed using any endpoint indicating a benefit to the patient, including, without limitation, (1) inhibition, to some extent, of disease progression, including slowing down and complete arrest; (2) reduction in lesion size; (3) inhibition (i.e., reduction, slowing down or complete stopping) of disease cell infiltration into adjacent peripheral organs and/or tissues; (4) inhibition (i.e. reduction, slowing down or complete stopping) of disease spread; (5) relief, to some extent, of one or more symptoms associated with the disorder; (6) increase in the length of disease-free presentation following treatment; and/or (8) decreased mortality at a given point of time following treatment.

The term “benefit” is used in the broadest sense and refers to any desirable effect and specifically includes clinical benefit as defined herein.

Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

The term “resistant cancer or “resistant tumor” refers to cancer, cancerous cells, or a tumor that does not respond completely, or loses or shows a reduced response over the course of cancer therapy to a cancer therapy comprising at least a VEGF antagonist. In certain embodiments, resistant tumor is a tumor that is resistant to anti-VEGF antibody therapy. In one embodiment, the anti-VEGF antibody is bevacizumab. In certain embodiments, a resistant tumor is a tumor that is unlikely to respond to a cancer therapy comprising at least a VEGF antagonist.

“Relapsed” refers to the regression of the patient's illness back to its former diseased state, especially the return of symptoms following an apparent recovery or partial recovery. Unless otherwise indicted, relapsed state refers to the process of returning to or the return to illness before the previous treatment including, but not limited to, VEGF antagonist and chemotherapy treatments. In certain embodiments, VEGF antagonist is an anti-VEGF antibody.

### III. Methods of the Invention

The present invention is based partly on the use of specific genes or biomarkers that correlate with efficacy of anti-angiogenic therapy or treatment other than or in addition to a VEGF antagonist. Suitable therapy or treatment other than or in addition to a VEGF antagonist include, but are not limited to a NRP1 antagonist, an EGFL7 antagonist, or a VEGF-C antagonist. Thus, the disclosed methods provide convenient, efficient, and potentially cost-effective means to obtain data and information useful in assessing appropriate or effective therapies for treating patients. For example, a cancer patient could have a biopsy performed to obtain a tissue or cell sample, and the sample could be examined by various in vitro assays to determine whether the expression level of one or more biomarkers has increased or decreased as compared to the expression level in a reference sample. If expression levels of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94 of the genes listed in Table 1 is increased or decreased, then the patient is likely to benefit from treatment with a therapy or treatment other than or in addition to a VEGF antagonist.

Expression levels/amount of a gene or a biomarker can be determined based on any suitable criterion known in the art, including but not limited to mRNA, cDNA, proteins, protein fragments and/or gene copy number.

Expression of various genes or biomarkers in a sample can be analyzed by a number of methodologies, many of which are known in the art and understood by the skilled artisan, including but not limited to, immunohistochemical and/or Western blot analysis, immunoprecipitation, molecular binding assays, ELISA, ELIFA, fluorescence activated cell sorting (FACS) and the like, quantitative blood based assays (as for example Serum ELISA) (to examine, for example, levels of protein expression), biochemical enzymatic activity assays, in situ hybridization, Northern analysis and/or PCR analysis of mRNAs, as well as any one of the wide variety of assays that can be performed by gene and/or tissue array analysis. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (MSD) may also be used.

In certain embodiments, expression/amount of a gene or biomarker in a sample is increased as compared to expression/amount in a reference sample if the expression level/amount of the gene or biomarker in the sample is greater than the expression level/amount of the gene or biomarker in reference sample. Similarly, expression/amount of a gene or biomarker in a sample is decreased as compared to expression/amount in a reference sample if the expression level/amount of the gene or biomarker in the ample is less than the expression level/amount of the gene or biomarker in the reference sample.

In certain embodiments, the samples are normalized for both differences in the amount of RNA or protein assayed and variability in the quality of the RNA or protein samples used, and variability between assay runs. Such normalization may be accomplished by measuring and incorporating the expression of certain normalizing genes, including well known housekeeping genes, such as ACTB. Alternatively, normalization can be based on the mean or median signal of all of the assayed genes or a large subset thereof (global normalization approach). On a gene-by-gene basis, measured normalized amount of a patient tumor mRNA or protein is compared to the amount found in a reference set. Normalized expression levels for each mRNA or protein per tested tumor per patient can be expressed as a percentage of the expression level measured in the reference set. The expression level measured in a particular patient sample to be analyzed will fall at some percentile within this range, which can be determined by methods well known in the art.

In certain embodiments, relative expression level of a gene is determined as follows:

Relative expression gene1ₛₐₘₚₗₑ₁=2exp(Ct_{housekeeping gene}−Ct_{gene1}) with Ct determined in a sample.

Relative expression gene1_{reference RNA}=2exp(Ct_{housekeeping gene}−Ct_{gene1}) with Ct determined in the reference sample.

Normalized relative expression gene1ₛₐₘₚₗₑ₁=(relative expression gene1ₛₐₘₚₗₑ₁/relative expression gene1_{reference RNA})×100

Ct is the threshold cycle. The Ct is the cycle number at which the fluorescence generated within a reaction crosses the threshold line.

All experiments are normalized to a reference RNA, which is a comprehensive mix of RNA from various tissue sources (e.g., reference RNA #636538 from Clontech, Mountain View, Calif.). Identical reference RNA is included in each qRT-PCR run, allowing comparison of results between different experimental runs.

A sample comprising a target gene or biomarker can be obtained by methods well known in the art, and that are appropriate for the particular type and location of the cancer of interest. See under Definitions. For instance, samples of cancerous lesions may be obtained by resection, bronchoscopy, fine needle aspiration, bronchial brushings, or from sputum, pleural fluid or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

Means for enriching a tissue preparation for cancer cells are known in the art. For example, the tissue may be isolated from paraffin or cryostat sections. Cancer cells may also be separated from normal cells by flow cytometry or laser capture microdissection. These, as well as other techniques for separating cancerous from normal cells, are well known in the art. If the cancer tissue is highly contaminated with normal cells, detection of signature gene or protein expression profile may be more difficult, although techniques for minimizing contamination and/or false positive/negative results are known, some of which are described herein below. For example, a sample may also be assessed for the presence of a biomarker known to be associated with a cancer cell of interest but not a corresponding normal cell, or vice versa.

In certain embodiments, the expression of proteins in a sample is examined using immunohistochemistry (“IHC”) and staining protocols. Immunohistochemical staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods.

The tissue sample may be fixed (i.e. preserved) by conventional methodology (See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the sample is to be histologically stained or otherwise analyzed. One of skill in the art will also appreciate that the length of fixation depends upon the size of the tissue sample and the fixative used. By way of example, neutral buffered formalin, Bouin's or paraformaldehyde, may be used to fix a sample.

Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., “Manual of Histological Staining Method of the Armed Forces Institute of Pathology”, supra). Examples of paraffin that may be used include, but are not limited to, Paraplast, Broloid, and Tissuemay. Once the tissue sample is embedded, the sample may be sectioned by a microtome or the like (See e.g., “Manual of Histological Staining Method of the Armed Forces Institute of Pathology”, supra). By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., “Manual of Histological Staining Method of the Armed Forces Institute of Pathology”, supra). Alternatively, commercially available deparaffinizing non-organic agents such as Hemo-De7 (CMS, Houston, Tex.) may be used.

In certain embodiments, subsequent to the sample preparation, a tissue section may be analyzed using IHC. IHC may be performed in combination with additional techniques such as morphological staining and/or fluorescence in-situ hybridization. Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Colloidal gold particles.

(c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. Fluorescence can be quantified using a fluorimeter.

(d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and

(iii) 13-D-galactosidase (β-D-Gal) with a chromogenic substrate (e.g., p-nitrophenyl-13-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980. Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired. For example, epitope retrieval methods, such as heating the tissue sample in citrate buffer may be carried out (see, e.g., Leong et al. Appl. Immunohistochem. 4(3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. In certain embodiments, the label is an enzymatic label (e.g. HRPO) which catalyzes a chemical alteration of the chromogenic substrate such as 3,3′-diaminobenzidine chromogen. In one embodiment, the enzymatic label is conjugated to antibody which binds specifically to the primary antibody (e.g. the primary antibody is rabbit polyclonal antibody and secondary antibody is goat anti-rabbit antibody).

Specimens thus prepared may be mounted and coverslipped. Slide evaluation is then determined, e.g., using a microscope, and staining intensity criteria, routinely used in the art, may be employed. Staining intensity criteria may be evaluated as follows:

| Staining Pattern | Score |
|---|---|
| No staining is observed in cells. | 0 |
| Faint/barely perceptible staining is detected in more | 1+ |
| than 10% of the cells. | |
| Weak to moderate staining is observed in more than | 2+ |
| 10% of the cells. | |
| Moderate to strong staining is observed in more than | 3+ |
| 10% of the cells. | |

In some embodiments, a staining pattern score of about 1+ or higher is diagnostic and/or prognostic. In certain embodiments, a staining pattern score of about 2+ or higher in an IHC assay is diagnostic and/or prognostic. In other embodiments, a staining pattern score of about 3 or higher is diagnostic and/or prognostic. In one embodiment, it is understood that when cells and/or tissue from a tumor or colon adenoma are examined using IHC, staining is generally determined or assessed in tumor cell and/or tissue (as opposed to stromal or surrounding tissue that may be present in the sample).

In alternative methods, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labelled antibody to a target biomarker.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labelled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, -galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

It is contemplated that the above described techniques may also be employed to detect expression of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94 of the target genes wherein the target genes are the genes set forth in Table 1.

Methods of the invention further include protocols which examine the presence and/or expression of mRNAs of the at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94 of the target genes set forth in Table 1, in a tissue or cell sample. Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like).

Tissue or cell samples from mammals can be conveniently assayed for mRNAs using Northern, dot blot or PCR analysis. For example, RT-PCR assays such as quantitative PCR assays are well known in the art. In an illustrative embodiment of the invention, a method for detecting a target mRNA in a biological sample comprises producing cDNA from the sample by reverse transcription using at least one primer; amplifying the cDNA so produced using a target polynucleotide as sense and antisense primers to amplify target cDNAs therein; and detecting the presence of the amplified target cDNA using polynucleotide probes. In some embodiments, primers and probes comprising the sequences set forth in Table 2 are used to detect expression of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94 of the target genes set forth in Table 1. In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined.

Optional methods of the invention include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlate with increased or reduced clinical benefit of anti-angiogenic therapy may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment. (see, e.g., WO 01/75166 published Oct. 11, 2001; (see, for example, U.S. Pat. No. 5,700,637, U.S. Pat. No. 5,445,934, and U.S. Pat. No. 5,807,522, Lockart, Nature Biotechnology, 14:1675-1680 (1996); Cheung, V. G. et al., Nature Genetics 21(Suppl):15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ).

The Affymetrix GeneChip® system is a commercially available microarray system which comprises arrays fabricated by direct synthesis of oligonucleotides on a glass surface. Probe/Gene Arrays: Oligonucleotides, usually 25 mers, are directly synthesized onto a glass wafer by a combination of semiconductor-based photolithography and solid phase chemical synthesis technologies. Each array contains up to 400,000 different oligos and each oligo is present in millions of copies. Since oligonucleotide probes are synthesized in known locations on the array, the hybridization patterns and signal intensities can be interpreted in terms of gene identity and relative expression levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligo. The Microarray Suite software subtracts the hybridization intensities of the mismatch probes from those of the perfect match probes to determine the absolute or specific intensity value for each probe set. Probes are chosen based on current information from Genbank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

Expression of a selected gene or biomarker in a tissue or cell sample may also be examined by way of functional or activity-based assays. For instance, if the biomarker is an enzyme, one may conduct assays known in the art to determine or detect the presence of the given enzymatic activity in the tissue or cell sample.

The kits of the invention have a number of embodiments. In certain embodiments, a kit comprises a container, a label on said container, and a composition contained within said container; wherein the composition includes one or more primary antibodies that bind to one or more target polypeptide sequences corresponding to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94 genes set forth in Table 1, the label on the container indicating that the composition can be used to evaluate the presence of one or more target proteins in at least one type of mammalian cell, and instructions for using the antibodies for evaluating the presence of one or more target proteins in at least one type of mammalian cell. The kit can further comprise a set of instructions and materials for preparing a tissue sample and applying antibody and probe to the same section of a tissue sample. The kit may include both a primary and secondary antibody, wherein the secondary antibody is conjugated to a label, e.g., an enzymatic label.

Another embodiment is a kit comprising a container, a label on said container, and a composition contained within said container; wherein the composition includes one or more polynucleotides that hybridize to the polynucleotide sequence of the at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94 genes set forth in Table 1, under stringent conditions, the label on said container indicates that the composition can be used to evaluate the presence of and/or expression levels of the one or more target genes in at least one type of mammalian cell, and instructions for using the polynucleotide for evaluating the presence of and/or expression levels of one or more target RNAs or DNAs in at least one type of mammalian cell. In some embodiments, the kits comprise polynucleotide primers and probes comprising the sequences set forth in Table 2

Other optional components in the kit include one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

### IV. Pharmaceutical Formulations

For the methods of the invention, therapeutic formulations of the anti-NRP1, anti-EGFL7antibody, anti-VEGF-C antibody, or anti-VEGF antibody are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### V. Therapeutic Uses

The present invention contemplates a method for treating an angiogenic disorder (e.g., a disorder characterized by abnormal angiogenesis or abnormal vascular leakage) in a patient comprising the steps of determining that a sample obtained from the patient has increased or decreased expression levels of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94 genes set forth in Table 1, and administering to the patient an effective amount of an anti-cancer therapy whereby the tumor, cancer or cell proliferative disorder is treated. The anticancer therapy may be, e.g., a NRP1 antagonist, an EGFL7 antagonist, or a VEGF-C antagonist.

Examples of angiogenic disorders to be treated herein include, but are not limited to cancer, especially vascularized solid tumors and metastatic tumors (including colon, lung cancer (especially small-cell lung cancer), or prostate cancer), diseases caused by ocular neovascularisation, especially diabetic blindness, retinopathies, primarily diabetic retinopathy or age-related macular degeneration, choroidal neovascularization (CNV), diabetic macular edema, pathological myopia, von Hippel-Lindau disease, histoplasmosis of the eye, Central Retinal Vein Occlusion (CRVO), corneal neovascularization, retinal neovascularization and rubeosis; psoriasis, psoriatic arthritis, haemangioblastoma such as haemangioma; inflammatory renal diseases, such as glomerulonephritis, especially mesangioproliferative glomerulonephritis, haemolytic uremic syndrome, diabetic nephropathy or hypertensive nephrosclerosis; various inflammatory diseases, such as arthritis, especially rheumatoid arthritis, inflammatory bowel disease, psoriasis, sarcoidosis, arterial arteriosclerosis and diseases occurring after transplants, endometriosis or chronic asthma and other conditions; disease states including, e.g., edema associated with tumors including, e.g., brain tumors; ascites associated with malignancies; Meigs' syndrome; lung inflammation; nephrotic syndrome; pericardial effusion; pleural effusion; permeability associated with cardiovascular diseases such as the condition following myocardial infarctions and strokes and the like.

Examples of cancer to be treated herein include, but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. More particularly, cancers that are amenable to treatment by the antibodies of the invention include breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. In some embodiments, the cancer may be a resistant cancer. In some embodiments, the cancer may be a relapsed cancer.

It is contemplated that when used to treat various diseases such as tumors, the NRP1 antagonist, EGFL7 antagonist, or VEGF-C antagonist can be combined with one or more other therapeutic agents suitable for the same or similar diseases. For example, when used for treating cancer, the NRP1 antagonist, EGFL7 antagonist, or VEGF-C antagonist may be used in combination with conventional anti-cancer therapies, such as surgery, radiotherapy, chemotherapy or combinations thereof.

In certain aspects, other therapeutic agents useful for combination cancer therapy with the NRP1 antagonist, EGFL7 antagonist, or VEGF-C antagonist include other anti-angiogenic agents. Many anti-angiogenic agents have been identified and are known in the arts, including those listed by Carmeliet and Jain (2000) Nature 407(6801):249-57.

In one aspect, the NRP1 antagonist, EGFL7 antagonist, or VEGF-C antagonist is used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof. Alternatively, or in addition, two or more NRP1 antagonists, EGFL7 antagonists, or VEGF-C antagonists may be co-administered to the patient. In a preferred embodiment, an anti-NRP1 antibody is used in combination with an anti-VEGF antibody to generate additive or synergistic effects. In another preferred embodiment, an anti-EGFL7 antibody is used in combination with an anti-VEGF antibody to generate additive or synergistic effects. In a further preferred embodiment, an anti-VEGF-C antibody is used in combination with an anti-VEGF antibody to generate additive or synergistic effects. Preferred anti-VEGF antibodies include those that bind to the same epitope as the anti-hVEGF antibody A4.6.1. More preferably the anti-VEGF antibody is bevacizumab or ranibizumab.

In some other aspects of the methods of the invention, other therapeutic agents useful for combination tumor therapy with the NRP1 antagonist, EGFL7 antagonist, or VEGF-C antagonist, include antagonists of other factors that are involved in tumor growth, such as EGFR, ErbB2 (also known as Her2) ErbB3, ErbB4, or TNF. Preferably, the anti-NRP1 antibody, anti-EGFL7 antibody, or VEGF-C antibody of the invention can be used in combination with small molecule receptor tyrosine kinase inhibitors (RTKIs) that target one or more tyrosine kinase receptors such as VEGF receptors, FGF receptors, EGF receptors and PDGF receptors. Many therapeutic small molecule RTKIs are known in the art, including, but are not limited to, vatalanib (PTK787), erlotinib (TARCEVA®), OSI-7904, ZD6474 (ZACTIMA®), ZD6126 (ANG453), ZD1839, sunitinib (SUTENT®), semaxanib (SU5416), AMG706, AG013736, Imatinib (GLEEVEC®), MLN-518, CEP-701, PKC-412, Lapatinib (GSK572016), VELCADE®, AZD2171, sorafenib (NEXAVAR®), XL880, and CHIR-265.

The methods of the invention can also include use of the NRP1 antagonist, EGFL7 antagonist, or VEGF-C antagonist, either alone or in combination with a second therapeutic agent (such as an anti-VEGF antibody) and further in combination with one or more chemotherapeutic agents. A variety of chemotherapeutic agents may be used in the combined treatment methods of the invention. An exemplary and non-limiting list of chemotherapeutic agents contemplated is provided herein above.

For the methods of the invention, when the NRP1 antagonist, EGFL7 antagonist, or VEGF-C antagonist is co-administered with a second therapeutic agent, the second therapeutic agent may be administered first, followed by the NRP1 antagonist, EGFL7 antagonist, or VEGF-C antagonist. However, simultaneous administration or administration of the NRP1 antagonist, EGFL7 antagonist, or VEGF-C antagonist first is also contemplated. Suitable dosages for the second therapeutic agent are those presently used and may be lowered due to the combined action (synergy) of the agent and NRP1 antagonist, EGFL7 antagonist, or VEGF-C antagonist.

Where the method of the invention contemplates administration of an antibody to a patient, depending on the type and severity of the disease, about 1 μg/kg to 50 mg/kg (e.g. 0.1-20 mg/kg) of antibody is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. In a preferred aspect, the antibody is administered every two to three weeks, at a dose ranged from about 5 mg/kg to about 15 mg/kg. In one aspect the antibody is administered every two to three weeks at a dose of about 5 mg/kg, 7.5 mg/kg, 10 mg/kg or 15 mg/kg. Such dosing regimen may be used in combination with a chemotherapy regimen. In some aspects, the chemotherapy regimen involves the traditional high-dose intermittent administration. In some other aspects, the chemotherapeutic agents are administered using smaller and more frequent doses without scheduled breaks (“metronomic chemotherapy”). The progress of the therapy of the invention is easily monitored by conventional techniques and assays.

The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat a disease or disorder. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as used hereinbefore or about from 1 to 99% of the heretofore employed dosages. Generally, alleviation or treatment of a disease or disorder involves the lessening of one or more symptoms or medical problems associated with the disease or disorder. In the case of cancer, the therapeutically effective amount of the drug can accomplish one or a combination of the following: reduce the number of cancer cells; reduce the tumor size; inhibit (i.e., to decrease to some extent and/or stop) cancer cell infiltration into peripheral organs; inhibit tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. In some embodiments, a composition of this invention can be used to prevent the onset or reoccurrence of the disease or disorder in a subject or mammal.

Although in the foregoing description the invention is illustrated with reference to certain embodiments, it is not so limited. Indeed, various modifications of the invention in addition to those shown and described herein will be apparent to those skilled in the art from the foregoing description and fall within the scope of the appended claims. All references cited throughout the specification, and the references cited therein, are hereby expressly incorporated by reference in their entirety for all purposes.

### EXAMPLES

### Example 1

### Identification of Agents with Tumor Inhibitory Activities

All studies are conducted in accordance with the Guide for the Care and Use of Laboratory Animals, published by the NIH (NIH Publication 85-23, revised 1985). An Institutional Animal Care and Use Committee (IACUC) approved all animal protocols.

Studies are conducted with a suitable tumor models including, for example, breast cancer models such as, e.g., MDA-MB231, MX1, BT474, MCF7, KPL-4, 66c14, Fo5, and MAXF583; colon cancer models such as, e.g., LS174t, DLD-1, HT29, SW620, SW480, HCT116, colo205, HM7, LoVo, LS180, CXF243, and CXF260; lung cancer models such as, e.g., A549, H460, SKMES, H1299, MV522, Calu-6, Lewis Lung carcinoma, H520, NCI-H2122, LXFE409, LXFL1674, LXFA629, LXFA737, LXFA1335, and 1050489; ovarian cancer models such as, e.g., OVCAR3, A2780, SKOV3, and IGROV-1; pancreatic cancer models such as, e.g., BxPC3, PANC1, MiaPaCa-2, KP4, and SU8686; prostate cancer models such as, e.g., PC3, DU145; brain cancer models such as, e.g., U87MG (glioblastoma), SF295 (glioblastoma), and SKNAS (neuroblastoma); liver cancer models such as, e.g., Hep3B, Huh-7, and JHH-7; melanoma models such as, e.g., A2058, A375, SKMEL-5, A2058, and MEXF989; renal cancer models such as, e.g., Caki-1, Caki-2, and 786-0; Ewing's sarcoma and bone cancer such as, e.g., MHH-ES-1; gastric cancer models such as, e.g., SNU5; rhabdomyosarcoma models such as, e.g., A673 and SXF463; myeloma models such as, e.g., OPM2-FcRH5; and B cell lymphoma such as, e.g., WSU-DLCL2; and urinary cancer bladder models such as, e.g., BXF1218 and BXF1352 using standardized techniques. Briefly, human tumor cells are implanted subcutaneously in the right flank of each test mouse. On the day of tumor implant, tumor cells are harvested and resuspended in PBS at a concentration of 5×10⁷ cells/mL. Each test mouse receives 1×10⁷ tumor cells implanted subcutaneously in the right flank, and tumor growth is monitored.

Tumor growth is monitored as the average size approached 120-180 mm³. On study day 1, the mice are sorted by tumor size into three test groups (one control group and two treatment groups). Tumor volume is calculated using the formula:

Tumor volume (mm³)==(w²×l)/2

where w=width and l=length in mm of the tumor.

All treatments are administered intra-peritoneally. Mice are treated twice weekly for up to 10-20 weeks with 5-10 mg/kg each of control antibody, an agent blocking VEGF activity, or the combination of an agent blocking VEGF activity and an test agent. For the combination treatment group, the anti-angiogenic agent is administered concurrently with the anti-VEGF antibody or sequentially with the anti-VEGF antibody. If the test agent and the anti-VEGF antibody are administered sequentially, the test agent is administered no earlier than 30 minutes prior to administration of the anti-VEGF antibody or no later than thirty minutes after administration of the anti-VEGF antibody. Each dose is delivered in a volume of 0.2 mL per 20 grams body weight (10 mL/kg), and is scaled to the body weight of the animal.

Tumor volume is recorded twice weekly using calipers. Each animal was euthanized when its tumor reached the endpoint size (generally 1000 mm³) or at the conclusion of the study, whichever occurs first. Tumor are harvested and either fixated overnight in 10% NBF, followed by 70% ethanol and subsequent embedding in paraffin, or within two minutes frozen in liquid nitrogen for subsequent storage at −80° C.

The time to endpoint (TTE) is calculated from the following equation:

TTE (days)=(log₁₀(endpoint volume,mm³−b)/m

where b is the intercept and m is the slope of the line obtained by linear regression of a log-transformed tumor growth data set.

Animals that reach the endpoint are assigned a TTE value equal to the last day of the study. Animals classified as NTR (non-treatment-related) deaths due to accident (NTRa) or due unknown causes (NTRu) are excluded from TTE calculations (and all further analyses). Animals classified as TR (treatment-related) deaths or NTRm (non-treatment-related death due to metastasis) are assigned a TTE value equal to the day of death.

Treatment outcome is evaluated by tumor growth delay (TGD), which is defined as the increase in the median time to endpoint (TTE) in a treatment group compared to the control group, which is calculated as follows:

TGD=T−C, expressed in days, or as a percentage of the median TTE of the control group, which is calculated as follows:

% TGD=[(T−C)/C]×100,

where T=median TTE for a treatment group and C=median TTE for the control group.

The Δ% TGD is calculated as above, with C=control group being the group receiving anti-VEGF-A treatment alone, and T=treatment group being the group receiving the combination of anti-VEGF and a test agent. The logrank test is employed to analyze the significance of the difference between the TTE values of two groups. Two-tailed statistical analyses are conducted at significance level p=0.05. A value of “1” indicates that treatment resulted in an additional delay in tumor progression. A value of “0” indicates that the treatment did not result in an additional delay in tumor progression.

### Example 2

### Identification of Biomarkers for Efficacy of Treatment

Gene expression analysis of at least one gene set forth in Table 1 below is performed using qRT-PCR on tumor samples obtained from the tumor model experiments described above in Example 1.

TABLE 1
Gene

18S rRNA
ACTB
RPS13
VEGFA
VEGFC
VEGFD
Bv8
PlGF
VEGFR1/Flt1
VEGFR2
VEGFR3
NRP1 (transmembrane and soluble)
Podoplanin
Prox1
VE-Cadherin (CD144, CDH5)
FGF2
IL8/CXCL8
HGF
THBS1/TSP1
Egfl7
NG3/Egfl8
ANG1
GM-CSF/CSF2
G-CSF/CSF3
FGF9
CXCL12/SDF1
TGFb1
TNFa
Alk1
BMP9
BMP10
HSPG2/perlecan
ESM1
Sema3a
Sema3b
Sema3c
Sema3e
Sema3f
NG2
ICAM1
CXCR4
TMEM100
PECAM/CD31
PDGFb
PDGFRb
RGS5
CXCL1
CXCL2
Robo4
LyPD6
VCAM1
collagen IV (a1, a2, or a3)
Spred-1
Hhex
ITGa5
LGALS1/Galectin1
LGALS7/Galectin7
MFAP5
Fibronectin
fibulin2
fibulin4/Efemp2
HMBS
SDHA
UBC
NRP2
CD34
DLL4
CLECSF5/CLEC5a
CCL2/MCP1
CCL5
CXCL5/ENA-78
ANG2
FGF8
FGF8b
PDGFC
cMet
JAG1
CD105/Endoglin
Notch1
EphB4
EphA3
EFNB2
TIE2/TEK
LAMA4
NID2
Map4k4
Bcl2A1
IGFBP4
VIM/vimentin
FGFR4
FRAS1
ANTXR2
CLECSF5/CLEC5a
Mincle/CLEC4E/CLECSF9
PTGS2
PDGFA

From frozen material, small cubes of maximal 3 mm side length are solubilized using commercially available reagents and equipment (RNeasy®, Tissuelyzer, both Qiagen Inc, Germany). After column purification RNA is eluted with H₂O, precipitated with ethanol after the addition of glycogen and Sodium acetate. RNA is pelleted by centrifugation for at least 30 min, washed twice with 80% ethanol, and the pellet resuspended in H₂0 after drying. RNA concentrations are assessed using a spectrophotometer or a bioanalyzer (Agilent, Foster City, Calif.), and 50 ng of total RNA is used per reaction in the subsequent gene expression analysis. Gene specific primer and probe sets were designed for qRT-PCR expression analysis. The primer and probe set sequences are set forth in Table 2 below.

TABLE 2

SEQ ID
NO:

human 18S rRNA

| | | |
|---|---|---|
| Forward primer | AGT CCC TGC CCT TTG TAC ACA | 1 |
| Reverse Primer | CCG AGG GCC TCA CTA AAC C | 2 |
| Probe | CGC CCG TCG CTA CTA CCG ATT GG | 3 |

human ACTB

| | | |
|---|---|---|
| Forward primer | GAAGGCTTTTGGTCTCCCTG | 4 |
| Reverse Primer | GGTGTGCACTTTTATTCAACTGG | 5 |
| Probe | AGGGCTTACCTGTACACTG | 6 |

murine ACTB

| | | |
|---|---|---|
| Forward primer | CCA TGA AAT AAG TGG TTA CAG GAA GTC | 7 |
| Reverse Primer | CAT GGA CGC GAC CAT CCT | 8 |
| Probe | TCC CAA AAG CCA CCC CCA CTC CTA AG | 9 |

human RPS13

| | | |
|---|---|---|
| Forward primer | CACCGTTTGGCTCGATATTA | 10 |
| Reverse Primer | GGCAGAGGCTGTAGATGATTC | 11 |
| Probe | ACCAAGCGAGTCCTCCCTCCC | 12 |

murine RPS13

| | | |
|---|---|---|
| Forward primer | CACCGATTGGCTCGATACTA | 13 |
| Reverse Primer | TAGAGCAGAGGCTGTGGATG | 14 |
| Probe | CGGGTGCTCCCACCTAATTGGA | 15 |

human VEGF-A

| | | |
|---|---|---|
| Forward primer | ATC ACC ATG CAG ATT ATG CG | 16 |
| Reverse Primer | TGC ATT CAC ATT TGT TGT GC | 17 |
| Probe | TCA AAC CTC ACC AAG GCC AGC A | 18 |

murine VEGF-A

| | | |
|---|---|---|
| Forward primer | GCAGAAGTCCCATGAAGTGA | 19 |
| Reverse Primer | CTCAATCGGACGGCAGTAG | 20 |
| Probe | TCAAGTTCATGGATGTCTACCAGCGAA | 21 |

human VEGF-C

| | | |
|---|---|---|
| Forward primer | CAGTGTCAGGCAGCGAACAA | 22 |
| Reverse Primer | CTTCCTGAGCCAGGCATCTG | 23 |
| Probe | CTGCCCCACCAATTACATGTGGAATAATCA | 24 |

murine VEGF-C

| | | |
|---|---|---|
| Forward primer | AAAGGGAAGAAGTTCCACCA | 25 |
| Reverse Primer | CAGTCCTGGATCACAATGCT | 26 |
| Probe | TCAGTCGATTCGCACACGGTCTT | 27 |
| human VEGF-D | | |
| Forward primer | CTGCCAGAAGCACAAGCTAT | 28 |
| Reverse Primer | ACATGGTCTGGTATGAAAGGG | 29 |
| Probe | CACCCAGACACCTGCAGCTGTG | 30 |

murine VEGF-D

| | | |
|---|---|---|
| Forward primer | TTG ACC TAG TGT CAT GGT AAA GC | 31 |
| Reverse Primer | TCA GTG AAC TGG GGA ATC AC | 32 |
| Probe | ACA TTT CCA TGC AAT GGC GGC T | 33 |

human Bv8

| | | |
|---|---|---|
| Forward primer | ATG GCA CGG AAG CTA GGA | 34 |
| Reverse Primer | GCA GAG CTG AAG TCC TCT TGA | 35 |
| Probe | TGC TGC TGG ACC CTT CCT AAA CCT | 36 |

murine Bv8

| | | |
|---|---|---|
| Forward primer | CGG AGG ATG CAC CAC ACC | 37 |
| Reverse Primer | CCG GTT GAA AGA AGT CCT TAA ACA | 38 |
| Probe | CCC CTG CCT GCC AGG CTT GG | 39 |

human P1GF all
isoforms

| | | |
|---|---|---|
| Forward primer | CAGCAGTGGGCCTTGTCT | 40 |
| Reverse Primer | AAGGGTACCACTTCCACCTC | 41 |
| Probe | TGACGAGCCGTTCCCAGC | 42 |

human P1GF,
isoforms 1 and 2

| | | |
|---|---|---|
| Forward primer | GAGCTGACGTTCTCTCAGCA | 43 |
| Reverse Primer | CTTTCCGGCTTCATCTTCTC | 44 |
| Probe | CTGCGAATGCCGGCCTCTG | 45 |

murine P1GF

| | | |
|---|---|---|
| Forward primer | TGCTTCTTACAGGTCCTAGCTG | 46 |
| Reverse Primer | AAAGGCACCACTTCCACTTC | 47 |
| Probe | CCCTGGGAATGCACAGCCAA | 48 |

human
VEGFR1/Flt1

| | | |
|---|---|---|
| Forward primer | CCGGCTTTCAGGAAGATAAA | 49 |
| Reverse Primer | TCCATAGTGATGGGCTCCTT | 50 |
| Probe | AACCGTCAGAATCCTCCTCTTCCTCA | 51 |

murine VEGFR1/Flt
(ECD)

| | | |
|---|---|---|
| Forward primer | GGCACCTGTACCAGACAAACTAT | 52 |
| Reverse Primer | GGCGTATTTGGACATCTAGGA | 53 |
| Probe | TGACCCATCGGCAGACCAATACA | 54 |

murine
VEGFR1/Flt1 (IC
Kinase Domain)

| | | |
|---|---|---|
| Forward primer | CGGAAACCTGTCCAACTACC | 55 |
| Reverse Primer | TGGTTCCAGGCTCTCTTTCT | 56 |
| Probe | CAACAAGGACGCAGCCTTGCA | 57 |

human VEGFR2

| | | |
|---|---|---|
| Forward primer | GGTCAGGCAGCTCACAGTCC | 58 |
| Reverse Primer | ACTTGTCGTCTGATTCTCCAGGTT | 59 |
| Probe | AGCGTGTGGCACCCACGATCAC | 60 |

murine VEGFR2

| | | |
|---|---|---|
| Forward primer | TCATTATCCTCGTCGGCACTG | 61 |
| Reverse Primer | CCTTCATTGGCCCGCTTAA | 62 |
| Probe | TTCTGGCTCCTTCTTGTCATTGTCCTACGG | 63 |

human VEGFR3

| | | |
|---|---|---|
| Forward primer | ACAGACAGTGGGATGGTGCTGGCC | 64 |
| Reverse Primer | CAAAGGCTCTGTGGACAACCA | 65 |
| Probe | TCTCTATCTGCTCAAACTCCTCCG | 66 |

murine VEGFR3

| | | |
|---|---|---|
| Forward primer | AGGAGCTAGAAAGCAGGCAT | 67 |
| Reverse Primer | CTGGGAATATCCATGTGCTG | 68 |
| Probe | CAGCTTCAGCTGTAAAGGTCCTGGC | 69 |

human NRP1
(transmembrane and
soluble)

| | | |
|---|---|---|
| Forward primer | CGGACCCATACCAGAGAATTA | 70 |
| Reverse Primer | CCATCGAAGACTTCCACGTA | 71 |
| Probe | TCAACCCTCACTTCGATTTGGAGGA | 72 |

human NRP1
(transmembrane)

| | | |
|---|---|---|
| Forward primer | AAACCAGCAGACCTGGATAAA | 73 |
| Reverse Primer | CACCTTCTCCTTCACCTTCG | 74 |
| Probe | TCCTGGCGTGCTCCCTGTTTC | 75 |

murine NRP1
(transmembrane and
soluble)

| | | |
|---|---|---|
| Forward primer | TTTCTCAGGAAGACTGTGCAA | 76 |
| Reverse Primer | TGGCTTCCTGGAGATGTTCT | 77 |
| Probe | CCTGGAGTGCTCCCTGTTTCATCA | 78 |

murine NRP1
(transmembrane)

| | | |
|---|---|---|
| Forward primer | CTGGAGATCTGGGATGGATT | 79 |
| Reverse Primer | TTTCTGCCCACAATAACGC | 80 |
| Probe | CCTGAAGTTGGCCCTCACATTGG | 81 |

human NRP1
(soluble, isoform 12)

| | | |
|---|---|---|
| Forward primer | CCACAGTGGAACAGGTGATG | 82 |
| Reverse Primer | CTGTCACATTTCGTATTTTATTTGA | 83 |
| Probe | GAAAAGCCCACGGTCATAGA | 84 |

human NRP1
(soluble, isoform 11)

| | | |
|---|---|---|
| Forward primer | CCACAGTGGAACAGGTGATG | 85 |
| Reverse Primer | ATGGTACAGCAATGGGATGA | 86 |
| Probe | CCAGCTCACAGGTGCAGAAACCA | 87 |

human NRP1
(soluble, isoform IV)

| | | |
|---|---|---|
| Forward primer | GACTGGGGCTCAGAATGG | 88 |
| Reverse Primer | CTATGACCGTGGGCTTTTCT | 89 |
| Probe | TGAAGTGGAAGGTGGCACCAC | 90 |

human Podoplanin

| | | |
|---|---|---|
| Forward primer | CCGCTATAAGTCTGGCTTGA | 91 |
| Reverse Primer | GATGCGAATGCCTGTTACAC | 92 |
| Probe | AACTCTGGTGGCAACAAGTGTCAACA | 93 |

murine Podoplanin

| | | |
|---|---|---|
| Forward primer | GGATGAAACGCAGACAACAG | 94 |
| Reverse Primer | GACGCCAACTATGATTCCAA | 95 |
| Probe | TGGCTTGCCAGTAGTCACCCTGG | 96 |

human Prox1

| | | |
|---|---|---|
| Forward primer | ACAAAAATGGTGGCACGGA | 97 |
| Reverse Primer | CCT GAT GTA CTT CGG AGC CTG | 98 |
| Probe | CCCAGTTTCCAAGCCAGCGGTCTCT | 99 |

murine Prox1

| | | |
|---|---|---|
| Forward primer | GCTGAAGACCTACTTCTCGGA | 100 |
| Reverse Primer | ACGGAAATTGCTGAACCACT | 101 |
| Probe | TTCAACAGATGCATTACCTCGCAGC | 102 |

human VE-Cadherin
(CD144, CDH5)

| | | |
|---|---|---|
| Forward primer | GAACAACTTTACCCTCACGGA | 103 |
| Reverse Primer | GGTCAAACTGCCCATACTTG | 104 |
| Probe | CACGATAACACGGCCAACATCACA | 105 |

murine VE-Cadherin
(CD144, CDH5)

| | | |
|---|---|---|
| Forward primer | TGAAGAACGAGGACAGCAAC | 106 |
| Reverse Primer | CCCGATTAAACTGCCCATAC | 107 |
| Probe | CACCGCCAACATCACGGTCA | 108 |

human robo4

| | | |
|---|---|---|
| Forward primer | GGGACCCACTAGACTGTCG | 109 |
| Reverse Primer | AGTGCTGGTGTCTGGAAGC | 110 |
| Probe | TCGCTCCTTGCTCTCCTGGGA | 111 |

human ICAM1

| | | |
|---|---|---|
| Forward primer | AACCAGAGCCAGGAGACACT | 112 |
| Reverse Primer | CGTCAGAATCACGTTGGG | 113 |
| Probe | TGACCATCTACAGCTTTCCGGCG | 114 |

murine ICAM1

| | | |
|---|---|---|
| Forward primer | CACGCTACCTCTGCTCCTG | 115 |
| Reverse Primer | CTTCTCTGGGATGGATGGAT | 116 |
| Probe | CACCAGGCCCAGGGATCACA | 117 |

human ESM1

| | | |
|---|---|---|
| Forward primer | TTCAGTAACCAAGTCTTCCAACA | 118 |
| Reverse Primer | TCACAATATTGCCATCTCCAG | 119 |
| Probe | TCTCACGGAGCATGACATGGCA | 120 |

murine ESM1

| | | |
|---|---|---|
| Forward primer | CAGTATGCAGCAGCCAAATC | 121 |
| Reverse Primer | CTCTTCTCTCACAGCGTTGC | 122 |
| Probe | TGCCTCCCACACAGAGCGTG | 123 |

human NG2

| | | |
|---|---|---|
| Forward primer | AGGCAGCTGAGATCAGAAGG | 124 |
| Reverse Primer | GATGTCTGCAGGTGGCACT | 125 |
| Probe | CTCCTGGGCTGCCTCCAGCT | 126 |

murine NG2

| | | |
|---|---|---|
| Forward primer | ACAGTGGGCTTGTGCTGTT | 127 |
| Reverse Primer | AGAGAGGTCGAAGTGGAAGC | 128 |
| Probe | TCCTTCCAGGGCTCCTCTGTGTG | 129 |

human FGF2

| | | |
|---|---|---|
| Forward primer | ACCCCGACGGCCGA | 130 |
| Reverse Primer | TCTTCTGCTTGAAGTTGTAGCTTGA | 131 |
| Probe | TCCGGGAGAAGAGCGACCCTCAC | 132 |

murine FGF2

| | | |
|---|---|---|
| Forward primer | ACCTTGCTATGAAGGAAGATGG | 133 |
| Reverse Primer | TTCCAGTCGTTCAAAGAAGAAA | 134 |
| Probe | AACACACTTAGAAGCCAGCAGCCGT | 135 |

human IL8/CXCL8

| | | |
|---|---|---|
| Forward primer | GGCAGCCTTCCTGATTTCT | 136 |
| Reverse Primer | TTCTTTAGCACTCCTTGGCA | 137 |
| Probe | AAACTGCACCTTCACACAGAGCTGC | 138 |

human HGF

| | | |
|---|---|---|
| Forward primer | TGGGACAAGAACATGGAAGA | 139 |
| Reverse Primer | GCATCATCATCTGGATTTCG | 140 |
| Probe | TCAGCTTACTTGCATCTGGTTCCCA | 141 |

murine HGF

| | | |
|---|---|---|
| Forward primer | GGACCAGCAGACACCACA | 142 |
| Reverse Primer | TATCATCAAAGCCCTTGTCG | 143 |
| Probe | CCGGCACAAGTTCTTGCCAGAA | 144 |

human THBS1/TSP1

| | | |
|---|---|---|
| Forward primer | TTTGGAACCACACCAGAAGA | 145 |
| Reverse Primer | GTCAAGGGTGAGGAGGACAC | 146 |
| Probe | CCTCAGGAACAAAGGCTGCTCCA | 147 |

murine
THBS1/TSP1

| | | |
|---|---|---|
| Forward primer | CGATGACAACGACAAGATCC | 148 |
| Reverse Primer | TCTCCCACATCATCTCTGTCA | 149 |
| Probe | CCATTCCATTACAACCCAGCCCA | 150 |

human ANG1

| | | |
|---|---|---|
| Forward primer | AGTTAATGGACTGGGAAGGG | 151 |
| Reverse Primer | GCTGTCCCAGTGTGACCTTT | 152 |
| Probe | ACCGAGCCTATTCACAGTATGACAGA | 153 |

human GM-
CSF/CSF2

| | | |
|---|---|---|
| Forward primer | TGCTGCTGAGATGAATGAAA | 154 |
| Reverse Primer | CCCTGCTTGTACAGCTCCA | 155 |
| Probe | CTCCAGGAGCCGACCTGCCT | 156 |

murine GM-
CSF/CSF2

| | | |
|---|---|---|
| Forward primer | AGCCAGCTACTACCAGACATACTG | 157 |
| Reverse Primer | GAAATCCGCATAGGTGGTAAC | 158 |
| Probe | AACTCCGGAAACGGACTGTGAAACAC | 159 |

human G-CSF/CSF3

| | | |
|---|---|---|
| Forward primer | GTCCCACCTTGGACACACT | 160 |
| Reverse Primer | TCCCAGTTCTTCCATCTGCT | 161 |
| Probe | CTGGACGTCGCCGACTTTGC | 162 |

murine G-CSF/CSF3

| | | |
|---|---|---|
| Forward primer | GAGTGGCTGCTCTAGCCAG | 163 |
| Reverse Primer | GACCTTGGTAGAGGCAGAGC | 164 |
| Probe | TGCAGCAGACACAGTGCCTAAGCC | 165 |

human FGF9

| | | |
|---|---|---|
| Forward primer | TATCCAGGGAACCAGGAAAG | 166 |
| Reverse Primer | CAGGCCCACTGCTATACTGA | 167 |
| Probe | CACAGCCGATTTGGCATTCTGG | 168 |

human
CXCL12/SDF1

| | | |
|---|---|---|
| Forward primer | ACACTCCAAACTGTGCCCTT | 169 |
| Reverse Primer | GGGTCAATGCACACTTGTCT | 170 |
| Probe | TGTAGCCCGGCTGAAGAACAACA | 171 |

murine
CXCL12/SDF1

| | | |
|---|---|---|
| Forward primer | CCAACGTCAAGCATCTGAAA | 172 |
| Reverse Primer | GGGTCAATGCACACTTGTCT | 173 |
| Probe | TGCCCTTCAGATTGTTGCACGG | 174 |

human TGFb1

| | | |
|---|---|---|
| Forward primer | CGTCTGCTGAGGCTCAAGT | 175 |
| Reverse Primer | GGAATTGTTGCTGTATTTCTGG | 176 |
| Probe | CAGCTCCACGTGCTGCTCCA | 177 |

murine TGFb1

| | | |
|---|---|---|
| Forward primer | CCCTATATTTGGAGCCTGGA | 178 |
| Reverse Primer | CGGGTTGTGTTGGTTGTAGA | 179 |
| Probe | CACAGTACAGCAAGGTCCTTGCCC | 180 |

human TNFa

| | | |
|---|---|---|
| Forward primer | TCAGATCATCTTCTCGAACCC | 181 |
| Reverse Primer | CAGCTTGAGGGTTTGCTACA | 182 |
| Probe | CGAGTGACAAGCCTGTAGCCCATG | 183 |

murine TNFa

| | | |
|---|---|---|
| Forward primer | AGTTCTATGGCCCAGACCCT | 184 |
| Reverse Primer | TCCACTTGGTGGTTTGCTAC | 185 |
| Probe | TCGAGTGACAAGCCTGTAGCCCA | 186 |

human BMP9

| | | |
|---|---|---|
| Forward primer | CAACATTGTGCGGAGCTT | 187 |
| Reverse Primer | GAGCAAGATGTGCTTCTGGA | 188 |
| Probe | CAGCATGGAAGATGCCATCTCCA | 189 |

human BMP10

| | | |
|---|---|---|
| Forward primer | CCTTGGTCCACCTCAAGAAT | 190 |
| Reverse Primer | GGAGATGGGCTCTAGCTTTG | 191 |
| Probe | CCAAAGCCTGCTGTGTGCCC | 192 |

human Sema3a

| | | |
|---|---|---|
| Forward primer | GAGGTTCTGCTGGAAGAAATG | 193 |
| Reverse Primer | CTGCTTAGTGGAAAGCTCCAT | 194 |
| Probe | CGGGAACCGACTGCTATTTCAGC | 195 |

murine Sema3a

| | | |
|---|---|---|
| Forward primer | TCCTCATGCTCACGCTATTT | 196 |
| Reverse Primer | AGTCAGTGGGTCTCCATTCC | 197 |
| Probe | CGTCTTGTGCGCCTCTTTGCA | 198 |

human Sema3b

| | | |
|---|---|---|
| Forward primer | ACCTGGACAACATCAGCAAG | 199 |
| Reverse Primer | GCCCAGTTGCACTCCTCT | 200 |
| Probe | CCGGCCAGGCCAGCTTCTT | 201 |

murine Sema3b

| | | |
|---|---|---|
| Forward primer | AGCTGCCGATGGACACTAC | 202 |
| Reverse Primer | GGGACTGAGATCACTTTCAGC | 203 |
| Probe | TGTGCCCACATCTGTACCAATGAAGA | 204 |

human Sema3c

| | | |
|---|---|---|
| Forward primer | CAGGGCAGAATTCCATATCC | 205 |
| Reverse Primer | CGCATATTGGGTGTAAATGC | 206 |
| Probe | CGCCCTGGAACTTGTCCAGGA | 207 |

murine Sema3c

| | | |
|---|---|---|
| Forward primer | ATGTGAGACATGGAAACCCA | 208 |
| Reverse Primer | TTCAGCTGCATTTCTGTATGC | 209 |
| Probe | TTGAACCCTCGGCATTGTGTCA | 210 |

human Sema3e

| | | |
|---|---|---|
| Forward primer | GCTCACGCAATTTACACCAG | 211 |
| Reverse Primer | TTCTCTGCCCTCCTACATCA | 212 |
| Probe | TTCACACAGAGTCGCCCGACC | 213 |

murine Sema3e

| | | |
|---|---|---|
| Forward primer | CCACTGGTCACTATATGAAGGAA | 214 |
| Reverse Primer | CTTGCCTCCGTTTACTTTGC | 215 |
| Probe | CAAGGCCTGGTTCCTGTGCCA | 216 |

human Sema3f

| | | |
|---|---|---|
| Forward primer | GGAACCCTGTCATTTACGCT | 217 |
| Reverse Primer | GTAGACACACACGGCAGAGC | 218 |
| Probe | CCTCTGGCTCCGTGTTCCGA | 219 |

murine Sema3f

| | | |
|---|---|---|
| Forward primer | CGTCAGGAACCCAGTCATTT | 220 |
| Reverse Primer | AGACACACACTGCAGACCCT | 221 |
| Probe | CTTTACCTCTTCAGGCTCTGTGTTCCG | 222 |

human
LGALS1/Galectin1

| | | |
|---|---|---|
| Forward primer | CTCAAACCTGGAGAGTGCCT | 223 |
| Reverse Primer | GGTTCAGCACGAAGCTCTTA | 224 |
| Probe | CGTCAGGAGCCACCTCGCCT | 225 |

murine
LGALS1/Galectin1

| | | |
|---|---|---|
| Forward primer | AATCATGGCCTGTGGTCTG | 226 |
| Reverse Primer | CCCGAACTTTGAGACATTCC | 227 |
| Probe | TCGCCAGCAACCTGAATCTCA | 228 |

human
LGALS7B/Galectin7

| | | |
|---|---|---|
| Forward primer | CCTTCGAGGTGCTCATCATC | 229 |
| Reverse Primer | GGCGGAAGTGGTGGTACT | 230 |
| Probe | ACCACGGCCTTGAAGCCGTC | 231 |

murine
LGALS7B/Galectin7

| | | |
|---|---|---|
| Forward primer | GAGAATTCGAGGCATGGTC | 232 |
| Reverse Primer | ATCTGCTCCTTGCTCCTCAC | 233 |
| Probe | CATGGAACCTGCCAGCCTGG | 234 |

human TMEM100

| | | |
|---|---|---|
| Forward primer | TGGTAATGGATTGCCTCTCTC | 235 |
| Reverse Primer | CAGTGCTTCTAAGCTGGGTTT | 236 |
| Probe | CGAGCTTTCACCCTGGTGAGACTG | 237 |

murine TMEM100

| | | |
|---|---|---|
| Forward primer | AGTCAAGTGGCCTCTCTGGT | 238 |
| Reverse Primer | CGCTTCACAGGCTAGATTTG | 239 |
| Probe | TGAGCTTGCATCCTGACCAGGC | 240 |

human Alk

| | | |
|---|---|---|
| Forward primer | AGGTGGTGTGTGTGGATCAG | 241 |
| Reverse Primer | CCGCATCATCTGAGCTAGG | 242 |
| Probe | CTGGCTGCAGACCCGGTCCT | 243 |

murine Alk1

| | | |
|---|---|---|
| Forward primer | CTTTGGCCTAGTGCTATGGG | 244 |
| Reverse Primer | GAAAGGTGGCCTGTAATCCT | 245 |
| Probe | CGGCGGACCATCATCAATGG | 246 |

human ITGa5

| | | |
|---|---|---|
| Forward primer | GCCTCAATGCTTCTGGAAA | 247 |
| Reverse Primer | CAGTCCAGCTGAAGTTCCAC | 248 |
| Probe | CGTTGCTGACTCCATTGGTTTCACA | 249 |

murine ITGa5

| | | |
|---|---|---|
| Forward primer | ACCGTCCTTAATGGCTCAGA | 250 |
| Reverse Primer | CCACAGCATAGCCGAAGTAG | 251 |
| Probe | CAACGTCTCAGGAGAACAGATGGCC | 252 |

human CXCR4

| | | |
|---|---|---|
| Forward primer | CTTCCTGCCCACCATCTACT | 253 |
| Reverse Primer | CATGACCAGGATGACCAATC | 254 |
| Probe | CATCTTCTTAACTGGCATTGTGGGCA | 255 |

human Egf17

| | | |
|---|---|---|
| Forward primer | GTGTACCAGCCCTTCCTCAC | 256 |
| Reverse Primer | CGGTCCTATAGATGGTTCGG | 257 |
| Probe | ACCGGGCCTGCAGCACCTA | 258 |

murine Egf17

| | | |
|---|---|---|
| Forward primer | GGCAGCAGATGGTACTACTGAG | 259 |
| Reverse Primer | GATGGAACCTCCGGAAATC | 260 |
| Probe | CCCACAGTACACACTCTACGGCTGG | 261 |

human NG3/Egf18

| | | |
|---|---|---|
| Forward primer | AAGCCCTACCTGACCTTGTG | 262 |
| Reverse Primer | ATAACGCGGTACATGGTCCT | 263 |
| Probe | AGTGCTGCAGATGCGCCTCC | 264 |

murine NG3/Egf18

| | | |
|---|---|---|
| Forward primer | CTGTCAGGGCTGGAAGAAG | 265 |
| Reverse Primer | CACCTCCATTAAGACAAGGCT | 266 |
| Probe | TCACCTGTGATGCCATCTGCTCC | 267 |

human
HSPG2/perlecan

| | | |
|---|---|---|
| Forward primer | CGGCCATGAGTCCTTCTACT | 268 |
| Reverse Primer | GGAGAGGGTGTATCGCAACT | 269 |
| Probe | CCGTAGGCCGCCACCTTGTC | 270 |

human Fibronectin

| | | |
|---|---|---|
| Forward primer | GGTTCGGGAAGAGGTTGTTA | 271 |
| Reverse Primer | TCATCCGTAGGTTGGTTCAA | 272 |
| Probe | CCGTGGGCAACTCTGTCAACG | 273 |

murine Fibronectin

| | | |
|---|---|---|
| Forward primer | AGAACCAGAGGAGGCACAAG | 272 |
| Reverse Primer | CATCTGTAGGCTGGTTCAGG | 275 |
| Probe | CCTTCGCTGACAGCGTTGCC | 276 |

murine LyPD6

| | | |
|---|---|---|
| Forward primer | CTCAGTCCCGAGACTTCACA | 277 |
| Reverse Primer | AAACACTTAAACCCACCAGGA | 278 |
| Probe | CCTCCACCCTTCAACCACTCCG | 279 |

murine Spred-1

| | | |
|---|---|---|
| Forward primer | CGAGGCATTCGAAGAGCTA | 280 |
| Reverse Primer | TCCTCCTTCAGCCTCAGTTT | 281 |
| Probe | TCTCTAGGGTGCCCAGCGTCAA | 282 |

murine MFAP5

| | | |
|---|---|---|
| Forward primer | CATCGGCCAGTCAGACAGT | 283 |
| Reverse Primer | AGTCGGGAACAGATCTCATTATT | 284 |
| Probe | CTGCTTCACCAGTTTACGGCGC | 285 |

murine MFAP5

| | | |
|---|---|---|
| Forward primer | GACACACTCAGCAGCCAGAG | 286 |
| Reverse Primer | CCAAGAACAGCATATTGTCTACAG | 287 |
| Probe | CCGGCAGACAGATCGCAGCT | 288 |

murine fibulin2

| | | |
|---|---|---|
| Forward primer | AGAATGGTGCCCAGAGTGA | 289 |
| Reverse Primer | TTCTCTTTCAAGTAGGAGATGCAG | 290 |
| Probe | CATTGCCTCTGGGCTATCCTACAGATG | 291 |

murine
fibulin4/Efemp2

| | | |
|---|---|---|
| Forward primer | CACCTGCCCTGATGGTTAC | 292 |
| Reverse Primer | CAATAGCGGTAACGACACTCA | 293 |
| Probe | TGTCCACACATTCGGGTCCAATTT | 294 |

murine collagen IV
(a1)

| | | |
|---|---|---|
| Forward primer | CGGCAGAGATGGTCTTGAA | 295 |
| Reverse Primer | TCTCTCCAGGCTCTCCCTTA | 296 |
| Probe | CCTTGTGGACCCGGCAATCC | 297 |

murine collagen IV
(a2)

| | | |
|---|---|---|
| Forward primer | TTCATTCCTCATGCACACTG | 298 |
| Reverse Primer | GCACGGAAGTCCTCTAGACA | 299 |
| Probe | ACTGGCCACCGCCTTCATCC | 300 |

murine collagen IV
(a3)

| | | |
|---|---|---|
| Forward primer | TTACCCTGCTGCTACTCCTG | 301 |
| Reverse Primer | GCATTGTCCTTTGCCTTTG | 302 |
| Probe | CACAGCCCTTGCTAGCCACAGG | 303 |

murine Hhex

| | | |
|---|---|---|
| Forward primer | GGCCAAGATGTTACAGCTCA | 304 |
| Reverse Primer | TTGCTTTGAGGATTCTCCTG | 305 |
| Probe | CCTGGTTTCAGAATCGCCGAGC | 306 |

murine robo4

| | | |
|---|---|---|
| Forward primer | CCTTTCTCTTCGTGGAGCTT | 307 |
| Reverse Primer | GTCAGAGGAGGGAGCTTGG | 308 |
| Probe | TCCACACACTGGCTCTGTGGGTC | 309 |

murine PDGFb

| | | |
|---|---|---|
| Forward primer | CATCTCGAGGGAGGAGGAG | 310 |
| Reverse Primer | CACTCGGCGATTACAGCA | 311 |
| Probe | TGCTGCTGCCAGGGACCCTA | 312 |

murine PDGFRb

| | | |
|---|---|---|
| Forward primer | CTTATGATAACTATGTCCCATCTGC | 313 |
| Reverse Primer | CTGGTGAGTCGTTGATTAAGGT | 314 |
| Probe | CCCTGAAAGGACCTATCGCGCC | 315 |

murine RGS5

| | | |
|---|---|---|
| Forward primer | GAGGAGGTCCTGCAGTGG | 316 |
| Reverse Primer | TGAAGCTGGCAAATCCATAG | 317 |
| Probe | CGCCAGTCCCTGGACAAGCTT | 318 |

murine CXCL1

| | | |
|---|---|---|
| Forward primer | CCGAAGTCATAGCCACACTC | 319 |
| Reverse Primer | TTTCTGAACCAAGGGAGCTT | 320 |
| Probe | AAGGCAAGCCTCGCGACCAT | 321 |

murine CXCL2

| | | |
|---|---|---|
| Forward primer | AAAGGCAAGGCTAACTGACC | 322 |
| Reverse Primer | CTTTGGTTCTTCCGTTGAGG | 323 |
| Probe | CAGCAGCCCAGGCTCCTCCT | 324 |

murine
PECAM/CD31

| | | |
|---|---|---|
| Forward primer | TCC CCG AAG CAG CAC TCT T | 325 |
| Reverse Primer | ACC GCA ATG AGC CCT TTC T | 326 |
| Probe | CAG TCA GAG TCT TCC TTG CCC CAT GG | 327 |

murine VCAM1

| | | |
|---|---|---|
| Forward primer | AACCCAAACAGAGGCAGAGT | 328 |
| Reverse Primer | CAGATGGTGGTTTCCTTGG | 329 |
| Probe | CAGCCTCTTTATGTCAACGTTGCCC | 330 |

Human HMBS

| | | |
|---|---|---|
| forward primer | CTTGATGACTGCCTTGCCTC | 331 |
| reverse primer | GGTTACATTCAAAGGCTGTTGCT | 332 |
| probe | TCTTTAGAGAAGTCC | 333 |

Human SDHA

| | | |
|---|---|---|
| forward primer | GGGAGCGTGGCACTTACCT | 334 |
| reverse primer | TGCCCAGTTTTATCATCTCACAA | 335 |
| probe | TGTCCCTTGCTTCATT | 336 |
| Human UBC | | |
| forward primer | TGCACTTGGTCCTGCGCTT | 337 |
| reverse primer | GGGAATGCAACAACTTTATTGAAA | 338 |
| probe | TGTCTAAGTTTCCCCTTTTA | 339 |

Human VEGFD

| | | |
|---|---|---|
| forward primer | ATTGACATGCTATGGGATAGCAACA | 340 |
| reverse primer | CTGGAGATGAGAGTGGTCTTCT | 341 |
| probe | TGTGTTTTGCAGGAGGAAAATCCACTTGCTGGA | 342 |

Human VEGFR1

| | | |
|---|---|---|
| forward primer | CTGGCAAGCGGTCTTACC | 343 |
| reverse primer | GCAGGTAACCCATCTTTTAACCATAC | 344 |
| probe | AAGTGAAGGCATTTCCCTCGCCGGAA | 345 |

Human VEGFR2

| | | |
|---|---|---|
| forward primer | AGG GAG TCT GTG GCA TCT G | 346 |
| reverse primer | GGA GTG ATA TCC GGA CTG GTA | 347 |
| probe | AGG CTC AAA CCA GAC AAG CGG C | 348 |

Human NRP2

| | | |
|---|---|---|
| forward primer | AGGACTGGATGGTGTACCG | 350 |
| reverse primer | TTCAGAACCACCTCAGTTGC | 351 |
| probe | CCACAAGGTATTTCAAGCCAACAACG | 352 |

Human Prox1

| | | |
|---|---|---|
| forward primer | TCAGATCACATTACGGGAGTTT | 352 |
| reverse primer | CAGCTTGCAGATGACCTTGT | 353 |
| probe | TCAATGCCATTATCGCAGGCAAA | 354 |

Human VE-Cadherin
(CD144, CDH5)

| | | |
|---|---|---|
| forward primer | ACA ATG TCC AAA CCC ACT CAT G | 355 |
| reverse primer | GAT GTG ACA ACA GCG AGG TGT AA | 356 |
| probe | TGC ATG ACG GAG CCG AGC CAT | 357 |

Human CD31/Pecam

| | | |
|---|---|---|
| forward primer | AGAAGCAAAATACTGACAGTCAGAG | 358 |
| reverse primer | GAG CAA TGA TCA CTC CGA TG | 359 |
| probe | CTGCAATAAGTCCTTTCTTCCATGG | 360 |
| Human Col4a1 | | |
| forward primer | CTGGAGGACAGGGACCAC | 361 |
| reverse primer | GGGAAACCCTTCTCTCCTTT | 362 |
| probe | CCAGGAGGGCCTGACAACCC | 363 |

Human Col4a2

| | | |
|---|---|---|
| forward primer | GCTACCCTGAGAAAGGTGGA | 364 |
| reverse primer | GGGAATCCTTGTAATCCTGGT | 365 |
| probe | CACTGGCCCAGGCTGACCAC | 366 |

Human Col4a3

| | | |
|---|---|---|
| forward primer | AGGAATCCCAGGAGTTGATG | 367 |
| reverse primer | CCTGGGATATAAGGGCACTG | 368 |
| probe | CCCAAAGGAGAACCAGGCCTCC | 369 |

Human Hhex

| | | |
|---|---|---|
| forward primer | CTCAGCGAGAGACAGGTCAA | 370 |
| reverse primer | TTTATTGCTTTGAGGGTTCTCC | 371 |
| probe | TCTCCTCCATTTAGCGCGTCGA | 372 |

Human DLL4

| | | |
|---|---|---|
| forward primer | AGGCCTGTTTTGTGACCAAGA | 373 |
| reverse primer | GAGCACGTTGCCCCATTCT | 374 |
| probe | ACTGCACCCACCACT | 375 |

Human PDGFRb

| | | |
|---|---|---|
| forward primer | CGGAAACGGCTCTACATCTT | 376 |
| reverse primer | AGTTCCTCGGCATCATTAGG | 377 |
| probe | CCAGATCCCACCGTGGGCTT | 378 |

Human RGS5

| | | |
|---|---|---|
| forward primer | ACCAGCCAAGACCCAGAAA | 379 |
| reverse primer | GCAAGTCCATAGTTGTTCTGC | 380 |
| probe | CACTGCAGGGCCTCGTCCAG | 381 |

Human CCL2/MCP1

| | | |
|---|---|---|
| forward primer | GAAGATCTCAGTGCAGAGGCT | 382 |
| reverse primer | TGAAGATCACAGCTTCTTTGG | 383 |
| probe | CGCGAGCTATAGAAGAATCACCAGCA | 384 |

Human CCL5

| | | |
|---|---|---|
| forward primer | TACACCAGTGGCAAGTGCTC | 385 |
| reverse primer | CACACTTGGCGGTTCTTTC | 386 |
| probe | CCCAGCAGTCGTCTTTGTCACCC | 387 |

Human
CXCL5/ENA-78

| | | |
|---|---|---|
| forward primer | GACGGTGGAAACAAGGAAA | 388 |
| reverse primer | TCTCTGCTGAAGACTGGGAA | 389 |
| probe | TCCATGCGTGCTCATTTCTCTTAATCA | 390 |

Human FGF8

| | | |
|---|---|---|
| forward primer | GGCCAACAAGCGCATCA | 391 |
| reverse primer | AAGGTGTCCGTCTCCACGAT | 392 |
| probe | CCTTCGCAAAGCT | 393 |

Human FGF8b

| | | |
|---|---|---|
| forward primer | GCTGGTCCTCTGCCTCCAA | 394 |
| reverse primer | TCCCTCACATGCTGTGTAAAATTAG | 395 |
| probe | CCCAGGTAACTGTTCAGT | 396 |

Human

| | | |
|---|---|---|
| CXCL12/SDF1 | | |
| forward primer | TCTCAACACTCCAAACTGTGC | 397 |
| reverse primer | GGGTCAATGCACACTTGTCT | 170 |
| probe | CCTTCAGATTGTAGCCCGGCTGA | 398 |

Human TGFb1

| | | |
|---|---|---|
| forward primer | TTTGATGTCACCGGAGTTGT | 399 |
| reverse primer | GCGAAAGCCCTCAATTTC | 400 |
| probe | TCCACGGCTCAACCACTGCC | 401 |

Human BMP9

| | | |
|---|---|---|
| forward primer | GGAGTAGAGGGAAGGAGCAG | 402 |
| reverse primer | CTGGGTTGTGGGAAATAACA | 403 |
| probe | CCGCGTGTCACACCCATCATT | 404 |

Human Sema3c

| | | |
|---|---|---|
| forward primer | GCCATTCCTGTTCCAGATTC | 405 |
| reverse primer | TCAGTGGGTTTCCATGTCTC | 406 |
| probe | TCGGCTCCTCCGTTTCCCAG | 407 |

Human cMet

| | | |
|---|---|---|
| forward primer | CACCATAGCTAATCTTGGGACAT | 408 |
| reverse primer | TGATGGTCCTGATCGAGAAA | 409 |
| probe | CCACAACCTGCATGAAGCGACC | 410 |

Human JAG1

| | | |
|---|---|---|
| forward primer | CGGGAACATACTGCCATGAA | 411 |
| reverse primer | GCAAGTGCCACCGTTTCTACA | 412 |
| probe | ATGACTGTGAGAGCAAC | 413 |

Human Notch1

| | | |
|---|---|---|
| forward primer | CACCTGCCTGGACCAGAT | 414 |
| reverse primer | GTCTGTGTTGACCTCGCAGT | 415 |
| probe | TCTGCATGCCCGGCTACGAG | 416 |

Human EphB4

| | | |
|---|---|---|
| forward primer | TCTGAAGTGGGTGACATTCC | 417 |
| reverse primer | CTGTGCTGTTCCTCATCCAG | 418 |
| probe | CTCCCACTGCCCGTCCACCT | 419 |

Human EFNB2

| | | |
|---|---|---|
| forward primer | ATCCAGGTTCTAGCACAGACG | 420 |
| reverse primer | TGAAGCAATCCCTGCAAATA | 421 |
| probe | TCCTCGGTTCCGAAGTGGCC | 422 |

Human FN1_EIIIA

| | | |
|---|---|---|
| forward primer | GAATCCAAGCGGAGAGAGTC | 423 |
| reverse primer | ACATCAGTGAATGCCAGTCC | 424 |
| probe | TGCAGTAACCAACATTGATCGCCC | 425 |

Human EFEMP2

| | | |
|---|---|---|
| forward primer | GATCAGCTTCTCCTCAGGATTC | 426 |
| reverse primer | TGTCTGGGTCCCACTCATAG | 427 |
| probe | CCCGACAGCTACACGGAATGCA | 428 |

Human FBLN2

| | | |
|---|---|---|
| forward primer | GAGCCAAGGAGGGTGAGAC | 429 |
| reverse primer | CCACAGCAGTCACAGCATT | 430 |
| probe | ACGACAGCTGCGGCATCTCC | 431 |

Human MFAP5

| | | |
|---|---|---|
| forward primer | AGGAGATCTGCTCTCGTCTTG | 432 |
| reverse primer | AGCCATCTGACGGCAAAG | 433 |
| probe | CTCATCTTTCATAGCTTCGTGTTCCTT | 434 |

Human LyPD6

| | | |
|---|---|---|
| forward primer | AGAGACTCCGAGCATGAAGG | 435 |
| reverse primer | GGGCAGTGGCAAGTTACAG | 436 |
| probe | CCACAAGGTCTGCACTTCTTGTTGTG | 437 |

Human Map4k4

| | | |
|---|---|---|
| forward primer | TTCTCCATCTAGCGGAACAACA | 438 |
| reverse primer | GGTCTCATCCCATCACAGGAA | 439 |
| probe | TGACATCTGTGGTGGGAT | 440 |

Human FRAS1

| | | |
|---|---|---|
| forward primer | TACTTGGAGAGCACTGGCAT | 441 |
| reverse primer | CTGTGCAGTTATGTGGGCTT | 442 |
| probe | TGTGAAGCTTGCCACCAGTCCTG | 443 |

Murine ACTB

| | | |
|---|---|---|
| forward primer | GCAAGCAGGAGTACGATGAG | 444 |
| reverse primer | TAACAGTCCGCCTAGAAGCA | 445 |
| probe | CCTCCATCGTGCACCGCAAG | 446 |

Murine HMBS

| | | |
|---|---|---|
| forward primer | CTCCCACTCAGAACCTCCTT | 447 |
| reverse primer | AGCAGCAACAGGACACTGAG | 448 |
| probe | CCCAAAGCCCAGCCTGGC | 449 |

Murine SDHA

| | | |
|---|---|---|
| forward primer | CTACAAGGGACAGGTGCTGA | 450 |
| reverse primer | GAGAGAATTTGCTCCAAGCC | 451 |
| probe | CCTGCGCCTCAGTGCATGGT | 452 |

Murine VEGFD

| | | |
|---|---|---|
| forward primer | ATG CTG TGG GAT AAC ACC AA | 453 |
| reverse primer | GTG GGT TCC TGG AGG TAA GA | 454 |
| probe | CGA GAC TCC ACT GCC TGG GAC A | 455 |

Murine Bv8

| | | |
|---|---|---|
| forward primer | AAAGTCATGTTGCAAATGGAAG | 456 |
| reverse primer | AATGGAACCTCCTTCTTCCTC | 457 |
| probe | TCTTCGCCCTTCTTCTTTCCTGC | 458 |

Murine NRP1

| | | |
|---|---|---|
| forward primer | CTCAGGTGGAGTGTGCTGAC | 459 |
| reverse primer | TTGCCATCTCCTGTATGGTC | 460 |
| probe | CTGAATCGGCCCTGTCTTGCTG | 461 |

Murine NRP1

| | | |
|---|---|---|
| forward primer | CTACTGGGCTGTGAAGTGGA | 462 |
| reverse primer | CACACTCATCCACTGGGTTC | 463 |
| probe | CAGCTGGACCAACCACACCCA | 464 |

Murine NRP2

| | | |
|---|---|---|
| forward primer | GCATTATCCTGCCCAGCTAT | 465 |
| reverse primer | GATCGTCCCTTCCCTATCAC | 466 |
| probe | TCCCTCGAACACGATCTGATACTCCA | 467 |

Murine Prox1

| | | |
|---|---|---|
| forward primer | CGGACGTGAAGTTCAACAGA | 468 |
| reverse primer | ACGCGCATACTTCTCCATCT | 469 |
| probe | CGCAGCTCATCAAGTGGTTCAGC | 470 |

Murine Murine
CD34

| | | |
|---|---|---|
| forward primer | CCTGGAAGTACCAGCCACTAC | 471 |
| reverse primer | GGGTAGCTGTAAAGTTGACCGT | 472 |
| probe | ACCACACCAGCCATCTCAGAGACC | 473 |

Murine FGF8

| | | |
|---|---|---|
| forward primer | CAGGTCTCTACATCTGCATGAAC | 474 |
| reverse primer | AATACGCAGTCCTTGCCTTT | 475 |
| probe | AAGCTAATTGCCAAGAGCAACGGC | 476 |

Murine FGF8b

| | | |
|---|---|---|
| forward primer | CTGCCTGCTGTTGCACTT | 477 |
| reverse primer | TTAGGTGAGGACTGAACAGTTACC | 478 |
| probe | CTGGTTCTCTGCCTCCAAGCCC | 479 |

Murine CXCL2

| | | |
|---|---|---|
| forward primer | ACATCCAGAGCTTGAGTGTGA | 480 |
| reverse primer | GCCCTTGAGAGTGGCTATG | 481 |
| probe | CCCACTGCGCCCAGACAGAA | 482 |

Murine CCL5

| | | |
|---|---|---|
| forward primer | GCCCACGTCAAGGAGTATTT | 483 |
| reverse primer | TCGAGTGACAAACACGACTG | 484 |
| probe | CACCAGCAGCAAGTGCTCCAATC | 485 |

Murine TNFa

| | | |
|---|---|---|
| forward primer | CAGACCCTCACACTCAGATCA | 486 |
| reverse primer | TCCACTTGGTGGTTTGCTAC | 185 |
| probe | TCGAGTGACAAGCCTGTAGCCCA | 186 |

Murine Sema3b

| | | |
|---|---|---|
| forward primer | AGTACCTGGAGTTGAGGGTGA | 487 |
| reverse primer | GTCTCGGGAGGACAGAAGG | 488 |
| probe | CACCCACTTTGACCAACTTCAGGATG | 489 |

Murine PDGFC

| | | |
|---|---|---|
| forward primer | CCATGAGGTCCTTCAGTTGAG | 490 |
| reverse primer | TCCTGCGTTTCCTCTACACA | 491 |
| probe | CCTCGTGGTGTTCCAGAGCCA | 492 |

Murine Ang1

| | | |
|---|---|---|
| forward primer | CACGAAGGATGCTGATAACG | 493 |
| reverse primer | ACCACCAACCTCCTGTTAGC | 494 |
| probe | CAACTGTATGTGCAAATGCGCTCTCA | 495 |

Murine Ang2

| | | |
|---|---|---|
| forward primer | CACAAAGGATTCGGACAATG | 496 |
| reverse primer | AAGTTGGAAGGACCACATGC | 497 |
| probe | CAAACCACCAGCCTCCTGAGAGC | 498 |

Murine BMP9

| | | |
|---|---|---|
| forward primer | CTTCAGCGTGGAAGATGCTA | 499 |
| reverse primer | TGGCAGGAGACATAGAGTCG | 500 |
| probe | CGACAGCTGCCACGGAGGAC | 501 |

Murine BMP10

| | | |
|---|---|---|
| forward primer | CCATGCCGTCTGCTAACAT | 502 |
| reverse primer | GATATTTCCGGAGCCCATTA | 503 |
| probe | CAGATCTTCGTTCTTGAAGCTCCGG | 504 |

Murine cMet

| | | |
|---|---|---|
| forward primer | ACGTCAGAAGGTCGCTTCA | 505 |
| reverse primer | ACATGAGGAGTGAGGTGTGC | 506 |
| probe | TGTTCGAGAGAGCACCACCTGCA | 507 |

Murine CXCR4

| | | |
|---|---|---|
| forward primer | TGTAGAGCGAGTGTTGCCA | 508 |
| reverse primer | CCAGAACCCACTTCTTCAGAG | 509 |
| probe | TGTATATACTCACACTGATCGGTTCCA | 510 |

Murine DLL4

| | | |
|---|---|---|
| forward primer | ATGCCTGGGAAGTATCCTCA | 511 |
| reverse primer | GGCTTCTCACTGTGTAACCG | 512 |
| probe | TGGCACCTTCTCTCCTAAGCTCTTGTC | 513 |

Murine JAG1

| | | |
|---|---|---|
| forward primer | ACATAGCCTGTGAGCCTTCC | 514 |
| reverse primer | CTTGACAGGGTTCCCATCAT | 515 |
| probe | CGTGGCCATCTCTGCAGAAGACA | 516 |

Murine EFNB2

| | | |
|---|---|---|
| forward primer | GTCCAACAAGACGTCCAGAG | 517 |
| reverse primer | CGGTGCTAGAACCTGGATTT | 518 |
| probe | TCAACAACAAGTCCCTTTGTGAAGCC | 519 |

Murine EFNB2

| | | |
|---|---|---|
| forward primer | TTGGACAAGATGCAAGTTCTG | 520 |
| reverse primer | TCTCCCATTTGTACCAGCTTC | 521 |
| probe | TCAGCCAGGAATCACGGTCCA | 522 |

Murine Notch1

| | | |
|---|---|---|
| forward primer | CACTGCATGGACAAGATCAA | 523 |
| reverse primer | TCATCCACATCATACTGGCA | 524 |
| probe | CCCAAAGGCTTCAACGGGCA | 525 |

Murine TIE2

| | | |
|---|---|---|
| forward primer | CACGAAGGATGCTGATAACG | 526 |
| reverse primer | ACCACCAACCTCCTGTTAGC | 527 |
| probe | CAACTGTATGTGCAAATGCGCTCTCA | 528 |

Murine EphA3

| | | |
|---|---|---|
| forward primer | TTGCAATGCTGGGTATGAAG | 529 |
| reverse primer | AGCCTTGTAGAAGCCTGGTC | 530 |
| probe | AACGAGGTTTCATATGCCAAGCTTGTC | 531 |

Murine Bc12A1

| | | |
|---|---|---|
| forward primer | CAGAATTCATAATGAATAACACAGGA | 532 |
| reverse primer | CAGCCAGCCAGATTTGG | 533 |
| probe | GAATGGAGGTTGGGAAGATGGCTTC | 534 |

Murine Map4k4

| | | |
|---|---|---|
| forward primer | TTGCCACGTACTATGGTGCT | 535 |
| reverse primer | CCATAACAAGCCAGAGTTGG | 536 |
| probe | TCATCATGTCCTGGAGGGCTCTTCT | 537 |

Murine ANTXR2

| | | |
|---|---|---|
| forward primer | TGGGAAGTCTGCTGTCTCAA | 538 |
| reverse primer | AATAGCTACGATGGCTGCAA | 539 |
| probe | CACAGCCACAGAATGTACCAATGGG | 540 |

Murine IGFBP4

| | | |
|---|---|---|
| forward primer | CCCTGCGTACATTGATGC | 541 |
| reverse primer | GCTCTCATCCTTGTCAGAGGT | 542 |
| probe | ACAGCTCCGTGCACACGCCT | 543 |

Murine FGFR4

| | | |
|---|---|---|
| forward primer | GAGGCATGCAGTATCTGGAG | 544 |
| reverse primer | CTCGGTCACCAGCACATTT | 545 |
| probe | CTCGGAAGTGCATCCACCGG | 546 |

Murine
CLECSF5/CLEC5a

| | | |
|---|---|---|
| forward primer | GTACGTCAGCCTGGAGAGAA | 547 |
| reverse primer | ATTGGTAACATTGCCATTGAAC | 548 |
| probe | AAAGTGGCGCTGGATCAACAACTCT | 549 |

Murine
Mincle/CLECSF9

| | | |
|---|---|---|
| forward primer | GAATGAATTCAACCAAATCGC | 550 |
| reverse primer | CAGGAGAGCACTTGGGAGTT | 551 |
| probe | TCCCACCACACAGAGAGAGGATGC | 552 |

Murine
FBLN2/fibulin2

| | | |
|---|---|---|
| forward primer | TTGTCCACCCAACTATGTCC | 553 |
| reverse primer | CGTGATATCCTGGCATGTG | 554 |
| probe | TGCGCTCGCACTTCGTTTCTG | 555 |

Murine Egf17

| | | |
|---|---|---|
| forward primer | AGCCTTACCTCACCACTTGC | 556 |
| reverse primer | ATAGGCAGTCCGGTAGATGG | 557 |
| probe | CGGACACAGAGCCTGCAGCA | 558 |

Murine LAMA4

| | | |
|---|---|---|
| forward primer | ATTCCCATGAGTGCTTGGAT | 559 |
| reverse primer | CACAGTGCTCTCCTGTTGTGT | 560 |
| probe | CTGTCTGCACTGCCAGCGGA | 561 |

Murine NID2

| | | |
|---|---|---|
| forward primer | GCAGATCACTTCTACCACACG | 562 |
| reverse primer | CTGGCCACTGTCCTTATTCA | 563 |
| probe | TGATATAACACCATCCCTCCGCCA | 564 |

Murine FRAS1

| | | |
|---|---|---|
| forward primer | GGC AAT AAA CCG AGG ACT TC | 565 |
| reverse primer | TCA AGT GCT GCT CTG TGA TG | 566 |
| probe | CGT GCT ACG GAC CCT GCT GAA A | 567 |

Murine PLC/HSPG2

| | | |
|---|---|---|
| forward primer | GAGACAAGGTGGCAGCCTAT | 568 |
| reverse primer | TGTTATTGCCCGTAATCTGG | 569 |
| probe | CGGGAAGCTGCGGTACACCC | 570 |

Human hPTGS2

| | | |
|---|---|---|
| forward primer | GCTGGAACATGGAATTACCC | 571 |
| reverse primer | GTACTGCGGGTGGAACATT | 572 |
| probe | ACCAGCAACCCTGCCAGCAA | 573 |

Human PDGFA

| | | |
|---|---|---|
| forward primer | GTCCATGCCACTAAGCATGT | 574 |
| reverse primer | ACAGCTTCCTCGATGCTTCT | 575 |
| probe | CCCTGCCCATTCGGAGGAAG | 576 |

### Example 3

### Tumor Inhibitory Activities of Anti-NRP1 Antibodies

All studies were conducted in accordance with the Guide for the Care and Use of Laboratory Animals, published by the NIH (NIH Publication 85-23, revised 1985). An Institutional Animal Care and Use Committee (IACUC) approved all animal protocols.

Studies were conducted with the following human tumor models using standardized techniques: LS174t, A549, H1299, MV522, MDA-MB231, HT29, SKMES. Human tumor cells were implanted subcutaneously in the right flank of each test mouse. For example, for H1299, xenografts were initiated from cultured H1299 human non-small cell lung carcinoma cells (grown to mid-log phase in RPMI-1640 medium containing 10% heat-inactivated fetal bovine serum, 100 units/mL penicillin G, 100 μg/mL streptomycin sulfate, 0.25 μg/mL amphotericin B, 1 mM sodium pyruvate, 2 mM glutamine, 10 mM HEPES, 0.075% sodium bicarbonate, and 25 μg/mL gentamicin) or from A549 human lung adenocarcinoma cells (cultured in Kaighn's modified Ham's F12 medium containing 10% heat-inactivated fetal bovine serum, 100 units/mL penicillin G, 100 μg/mL streptomycin sulfate, 0.25 μg/mL amphotericin B, 2 mM glutamine, 1 mM sodium pyruvate, and 25 μg/mL gentamicin). On the day of tumor implant, H1299 cells were harvested and resuspended in PBS at a concentration of 5×10⁷ cells/mL. Each test mouse received 1×10⁷H1299 tumor cells implanted subcutaneously in the right flank. For A549 tumors, A549 cells were resuspended in 100% Matrigel™ matrix (BD Biosciences, San Jose, Calif.) at a concentration of 5×10⁷ cells/mL. A549 cells (1×10⁷ in 0.2 mL) were implanted subcutaneously in the right flank of each test mouse, and tumor growth was monitored. As an alternate example, a fragment of a LXFA629 tumor was implanted into the right flank of each test mouse and tumor growth was monitored.

Tumor growth was monitored as the average size approached 120-180 mm³. On study day 1, individual tumors sizes ranged from 126 to 196 mm³ and the animals were sorted by tumor size into three test groups (one control group and two treatment groups). Tumor volume was calculated using the formula:

Tumor volume (mm³)==(w²×l)/2

where w=width and l=length in mm of the tumor.

All treatments were administered intra-peritoneally. Tumors were treated twice weekly for up to 10-20 weeks with 5-10 mg/kg each of control antibody, an agent blocking VEGF-A activity (anti-VEGF-A antibody B20-4.1 at 5 mg/kg), or the combination of an agent blocking VEGF-A activity and an agent blocking NRP1 activity (anti-NRP1 antibody at 10 mg/kg). For the combination treatment group, anti-NRP1 antibody was administered no later than thirty minutes after administration of the anti-VEGF-A antibody. Each dose was delivered in a volume of 0.2 mL per 20 grams body weight (10 mL/kg), and was scaled to the body weight of the animal.

Tumor volume was recorded twice weekly using calipers. Each animal was euthanized when its tumor reached the endpoint size (generally 1000 mm³) or at the conclusion of the study, whichever occurred first.

The time to endpoint (TTE) was calculated from the following equation:

TTE (days)=(log₁₀(endpoint volume,mm³−b)/m

where b is the intercept and m is the slope of the line obtained by linear regression of a log-transformed tumor growth data set.

Animals that did reach the endpoint were assigned a TTE value equal to the last day of the study. Animals classified as NTR (non-treatment-related) deaths due to accident (NTRa) or due unknown causes (NTRu) were excluded from TTE calculations (and all further analyses). Animals classified as TR (treatment-related) deaths or NTRm (non-treatment-related death due to metastasis) were assigned a TTE value equal to the day of death. Tumor were harvested and either fixated overnight in 10% NBF, followed by 70% ethanol and subsequent embedding in paraffin, or within two minutes frozen in liquid nitrogen for subsequent storage at −80° C.

Treatment outcome was evaluated by tumor growth delay (TGD), which is defined as the increase in the median time to endpoint (TTE) in a treatment group compared to the control group, which was calculated as follows:

TGD=T−C, expressed in days, or as a percentage of the median TTE of the control group, which was calculated as follows:

% TGD=[(T−C)/C]×100,

where T=median TTE for a treatment group and C=median TTE for the control group.

The Δ% TGD was calculated as above, with C=control group being the group receiving anti-VEGF-A treatment alone, and T=treatment group being the group receiving the combination of anti-VEGF-A and anti-NRP1 treatment. The logrank test was employed to analyze the significance of the difference between the TTE values of two groups. Two-tailed statistical analyses were conducted at significance level p=0.05. A value of “1” indicates that treatment resulted in an additional delay in tumor progression. A value of “0” indicates that the treatment did not result in an additional delay in tumor progression.

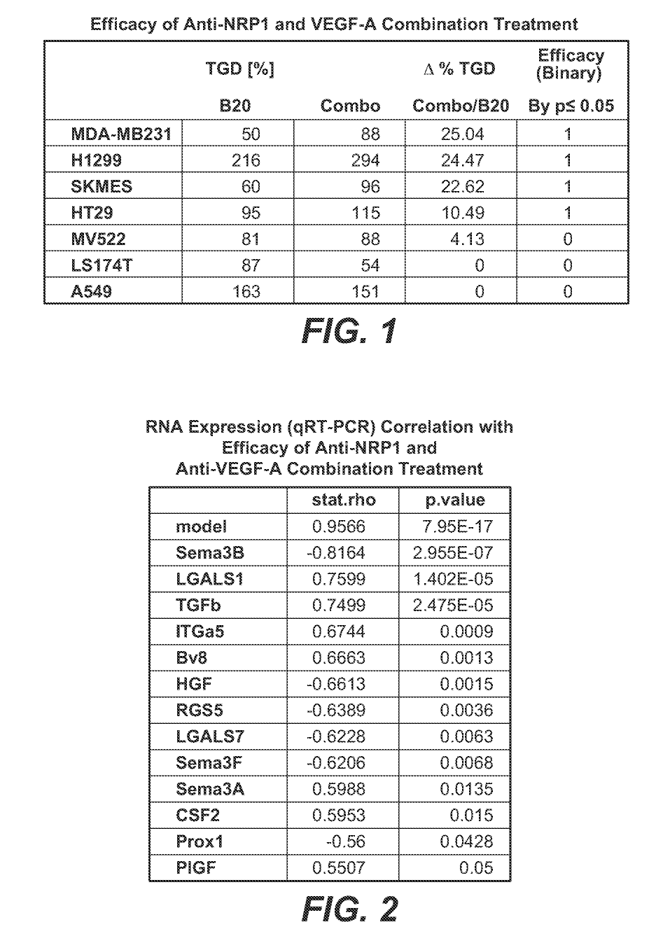

Treatment with the combination of anti-NRP1 antibody and anti-VEGF-A antibody resulted in additional delay in tumor progression in MDA-MB231, HT29, SKMES and H1299 tumors, compared to anti-VEGF treatment alone (FIG. 1).

### Example 4

### Identification of Biomarkers for Efficacy of Anti-NRP1 Antibody Treatment

Gene expression analysis was performed using qRT-PCR on frozen tumor samples obtained from the tumor model experiments described above in Example 3. From frozen material, small cubes of maximal 3 mm side length were solubilized using commercially available reagents and equipment (RNeasy®, Tissuelyzer, both Qiagen Inc, Germany). After column purification RNA was eluted with H₂O, precipitated with ethanol after the addition of glycogen and Sodium acetate. RNA was pelleted by centrifugation for at least 30 min, washed twice with 80% ethanol, and the pellet resuspended in H₂0 after drying. RNA concentrations were assessed using a spectrophotometer or a bioanalyzer (Agilent, Foster City, Calif.), and 50 ng of total RNA used per reaction in the subsequent gene expression analysis.

Gene specific primer and probe sets set forth in Example 1 above were used for qRT-PCR expression analysis of 18SrRNA, human and mouse RPS13 (housekeeping gene), NRP1 (transmembrane form only, and transmembrane and soluble form), Sema3A, Sema3B, Sema3F, PlGF, TGFβ1, HGF, Bv8, RGS5, Prox1, CSF2, LGALS1, LGALS7, and ITGa5.

Relative expression levels of NRP1, Sema3A, Sema3B, Sema3F, PlGF, TGFβ1, HGF, Bv8, RGS5, Prox1, CSF2, LGALS1, LGALS7 and ITGa5 was determined. For example, relative expression level of NRP1 was calculated as follows:

Relative expression NRP1ₛₐₘₚₗₑ=2exp(Ct_{[(18SrRNA+RPS13)/2]}−Ct_{NRP1}) with Ct determined in the sample, where Ct is the threshold cycle. The Ct is the cycle number at which the fluorescence generated within a reaction crosses the threshold line.

To allow comparison of results from different reaction plates, relative expression was then calculated as a fraction to the relative expression to an internal reference RNA that was identical in all experimental runs, multiplied by 100:

Normalized relative expression NRP1ₛₐₘₚₗₑ=(relative expression NRP1ₛₐₘₚₗₑ/relative expression NRP1_{reference RNA})×100, where relative expression NRP1_{reference RNA}=2exp(Ct_{[(18SrRNA+RPS13)/2]}−Ct_{NRP1}) with Ct determined in the reference RNA

Using this calculation, samples that had any signal in the qRT-PCR reaction had values above ‘1’, samples with values below ‘1’ were classed as ‘negative’ for the particular analyte.

The p- and r-values for the correlation of marker RNA expression (qPCR) and combination treatment efficacy are shown in FIG. 2.

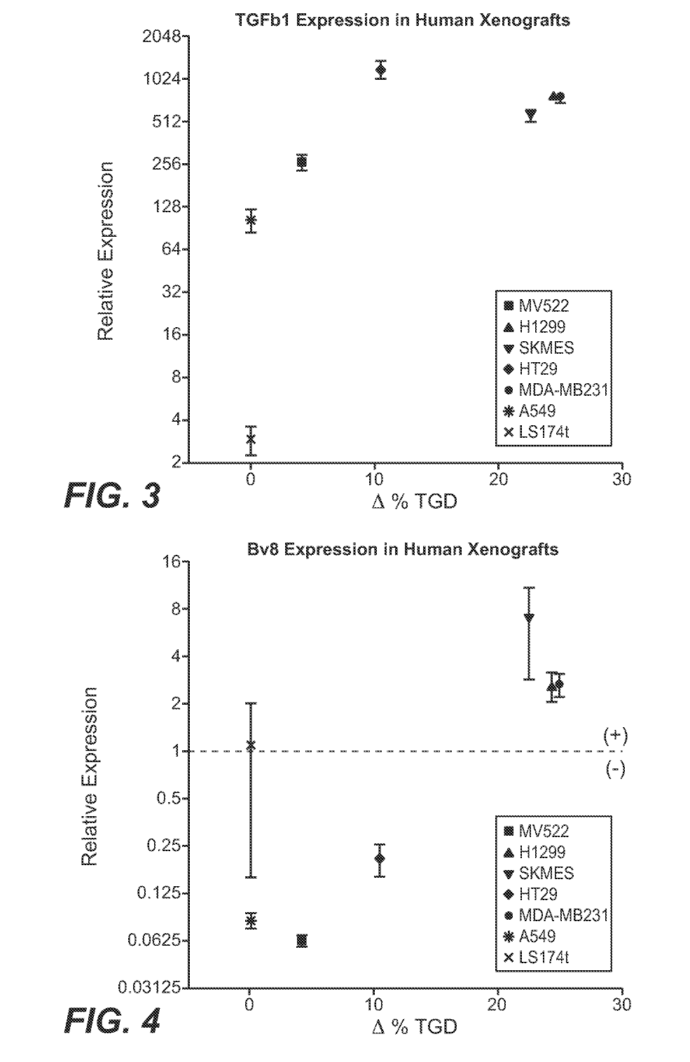

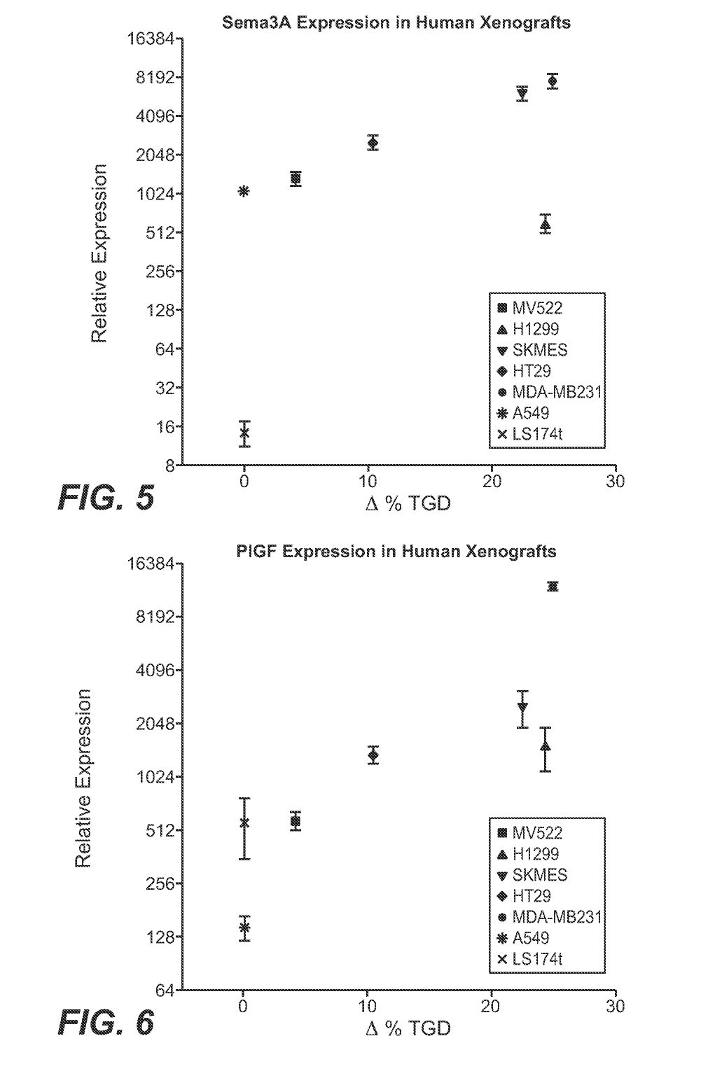

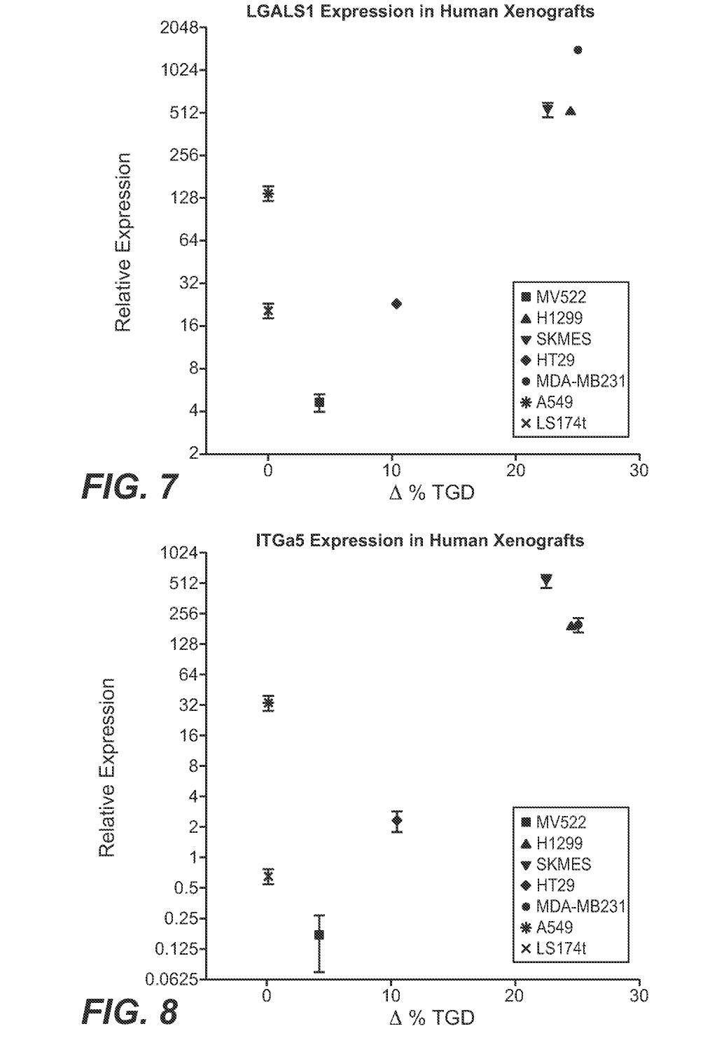

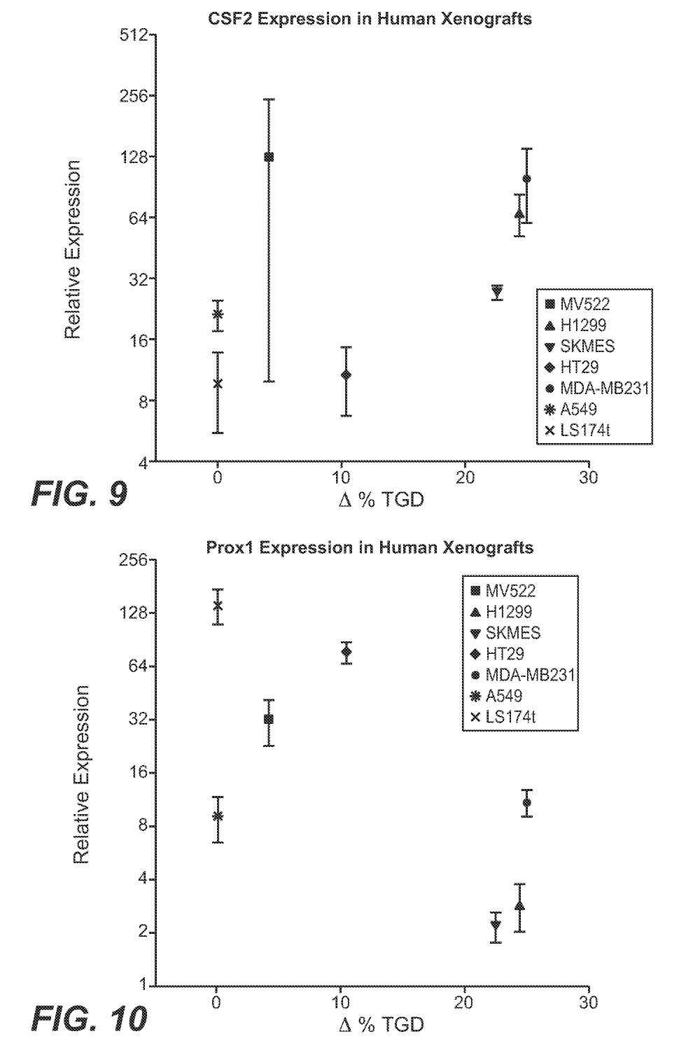

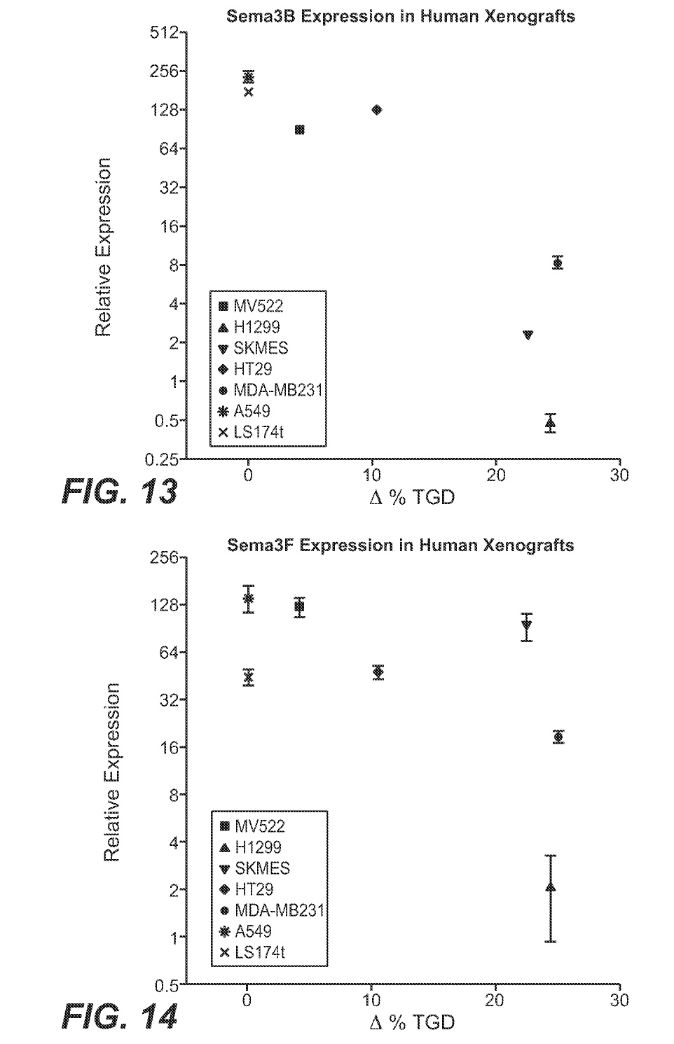

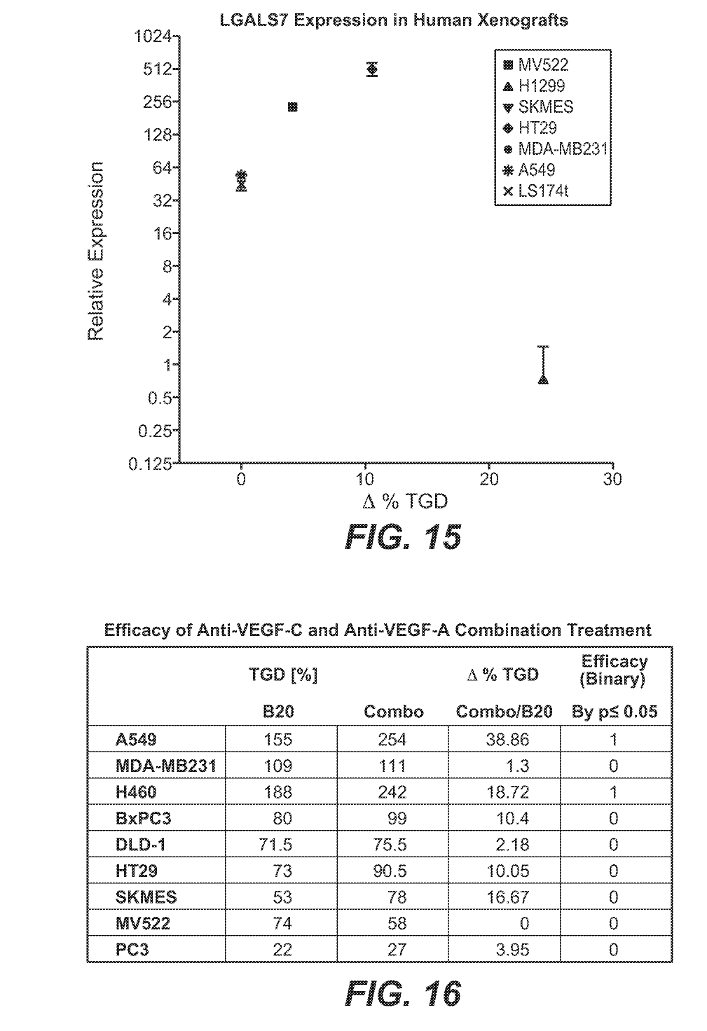

Results from the gene expression analysis are shown in FIGS. 3-15. In each of FIGS. 3-15, the relative expression of the gene assayed is compared to the percent change in tumor growth delay (Δ% TGD) exhibited by the seven different tumor models examined.

Tumor models that responded to treatment with anti-NRP1 antibody in combination with anti-VEGF-A antibody expressed higher levels of TGFβ1, Bv8, Sema3A, PlGF, LGALS1, ITGa5 and CSF2 compared to tumor models that did not respond to the combination treatment (see FIGS. 3-9).

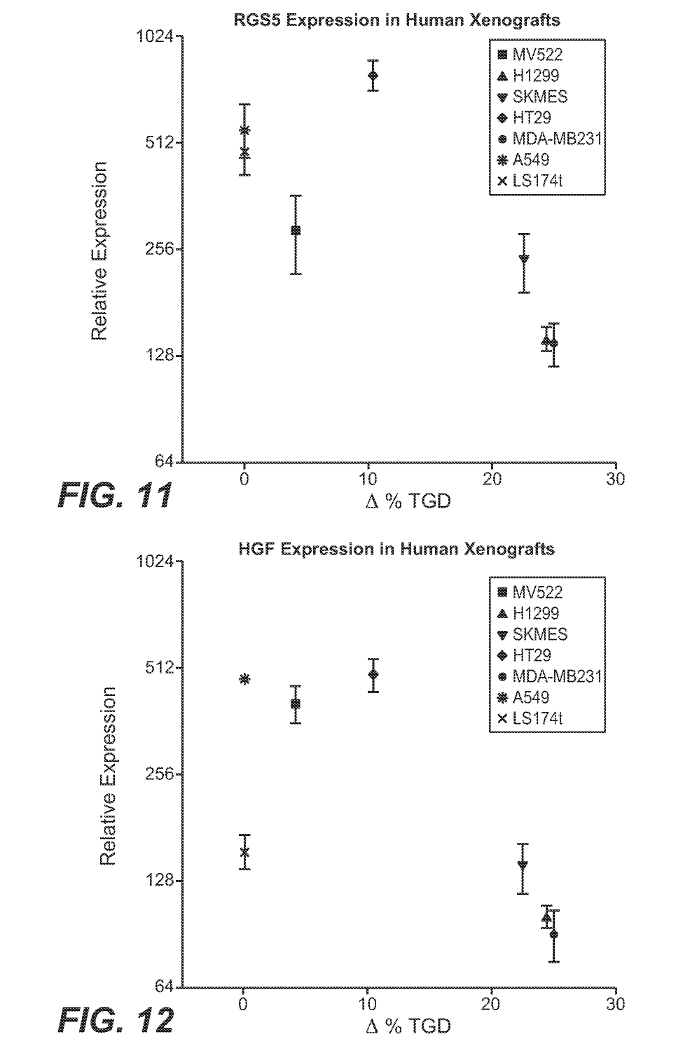

Tumor models responsive to the combination treatment with anti-NRP1 antibody and anti-VEGF-A antibody also expressed lower levels of Prox1, RGS5, HGF, Sema3B, Sema3F and LGALS7 as compared to the tumor models that did not respond to the combination treatment (see FIGS. 10-15).

### Example 5

### Tumor Inhibitory Activities of Anti-VEGF-C Antibodies

All studies were conducted in accordance with the Guide for the Care and Use of Laboratory Animals, published by the NIH (NIH Publication 85-23, revised 1985). An Institutional Animal Care and Use Committee (IACUC) approved all animal protocols.

Studies were conducted with the following human tumor models using standardized techniques: A549, MDA-MB231, H460, BxPC3, DLD-1, HT29, SKMES, MV522 and PC3. Human tumor cells were implanted subcutaneously in the right flank of each test mouse. For example, for A549, xenografts were initiated from cultured A549 human non-small cell lung carcinoma cells (grown to mid-log phase in RPMI-1640 medium containing 10% heat-inactivated fetal bovine serum, 100 units/mL penicillin G, 100 μg/mL streptomycin sulfate, 0.25 μg/mL amphotericin B, 1 mM sodium pyruvate, 2 mM glutamine, 10 mM HEPES, 0.075% sodium bicarbonate, and 25 μg/mL gentamicin) or from A549 human lung adenocarcinoma cells (cultured in Kaighn's modified Ham's F12 medium containing 10% heat-inactivated fetal bovine serum, 100 units/mL penicillin G, 100 μg/mL streptomycin sulfate, 0.25 μg/mL amphotericin B, 2 mM glutamine, 1 mM sodium pyruvate, and 25 μg/mL gentamicin). On the day of tumor implant, A549 cells were harvested and resuspended in PBS at a concentration of 5×10⁷ cells/mL. Each test mouse received 1×10⁷ A549 tumor cells implanted subcutaneously in the right flank. For A549 tumors, A549 cells were resuspended in 100% Matrigel™ matrix (BD Biosciences, San Jose, Calif.) at a concentration of 5×10⁷ cells/mL. A549 cells (1×10⁷ in 0.2 mL) were implanted subcutaneously in the right flank of each test mouse, and tumor growth was monitored.

Tumor growth was monitored as the average size approached 120-180 mm³. On study day 1, individual tumors sizes ranged from 126 to 196 mm³ and the animals were sorted by tumor size into three test groups (one control group and two treatment groups). Tumor volume was calculated using the formula:

Tumor volume (mm³)==(w²×l)/2

where w=width and l=length in mm of the tumor.

All treatments were administered intra-peritoneally. Tumors were treated twice weekly for up to 10-20 weeks with 5-10 mg/kg each of control antibody, an agent blocking VEGF-A activity (anti-VEGF-A antibody B20-4.1 at 5 mg/kg), or the combination of an agent blocking VEGF-A activity and an agent blocking VEGF-C activity (anti-VEGF-C antibody at 10 mg/kg. For the combination treatment group, anti-VEGF-C antibody was administered no later than thirty minutes after administration of the anti-VEGF-A antibody. Each dose was delivered in a volume of 0.2 mL per 20 grams body weight (10 mL/kg), and was scaled to the body weight of the animal.

Tumor volume was recorded twice weekly using calipers. Each animal was euthanized when its tumor reached the endpoint size (generally 1000 mm³) or at the conclusion of the study, whichever came first. Tumors were harvested and either fixated overnight in 10% NBF, followed by 70% ethanol and subsequent embedding in paraffin, or within two minutes frozen in liquid nitrogen for subsequent storage at −80° C.

The time to endpoint (TTE) was calculated from the following equation:

TTE (days)=(log₁₀(endpoint volume,mm³−b)/m

where b is the intercept and m is the slope of the line obtained by linear regression of a log-transformed tumor growth data set.

Animals that did reach the endpoint were assigned a TTE value equal to the last day of the study. Animals classified as NTR (non-treatment-related) deaths due to accident (NTRa) or due unknown causes (NTRu) were excluded from TTE calculations (and all further analyses). Animals classified as TR (treatment-related) deaths or NTRm (non-treatment-related death due to metastasis) were assigned a TTE value equal to the day of death.

Treatment outcome was evaluated by tumor growth delay (TGD), which is defined as the increase in the median time to endpoint (TTE) in a treatment group compared to the control group, which was calculated as follows:

TGD=T−C, expressed in days, or as a percentage of the median TTE of the control group, which was calculated as follows:

% TGD=[(T−C)/C]×100,

where T=median TTE for a treatment group and C=median TTE for the control group.

The Δ% TGD was calculated as above, with C=control group being the group receiving anti-VEGF-A antibody treatment alone, and T=treatment group being the group receiving the combination of anti-VEGF-A antibody and anti-VEGF-C antibody treatment. The logrank test was employed to analyze the significance of the difference between the TTE values of two groups. Two-tailed statistical analyses were conducted at significance level p=0.05. A value of “1” indicates that treatment resulted in an additional delay in tumor progression. A value of “0” indicates that the treatment did not result in an additional delay in tumor progression.

Treatment with the combination of anti-VEGF-C antibody and anti-VEGF-A antibody resulted in additional delay in tumor progression in A549 and H460 tumors, compared to anti-VEGF-A antibody treatment alone (FIG. 16).

### Example 6

### Identification of Biomarkers for Efficacy of Anti-VEGF-C Antibody Treatment

Gene expression analysis was performed using qRT-PCR on frozen tumor samples obtained from the tumor model experiments described above in Example 5. From frozen material, small cubes of maximal 3 mm side length were solubilized using commercially available reagents and equipment (RNeasy®, Tissuelyzer, both Qiagen Inc., Germany). After column purification RNA was eluted with H₂O, precipitated with ethanol after the addition of glycogen and Sodium acetate. RNA was pelleted by centrifugation for at least 30 min, washed twice with 80% ethanol, and the pellet resuspended in H20 after drying. RNA concentrations were assessed using a spectrophotometer or a bioanalyzer (Agilent, Foster City, Calif.), and 50 ng of total RNA used per reaction in the subsequent gene expression analysis.

Gene specific primer and probe sets were designed for qRT-PCR expression analysis of 18SrRNA, human and mouse RPS13 (housekeeping gene), VEGF-C, VEGF-A, VEGF-D, VEGFR3, FGF2, CSF2, ICAM1, RGS5/CDH5, ESM1, Prox1, PlGF, ITGa5 and TGF-β. The primer and probe set sequences are listed in Table 2.

Relative expression levels of VEGF-C, VEGF-A, VEGF-D, VEGFR3, FGF2, CSF2, ICAM1, RGS5/CDH5, ESM1, Prox1, PlGF, ITGa5 and TGF-β were determined. For example, relative expression level of VEGF-C was calculated as follows:

Relative expression VEGF-Cₛₐₘₚₗₑ=2exp(Ct_{[(18SrRNA+RPS13)/2]}−Ct_{VEGF-C})

with Ct determined in the sample, where Ct is the threshold cycle. The Ct is the cycle number at which the fluorescence generated within a reaction crosses the threshold line.

To allow comparison of results from different reaction plates, relative expression was then calculated as a fraction to the relative expression to an internal reference RNA that was identical in all experimental runs, multiplied by 100:

Normalized relative expression VEGF-Cₛₐₘₚₗₑ=(relative expression VEGF-Cₛₐₘₚₗₑ/relative expression VEGF-C_{reference RNA})×100, where relative expression VEGF-C_{reference RNA}=2exp(Ct_{[(18SrRNA+RPS13)/2]}−Ct_{VEGF-C}) with Ct determined in the reference RNA

Using this calculation, samples that had any signal in the qRT-PCR reaction had values above ‘1’, samples with values below ‘1’ were classed as ‘negative’ for the particular analyte.

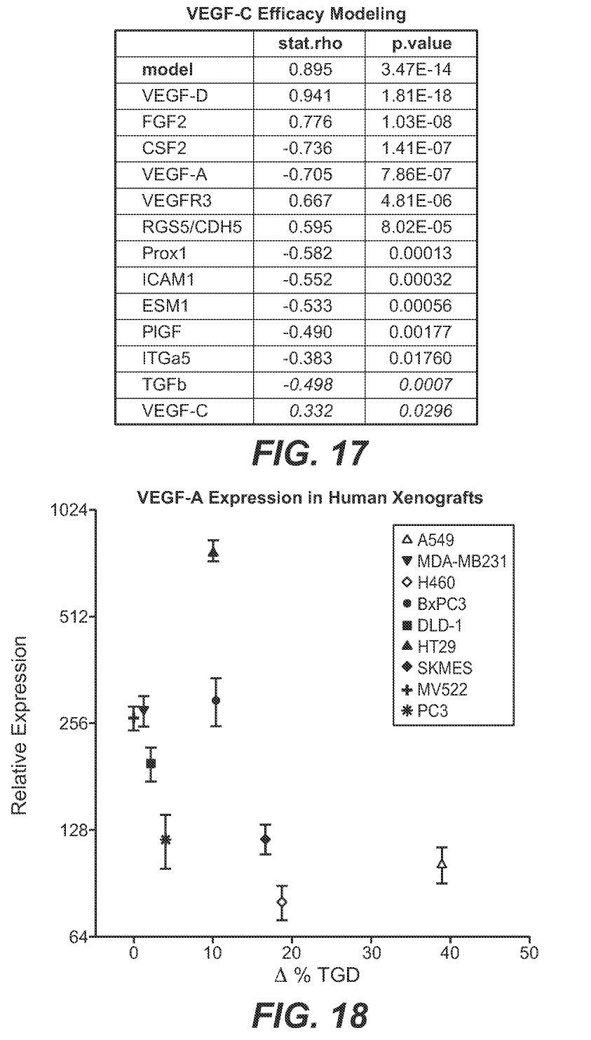

The p- and r-values for the correlation of marker RNA expression (qPCR) and combination treatment efficacy are shown in FIG. 17.

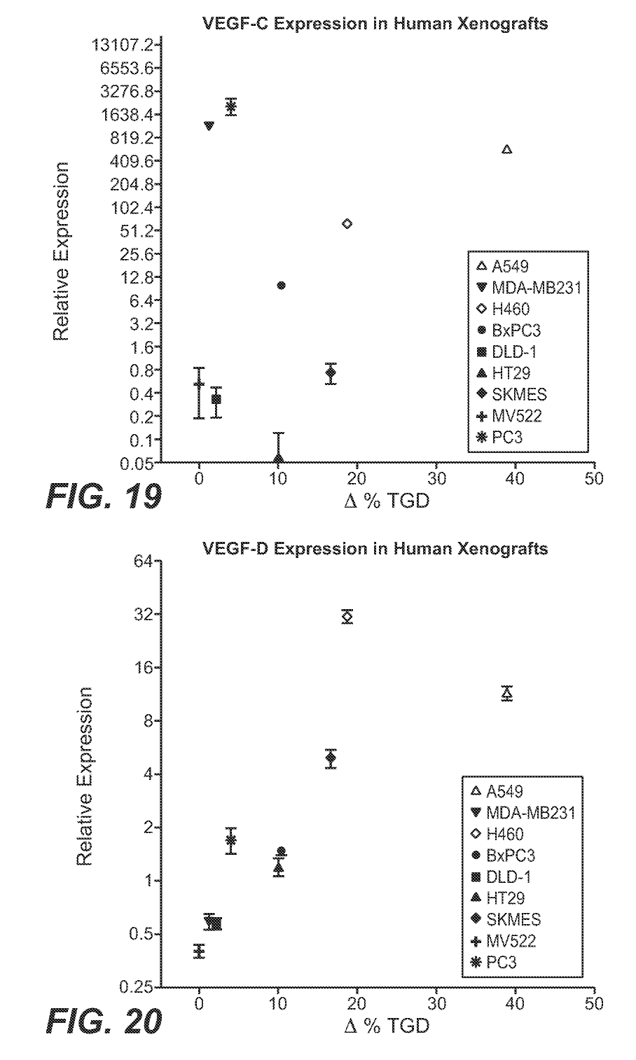

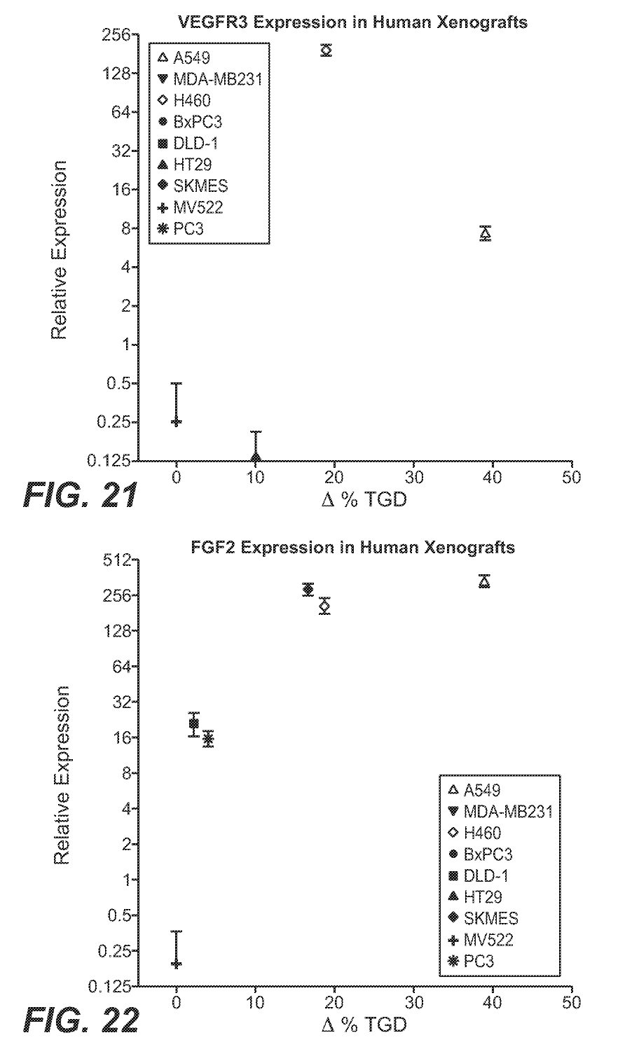

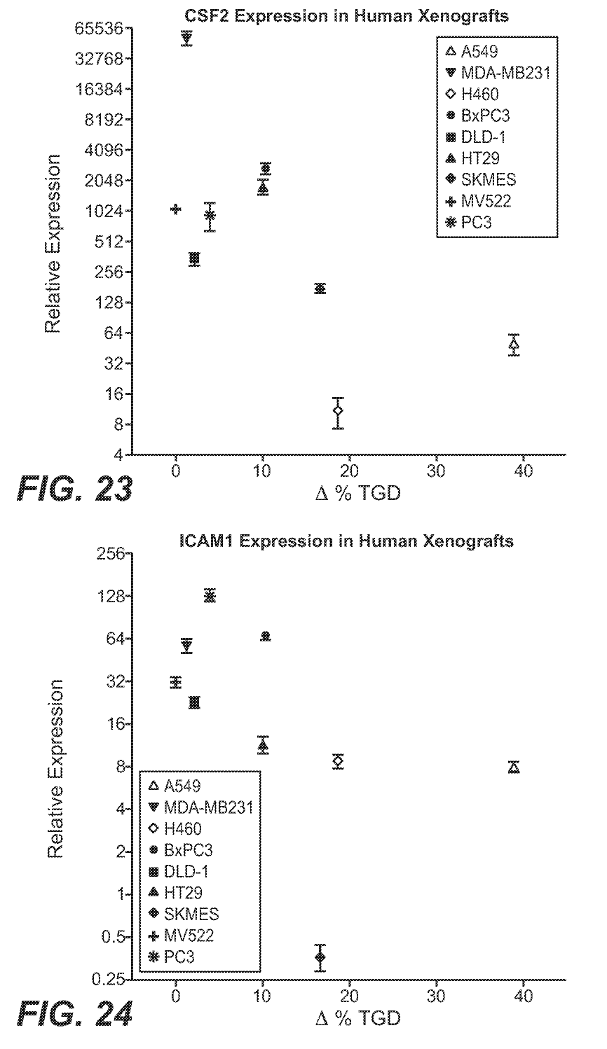

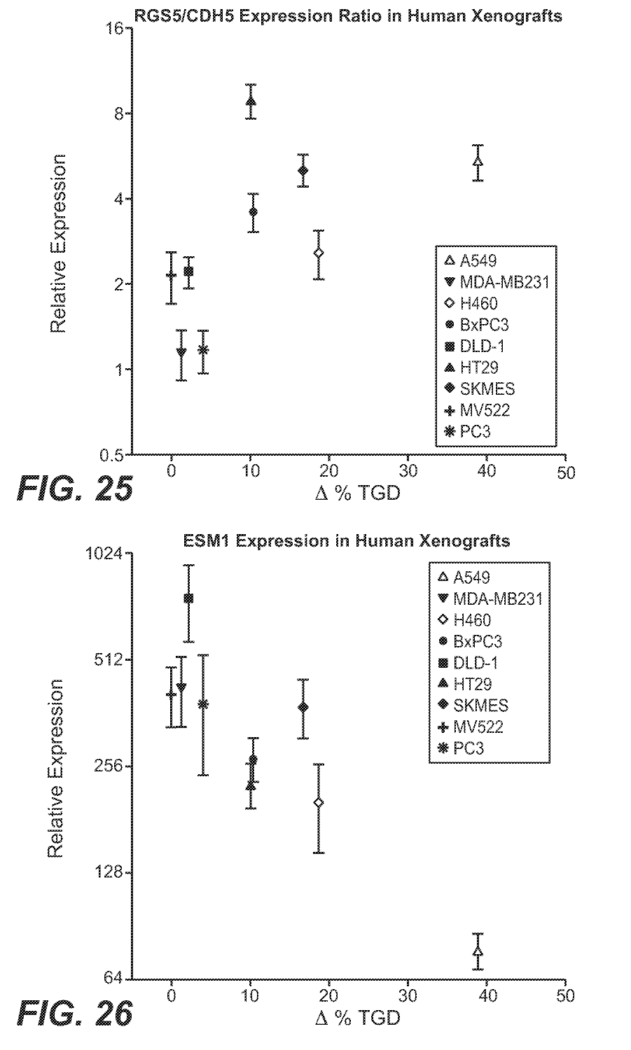

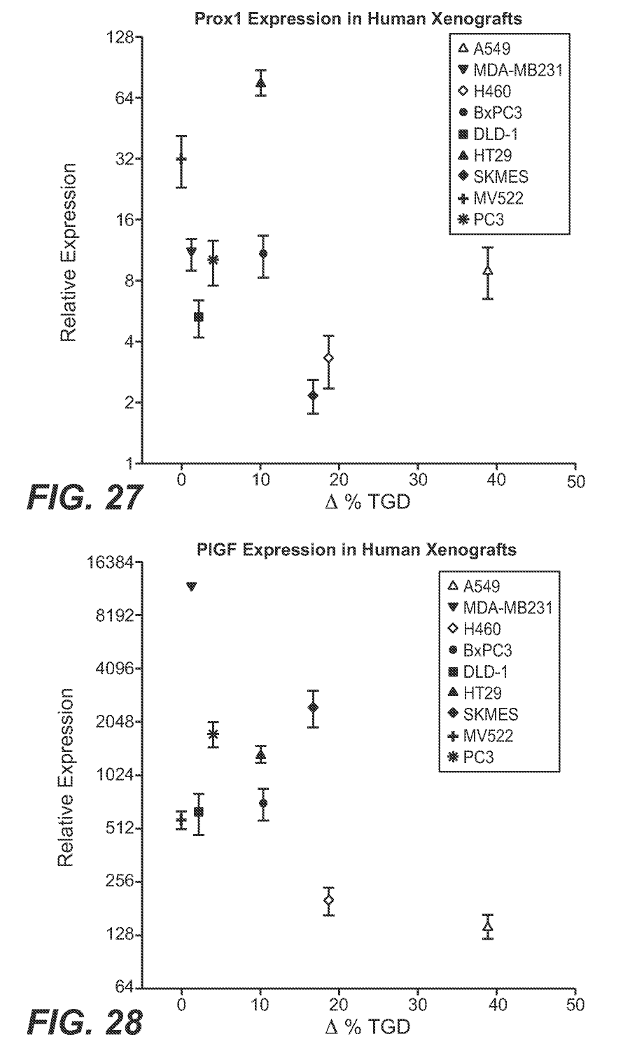

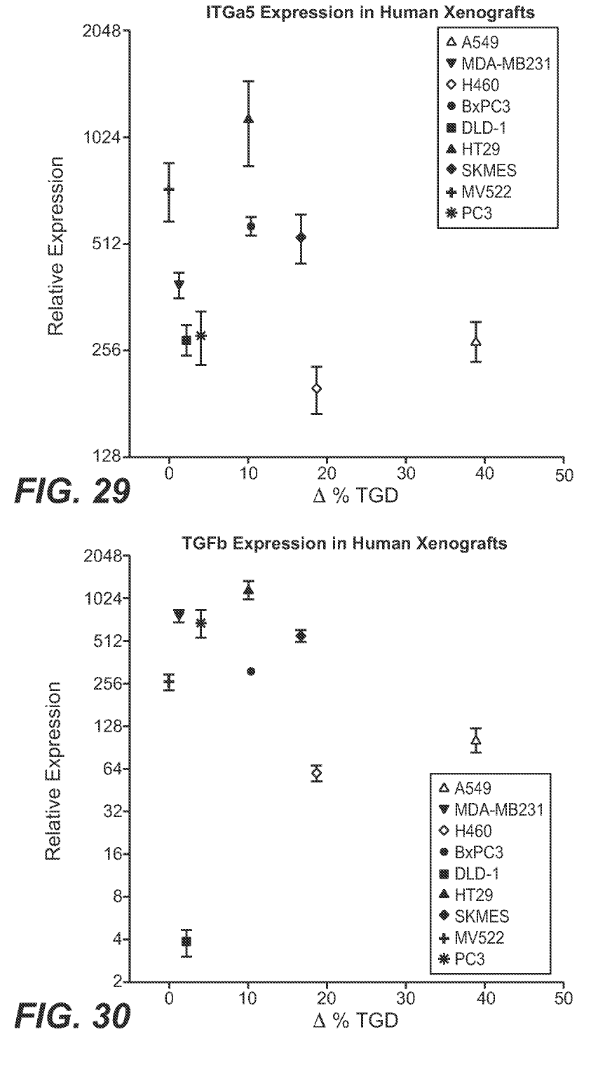

Results from the gene expression analysis are shown in FIGS. 18-30. In each of FIGS. 18-30, the relative expression of the gene assayed is compared to the percent change in tumor growth delay (Δ% TGD) exhibited by the seven different tumor models examined. Tumor models that responded to treatment with anti-VEGF-C antibody in combination with anti-VEGF-A antibody expressed higher levels of VEGF-C, VEGF-D, VEGFR3, FGF2 and RGS5/CDH5 compared to tumor models that did not respond to the combination treatment (see FIGS. 19-22 and 25).

Tumor models responsive to the combination treatment with anti-VEGF-C antibody and anti-VEGF-A antibody also expressed lower levels of VEGF-A, CSF2, Prox1, ICAM1, ESM1, PlGF, ITGa5 and TGFβ as compared to the tumor models that did not respond to the combination treatment (see FIGS. 18, 23-24, and 26-30).

### Example 7

### Tumor Inhibitory Activities of Anti-EGFL7 Antibodies

All studies were conducted in accordance with the Guide for the Care and Use of Laboratory Animals, published by the NIH (NIH Publication 85-23, revised 1985). An Institutional Animal Care and Use Committee (IACUC) approved all animal protocols.

Studies were conducted with the following human tumor models using standardized techniques: A549, MDA-MB231, H460, BxPC3, SKMES, SW620, H1299, MV522 and PC3. Human tumor cells were implanted subcutaneously in the right flank of each test mouse. For example, for A549, xenografts were initiated from cultured A549 human non-small cell lung carcinoma cells (grown to mid-log phase in RPMI-1640 medium containing 10% heat-inactivated fetal bovine serum, 100 units/mL penicillin G, 100 μg/mL streptomycin sulfate, 0.25 μg/mL amphotericin B, 1 mM sodium pyruvate, 2 mM glutamine, 10 mM HEPES, 0.075% sodium bicarbonate, and 25 μg/mL gentamicin) or from A549 human lung adenocarcinoma cells (cultured in Kaighn's modified Ham's F12 medium containing 10% heat-inactivated fetal bovine serum, 100 units/mL penicillin G, 100 μg/mL streptomycin sulfate, 0.25 μg/mL amphotericin B, 2 mM glutamine, 1 mM sodium pyruvate, and 25 μg/mL gentamicin). On the day of tumor implant, A549 cells were harvested and resuspended in PBS at a concentration of 5×10⁷ cells/mL. Each test mouse received 1×10⁷ A549 tumor cells implanted subcutaneously in the right flank. For A549 tumors, A549 cells were resuspended in 100% Matrigel™ matrix (BD Biosciences, San Jose, Calif.) at a concentration of 5×10⁷ cells/mL. A549 cells (1×10⁷ in 0.2 mL) were implanted subcutaneously in the right flank of each test mouse, and tumor growth was monitored.

Tumor growth was monitored as the average size approached 120-180 mm³. On study day 1, individual tumors sizes ranged from 126 to 196 mm³ and the animals were sorted by tumor size into three test groups (one control group and two treatment groups). Tumor volume was calculated using the formula:

Tumor volume (mm³)=(w²×l)/2

where w=width and l=length in mm of the tumor.

All treatments were administered intra-peritoneally. Tumors were treated twice weekly for up to 10-20 weeks with 5-10 mg/kg each of control antibody, an agent blocking VEGF-A activity (anti-VEGF-A antibody B20-4.1 at 5 mg/kg), or the combination of an agent blocking VEGF-A activity and an agent blocking EGFL7 activity (anti-EGFL7 antibody at 10 mg/kg). For the combination treatment group, anti-EGFL7 antibody was administered no later than thirty minutes after administration of the anti-VEGF-A antibody. Each dose was delivered in a volume of 0.2 mL per 20 grams body weight (10 mL/kg), and was scaled to the body weight of the animal.

Tumor volume was recorded twice weekly using calipers. Each animal was euthanized when its tumor reached the endpoint size (generally 1000 mm³) or at the conclusion of the study, whichever came first. Tumors were harvested and either fixated overnight in 10% NBF, followed by 70% ethanol and subsequent embedding in paraffin, or within two minutes frozen in liquid nitrogen for subsequent storage at −80° C.

The time to endpoint (TTE) was calculated from the following equation:

TTE (days)=(log₁₀(endpoint volume,mm³−b)/m

where b is the intercept and m is the slope of the line obtained by linear regression of a log-transformed tumor growth data set.

Animals that did reach the endpoint were assigned a TTE value equal to the last day of the study. Animals classified as NTR (non-treatment-related) deaths due to accident (NTRa) or due unknown causes (NTRu) were excluded from TTE calculations (and all further analyses). Animals classified as TR (treatment-related) deaths or NTRm (non-treatment-related death due to metastasis) were assigned a TTE value equal to the day of death.

Treatment outcome was evaluated by tumor growth delay (TGD), which is defined as the increase in the median time to endpoint (TTE) in a treatment group compared to the control group, which was calculated as follows:

TGD=T−C, expressed in days, or as a percentage of the median TTE of the control group, which was calculated as follows:

% TGD=[(T−C)/C]×100,

where T=median TTE for a treatment group and C=median TTE for the control group.

The Δ% TGD was calculated as above, with C=control group being the group receiving anti-VEGF-A antibody treatment alone, and T=treatment group being the group receiving the combination of anti-VEGF-A antibody and anti-VEGF-C antibody treatment. The logrank test was employed to analyze the significance of the difference between the TTE values of two groups. Two-tailed statistical analyses were conducted at significance level p=0.05. A value of “1” indicates that treatment resulted in an additional delay in tumor progression. A value of “0” indicates that the treatment did not result in an additional delay in tumor progression.

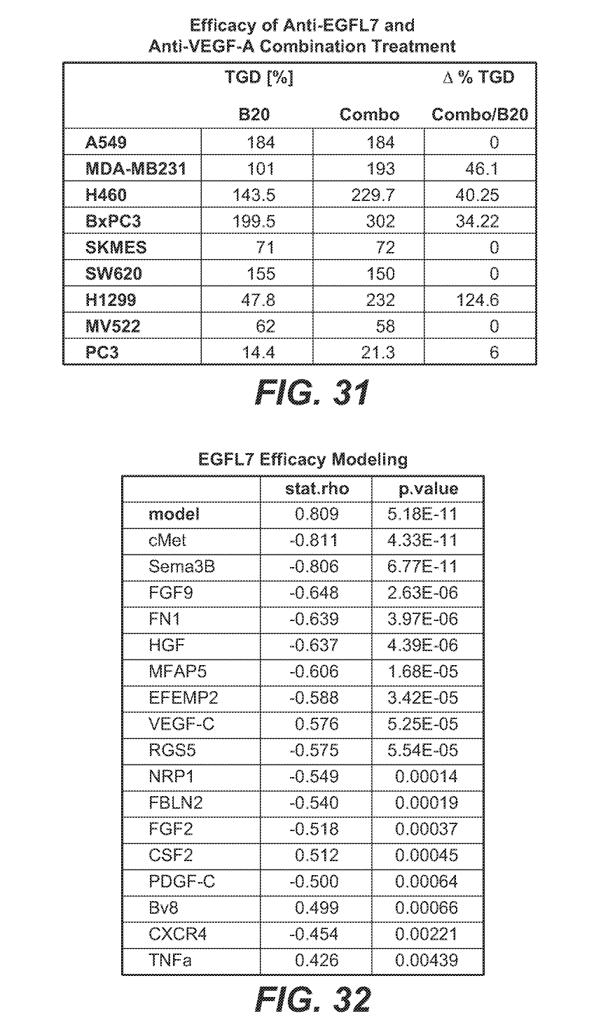

Treatment with the combination of anti-EGFL7 antibody and anti-VEGF-A antibody resulted in additional delay in tumor progression in MDA-MB231, H460, and H1299 tumors, compared to anti-VEGF-A antibody treatment alone (FIG. 31).

### Example 8

### Identification of Biomarkers for Efficacy of Anti-EGFL7 Antibody Treatment

Gene expression analysis was performed using qRT-PCR on frozen tumor samples obtained from the tumor model experiments described above in Example 7. From frozen material, small cubes of maximal 3 mm side length were solubilized using commercially available reagents and equipment (RNeasy®, TissueLyzer, both Qiagen Inc., Germany). After column purification RNA was eluted with H₂O, precipitated with ethanol after the addition of glycogen and sodium acetate. RNA was pelleted by centrifugation for at least 30 min, washed twice with 80% ethanol, and the pellet resuspended in H20 after drying. RNA concentrations were assessed using a spectrophotometer or a bioanalyzer (Agilent, Foster City, Calif.), and 50 ng of total RNA used per reaction in the subsequent gene expression analysis.

Gene specific primer and probe sets were designed for qRT-PCR expression analysis of 18SrRNA, human and mouse RPS13 (housekeeping gene), cMet, Sema3B, FGF9, FN1, HGF, MFAP5, EFEMP2/fibulin4, VEGF-C, RGS5, NRP1, FBLN2, FGF2, CSF2, PDGF-C, BV8, CXCR4, and TNFa. The primer and probe set sequences are listed in Table 2.

Relative expression levels of cMet, Sema3B, FGF9, FN1, HGF, MFAP5, EFEMP2/fibulin4, VEGF-C, RGS5, NRP1, FBLN2, FGF2, CSF2, PDGF-C, BV8, CXCR4, and TNFa were determined. For example, relative expression level of VEGF-C was calculated as follows:

Relative expression VEGF-Cₛₐₘₚₗₑ=2exp(Ct_{[(18SrRNA+RPS13)/2]}−Ct_{VEGF-C})

with Ct determined in the sample, where Ct is the threshold cycle. The Ct is the cycle number at which the fluorescence generated within a reaction crosses the threshold line.

To allow comparison of results from different reaction plates, relative expression was then calculated as a fraction to the relative expression to an internal reference RNA that was identical in all experimental runs, multiplied by 100:

Normalized relative expression VEGF-Cₛₐₘₚₗₑ=(relative expression VEGF-Cₛₐₘₚₗₑ/relative expression VEGF-C_{reference RNA})×100, where relative expression VEGF-C_{reference RNA}=2exp(Ct_{[(18SrRNA+RPS13)/2]}−Ct_{VEGF-C}) with Ct determined in the reference RNA

Using this calculation, samples that had any signal in the qRT-PCR reaction had values above ‘1’, samples with values below ‘1’ were classed as ‘negative’ for the particular analyte.

The p- and r-values for the correlation of marker RNA expression (qPCR) and combination treatment efficacy are shown in FIG. 32.

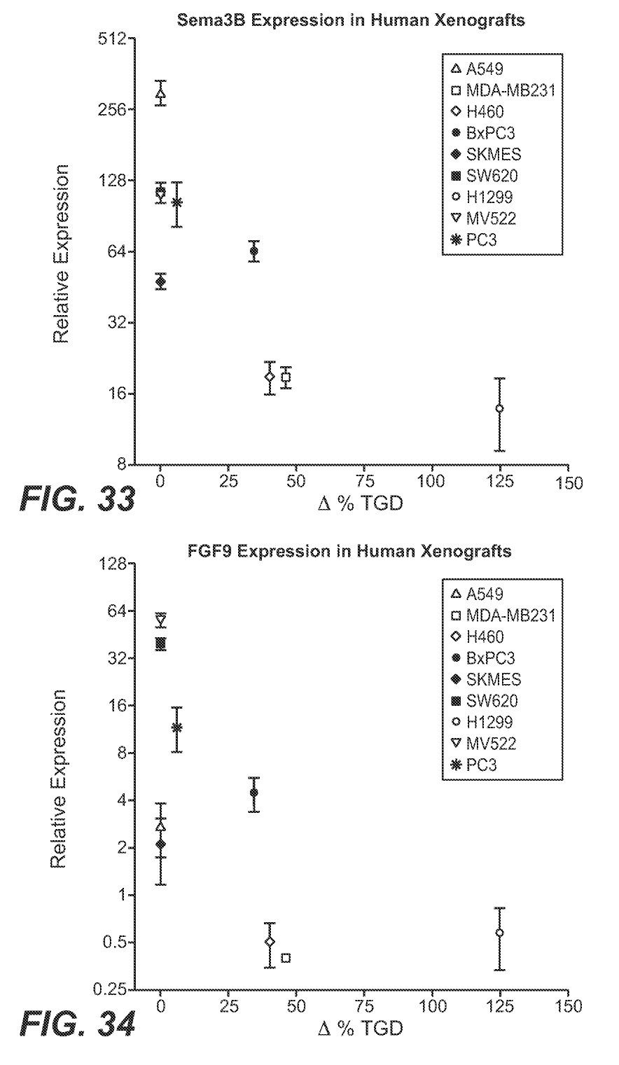

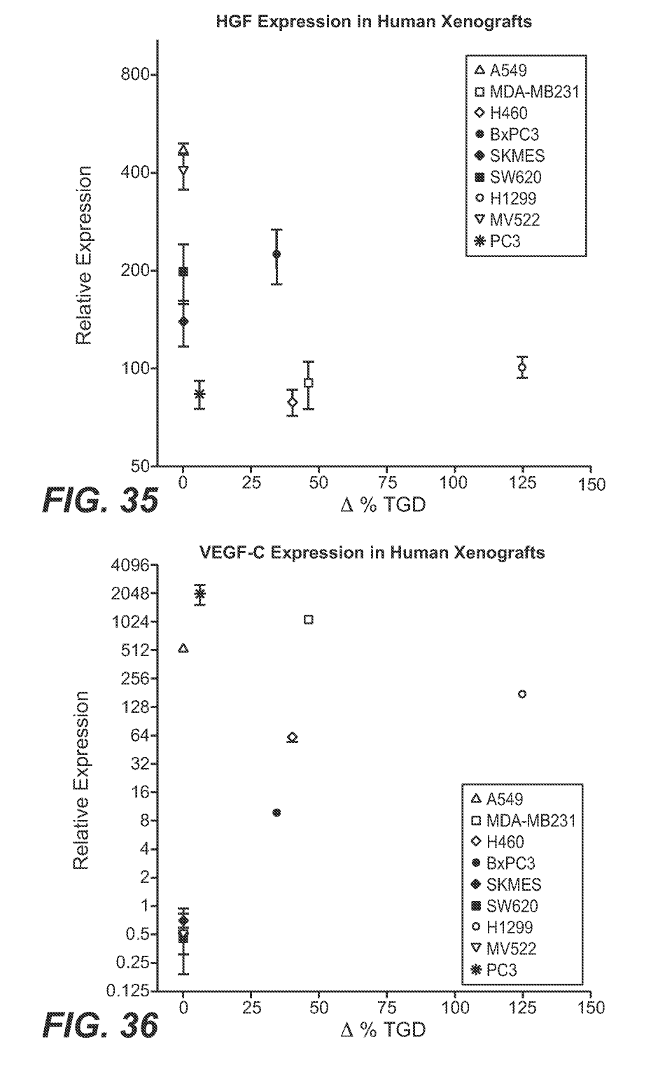

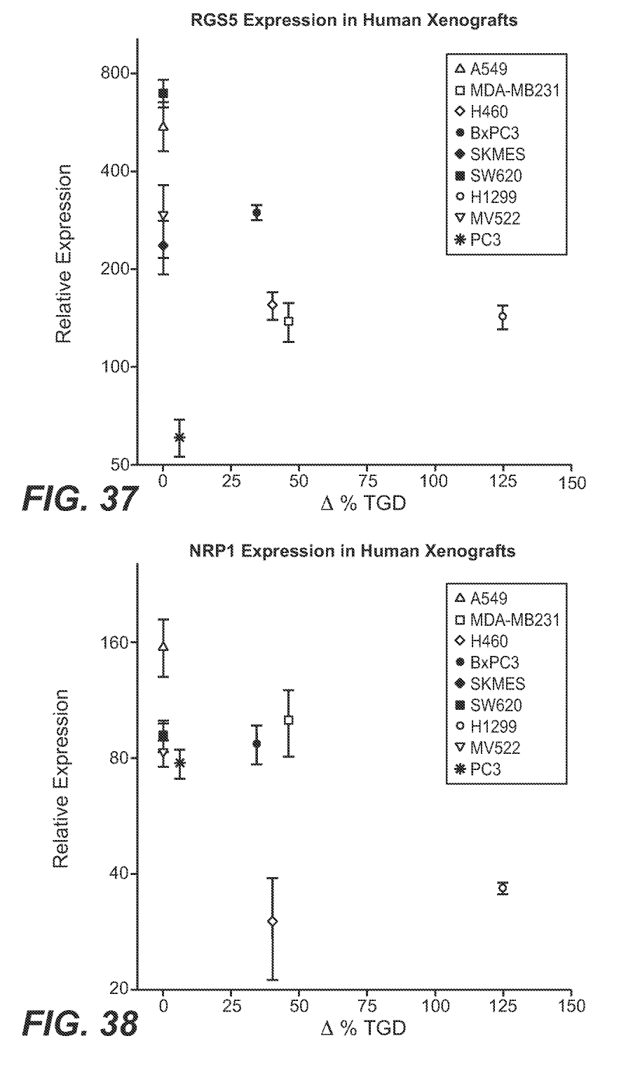

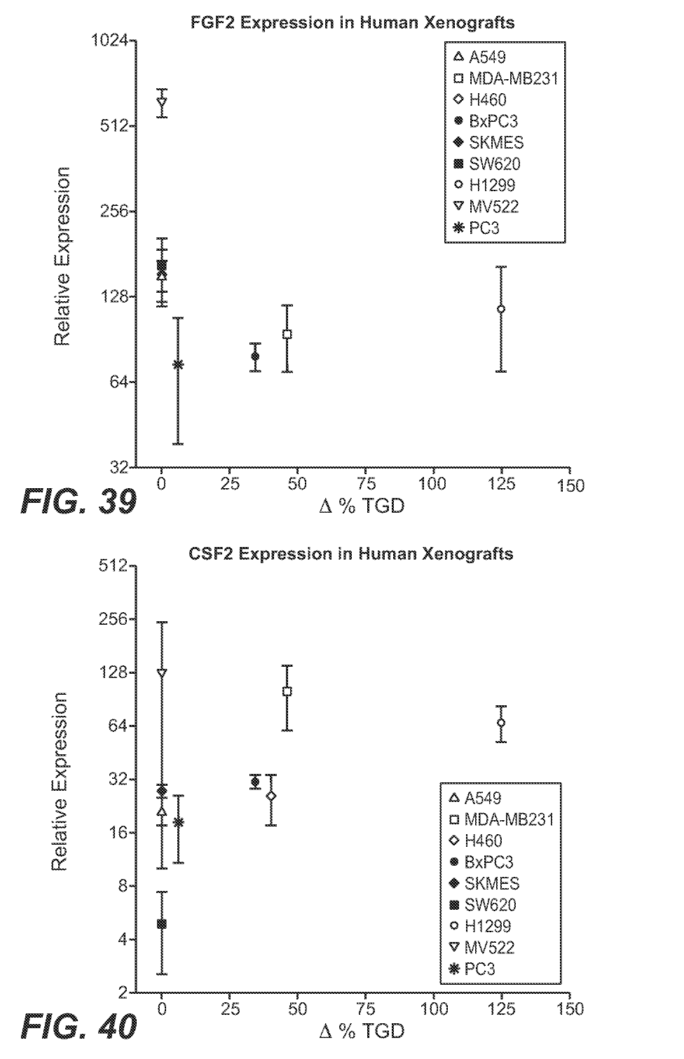

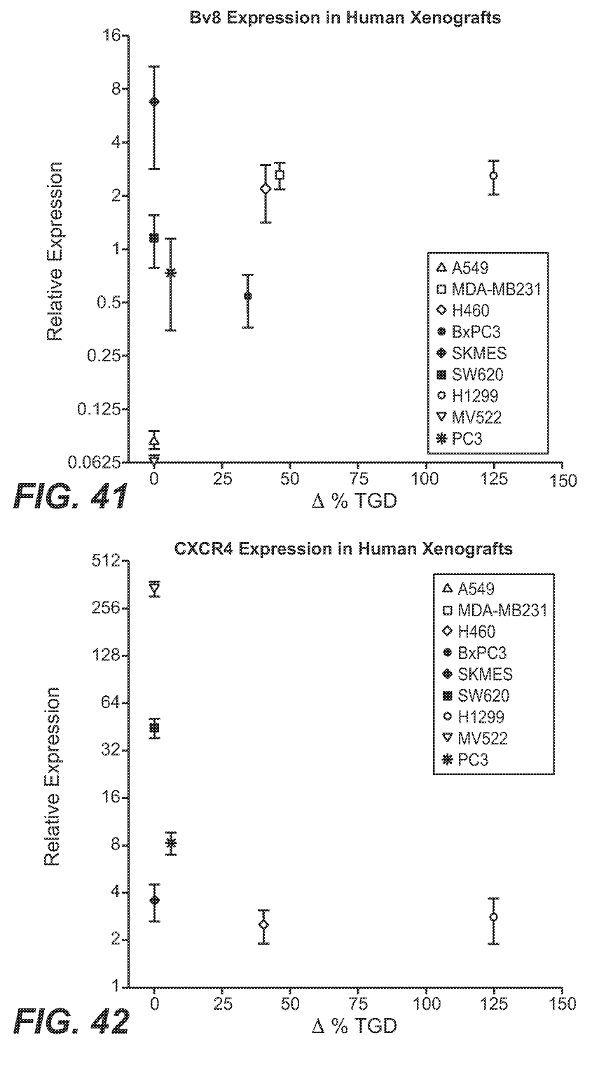

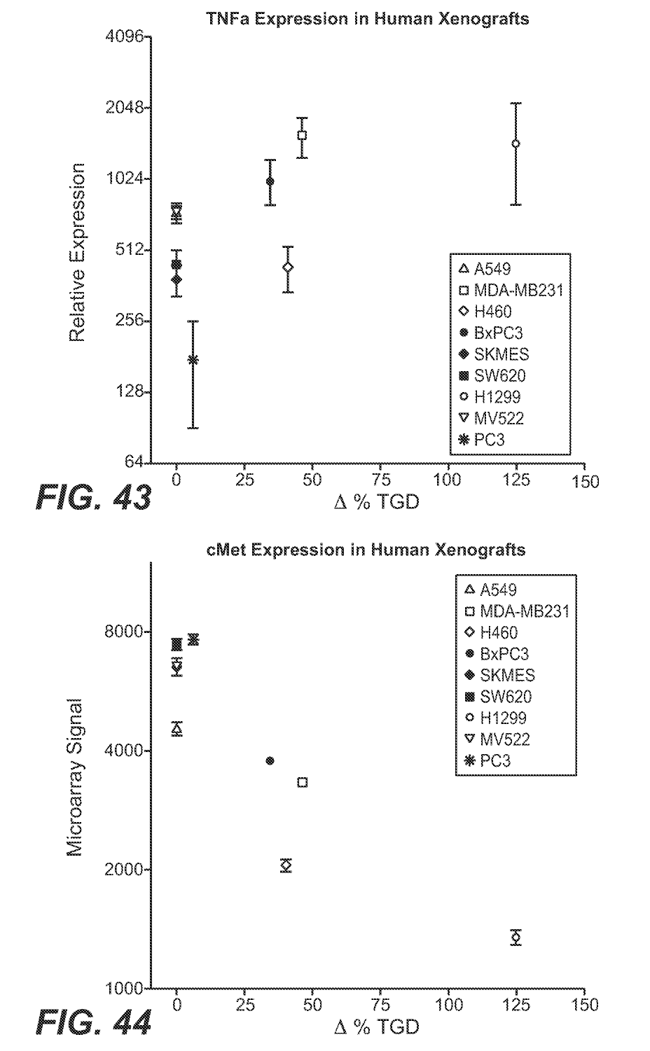

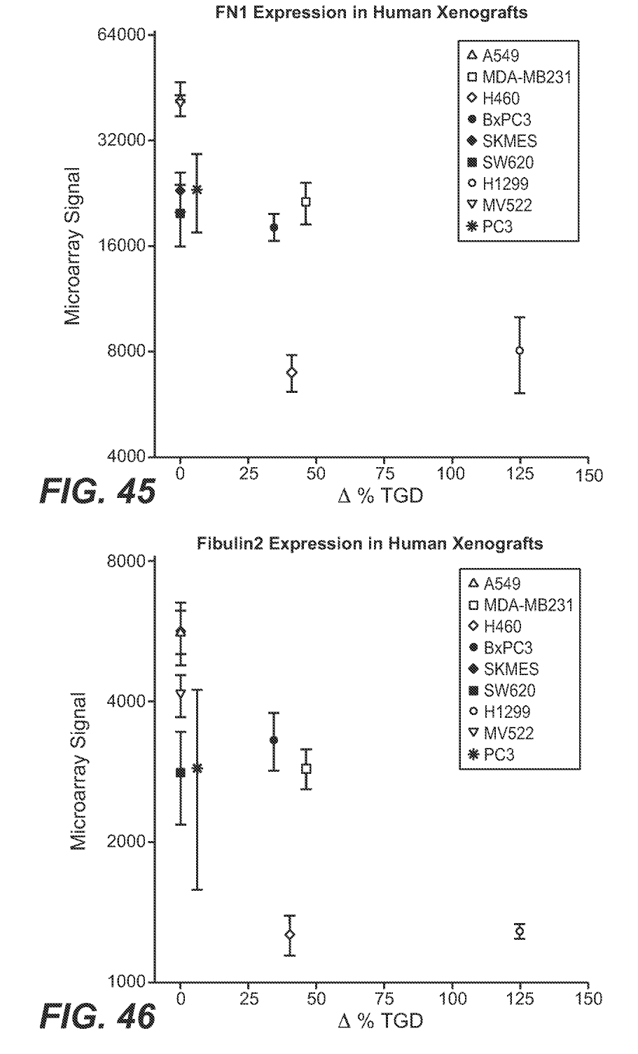

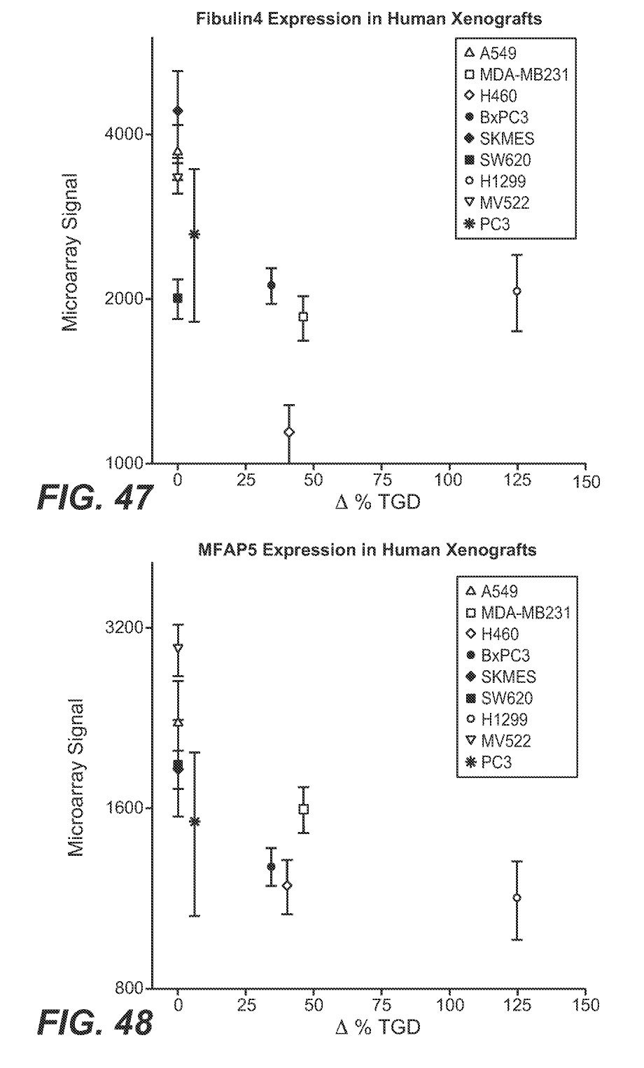

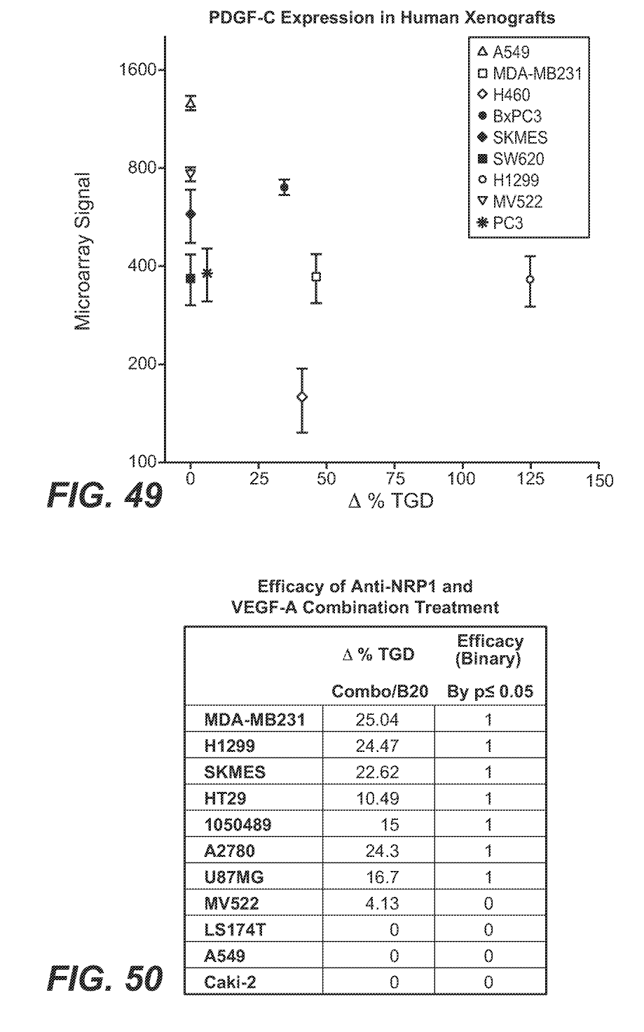

Results from the gene expression analysis are shown in FIGS. 33-49. In each of FIGS. 33-49, the relative expression of the gene assayed is compared to the percent change in tumor growth delay (Δ% TGD) exhibited by the nine different tumor models examined. Tumor models that responded to treatment with anti-EGFL7 antibody in combination with anti-VEGF-A antibody expressed higher levels of VEGF-C, BV8, CSF2 and TNFa compared to tumor models that did not respond to the combination treatment (see FIGS. 36, 40, 41, and 43).

Tumor models responsive to the combination treatment with anti-VEGF-C antibody and anti-EGFL7 antibody also expressed lower levels of Sema3B, FGF9, HGF, RGS5, NRP1, FGF2, CXCR4, cMet, FN1, Fibulin 2, Fibulin4, MFAP5, PDGF-C and Sema3F as compared to the tumor models that did not respond to the combination treatment (see FIGS. 33-35, 37-39, 42, and 44-49).

### Example 9

### Tumor Inhibitory Activities of Anti-NRP1 Antibodies

All studies were conducted in accordance with the Guide for the Care and Use of Laboratory Animals, published by the NIH (NIH Publication 85-23, revised 1985). An Institutional Animal Care and Use Committee (IACUC) approved all animal protocols.

Studies were conducted with the following human tumor models using standardized techniques: MDA-MB231, H1299, SKMES, HT29, 1050489, A2780, U87MG, MV522, LS174t, A549, and Caki-2. Human tumor cells were implanted subcutaneously in the right flank of each test mouse. For example, for H1299, xenografts were initiated from cultured H1299 human non-small cell lung carcinoma cells (grown to mid-log phase in RPMI-1640 medium containing 10% heat-inactivated fetal bovine serum, 100 units/mL penicillin G, 100 μg/mL streptomycin sulfate, 0.25 μg/mL amphotericin B, 1 mM sodium pyruvate, 2 mM glutamine, 10 mM HEPES, 0.075% sodium bicarbonate, and 25 μg/mL gentamicin) or from A549 human lung adenocarcinoma cells (cultured in Kaighn's modified Ham's F12 medium containing 10% heat-inactivated fetal bovine serum, 100 units/mL penicillin G, 100 μg/mL streptomycin sulfate, 0.25 μg/mL amphotericin B, 2 mM glutamine, 1 mM sodium pyruvate, and 25 μg/mL gentamicin). On the day of tumor implant, H1299 cells were harvested and resuspended in PBS at a concentration of 5×10⁷ cells/mL. Each test mouse received 1×10⁷H1299 tumor cells implanted subcutaneously in the right flank. For A549 tumors, A549 cells were resuspended in 100% Matrigel™ matrix (BD Biosciences, San Jose, Calif.) at a concentration of 5×10⁷ cells/mL. A549 cells (1×10⁷ in 0.2 mL) were implanted subcutaneously in the right flank of each test mouse, and tumor growth was monitored. As another example, a fragment of a 1050489 tumor was implanted into the right flank of each test mouse and tumor growth was monitored.

Tumor growth was monitored as the average size approached 120-180 mm³. On study day 1, individual tumors sizes ranged from 126 to 196 mm³ and the animals were sorted by tumor size into three test groups (one control group and two treatment groups). Tumor volume was calculated using the formula:

Tumor volume (mm³)==(w²×l)/2

where w=width and l=length in mm of the tumor.

All treatments were administered intra-peritoneally. Tumors were treated twice weekly for up to 10-20 weeks with 5-10 mg/kg each of control antibody, an agent blocking VEGF-A activity (anti-VEGF-A antibody B20-4.1 at 5 mg/kg), or the combination of an agent blocking VEGF-A activity and an agent blocking NRP1 activity (anti-NRP1 antibody at 10 mg/kg). For the combination treatment group, anti-NRP1 antibody was administered no later than thirty minutes after administration of the anti-VEGF-A antibody. Each dose was delivered in a volume of 0.2 mL per 20 grams body weight (10 mL/kg), and was scaled to the body weight of the animal.

Tumor volume was recorded twice weekly using calipers. Each animal was euthanized when its tumor reached the endpoint size (generally 1000 mm³) or at the conclusion of the study, whichever occurred first.

The time to endpoint (TTE) was calculated from the following equation:

TTE (days)=(log₁₀(endpoint volume,mm³−b)/m

where b is the intercept and m is the slope of the line obtained by linear regression of a log-transformed tumor growth data set.

Animals that did reach the endpoint were assigned a TTE value equal to the last day of the study. Animals classified as NTR (non-treatment-related) deaths due to accident (NTRa) or due unknown causes (NTRu) were excluded from TTE calculations (and all further analyses). Animals classified as TR (treatment-related) deaths or NTRm (non-treatment-related death due to metastasis) were assigned a TTE value equal to the day of death. Tumor were harvested and either fixated overnight in 10% NBF, followed by 70% ethanol and subsequent embedding in paraffin, or within two minutes frozen in liquid nitrogen for subsequent storage at −80° C.

Treatment outcome was evaluated by tumor growth delay (TGD), which is defined as the increase in the median time to endpoint (TTE) in a treatment group compared to the control group, which was calculated as follows:

TGD=T−C, expressed in days, or as a percentage of the median TTE of the control group, which was calculated as follows:

% TGD=[(T−C)/C]×100,

where T=median TTE for a treatment group and C=median TTE for the control group.

The Δ% TGD was calculated as above, with C=control group being the group receiving anti-VEGF-A treatment alone, and T=treatment group being the group receiving the combination of anti-VEGF-A and anti-NRP1 treatment. The logrank test was employed to analyze the significance of the difference between the TTE values of two groups. Two-tailed statistical analyses were conducted at significance level p=0.05. A value of “1” indicates that treatment resulted in an additional delay in tumor progression. A value of “0” indicates that the treatment did not result in an additional delay in tumor progression.

Treatment with the combination of anti-NRP1 antibody and anti-VEGF-A antibody resulted in additional delay in tumor progression in MDA-MB231, H1299, SKMES, HT29, 1050489, A2780, and U87MG tumors, compared to anti-VEGF treatment alone (FIG. 50).

### Example 10

### Identification of Biomarkers for Efficacy of Anti-NRP1 Antibody Treatment

Gene expression analysis was performed using qRT-PCR on frozen tumor samples obtained from the tumor model experiments described above in Example 9. From frozen material, small cubes of maximal 3 mm side length were solubilized using commercially available reagents and equipment (RNeasy®, Tissuelyzer, both Qiagen Inc, Germany). After column purification RNA was eluted with H₂O, precipitated with ethanol after the addition of glycogen and Sodium acetate. RNA was pelleted by centrifugation for at least 30 min, washed twice with 80% ethanol, and the pellet resuspended in H₂0 after drying. RNA concentrations were assessed using a spectrophotometer or a bioanalyzer (Agilent, Foster City, Calif.), and 50 ng of total RNA used per reaction in the subsequent gene expression analysis.

Gene specific primer and probe sets set forth in Example 1 above were used for qRT-PCR expression analysis of 18SrRNA, RPS13, HMBS, ACTB, and SDHA (housekeeping genes) and SEMA3B, TGFB1, FGFR4, Vimentin, SEMA3A, PLC, CXCL5, ITGa5, PLGF, CCL2, IGFBP4, LGALS1, HGF, TSP1, CXCL1, CXCL2, Alk1, and FGF8.

Relative expression levels of SEMA3B, TGFB1, FGFR4, Vimentin, SEMA3A, PLC, CXCL5, ITGa5, PLGF, CCL2, IGFBP4, LGALS1, HGF, TSP1, CXCL1, CXCL2, Alk1, and FGF8 was determined. For example, relative expression level of SEMA3B was calculated as follows:

Relative expression SEMA3Bₛₐₘₚₗₑ=2exp(Ct_{[(HK1+HK2+HKx)/x]}−Ct_{SEMA3B});

where HK is a housekeeping gene (e.g., 18sRNA, ACTB, RPS13, HMBS, SDHA, OR UBC), and x is the total number of housekeeping genes used to normalize the data with Ct determined in the sample, where Ct is the threshold cycle. The Ct is the cycle number at which the fluorescence generated within a reaction crosses the threshold line.

To allow comparison of results from different reaction plates, relative expression was then calculated as a fraction to the relative expression to an internal reference RNA that was identical in all experimental runs:

Normalized relative expression SEMA3Bₛₐₘₚₗₑ=(relative expression SEMA3Bₛₐₘₚₗₑ/relative expression SEMA3B_{reference RNA}), where relative expression SEMA3Bₛₐₘₚₗₑ=2exp(Ct_{[(HK1+HK2+HKx)/x]}−Ct_{SEMA3B}) with Ct determined in the reference RNA.

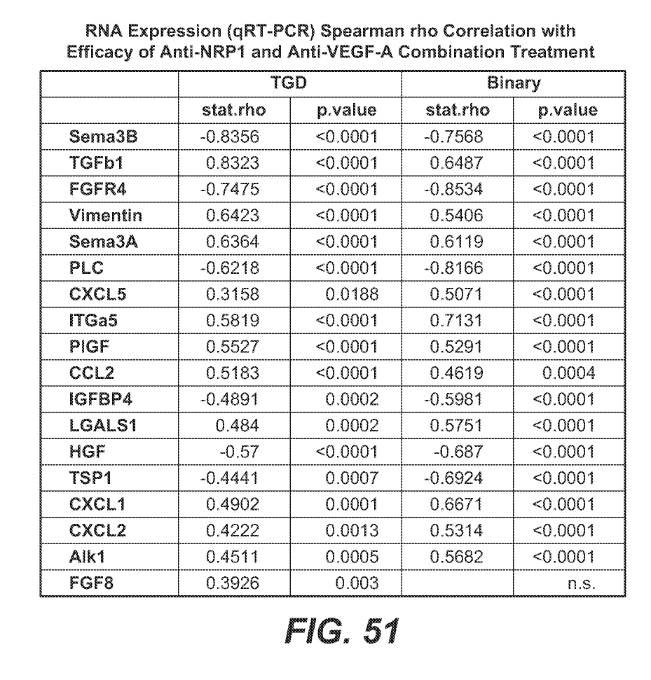

The p- and r-values for the correlation of marker RNA expression (qPCR) and combination treatment efficacy are shown in FIG. 51.

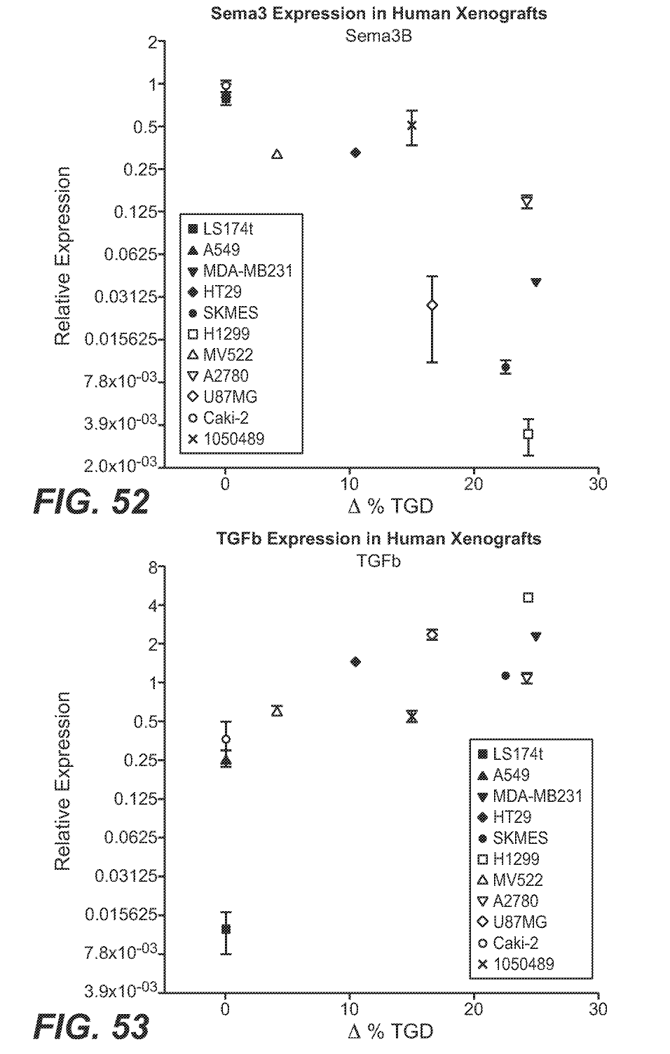

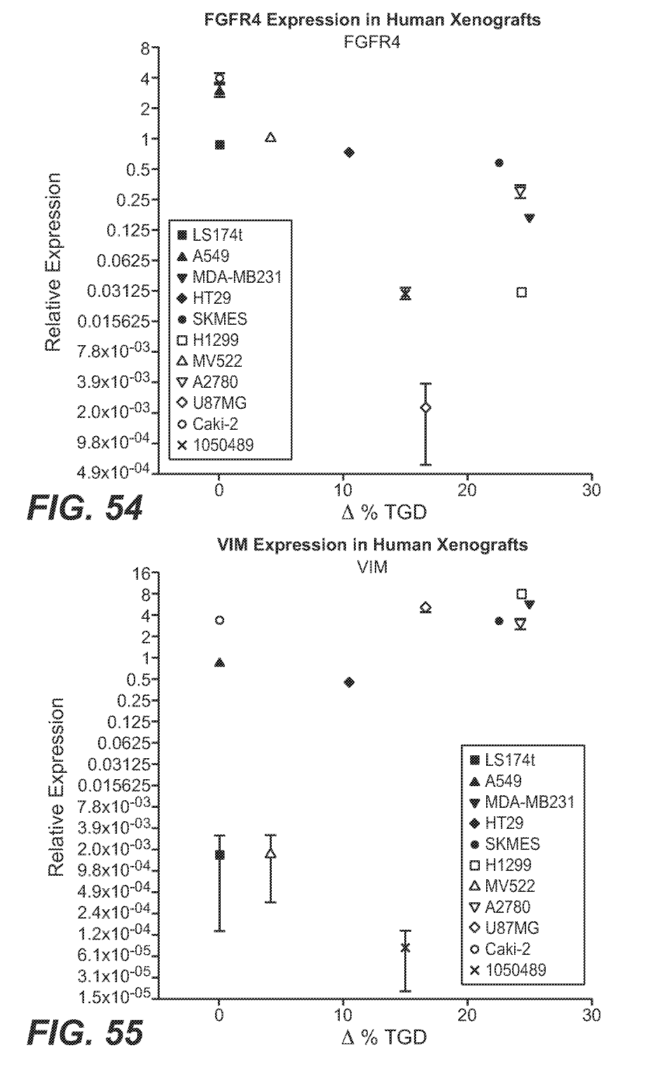

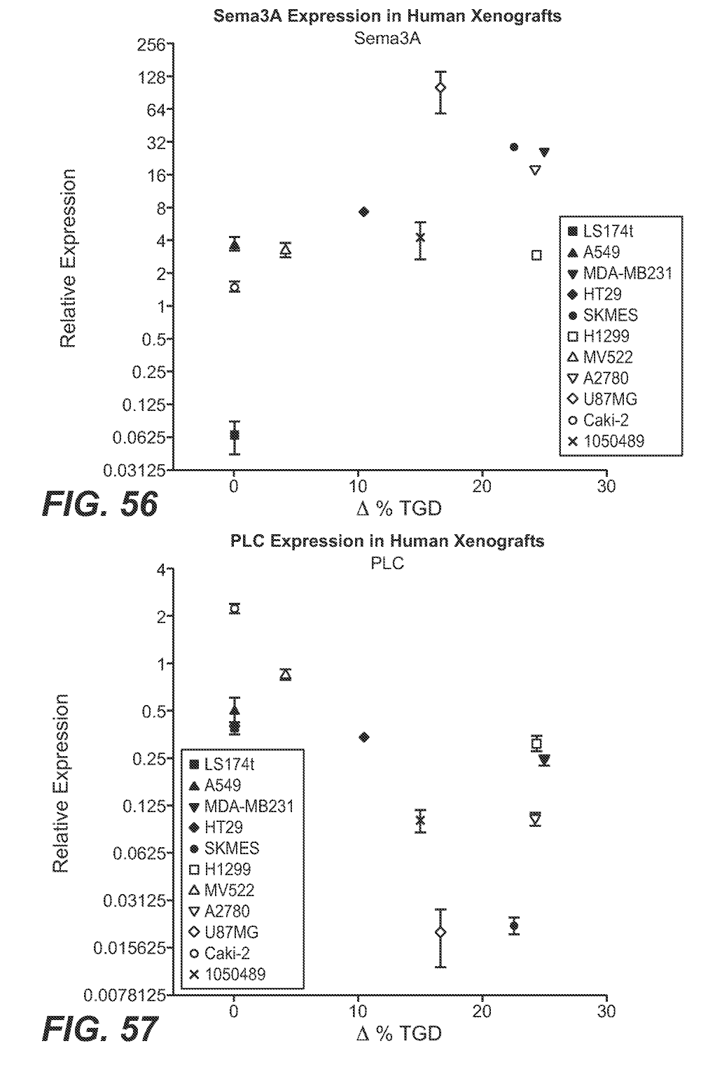

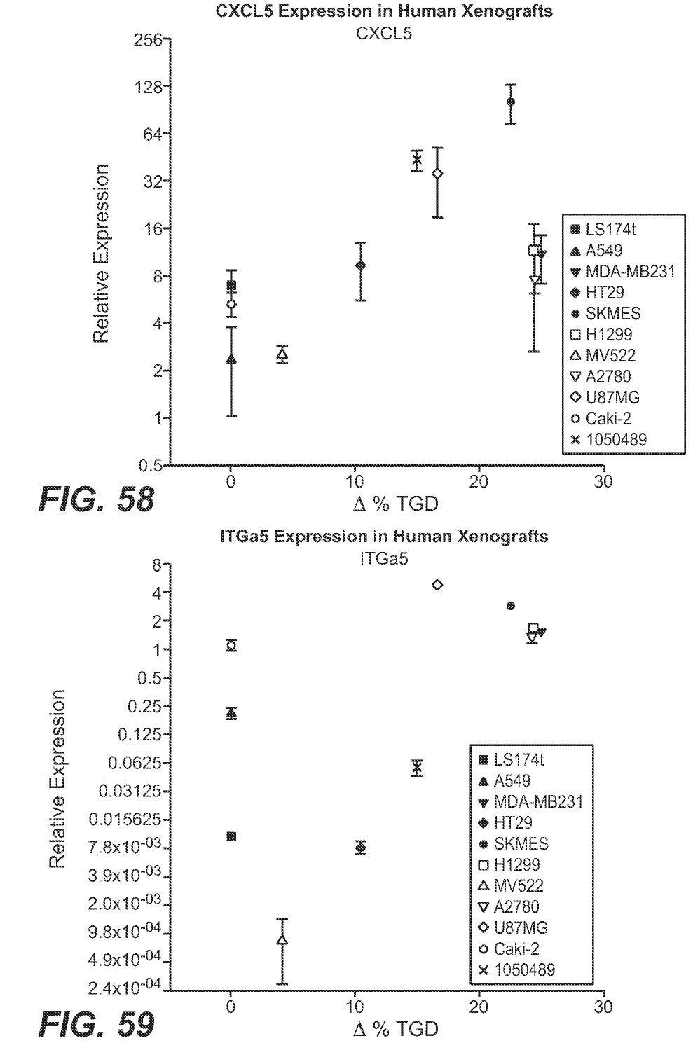

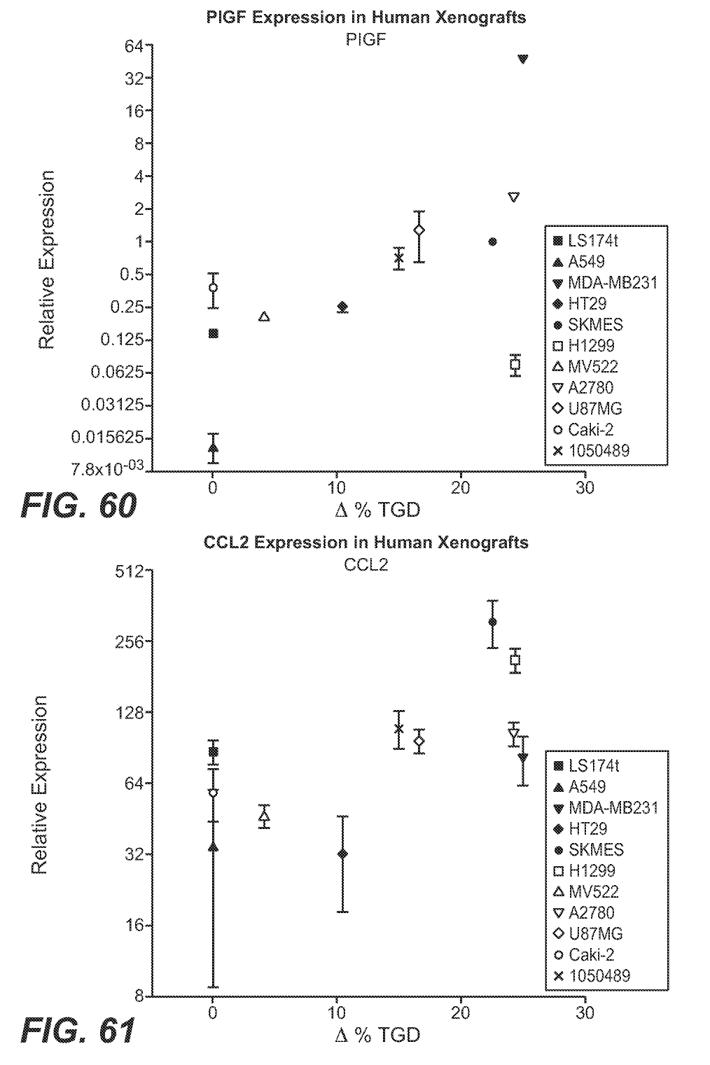

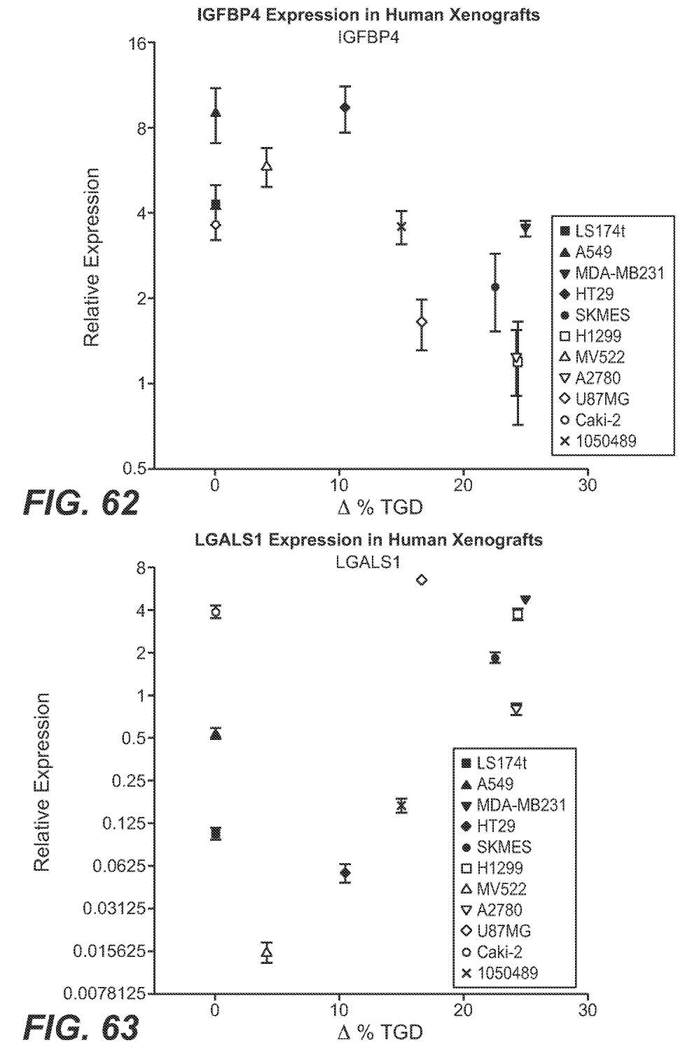

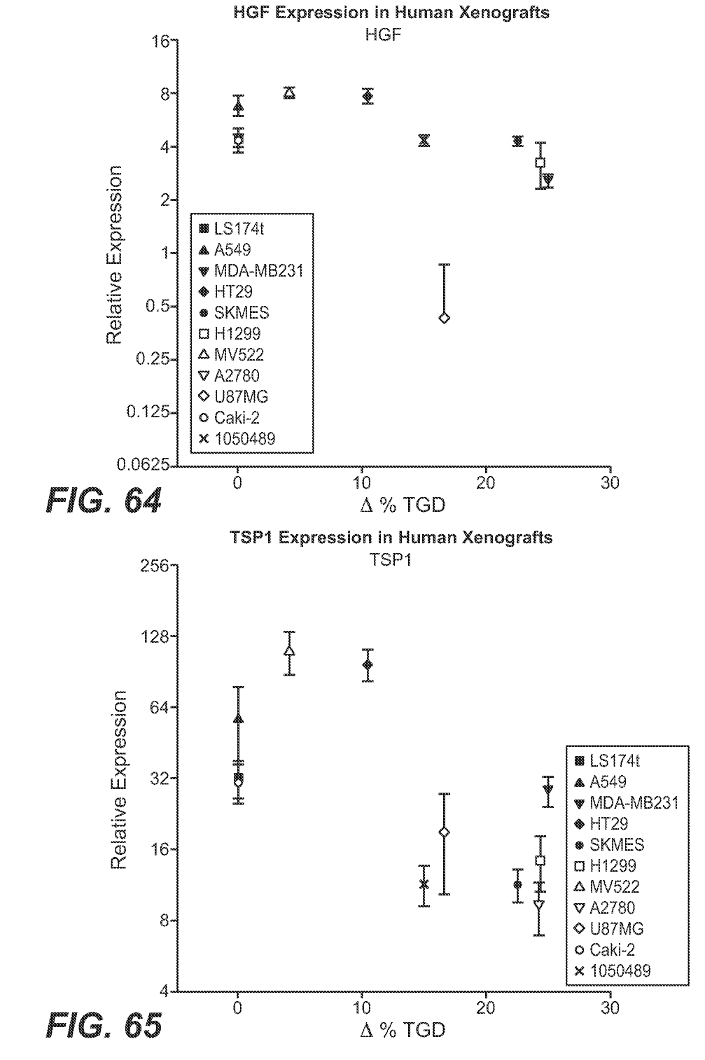

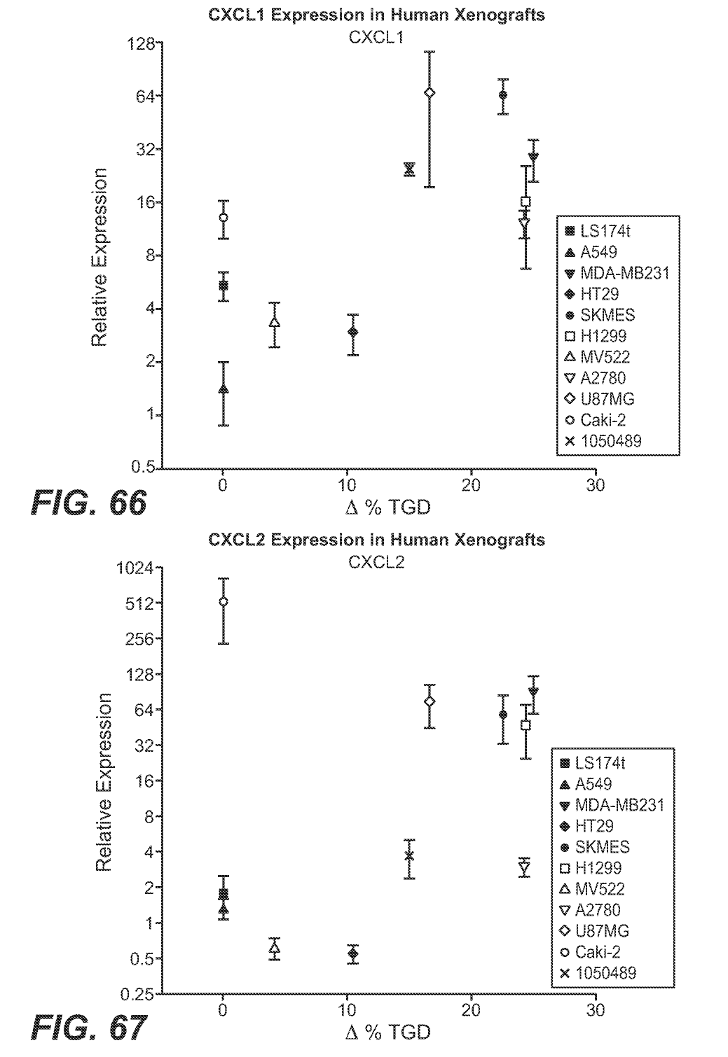

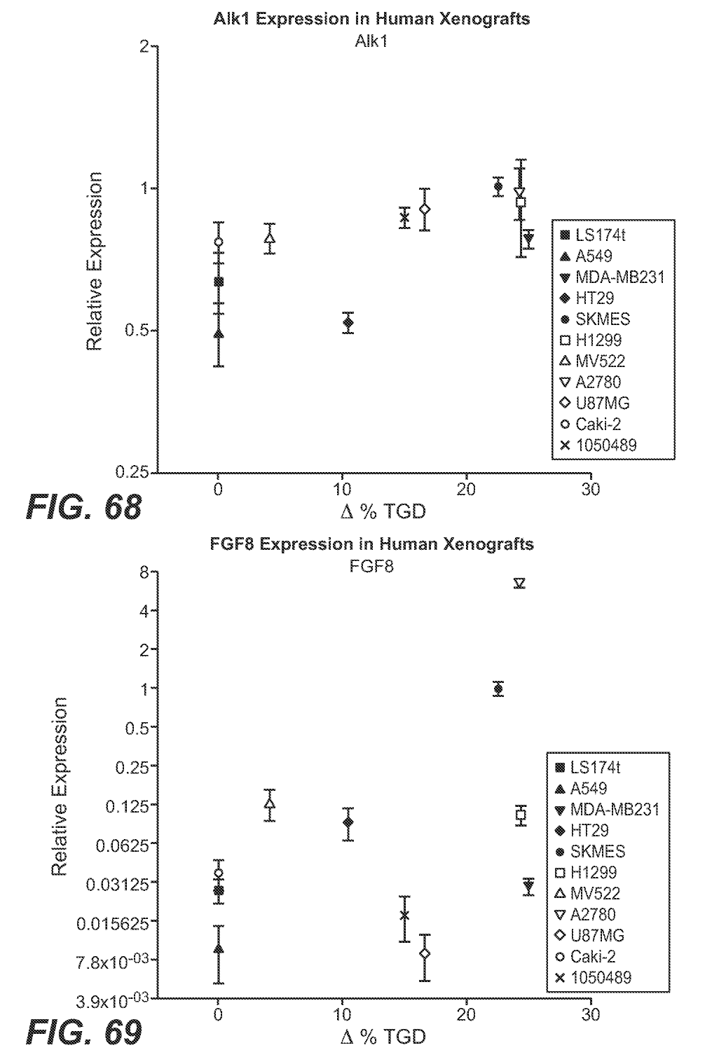

Results from the gene expression analysis are shown in FIGS. 52-69. In each of FIGS. 52-69, the relative expression of the gene assayed is compared to the percent change in tumor growth delay (Δ% TGD) exhibited by the seven different tumor models examined.

Tumor models that responded to treatment with anti-NRP1 antibody in combination with anti-VEGF-A antibody expressed higher levels of TGFβ1, Vimentin, Sema3A, CXCL5, ITGa5, PlGF, CCL2, LGALS1, CXCL2, Alk1, and FGF8 compared to tumor models that did not respond to the combination treatment (see FIGS. 53, 55-56, 58-61, 63, and 66-69).

Tumor models responsive to the combination treatment with anti-NRP1 antibody and anti-VEGF-A antibody also expressed lower levels of Sema3B, FGRF4, PLC, IGFB4, HGF, and TSP1 as compared to the tumor models that did not respond to the combination treatment (see FIGS. 52, 54, 57, 62, and 64-65).

### Example 11

### Tumor Inhibitory Activities of Anti-VEGF-C Antibodies

All studies were conducted in accordance with the Guide for the Care and Use of Laboratory Animals, published by the NIH (NIH Publication 85-23, revised 1985). An Institutional Animal Care and Use Committee (IACUC) approved all animal protocols.

Studies were conducted with the following human tumor models using standardized techniques: A549, MDA-MB231, H460, BxPC3, DLD-1, HT29, SKMES, MV522, PC3, LXFE409, LXFL1674, LXFA629, LXFA737, LXFA1335, CXF243, CXF260, MAXF583, MEXF989, BXF1218, BXF1352, and SXF463. Human tumor cells were implanted subcutaneously in the right flank of each test mouse. For example, for A549, xenografts were initiated from cultured A549 human non-small cell lung carcinoma cells (grown to mid-log phase in RPMI-1640 medium containing 10% heat-inactivated fetal bovine serum, 100 units/mL penicillin G, 100 μg/mL streptomycin sulfate, 0.25 μg/mL amphotericin B, 1 mM sodium pyruvate, 2 mM glutamine, 10 mM HEPES, 0.075% sodium bicarbonate, and 25 μg/mL gentamicin) or from A549 human lung adenocarcinoma cells (cultured in Kaighn's modified Ham's F12 medium containing 10% heat-inactivated fetal bovine serum, 100 units/mL penicillin G, 100 μg/mL streptomycin sulfate, 0.25 μg/mL amphotericin B, 2 mM glutamine, 1 mM sodium pyruvate, and 25 μg/mL gentamicin). On the day of tumor implant, A549 cells were harvested and resuspended in PBS at a concentration of 5×10⁷ cells/mL. Each test mouse received 1×10⁷ A549 tumor cells implanted subcutaneously in the right flank. For A549 tumors, A549 cells were resuspended in 100% Matrigel™ matrix (BD Biosciences, San Jose, Calif.) at a concentration of 5×10⁷ cells/mL. A549 cells (1×10⁷ in 0.2 mL) were implanted subcutaneously in the right flank of each test mouse, and tumor growth was monitored. As another example, a fragment of a LXFA629 tumor was implanted into the right flank of each test mouse and tumor growth was monitored.

Tumor growth was monitored as the average size approached 120-180 mm³. On study day 1, individual tumors sizes ranged from 126 to 196 mm³ and the animals were sorted by tumor size into three test groups (one control group and two treatment groups). Tumor volume was calculated using the formula:

Tumor volume (mm³)==(w²×l)/2

where w=width and l=length in mm of the tumor.

All treatments were administered intra-peritoneally. Tumors were treated twice weekly for up to 10-20 weeks with 5-10 mg/kg each of control antibody, an agent blocking VEGF-A activity (anti-VEGF-A antibody B20-4.1 at 5 mg/kg), or the combination of an agent blocking VEGF-A activity and an agent blocking VEGF-C activity (anti-VEGF-C antibody at 10 mg/kg. For the combination treatment group, anti-VEGF-C antibody was administered no later than thirty minutes after administration of the anti-VEGF-A antibody. Each dose was delivered in a volume of 0.2 mL per 20 grams body weight (10 mL/kg), and was scaled to the body weight of the animal.

Tumor volume was recorded twice weekly using calipers. Each animal was euthanized when its tumor reached the endpoint size (generally 1000 mm³) or at the conclusion of the study, whichever came first. Tumors were harvested and either fixated overnight in 10% NBF, followed by 70% ethanol and subsequent embedding in paraffin, or within two minutes frozen in liquid nitrogen for subsequent storage at −80° C.

The time to endpoint (TTE) was calculated from the following equation:

TTE (days)=(log₁₀(endpoint volume,mm³−b)/m

where b is the intercept and m is the slope of the line obtained by linear regression of a log-transformed tumor growth data set.

Animals that did reach the endpoint were assigned a TTE value equal to the last day of the study. Animals classified as NTR (non-treatment-related) deaths due to accident (NTRa) or due unknown causes (NTRu) were excluded from TTE calculations (and all further analyses). Animals classified as TR (treatment-related) deaths or NTRm (non-treatment-related death due to metastasis) were assigned a TTE value equal to the day of death.

Treatment outcome was evaluated by tumor growth delay (TGD), which is defined as the increase in the median time to endpoint (TTE) in a treatment group compared to the control group, which was calculated as follows:

TGD=T−C, expressed in days, or as a percentage of the median TTE of the control group, which was calculated as follows:

% TGD=[(T−C)/C]×100,

where T=median TTE for a treatment group and C=median TTE for the control group.

The Δ% TGD was calculated as above, with C=control group being the group receiving anti-VEGF-A antibody treatment alone, and T=treatment group being the group receiving the combination of anti-VEGF-A antibody and anti-VEGF-C antibody treatment. The logrank test was employed to analyze the significance of the difference between the TTE values of two groups. Two-tailed statistical analyses were conducted at significance level p=0.05. A value of “1” indicates that treatment resulted in an additional delay in tumor progression. A value of “0” indicates that the treatment did not result in an additional delay in tumor progression.

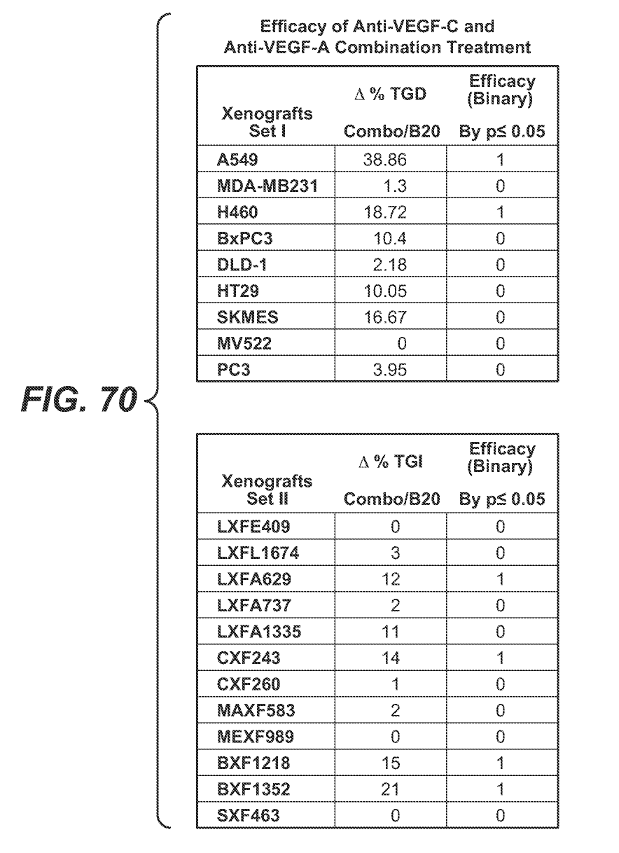

Treatment with the combination of anti-VEGF-C antibody and anti-VEGF-A antibody resulted in additional delay in tumor progression in A549, H460, LXFA629, CXF243, BXF1218, and BXF1352 tumors, compared to anti-VEGF-A antibody treatment alone (FIG. 70).

### Example 12

### Identification of Biomarkers for Efficacy of Anti-VEGF-C Antibody Treatment

Gene expression analysis was performed using qRT-PCR on frozen tumor samples obtained from the tumor model experiments described above in Example 11. From frozen material, small cubes of maximal 3 mm side length were solubilized using commercially available reagents and equipment (RNeasy®, Tissuelyzer, both Qiagen Inc., Germany). After column purification RNA was eluted with H₂O, precipitated with ethanol after the addition of glycogen and Sodium acetate. RNA was pelleted by centrifugation for at least 30 min, washed twice with 80% ethanol, and the pellet resuspended in H₂0 after drying. RNA concentrations were assessed using a spectrophotometer or a bioanalyzer (Agilent, Foster City, Calif.), and 50 ng of total RNA used per reaction in the subsequent gene expression analysis.

Gene specific primer and probe sets were designed for qRT-PCR expression analysis of 18SrRNA, RPS13, HMBS, ACTB, and SDHA (housekeeping genes) and VEGF-A, PLGF, VEGF-C, VEGF-D, VEGFR3, IL-8, CXCL1, CXCL2, Hhex, Col4a1, Col4a2, Alk1, ESM1, and Mincle. The primer and probe set sequences are listed in Table 2.

Relative expression levels of VEGF-A, PLGF, VEGF-C, VEGF-D, VEGFR3, IL-8, CXCL1, CXCL2, Hhex, Col4a1, Col4a2, Alk1, ESM1, and Mincle were determined. For example, relative expression level of VEGF-C was calculated as follows:

Relative expression VEGF-Cₛₐₘₚₗₑ=2exp(Ct_{[(HK1+HK2+HKx)/x]}−Ct_{VEGF-C}),

where HK is a housekeeping gene (e.g., 18SrRNA, RPS13, HMBS, ACTB, and SDHA) and x is the total number of housekeeping genes used to normalize the data, with Ct determined in the sample, where Ct is the threshold cycle. The Ct is the cycle number at which the fluorescence generated within a reaction crosses the threshold line.

To allow comparison of results from different reaction plates, relative expression was then calculated as a fraction to the relative expression to an internal reference RNA that was identical in all experimental runs:

Normalized relative expression VEGF-Cₛₐₘₚₗₑ=(relative expression VEGF-Cₛₐₘₚₗₑ/relative expression VEGF-C_{reference RNA}), where relative expression VEGF-Cₛₐₘₚₗₑ=2exp(Ct_{[(HK1+HK2+HKx)/x]]}−Ct_{VEGF-C}) with Ct determined in the reference RNA

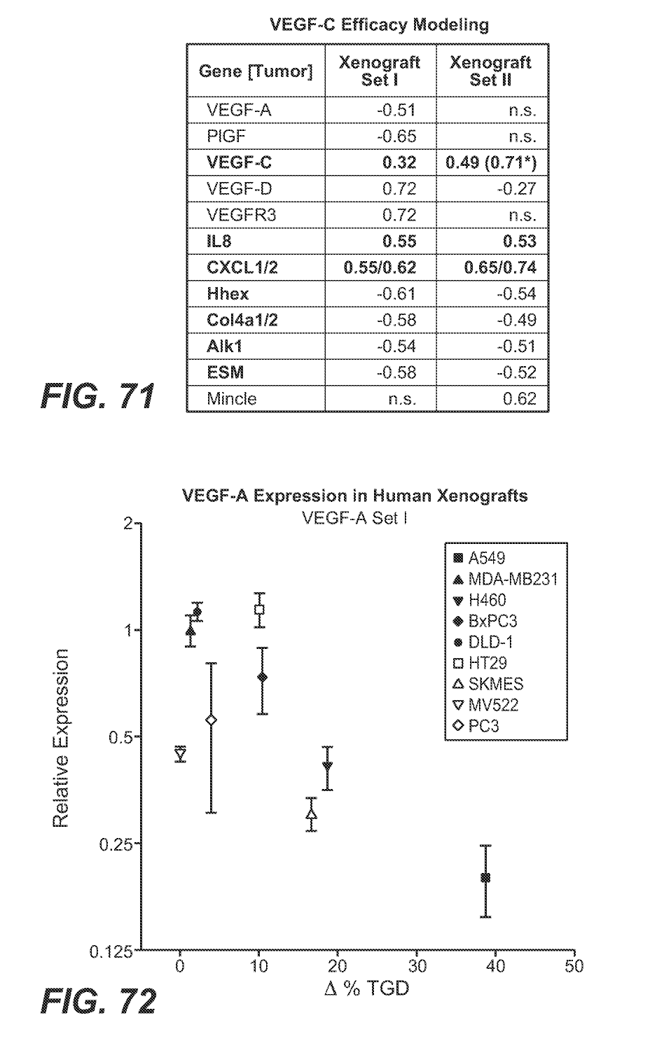

The values for the correlation of marker RNA expression (qPCR) and combination treatment efficacy are shown in FIG. 71.

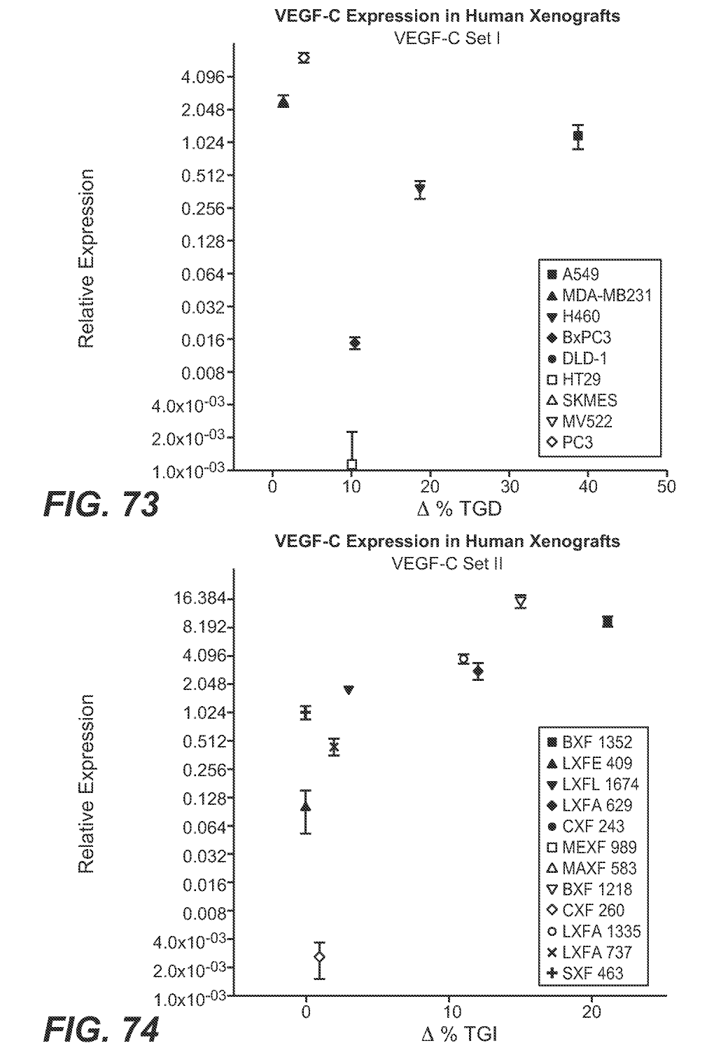

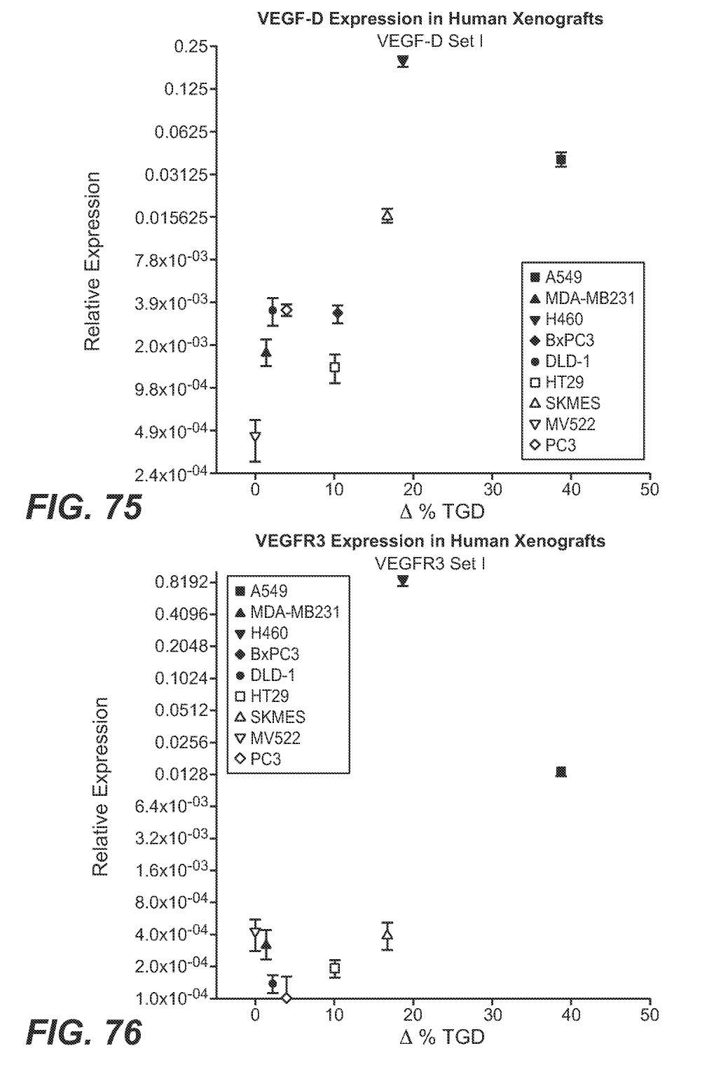

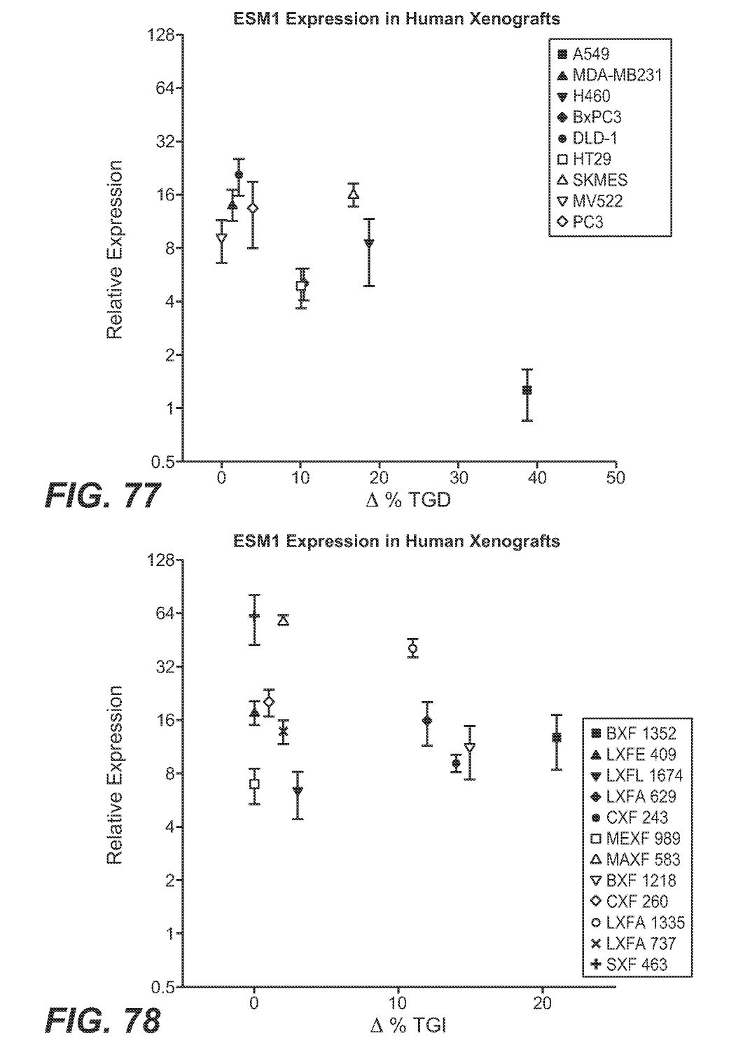

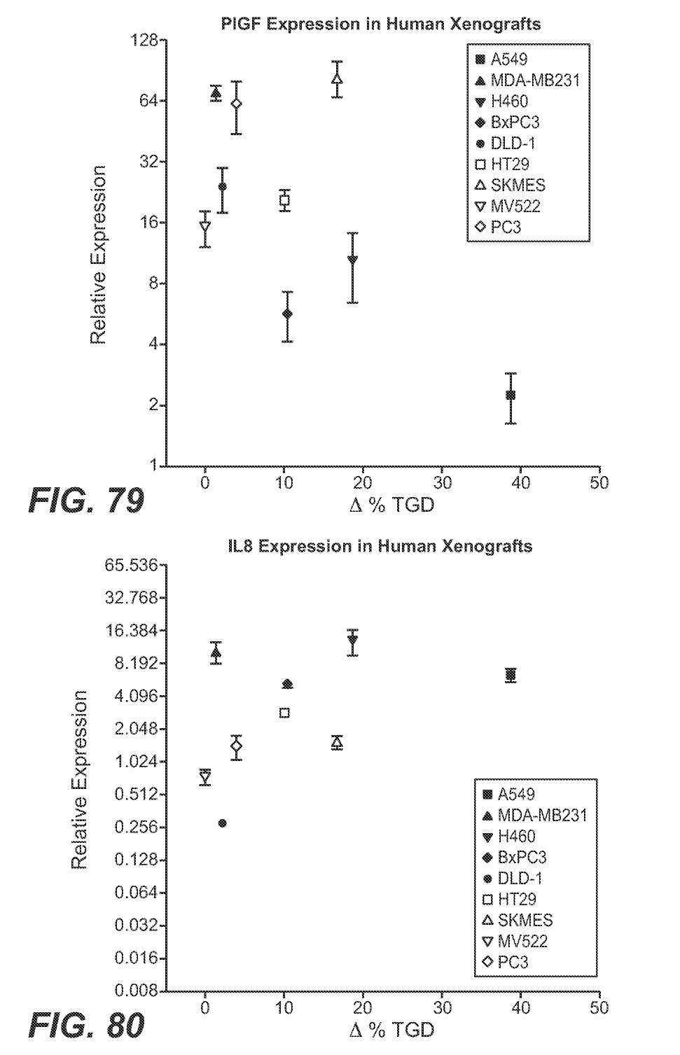

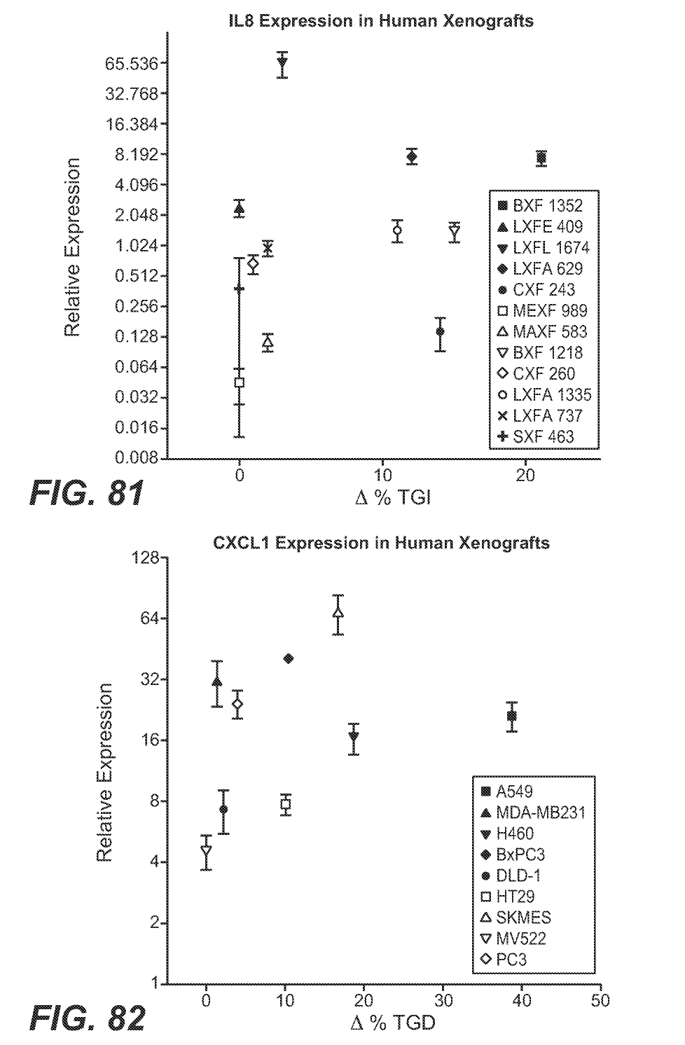

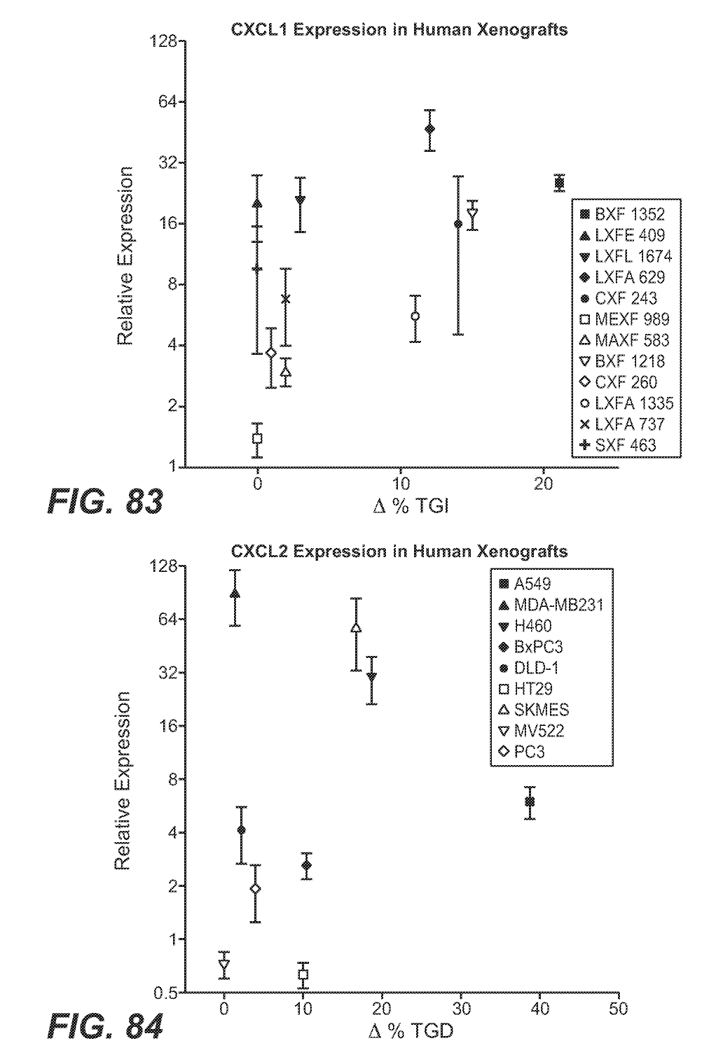

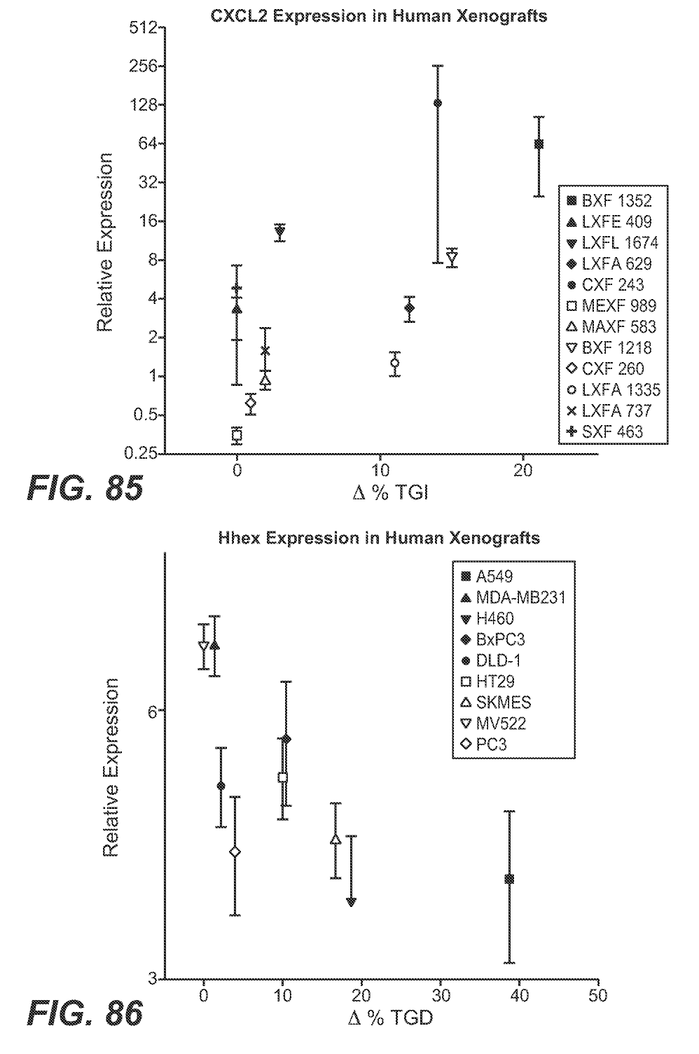

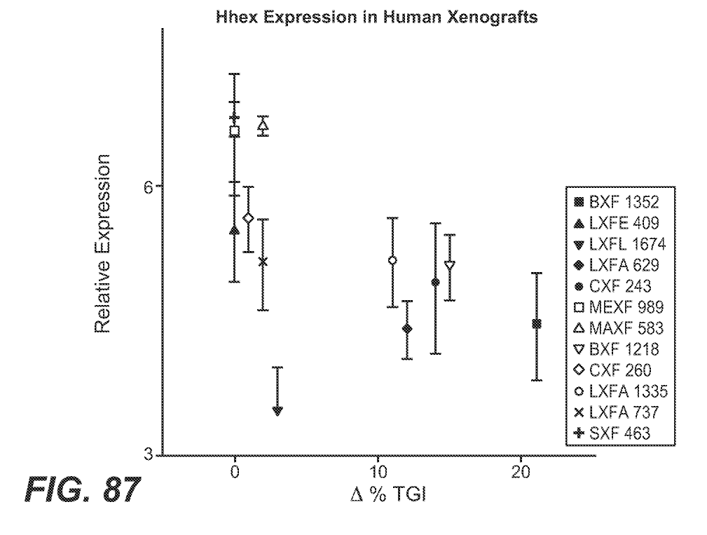

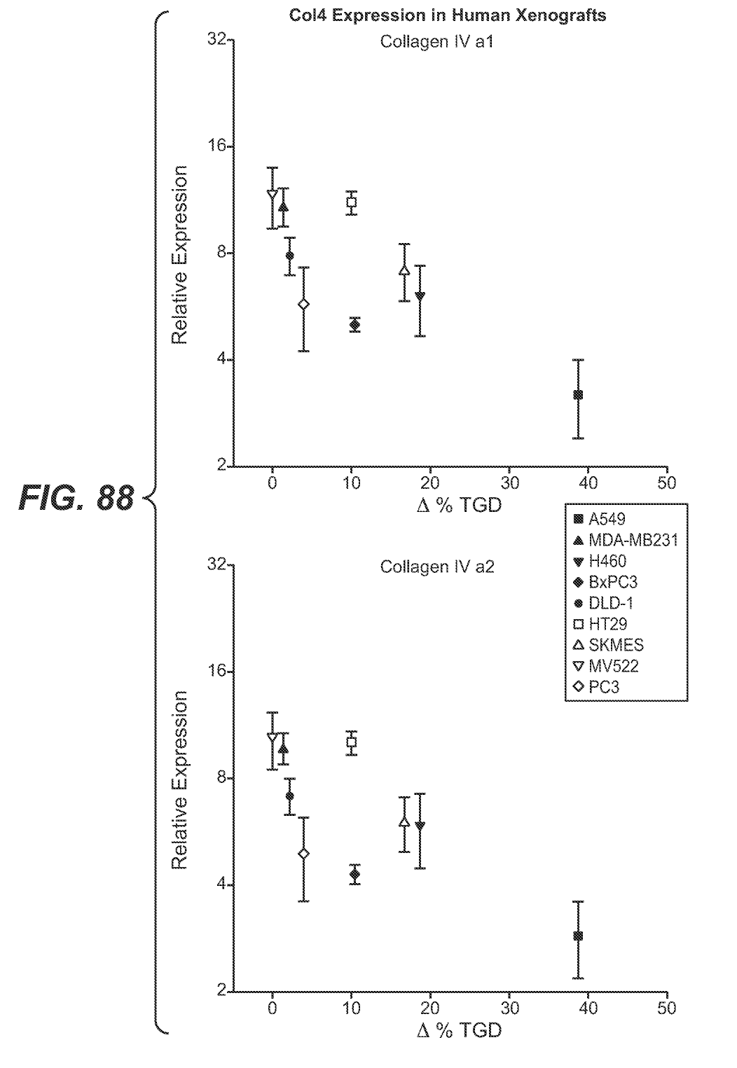

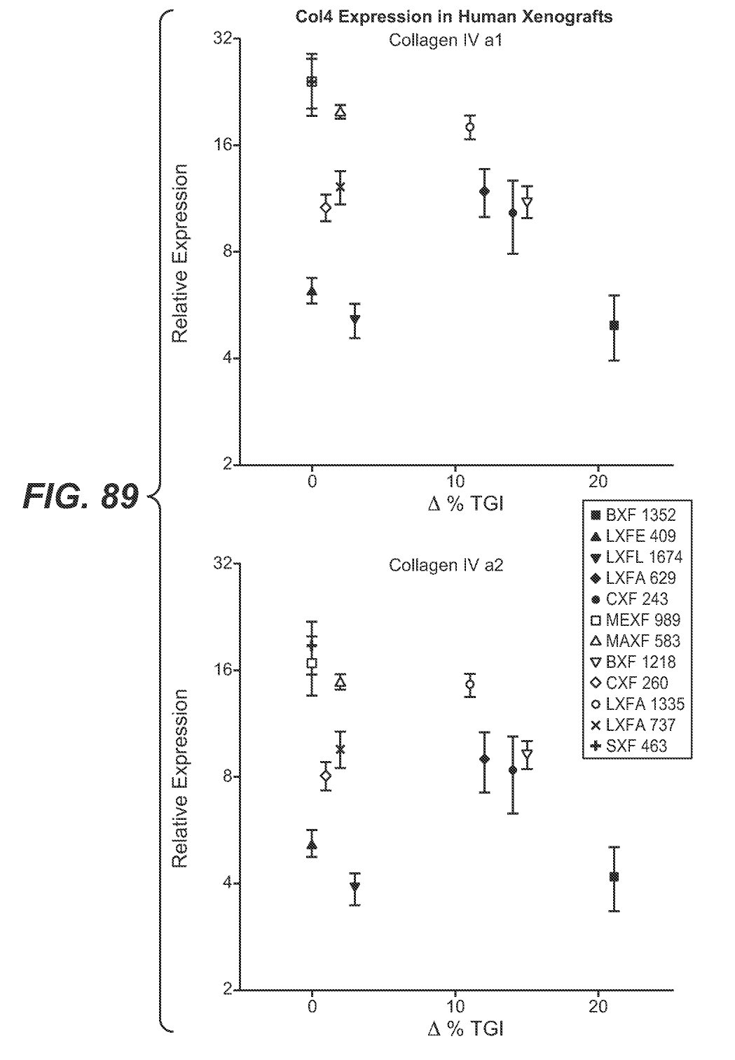

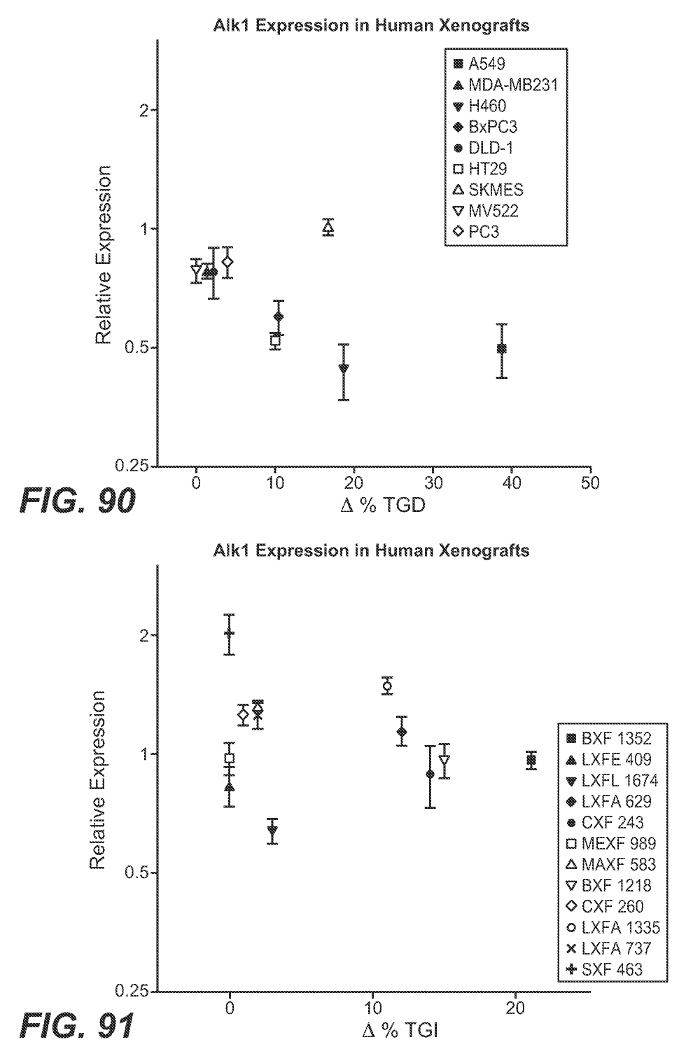

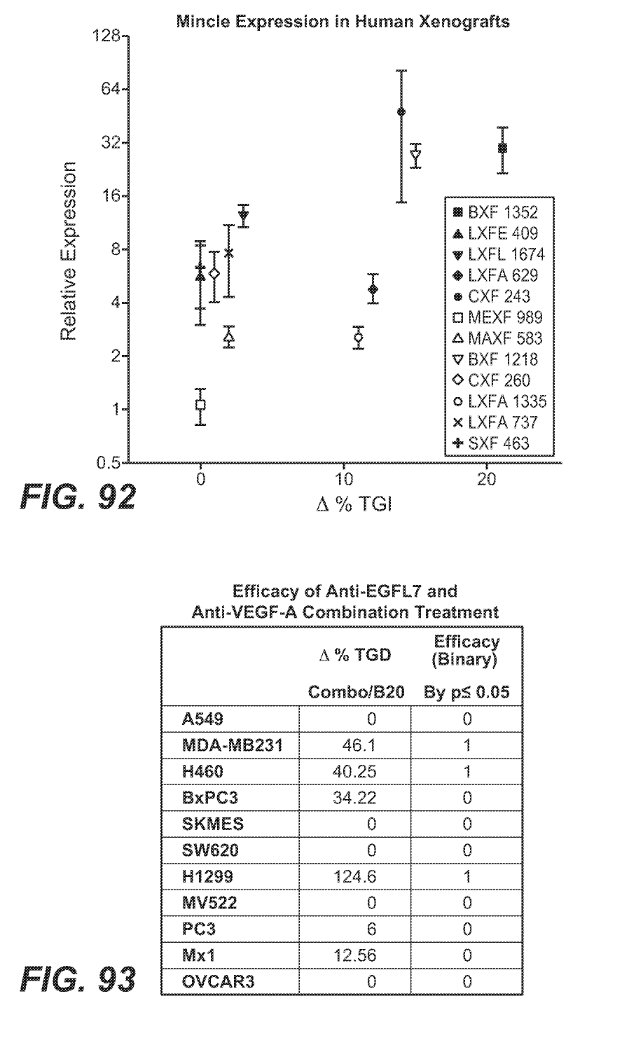

Results from the gene expression analysis are shown in FIGS. 72-92. In each of FIGS. 72-92, the relative expression of the gene assayed is compared to the percent change in tumor growth delay (Δ% TGD) exhibited by the seven different tumor models examined. Tumor models that responded to treatment with anti-VEGF-C antibody in combination with anti-VEGF-A antibody expressed higher levels of VEGF-C, VEGF-D, VEGFR3, IL-8, CXCL1, and CXCL2 compared to tumor models that did not respond to the combination treatment (see FIGS. 73-76 and 80-85).

Tumor models responsive to the combination treatment with anti-VEGF-C antibody and anti-VEGF-A antibody also expressed lower levels of VEGF-A, PlGF, Hhex, Col4a1, Col4a2, Alk1, and ESM1 as compared to the tumor models that did not respond to the combination treatment (see FIGS. 72, 77-79, and 86-92).

### Example 13

### Tumor Inhibitory Activities of Anti-EGFL7 Antibodies

All studies were conducted in accordance with the Guide for the Care and Use of Laboratory Animals, published by the NIH (NIH Publication 85-23, revised 1985). An Institutional Animal Care and Use Committee (IACUC) approved all animal protocols.

Studies were conducted with the following human tumor models using standardized techniques: A549, MDA-MB231, H460, BxPC3, SKMES, SW620, H1299, MV522 and PC3. Human tumor cells were implanted subcutaneously in the right flank of each test mouse. For example, for A549, xenografts were initiated from cultured A549 human non-small cell lung carcinoma cells (grown to mid-log phase in RPMI-1640 medium containing 10% heat-inactivated fetal bovine serum, 100 units/mL penicillin G, 100 μg/mL streptomycin sulfate, 0.25 μg/mL amphotericin B, 1 mM sodium pyruvate, 2 mM glutamine, 10 mM HEPES, 0.075% sodium bicarbonate, and 25 μg/mL gentamicin) or from A549 human lung adenocarcinoma cells (cultured in Kaighn's modified Ham's F12 medium containing 10% heat-inactivated fetal bovine serum, 100 units/mL penicillin G, 100 μg/mL streptomycin sulfate, 0.25 μg/mL amphotericin B, 2 mM glutamine, 1 mM sodium pyruvate, and 25 μg/mL gentamicin). On the day of tumor implant, A549 cells were harvested and resuspended in PBS at a concentration of 5×10⁷ cells/mL. Each test mouse received 1×10⁷ A549 tumor cells implanted subcutaneously in the right flank. For A549 tumors, A549 cells were resuspended in 100% Matrigel™ matrix (BD Biosciences, San Jose, Calif.) at a concentration of 5×10⁷ cells/mL. A549 cells (1×10⁷ in 0.2 mL) were implanted subcutaneously in the right flank of each test mouse, and tumor growth was monitored.

Tumor growth was monitored as the average size approached 120-180 mm³. On study day 1, individual tumors sizes ranged from 126 to 196 mm³ and the animals were sorted by tumor size into three test groups (one control group and two treatment groups). Tumor volume was calculated using the formula:

Tumor volume (mm³)=(w²×l)/2

where w=width and l=length in mm of the tumor.

All treatments were administered intra-peritoneally. Tumors were treated twice weekly for up to 10-20 weeks with 5-10 mg/kg each of control antibody, an agent blocking VEGF-A activity (anti-VEGF-A antibody B20-4.1 at 5 mg/kg), or the combination of an agent blocking VEGF-A activity and an agent blocking EGFL7 activity (anti-EGFL7 antibody at 10 mg/kg). For the combination treatment group, anti-EGFL7 antibody was administered no later than thirty minutes after administration of the anti-VEGF-A antibody. Each dose was delivered in a volume of 0.2 mL per 20 grams body weight (10 mL/kg), and was scaled to the body weight of the animal.

Tumor volume was recorded twice weekly using calipers. Each animal was euthanized when its tumor reached the endpoint size (generally 1000 mm³) or at the conclusion of the study, whichever came first. Tumors were harvested and either fixated overnight in 10% NBF, followed by 70% ethanol and subsequent embedding in paraffin, or within two minutes frozen in liquid nitrogen for subsequent storage at −80° C.

The time to endpoint (TTE) was calculated from the following equation:

TTE (days)=(log₁₀(endpoint volume,mm³−b)/m

where b is the intercept and m is the slope of the line obtained by linear regression of a log-transformed tumor growth data set.

Animals that did reach the endpoint were assigned a TTE value equal to the last day of the study. Animals classified as NTR (non-treatment-related) deaths due to accident (NTRa) or due unknown causes (NTRu) were excluded from TTE calculations (and all further analyses). Animals classified as TR (treatment-related) deaths or NTRm (non-treatment-related death due to metastasis) were assigned a TTE value equal to the day of death.

Treatment outcome was evaluated by tumor growth delay (TGD), which is defined as the increase in the median time to endpoint (TTE) in a treatment group compared to the control group, which was calculated as follows:

TGD=T−C, expressed in days, or as a percentage of the median TTE of the control group, which was calculated as follows:

% TGD=[(T−C)/C]×100,

where T=median TTE for a treatment group and C=median TTE for the control group.

The Δ% TGD was calculated as above, with C=control group being the group receiving anti-VEGF-A antibody treatment alone, and T=treatment group being the group receiving the combination of anti-VEGF-A antibody and anti-VEGF-C antibody treatment. The logrank test was employed to analyze the significance of the difference between the TTE values of two groups. Two-tailed statistical analyses were conducted at significance level p=0.05. A value of “1” indicates that treatment resulted in an additional delay in tumor progression. A value of “0” indicates that the treatment did not result in an additional delay in tumor progression.

Treatment with the combination of anti-EGFL7 antibody and anti-VEGF-A antibody resulted in additional delay in tumor progression in MDA-MB231, H460, and H1299 tumors, compared to anti-VEGF-A antibody treatment alone (FIG. 93).

### Example 14

### Identification of Biomarkers for Efficacy of Anti-EGFL7 Antibody Treatment

Gene expression analysis was performed using qRT-PCR on frozen tumor samples obtained from the tumor model experiments described above in Example 13. From frozen material, small cubes of maximal 3 mm side length were solubilized using commercially available reagents and equipment (RNeasy®, TissueLyzer, both Qiagen Inc., Germany). After column purification RNA was eluted with H₂O, precipitated with ethanol after the addition of glycogen and sodium acetate. RNA was pelleted by centrifugation for at least 30 min, washed twice with 80% ethanol, and the pellet resuspended in H20 after drying. RNA concentrations were assessed using a spectrophotometer or a bioanalyzer (Agilent, Foster City, Calif.), and 50 ng of total RNA used per reaction in the subsequent gene expression analysis.

Gene specific primer and probe sets were designed for qRT-PCR expression analysis of 18SrRNA, RPS13, ACTB, HNBS, and SDHA (housekeeping genes) and FRAS1, cMet, Sema3B, FGF9, FN1, HGF, MFAP5, EFEMP2/fibulin4, VEGF-C, CXCL2, FBLN2, FGF2, PDGF-C, BV8, TNFa, and Mincle. The primer and probe set sequences are listed in Table 2.

Relative expression levels of FRAS1, cMet, Sema3B, FGF9, FN1, HGF, MFAP5, EFEMP2/fibulin4, VEGF-C, CXCL2, FBLN2, FGF2, PDGF-C, BV8, TNFa, and Mincle were determined. For example, relative expression level of VEGF-C was calculated as follows:

Relative expression VEGF-Cₛₐₘₚₗₑ=2exp(Ct_{[(HK1+HK2+HKx)/x]}−Ct_{VEGF-C}),

where HK is a housekeeping gene (e.g., 18SrRNA, RPS13, HMBS, ACTB, and SDHA) and x is the total number of housekeeping genes used to normalize the data, with Ct determined in the sample, where Ct is the threshold cycle. The Ct is the cycle number at which the fluorescence generated within a reaction crosses the threshold line.

To allow comparison of results from different reaction plates, relative expression was then calculated as a fraction to the relative expression to an internal reference RNA that was identical in all experimental runs, multiplied by 100:

Normalized relative expression VEGF-Cₛₐₘₚₗₑ=(relative expression VEGF-Cₛₐₘₚₗₑ/relative expression VEGF-C_{reference RNA})×100, where relative expression VEGF-Cₛₐₘₚₗₑ=2exp(Ct_{[(HK1+HK2+HKx)/x]}−Ct_{VEGF-C}) with Ct determined in the reference RNA

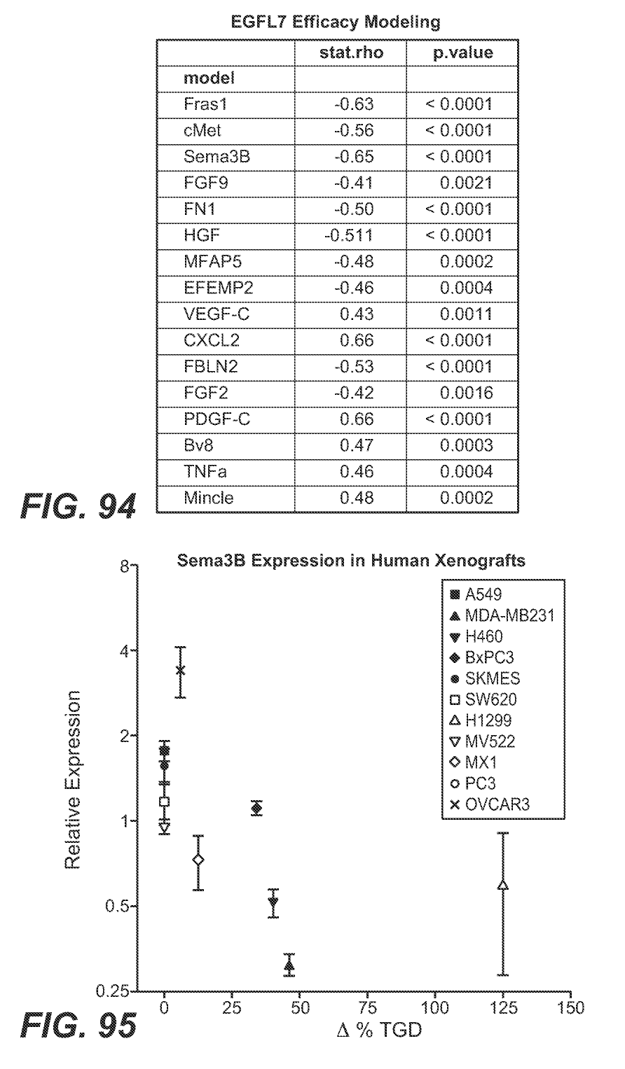

The p- and r-values for the correlation of marker RNA expression (qPCR) and combination treatment efficacy are shown in FIG. 94.

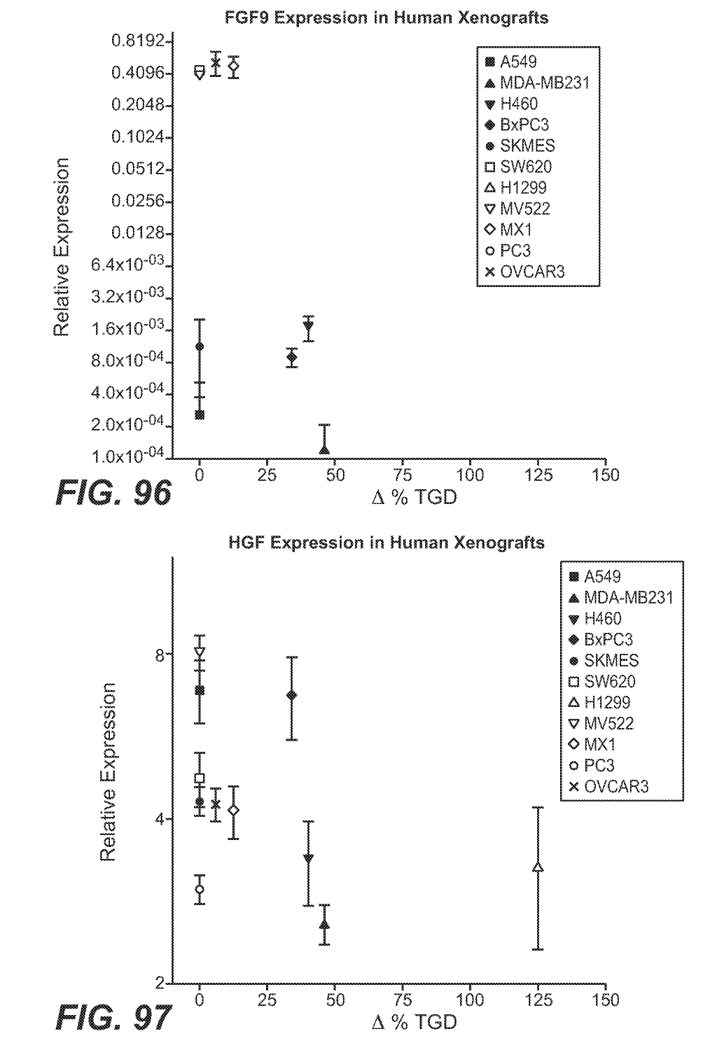

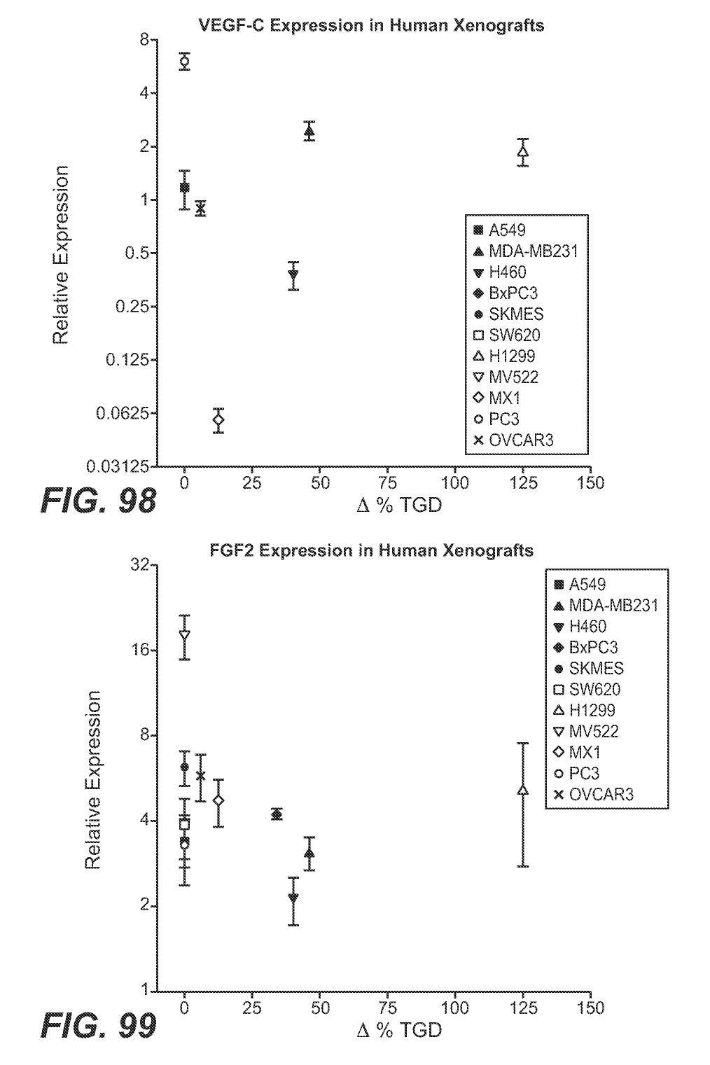

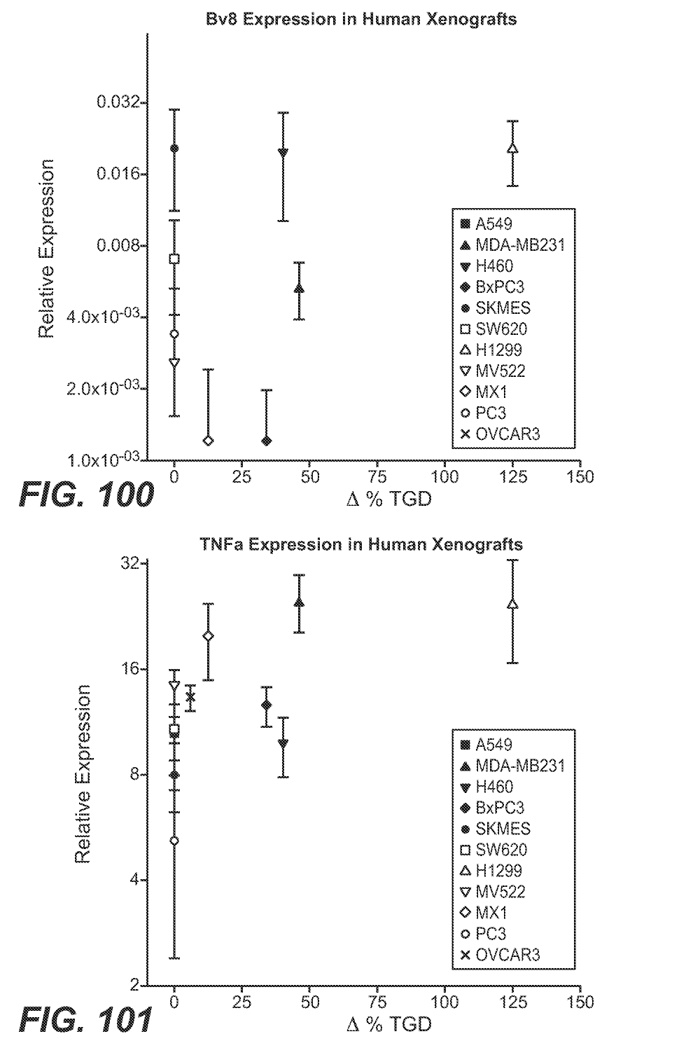

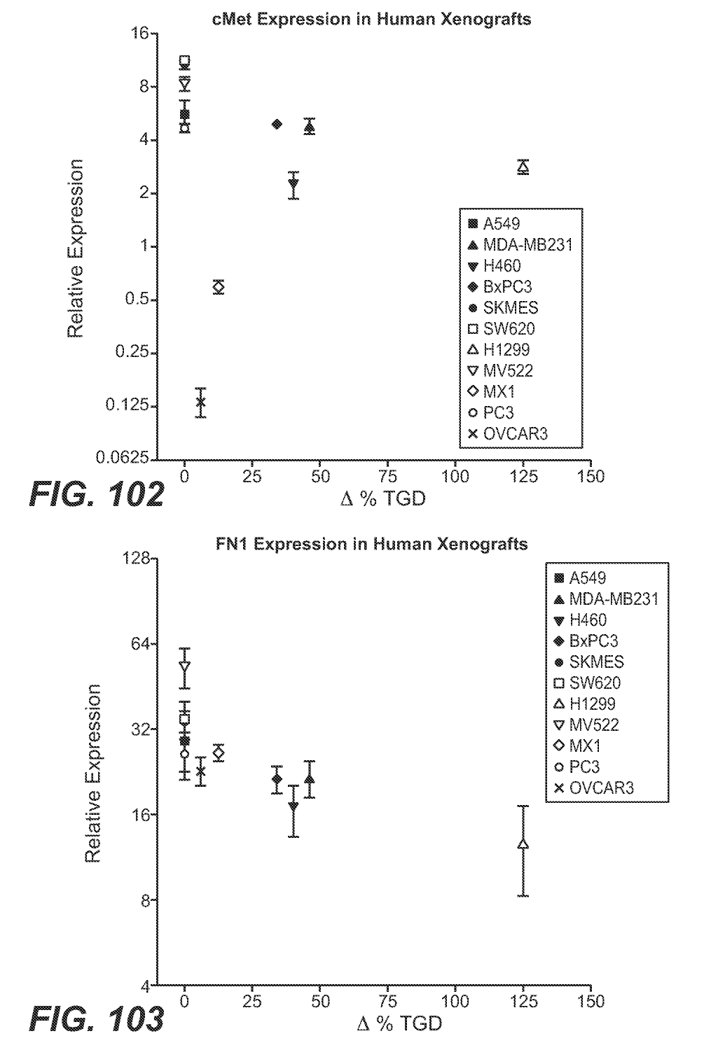

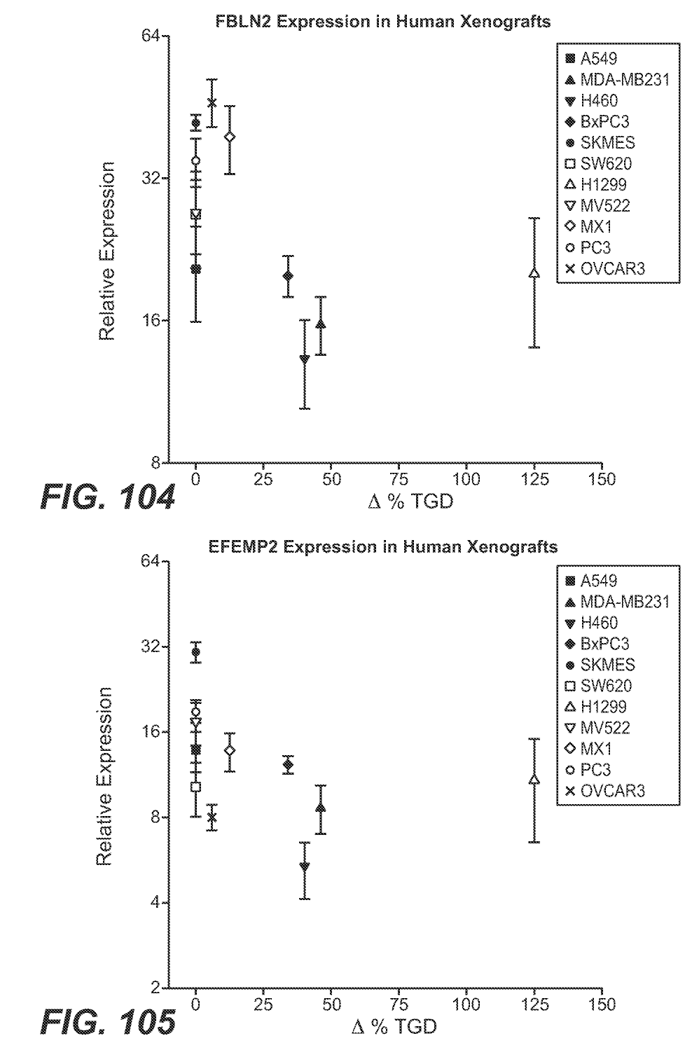

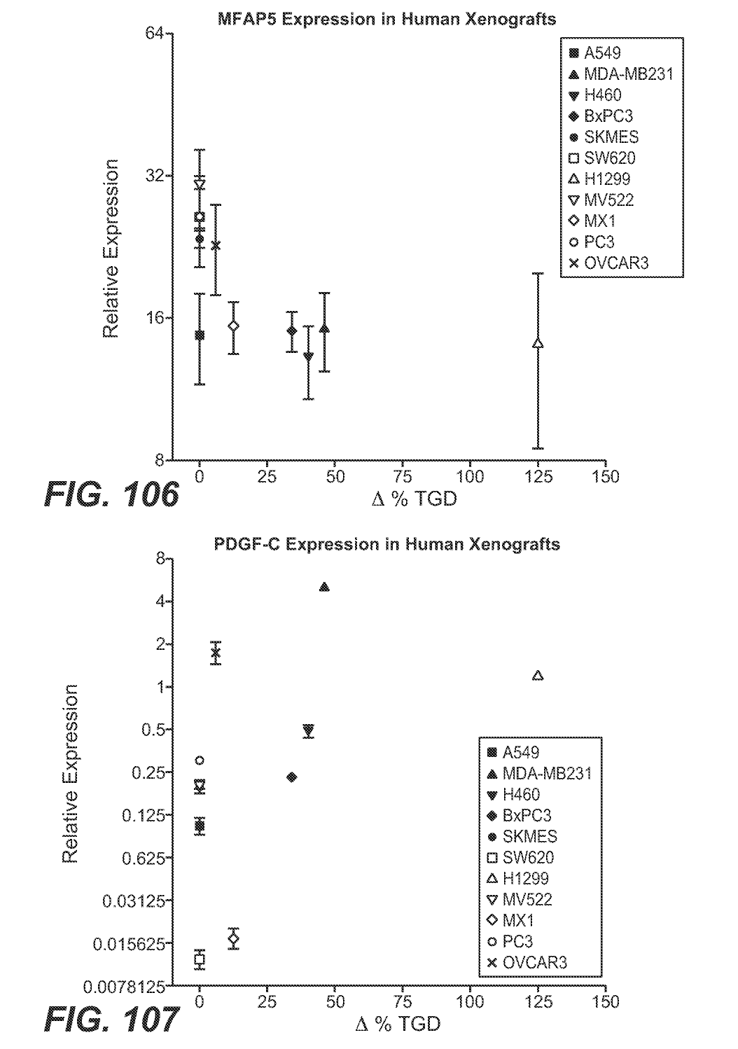

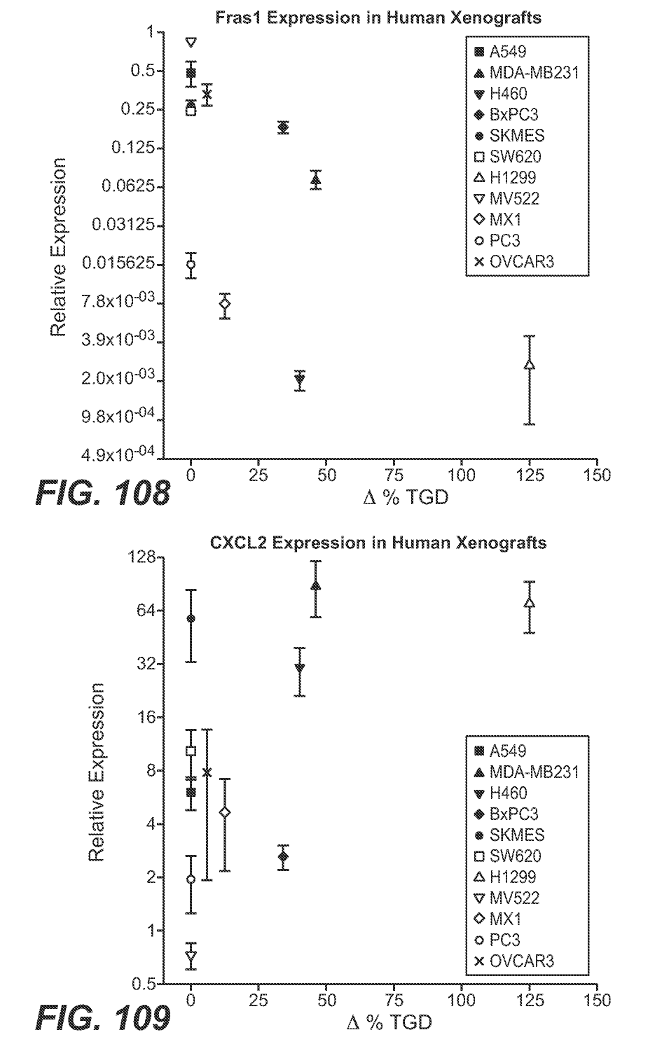

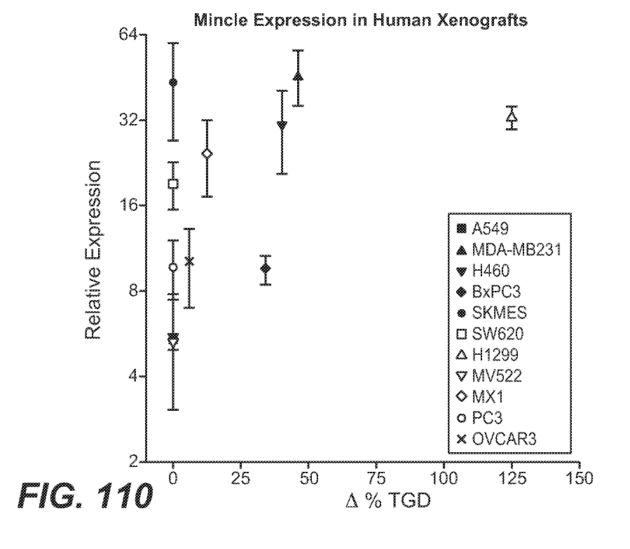

Results from the gene expression analysis are shown in FIGS. 95-110. In each of FIGS. 95-110, the relative expression of the gene assayed is compared to the percent change in tumor growth delay (Δ% TGD) exhibited by the nine different tumor models examined. Tumor models that responded to treatment with anti-EGFL7 antibody in combination with anti-VEGF-A antibody expressed higher levels of VEGF-C, CXCL2, PDGF-C, BV8, TNFα, and Mincle compared to tumor models that did not respond to the combination treatment (see FIGS. 98, 100, 101, 107, 109-110)

Tumor models responsive to the combination treatment with anti-VEGF-A antibody and anti-EGFL7 antibody also expressed lower levels of FRAS1, cMet, Sema3B, FGF9, FN1, HGF, MFAP5, EFEMP2/fibulin4, Fibulin 2, and FGF2 as compared to the tumor models that did not respond to the combination treatment (see FIGS. 95-97, 99, 102-106, and 108)

1. A method of identifying a patient who may benefit from treatment with an anti-cancer therapy other than or in addition to a VEGF-A antagonist, the method comprising:
determining expression levels of at least one gene set forth in Table 1 in a sample obtained from the patient, wherein an increased expression level of said at least one gene in the sample as compared to a reference sample indicates that the patient may benefit from treatment with the anti-cancer therapy.

2. A method of identifying a patient who may benefit from treatment with an anti-cancer therapy other than or in addition to a VEGF-A antagonist, the method comprising:
determining expression levels of at least one gene set forth in Table 1 in a sample obtained from the patient, wherein a decreased expression level of said at least one gene in the sample as compared to a reference sample indicates that the patient may benefit from treatment with the anti-cancer therapy.

3-6. (canceled)

7. A method of optimizing therapeutic efficacy for treatment of cancer, the method comprising
determining expression levels of at least one gene set forth in Table 1 in a sample obtained from the patient, wherein an increased expression level of said at least one gene in the sample as compared to a reference sample indicates that the patient has increased likelihood of benefit from an anti-cancer therapy other than or in addition to a VEGF-A antagonist. 8.

8. A method of optimizing therapeutic efficacy for treatment of cancer, the method comprising
determining expression levels of at least one gene set forth in Table 1 in a sample obtained from the patient, wherein a decreased expression level of said at least one gene in the sample as compared to a reference sample indicates that the patient has increased likelihood of benefit from an anti-cancer therapy other than or in addition to a VEGF-A antagonist.

9-10. (canceled)

11. The method of any one of claims 1, 2, 7, or 8, wherein the sample obtained from the patient is a member selected from the group consisting of: tissue, whole blood, blood-derived cells, plasma, serum, and combinations thereof.

12. The method of any one of claims 1, 2, 7, or 8, wherein the expression level is selected from mRNA expression level, protein expression level, and combinations thereof.

13. (canceled)

14. The method of any one of claims 1, 2, 7, or 8, further comprising detecting the expression of at least a second, third, fourth, fifth, sixth, seventh, eighth, ninth, or tenth gene set forth in Table 1.

15-22. (canceled)

23. The method of any one of claim 1, 2, 7 or 8, further comprising administering an effective amount of the anti-cancer therapy other than a VEGF-A antagonist to said patient.

24. The method of claim 23, wherein the anti-cancer therapy is a member selected from the group consisting of: an antibody, a small molecule, and an siRNA.

25. The method of claim 23, wherein the anti-cancer therapy is a member selected from the group consisting of: an EGFL7 antagonist, a NRP1 antagonist, and a VEGF-C antagonist.

26. The method of claim 25, wherein the EGFL7 antagonist is an antibody.

27. The method of claim 25, wherein the NRP1 antagonist is an antibody.

28. The method of claim 25, wherein the VEGF-C antagonist is an antibody.

29. The method of claim 23 further comprising administering the VEGF-A antagonist to said patient.

30. The method of claim 29, wherein the VEGF-A antagonist is an anti-VEGF-A antibody.

31. The method of claim 30, wherein the anti-VEGF-A antibody is bevacizumab.

32. The method of claim 29, wherein the anti-cancer therapy and the VEGF-A antagonist are administered concurrently.

33. The method of claim 29, wherein the anti-cancer therapy and the VEGF-A antagonist are administered sequentially.

34-286. (canceled)


SEQUENCE LISTING (576 sequences)

SEQ ID NO: 1 - DNA, 21 bp, Artificial sequence
  sequence is synthesized
agtccctgccctttgtacaca

SEQ ID NO: 2 - DNA, 19 bp, Artificial sequence
  sequence is synthesized
ccgagggcctcactaaacc

SEQ ID NO: 3 - DNA, 23 bp, Artificial sequence
  sequence is synthesized
cgcccgtcgctactaccgattgg

SEQ ID NO: 4 - DNA, 20 bp, Artificial sequence
  sequence is synthesized
gaaggcttttggtctccctg

SEQ ID NO: 5 - DNA, 23 bp, Artificial sequence
  sequence is synthesized
ggtgtgcacttttattcaactgg

SEQ ID NO: 6 - DNA, 19 bp, Artificial sequence
  sequence is synthesized
agggcttacctgtacactg

SEQ ID NO: 7 - DNA, 27 bp, Artificial sequence
  sequence is synthesized
ccatgaaataagtggttacaggaagtc

SEQ ID NO: 8 - DNA, 18 bp, Artificial sequence
  sequence is synthesized
catggacgcgaccatcct

SEQ ID NO: 9 - DNA, 26 bp, Artificial sequence
  sequence is synthesized
tcccaaaagccacccccactcctaag

SEQ ID NO: 10 - DNA, 20 bp, Artificial sequence
  sequence is synthesized
caccgtttggctcgatatta

SEQ ID NO: 11 - DNA, 21 bp, Artificial sequence
  sequence is synthesized
ggcagaggctgtagatgattc

SEQ ID NO: 12 - DNA, 21 bp, Artificial sequence
  sequence is synthesized
accaagcgagtcctccctccc

SEQ ID NO: 13 - DNA, 20 bp, Artificial sequence
  sequence is synthesized
caccgattggctcgatacta

SEQ ID NO: 14 - DNA, 20 bp, Artificial sequence
  sequence is synthesized
tagagcagaggctgtggatg

SEQ ID NO: 15 - DNA, 22 bp, Artificial sequence
  sequence is synthesized
cgggtgctcccacctaattgga

SEQ ID NO: 16 - DNA, 20 bp, Artificial sequence
  sequence is synthesized
atcaccatgcagattatgcg

SEQ ID NO: 17 - DNA, 20 bp, Artificial sequence
  sequence is synthesized
tgcattcacatttgttgtgc

SEQ ID NO: 18 - DNA, 22 bp, Artificial sequence
  sequence is synthesized
tcaaacctcaccaaggccagca

SEQ ID NO: 19 - DNA, 20 bp, Artificial sequence
  sequence is synthesized
gcagaagtcccatgaagtga

SEQ ID NO: 20 - DNA, 19 bp, Artificial sequence
  sequence is synthesized
ctcaatcggacggcagtag

SEQ ID NO: 21 - DNA, 27 bp, Artificial sequence
  sequence is synthesized
tcaagttcatggatgtctaccagcgaa

SEQ ID NO: 22 - DNA, 20 bp, Artificial sequence
  sequence is synthesized
cagtgtcaggcagcgaacaa

SEQ ID NO: 23 - DNA, 20 bp, Artificial sequence
  sequence is synthesized
cttcctgagccaggcatctg

SEQ ID NO: 24 - DNA, 30 bp, Artificial sequence
  sequence is synthesized
ctgccccaccaattacatgtggaataatca

SEQ ID NO: 25 - DNA, 20 bp, Artificial sequence
  sequence is synthesized
aaagggaagaagttccacca

SEQ ID NO: 26 - DNA, 20 bp, Artificial sequence
  sequence is synthesized
cagtcctggatcacaatgct

SEQ ID NO: 27 - DNA, 23 bp, Artificial sequence
  sequence is synthesized
tcagtcgattcgcacacggtctt

SEQ ID NO: 28 - DNA, 20 bp, Artificial sequence
  sequence is synthesized
ctgccagaagcacaagctat

SEQ ID NO: 29 - DNA, 21 bp, Artificial sequence
  sequence is synthesized
acatggtctggtatgaaaggg

SEQ ID NO: 30 - DNA, 22 bp, Artificial sequence
  sequence is synthesized
cacccagacacctgcagctgtg

SEQ ID NO: 31 - DNA, 23 bp, Artificial sequence
  sequence is synthesized
ttgacctagtgtcatggtaaagc

SEQ ID NO: 32 - DNA, 20 bp, Artificial sequence
  sequence is synthesized
tcagtgaactggggaatcac

SEQ ID NO: 33 - DNA, 22 bp, Artificial sequence
  sequence is synthesized
acatttccatgcaatggcggct

SEQ ID NO: 34 - DNA, 18 bp, Artificial sequence
  sequence is synthesized
atggcacggaagctagga

SEQ ID NO: 35 - DNA, 21 bp, Artificial sequence
  sequence is synthesized
gcagagctgaagtcctcttga

SEQ ID NO: 36 - DNA, 24 bp, Artificial sequence
  sequence is synthesized
tgctgctggacccttcctaaacct

SEQ ID NO: 37 - DNA, 18 bp, Artificial sequence
  sequence is synthesized
cggaggatgcaccacacc

SEQ ID NO: 38 - DNA, 24 bp, Artificial sequence
  sequence is synthesized
ccggttgaaagaagtccttaaaca

SEQ ID NO: 39 - DNA, 20 bp, Artificial sequence
  sequence is synthesized
cccctgcctgccaggcttgg

SEQ ID NO: 40 - DNA, 18 bp, Artificial sequence
  sequence is synthesized
cagcagtgggccttgtct

SEQ ID NO: 41 - DNA, 20 bp, Artificial sequence
  sequence is synthesized
aagggtaccacttccacctc

SEQ ID NO: 42 - DNA, 18 bp, Artificial sequence
  sequence is synthesized
tgacgagccgttcccagc

SEQ ID NO: 43 - DNA, 20 bp, Artificial sequence
  sequence is synthesized
gagctgacgttctctcagca

SEQ ID NO: 44 - DNA, 20 bp, Artificial sequence
  sequence is synthesized
ctttccggcttcatcttctc

SEQ ID NO: 45 - DNA, 19 bp, Artificial sequence
  sequence is synthesized
ctgcgaatgccggcctctg

SEQ ID NO: 46 - DNA, 22 bp, Artificial sequence
  sequence is synthesized
tgcttcttacaggtcctagctg

SEQ ID NO: 47 - DNA, 20 bp, Artificial sequence
  sequence is synthesized
aaaggcaccacttccacttc

SEQ ID NO: 48 - DNA, 20 bp, Artificial sequence
  sequence is synthesized
ccctgggaatgcacagccaa

SEQ ID NO: 49 - DNA, 20 bp, Artificial sequence
  sequence is synthesized
ccggctttcaggaagataaa

SEQ ID NO: 50 - DNA, 20 bp, Artificial sequence
  sequence is synthesized
tccatagtgatgggctcctt

SEQ ID NO: 51 - DNA, 26 bp, Artificial sequence
  sequence is synthesized
aaccgtcagaatcctcctcttcctca

SEQ ID NO: 52 - DNA, 23 bp, Artificial sequence
  sequence is synthesized
ggcacctgtaccagacaaactat

SEQ ID NO: 53 - DNA, 21 bp, Artificial sequence
  sequence is synthesized
ggcgtatttggacatctagga

SEQ ID NO: 54 - DNA, 23 bp, Artificial sequence
  sequence is synthesized
tgacccatcggcagaccaataca

SEQ ID NO: 55 - DNA, 20 bp, Artificial sequence
  sequence is synthesized
cggaaacctgtccaactacc

SEQ ID NO: 56 - DNA, 20 bp, Artificial sequence
  sequence is synthesized
tggttccaggctctctttct

SEQ ID NO: 57 - DNA, 21 bp, Artificial sequence
  sequence is synthesized
caacaaggacgcagccttgca

SEQ ID NO: 58 - DNA, 20 bp, Artificial sequence
  sequence is synthesized
ggtcaggcagctcacagtcc

SEQ ID NO: 59 - DNA, 24 bp, Artificial sequence
  sequence is synthesized
acttgtcgtctgattctccaggtt

SEQ ID NO: 60 - DNA, 22 bp, Artificial sequence
  sequence is synthesized
agcgtgtggcacccacgatcac

SEQ ID NO: 61 - DNA, 21 bp, Artificial sequence
  sequence is synthesized
tcattatcctcgtcggcactg

SEQ ID NO: 62 - DNA, 19 bp, Artificial sequence
  sequence is synthesized
ccttcattggcccgcttaa

SEQ ID NO: 63 - DNA, 30 bp, Artificial sequence
  sequence is synthesized
ttctggctccttcttgtcattgtcctacgg

SEQ ID NO: 64 - DNA, 24 bp, Artificial sequence
  sequence is synthesized
acagacagtgggatggtgctggcc

SEQ ID NO: 65 - DNA, 21 bp, Artificial sequence
  sequence is synthesized
caaaggctctgtggacaacca

SEQ ID NO: 66 - DNA, 24 bp, Artificial sequence
  sequence is synthesized
tctctatctgctcaaactcctccg

SEQ ID NO: 67 - DNA, 20 bp, Artificial sequence
  sequence is synthesized
aggagctagaaagcaggcat

SEQ ID NO: 68 - DNA, 20 bp, Artificial sequence
  sequence is synthesized
ctgggaatatccatgtgctg

SEQ ID NO: 69 - DNA, 25 bp, Artificial sequence
  sequence is synthesized
cagcttcagctgtaaaggtcctggc

SEQ ID NO: 70 - DNA, 21 bp, Artificial sequence
  sequence is synthesized
cggacccataccagagaatta

SEQ ID NO: 71 - DNA, 20 bp, Artificial sequence
  sequence is synthesized
ccatcgaagacttccacgta

SEQ ID NO: 72 - DNA, 25 bp, Artificial sequence
  sequence is synthesized
tcaaccctcacttcgatttggagga

SEQ ID NO: 73 - DNA, 21 bp, Artificial sequence
  sequence is synthesized
aaaccagcagacctggataaa

SEQ ID NO: 74 - DNA, 20 bp, Artificial sequence
  sequence is synthesized
caccttctccttcaccttcg

SEQ ID NO: 75 - DNA, 21 bp, Artificial sequence
  sequence is synthesized
tcctggcgtgctccctgtttc

SEQ ID NO: 76 - DNA, 21 bp, Artificial sequence
  sequence is synthesized
tttctcaggaagactgtgcaa

SEQ ID NO: 77 - DNA, 20 bp, Artificial sequence
  sequence is synthesized
tggcttcctggagatgttct

SEQ ID NO: 78 - DNA, 24 bp, Artificial sequence
  sequence is synthesized
cctggagtgctccctgtttcatca

SEQ ID NO: 79 - DNA, 20 bp, Artificial sequence
  sequence is synthesized
ctggagatctgggatggatt

SEQ ID NO: 80 - DNA, 19 bp, Artificial sequence
  sequence is synthesized
tttctgcccacaataacgc

SEQ ID NO: 81 - DNA, 23 bp, Artificial sequence
  sequence is synthesized
cctgaagttggccctcacattgg

SEQ ID NO: 82 - DNA, 20 bp, Artificial sequence
  sequence is synthesized
ccacagtggaacaggtgatg

SEQ ID NO: 83 - DNA, 25 bp, Artificial sequence
  sequence is synthesized
ctgtcacatttcgtattttatttga

SEQ ID NO: 84 - DNA, 20 bp, Artificial sequence
  sequence is synthesized
gaaaagcccacggtcataga

SEQ ID NO: 85 - DNA, 20 bp, Artificial sequence
  sequence is synthesized
ccacagtggaacaggtgatg

SEQ ID NO: 86 - DNA, 20 bp, Artificial sequence
  sequence is synthesized
atggtacagcaatgggatga

SEQ ID NO: 87 - DNA, 23 bp, Artificial sequence
  sequence is synthesized
ccagctcacaggtgcagaaacca

SEQ ID NO: 88 - DNA, 18 bp, Artificial sequence
  sequence is synthesized
gactggggctcagaatgg

SEQ ID NO: 89 - DNA, 20 bp, Artificial sequence
  sequence is synthesized
ctatgaccgtgggcttttct

SEQ ID NO: 90 - DNA, 21 bp, Artificial sequence
  sequence is synthesized
tgaagtggaaggtggcaccac

SEQ ID NO: 91 - DNA, 20 bp, Artificial sequence
  sequence is synthesized
ccgctataagtctggcttga

SEQ ID NO: 92 - DNA, 20 bp, Artificial sequence
  sequence is synthesized
gatgcgaatgcctgttacac

SEQ ID NO: 93 - DNA, 26 bp, Artificial sequence
  sequence is synthesized
aactctggtggcaacaagtgtcaaca

SEQ ID NO: 94 - DNA, 20 bp, Artificial sequence
  sequence is synthesized
ggatgaaacgcagacaacag

SEQ ID NO: 95 - DNA, 20 bp, Artificial sequence
  sequence is synthesized
gacgccaactatgattccaa

SEQ ID NO: 96 - DNA, 23 bp, Artificial sequence
  sequence is synthesized
tggcttgccagtagtcaccctgg

SEQ ID NO: 97 - DNA, 19 bp, Artificial sequence
  sequence is synthesized
acaaaaatggtggcacgga

SEQ ID NO: 98 - DNA, 21 bp, Artificial sequence
  sequence is synthesized
cctgatgtacttcggagcctg

SEQ ID NO: 99 - DNA, 25 bp, Artificial sequence
  sequence is synthesized
cccagtttccaagccagcggtctct

SEQ ID NO: 100 - DNA, 21 bp, Artificial sequence
  sequence is synthesized
gctgaagacctacttctcgga

SEQ ID NO: 101 - DNA, 20 bp, Artificial sequence
  sequence is synthesized
acggaaattgctgaaccact

SEQ ID NO: 102 - DNA, 25 bp, Artificial sequence
  sequence is synthesized
ttcaacagatgcattacctcgcagc

SEQ ID NO: 103 - DNA, 21 bp, Artificial sequence
  sequence is synthesized
gaacaactttaccctcacgga

SEQ ID NO: 104 - DNA, 20 bp, Artificial sequence
  sequence is synthesized
ggtcaaactgcccatacttg

SEQ ID NO: 105 - DNA, 24 bp, Artificial sequence
  sequence is synthesized
cacgataacacggccaacatcaca

SEQ ID NO: 106 - DNA, 20 bp, Artificial sequence
  sequence is synthesized
tgaagaacgaggacagcaac

SEQ ID NO: 107 - DNA, 20 bp, Artificial sequence
  sequence is synthesized
cccgattaaactgcccatac

SEQ ID NO: 108 - DNA, 20 bp, Artificial sequence
  sequence is synthesized
caccgccaacatcacggtca

SEQ ID NO: 109 - DNA, 19 bp, Artificial sequence
  sequence is synthesized
gggacccactagactgtcg

SEQ ID NO: 110 - DNA, 19 bp, Artificial sequence
  sequence is synthesized
agtgctggtgtctggaagc

SEQ ID NO: 111 - DNA, 21 bp, Artificial sequence
  sequence is synthesized
tcgctccttgctctcctggga

SEQ ID NO: 112 - DNA, 20 bp, Artificial sequence
  sequence is synthesized
aaccagagccaggagacact

SEQ ID NO: 113 - DNA, 18 bp, Artificial sequence
  sequence is synthesized
cgtcagaatcacgttggg

SEQ ID NO: 114 - DNA, 23 bp, Artificial sequence
  sequence is synthesized
tgaccatctacagctttccggcg

SEQ ID NO: 115 - DNA, 19 bp, Artificial sequence
  sequence is synthesized
cacgctacctctgctcctg

SEQ ID NO: 116 - DNA, 20 bp, Artificial sequence
  sequence is synthesized
cttctctgggatggatggat

SEQ ID NO: 117 - DNA, 20 bp, Artificial sequence
  sequence is synthesized
caccaggcccagggatcaca

SEQ ID NO: 118 - DNA, 23 bp, Artificial sequence
  sequence is synthesized
ttcagtaaccaagtcttccaaca

SEQ ID NO: 119 - DNA, 21 bp, Artificial sequence
  sequence is synthesized
tcacaatattgccatctccag

SEQ ID NO: 120 - DNA, 22 bp, Artificial sequence
  sequence is synthesized
tctcacggagcatgacatggca

SEQ ID NO: 121 - DNA, 20 bp, Artificial sequence
  sequence is synthesized
cagtatgcagcagccaaatc

SEQ ID NO: 122 - DNA, 20 bp, Artificial sequence
  sequence is synthesized
ctcttctctcacagcgttgc

SEQ ID NO: 123 - DNA, 20 bp, Artificial sequence
  sequence is synthesized
tgcctcccacacagagcgtg

SEQ ID NO: 124 - DNA, 20 bp, Artificial sequence
  sequence is synthesized
aggcagctgagatcagaagg

SEQ ID NO: 125 - DNA, 19 bp, Artificial sequence
  sequence is synthesized
gatgtctgcaggtggcact

SEQ ID NO: 126 - DNA, 20 bp, Artificial sequence
  sequence is synthesized
ctcctgggctgcctccagct

SEQ ID NO: 127 - DNA, 19 bp, Artificial sequence
  sequence is synthesized
acagtgggcttgtgctgtt

SEQ ID NO: 128 - DNA, 20 bp, Artificial sequence
  sequence is synthesized
agagaggtcgaagtggaagc

SEQ ID NO: 129 - DNA, 23 bp, Artificial sequence
  sequence is synthesized
tccttccagggctcctctgtgtg

SEQ ID NO: 130 - DNA, 14 bp, Artificial sequence
  sequence is synthesized
accccgacggccga

SEQ ID NO: 131 - DNA, 25 bp, Artificial sequence
  sequence is synthesized
tcttctgcttgaagttgtagcttga

SEQ ID NO: 132 - DNA, 23 bp, Artificial sequence
  sequence is synthesized
tccgggagaagagcgaccctcac

SEQ ID NO: 133 - DNA, 22 bp, Artificial sequence
  sequence is synthesized
accttgctatgaaggaagatgg

SEQ ID NO: 134 - DNA, 22 bp, Artificial sequence
  sequence is synthesized
ttccagtcgttcaaagaagaaa

SEQ ID NO: 135 - DNA, 25 bp, Artificial sequence
  sequence is synthesized
aacacacttagaagccagcagccgt

SEQ ID NO: 136 - DNA, 19 bp, Artificial sequence
  sequence is synthesized
ggcagccttcctgatttct

SEQ ID NO: 137 - DNA, 20 bp, Artificial sequence
  sequence is synthesized
ttctttagcactccttggca

SEQ ID NO: 138 - DNA, 25 bp, Artificial sequence
  sequence is synthesized
aaactgcaccttcacacagagctgc

SEQ ID NO: 139 - DNA, 20 bp, Artificial sequence
  sequence is synthesized
tgggacaagaacatggaaga

SEQ ID NO: 140 - DNA, 20 bp, Artificial sequence
  sequence is synthesized
gcatcatcatctggatttcg

SEQ ID NO: 141 - DNA, 25 bp, Artificial sequence
  sequence is synthesized
tcagcttacttgcatctggttccca

SEQ ID NO: 142 - DNA, 18 bp, Artificial sequence
  sequence is synthesized
ggaccagcagacaccaca

SEQ ID NO: 143 - DNA, 20 bp, Artificial sequence
  sequence is synthesized
tatcatcaaagcccttgtcg

SEQ ID NO: 144 - DNA, 22 bp, Artificial sequence
  sequence is synthesized
ccggcacaagttcttgccagaa

SEQ ID NO: 145 - DNA, 20 bp, Artificial sequence
  sequence is synthesized
tttggaaccacaccagaaga

SEQ ID NO: 146 - DNA, 20 bp, Artificial sequence
  sequence is synthesized
gtcaagggtgaggaggacac

SEQ ID NO: 147 - DNA, 23 bp, Artificial sequence
  sequence is synthesized
cctcaggaacaaaggctgctcca

SEQ ID NO: 148 - DNA, 20 bp, Artificial sequence
  sequence is synthesized
cgatgacaacgacaagatcc

SEQ ID NO: 149 - DNA, 21 bp, Artificial sequence
  sequence is synthesized
tctcccacatcatctctgtca

SEQ ID NO: 150 - DNA, 23 bp, Artificial sequence
  sequence is synthesized
ccattccattacaacccagccca

SEQ ID NO: 151 - DNA, 20 bp, Artificial sequence
  sequence is synthesized
agttaatggactgggaaggg

SEQ ID NO: 152 - DNA, 20 bp, Artificial sequence
  sequence is synthesized
gctgtcccagtgtgaccttt

SEQ ID NO: 153 - DNA, 26 bp, Artificial sequence
  sequence is synthesized
accgagcctattcacagtatgacaga

SEQ ID NO: 154 - DNA, 20 bp, Artificial sequence
  sequence is synthesized
tgctgctgagatgaatgaaa

SEQ ID NO: 155 - DNA, 19 bp, Artificial sequence
  sequence is synthesized
ccctgcttgtacagctcca

SEQ ID NO: 156 - DNA, 20 bp, Artificial sequence
  sequence is synthesized
ctccaggagccgacctgcct

SEQ ID NO: 157 - DNA, 24 bp, Artificial sequence
  sequence is synthesized
agccagctactaccagacatactg

SEQ ID NO: 158 - DNA, 21 bp, Artificial sequence
  sequence is synthesized
gaaatccgcataggtggtaac

SEQ ID NO: 159 - DNA, 26 bp, Artificial sequence
  sequence is synthesized
aactccggaaacggactgtgaaacac

SEQ ID NO: 160 - DNA, 19 bp, Artificial sequence
  sequence is synthesized
gtcccaccttggacacact

SEQ ID NO: 161 - DNA, 20 bp, Artificial sequence
  sequence is synthesized
tcccagttcttccatctgct

SEQ ID NO: 162 - DNA, 20 bp, Artificial sequence
  sequence is synthesized
ctggacgtcgccgactttgc

SEQ ID NO: 163 - DNA, 19 bp, Artificial sequence
  sequence is synthesized
gagtggctgctctagccag

SEQ ID NO: 164 - DNA, 20 bp, Artificial sequence
  sequence is synthesized
gaccttggtagaggcagagc

SEQ ID NO: 165 - DNA, 24 bp, Artificial sequence
  sequence is synthesized
tgcagcagacacagtgcctaagcc

SEQ ID NO: 166 - DNA, 20 bp, Artificial sequence
  sequence is synthesized
tatccagggaaccaggaaag

SEQ ID NO: 167 - DNA, 20 bp, Artificial sequence
  sequence is synthesized
caggcccactgctatactga

SEQ ID NO: 168 - DNA, 22 bp, Artificial sequence
  sequence is synthesized
cacagccgatttggcattctgg

SEQ ID NO: 169 - DNA, 20 bp, Artificial sequence
  sequence is synthesized
acactccaaactgtgccctt

SEQ ID NO: 170 - DNA, 20 bp, Artificial sequence
  sequence is synthesized
gggtcaatgcacacttgtct

SEQ ID NO: 171 - DNA, 23 bp, Artificial sequence
  sequence is synthesized
tgtagcccggctgaagaacaaca

SEQ ID NO: 172 - DNA, 20 bp, Artificial sequence
  sequence is synthesized
ccaacgtcaagcatctgaaa

SEQ ID NO: 173 - DNA, 20 bp, Artificial sequence
  sequence is synthesized
gggtcaatgcacacttgtct

SEQ ID NO: 174 - DNA, 22 bp, Artificial sequence
  sequence is synthesized
tgcccttcagattgttgcacgg

SEQ ID NO: 175 - DNA, 19 bp, Artificial sequence
  sequence is synthesized
cgtctgctgaggctcaagt

SEQ ID NO: 176 - DNA, 22 bp, Artificial sequence
  sequence is synthesized
ggaattgttgctgtatttctgg

SEQ ID NO: 177 - DNA, 20 bp, Artificial sequence
  sequence is synthesized
cagctccacgtgctgctcca

SEQ ID NO: 178 - DNA, 20 bp, Artificial sequence
  sequence is synthesized
ccctatatttggagcctgga

SEQ ID NO: 179 - DNA, 20 bp, Artificial sequence
  sequence is synthesized
cgggttgtgttggttgtaga

SEQ ID NO: 180 - DNA, 24 bp, Artificial sequence
  sequence is synthesized
cacagtacagcaaggtccttgccc

SEQ ID NO: 181 - DNA, 21 bp, Artificial sequence
  sequence is synthesized
tcagatcatcttctcgaaccc

SEQ ID NO: 182 - DNA, 20 bp, Artificial sequence
  sequence is synthesized
cagcttgagggtttgctaca

SEQ ID NO: 183 - DNA, 24 bp, Artificial sequence
  sequence is synthesized
cgagtgacaagcctgtagcccatg

SEQ ID NO: 184 - DNA, 20 bp, Artificial sequence
  sequence is synthesized
agttctatggcccagaccct

SEQ ID NO: 185 - DNA, 20 bp, Artificial sequence
  sequence is synthesized
tccacttggtggtttgctac

SEQ ID NO: 186 - DNA, 23 bp, Artificial sequence
  sequence is synthesized
tcgagtgacaagcctgtagccca

SEQ ID NO: 187 - DNA, 18 bp, Artificial sequence
  sequence is synthesized
caacattgtgcggagctt

SEQ ID NO: 188 - DNA, 20 bp, Artificial sequence
  sequence is synthesized
gagcaagatgtgcttctgga

SEQ ID NO: 189 - DNA, 23 bp, Artificial sequence
  sequence is synthesized
cagcatggaagatgccatctcca

SEQ ID NO: 190 - DNA, 20 bp, Artificial sequence
  sequence is synthesized
ccttggtccacctcaagaat

SEQ ID NO: 191 - DNA, 20 bp, Artificial sequence
  sequence is synthesized
ggagatgggctctagctttg

SEQ ID NO: 192 - DNA, 20 bp, Artificial sequence
  sequence is synthesized
ccaaagcctgctgtgtgccc

SEQ ID NO: 193 - DNA, 21 bp, Artificial sequence
  sequence is synthesized
gaggttctgctggaagaaatg

SEQ ID NO: 194 - DNA, 21 bp, Artificial sequence
  sequence is synthesized
ctgcttagtggaaagctccat

SEQ ID NO: 195 - DNA, 23 bp, Artificial sequence
  sequence is synthesized
cgggaaccgactgctatttcagc

SEQ ID NO: 196 - DNA, 20 bp, Artificial sequence
  sequence is synthesized
tcctcatgctcacgctattt

SEQ ID NO: 197 - DNA, 20 bp, Artificial sequence
  sequence is synthesized
agtcagtgggtctccattcc

SEQ ID NO: 198 - DNA, 21 bp, Artificial sequence
  sequence is synthesized
cgtcttgtgcgcctctttgca

SEQ ID NO: 199 - DNA, 20 bp, Artificial sequence
  sequence is synthesized
acctggacaacatcagcaag

SEQ ID NO: 200 - DNA, 18 bp, Artificial sequence
  sequence is synthesized
gcccagttgcactcctct

SEQ ID NO: 201 - DNA, 19 bp, Artificial sequence
  sequence is synthesized
ccggccaggccagcttctt

SEQ ID NO: 202 - DNA, 19 bp, Artificial sequence
  sequence is synthesized
agctgccgatggacactac

SEQ ID NO: 203 - DNA, 21 bp, Artificial sequence
  sequence is synthesized
gggactgagatcactttcagc

SEQ ID NO: 204 - DNA, 26 bp, Artificial sequence
  sequence is synthesized
tgtgcccacatctgtaccaatgaaga

SEQ ID NO: 205 - DNA, 20 bp, Artificial sequence
  sequence is synthesized
cagggcagaattccatatcc

SEQ ID NO: 206 - DNA, 20 bp, Artificial sequence
  sequence is synthesized
cgcatattgggtgtaaatgc

SEQ ID NO: 207 - DNA, 21 bp, Artificial sequence
  sequence is synthesized
cgccctggaacttgtccagga

SEQ ID NO: 208 - DNA, 20 bp, Artificial sequence
  sequence is synthesized
atgtgagacatggaaaccca

SEQ ID NO: 209 - DNA, 21 bp, Artificial sequence
  sequence is synthesized
ttcagctgcatttctgtatgc

SEQ ID NO: 210 - DNA, 22 bp, Artificial sequence
  sequence is synthesized
ttgaaccctcggcattgtgtca

SEQ ID NO: 211 - DNA, 20 bp, Artificial sequence
  sequence is synthesized
gctcacgcaatttacaccag

SEQ ID NO: 212 - DNA, 20 bp, Artificial sequence
  sequence is synthesized
ttctctgccctcctacatca

SEQ ID NO: 213 - DNA, 21 bp, Artificial sequence
  sequence is synthesized
ttcacacagagtcgcccgacc

SEQ ID NO: 214 - DNA, 23 bp, Artificial sequence
  sequence is synthesized
ccactggtcactatatgaaggaa

SEQ ID NO: 215 - DNA, 20 bp, Artificial sequence
  sequence is synthesized
cttgcctccgtttactttgc

SEQ ID NO: 216 - DNA, 21 bp, Artificial sequence
  sequence is synthesized
caaggcctggttcctgtgcca

SEQ ID NO: 217 - DNA, 20 bp, Artificial sequence
  sequence is synthesized
ggaaccctgtcatttacgct

SEQ ID NO: 218 - DNA, 20 bp, Artificial sequence
  sequence is synthesized
gtagacacacacggcagagc

SEQ ID NO: 219 - DNA, 20 bp, Artificial sequence
  sequence is synthesized
cctctggctccgtgttccga

SEQ ID NO: 220 - DNA, 20 bp, Artificial sequence
  sequence is synthesized
cgtcaggaacccagtcattt

SEQ ID NO: 221 - DNA, 20 bp, Artificial sequence
  sequence is synthesized
agacacacactgcagaccct

SEQ ID NO: 222 - DNA, 27 bp, Artificial sequence
  sequence is synthesized
ctttacctcttcaggctctgtgttccg

SEQ ID NO: 223 - DNA, 20 bp, Artificial sequence
  sequence is synthesized
ctcaaacctggagagtgcct

SEQ ID NO: 224 - DNA, 20 bp, Artificial sequence
  sequence is synthesized
ggttcagcacgaagctctta

SEQ ID NO: 225 - DNA, 20 bp, Artificial sequence
  sequence is synthesized
cgtcaggagccacctcgcct

SEQ ID NO: 226 - DNA, 19 bp, Artificial sequence
  sequence is synthesized
aatcatggcctgtggtctg

SEQ ID NO: 227 - DNA, 20 bp, Artificial sequence
  sequence is synthesized
cccgaactttgagacattcc

SEQ ID NO: 228 - DNA, 21 bp, Artificial sequence
  sequence is synthesized
tcgccagcaacctgaatctca

SEQ ID NO: 229 - DNA, 20 bp, Artificial sequence
  sequence is synthesized
ccttcgaggtgctcatcatc

SEQ ID NO: 230 - DNA, 18 bp, Artificial sequence
  sequence is synthesized
ggcggaagtggtggtact

SEQ ID NO: 231 - DNA, 20 bp, Artificial sequence
  sequence is synthesized
accacggccttgaagccgtc

SEQ ID NO: 232 - DNA, 19 bp, Artificial sequence
  sequence is synthesized
gagaattcgaggcatggtc

SEQ ID NO: 233 - DNA, 20 bp, Artificial sequence
  sequence is synthesized
atctgctccttgctcctcac

SEQ ID NO: 234 - DNA, 20 bp, Artificial sequence
  sequence is synthesized
catggaacctgccagcctgg

SEQ ID NO: 235 - DNA, 21 bp, Artificial sequence
  sequence is synthesized
tggtaatggattgcctctctc

SEQ ID NO: 236 - DNA, 21 bp, Artificial sequence
  sequence is synthesized
cagtgcttctaagctgggttt

SEQ ID NO: 237 - DNA, 24 bp, Artificial sequence
  sequence is synthesized
cgagctttcaccctggtgagactg

SEQ ID NO: 238 - DNA, 20 bp, Artificial sequence
  sequence is synthesized
agtcaagtggcctctctggt

SEQ ID NO: 239 - DNA, 20 bp, Artificial sequence
  sequence is synthesized
cgcttcacaggctagatttg

SEQ ID NO: 240 - DNA, 22 bp, Artificial sequence
  sequence is synthesized
tgagcttgcatcctgaccaggc

SEQ ID NO: 241 - DNA, 20 bp, Artificial sequence
  sequence is synthesized
aggtggtgtgtgtggatcag

SEQ ID NO: 242 - DNA, 19 bp, Artificial sequence
  sequence is synthesized
ccgcatcatctgagctagg

SEQ ID NO: 243 - DNA, 20 bp, Artificial sequence
  sequence is synthesized
ctggctgcagacccggtcct

SEQ ID NO: 244 - DNA, 20 bp, Artificial sequence
  sequence is synthesized
ctttggcctagtgctatggg

SEQ ID NO: 245 - DNA, 20 bp, Artificial sequence
  sequence is synthesized
gaaaggtggcctgtaatcct

SEQ ID NO: 246 - DNA, 20 bp, Artificial sequence
  sequence is synthesized
cggcggaccatcatcaatgg

SEQ ID NO: 247 - DNA, 19 bp, Artificial sequence
  sequence is synthesized
gcctcaatgcttctggaaa

SEQ ID NO: 248 - DNA, 20 bp, Artificial sequence
  sequence is synthesized
cagtccagctgaagttccac

SEQ ID NO: 249 - DNA, 25 bp, Artificial sequence
  sequence is synthesized
cgttgctgactccattggtttcaca

SEQ ID NO: 250 - DNA, 20 bp, Artificial sequence
  sequence is synthesized
accgtccttaatggctcaga

SEQ ID NO: 251 - DNA, 20 bp, Artificial sequence
  sequence is synthesized
ccacagcatagccgaagtag

SEQ ID NO: 252 - DNA, 25 bp, Artificial sequence
  sequence is synthesized
caacgtctcaggagaacagatggcc

SEQ ID NO: 253 - DNA, 20 bp, Artificial sequence
  sequence is synthesized
cttcctgcccaccatctact

SEQ ID NO: 254 - DNA, 20 bp, Artificial sequence
  sequence is synthesized
catgaccaggatgaccaatc

SEQ ID NO: 255 - DNA, 26 bp, Artificial sequence
  sequence is synthesized
catcttcttaactggcattgtgggca

SEQ ID NO: 256 - DNA, 20 bp, Artificial sequence
  sequence is synthesized
gtgtaccagcccttcctcac

SEQ ID NO: 257 - DNA, 20 bp, Artificial sequence
  sequence is synthesized
cggtcctatagatggttcgg

SEQ ID NO: 258 - DNA, 19 bp, Artificial sequence
  sequence is synthesized
accgggcctgcagcaccta

SEQ ID NO: 259 - DNA, 22 bp, Artificial sequence
  sequence is synthesized
ggcagcagatggtactactgag

SEQ ID NO: 260 - DNA, 19 bp, Artificial sequence
  sequence is synthesized
gatggaacctccggaaatc

SEQ ID NO: 261 - DNA, 25 bp, Artificial sequence
  sequence is synthesized
cccacagtacacactctacggctgg

SEQ ID NO: 262 - DNA, 20 bp, Artificial sequence
  sequence is synthesized
aagccctacctgaccttgtg

SEQ ID NO: 263 - DNA, 20 bp, Artificial sequence
  sequence is synthesized
ataacgcggtacatggtcct

SEQ ID NO: 264 - DNA, 20 bp, Artificial sequence
  sequence is synthesized
agtgctgcagatgcgcctcc

SEQ ID NO: 265 - DNA, 19 bp, Artificial sequence
  sequence is synthesized
ctgtcagggctggaagaag

SEQ ID NO: 266 - DNA, 21 bp, Artificial sequence
  sequence is synthesized
cacctccattaagacaaggct

SEQ ID NO: 267 - DNA, 23 bp, Artificial sequence
  sequence is synthesized
tcacctgtgatgccatctgctcc

SEQ ID NO: 268 - DNA, 20 bp, Artificial sequence
  sequence is synthesized
cggccatgagtccttctact

SEQ ID NO: 269 - DNA, 20 bp, Artificial sequence
  sequence is synthesized
ggagagggtgtatcgcaact

SEQ ID NO: 270 - DNA, 20 bp, Artificial sequence
  sequence is synthesized
ccgtaggccgccaccttgtc

SEQ ID NO: 271 - DNA, 20 bp, Artificial sequence
  sequence is synthesized
ggttcgggaagaggttgtta

SEQ ID NO: 272 - DNA, 20 bp, Artificial sequence
  sequence is synthesized
tcatccgtaggttggttcaa

SEQ ID NO: 273 - DNA, 21 bp, Artificial sequence
  sequence is synthesized
ccgtgggcaactctgtcaacg

SEQ ID NO: 274 - DNA, 20 bp, Artificial sequence
  sequence is synthesized
agaaccagaggaggcacaag

SEQ ID NO: 275 - DNA, 20 bp, Artificial sequence
  sequence is synthesized
catctgtaggctggttcagg

SEQ ID NO: 276 - DNA, 20 bp, Artificial sequence
  sequence is synthesized
ccttcgctgacagcgttgcc

SEQ ID NO: 277 - DNA, 20 bp, Artificial sequence
  sequence is synthesized
ctcagtcccgagacttcaca

SEQ ID NO: 278 - DNA, 21 bp, Artificial sequence
  sequence is synthesized
aaacacttaaacccaccagga

SEQ ID NO: 279 - DNA, 22 bp, Artificial sequence
  sequence is synthesized
cctccacccttcaaccactccg

SEQ ID NO: 280 - DNA, 19 bp, Artificial sequence
  sequence is synthesized
cgaggcattcgaagagcta

SEQ ID NO: 281 - DNA, 20 bp, Artificial sequence
  sequence is synthesized
tcctccttcagcctcagttt

SEQ ID NO: 282 - DNA, 22 bp, Artificial sequence
  sequence is synthesized
tctctagggtgcccagcgtcaa

SEQ ID NO: 283 - DNA, 19 bp, Artificial sequence
  sequence is synthesized
catcggccagtcagacagt

SEQ ID NO: 284 - DNA, 23 bp, Artificial sequence
  sequence is synthesized
agtcgggaacagatctcattatt

SEQ ID NO: 285 - DNA, 22 bp, Artificial sequence
  sequence is synthesized
ctgcttcaccagtttacggcgc

SEQ ID NO: 286 - DNA, 20 bp, Artificial sequence
  sequence is synthesized
gacacactcagcagccagag

SEQ ID NO: 287 - DNA, 24 bp, Artificial sequence
  sequence is synthesized
ccaagaacagcatattgtctacag

SEQ ID NO: 288 - DNA, 20 bp, Artificial sequence
  sequence is synthesized
ccggcagacagatcgcagct

SEQ ID NO: 289 - DNA, 19 bp, Artificial sequence
  sequence is synthesized
agaatggtgcccagagtga

SEQ ID NO: 290 - DNA, 24 bp, Artificial sequence
  sequence is synthesized
ttctctttcaagtaggagatgcag

SEQ ID NO: 291 - DNA, 27 bp, Artificial sequence
  sequence is synthesized
cattgcctctgggctatcctacagatg

SEQ ID NO: 292 - DNA, 19 bp, Artificial sequence
  sequence is synthesized
cacctgccctgatggttac

SEQ ID NO: 293 - DNA, 21 bp, Artificial sequence
  sequence is synthesized
caatagcggtaacgacactca

SEQ ID NO: 294 - DNA, 24 bp, Artificial sequence
  sequence is synthesized
tgtccacacattcgggtccaattt

SEQ ID NO: 295 - DNA, 19 bp, Artificial sequence
  sequence is synthesized
cggcagagatggtcttgaa

SEQ ID NO: 296 - DNA, 20 bp, Artificial sequence
  sequence is synthesized
tctctccaggctctccctta

SEQ ID NO: 297 - DNA, 20 bp, Artificial sequence
  sequence is synthesized
ccttgtggacccggcaatcc

SEQ ID NO: 298 - DNA, 20 bp, Artificial sequence
  sequence is synthesized
ttcattcctcatgcacactg

SEQ ID NO: 299 - DNA, 20 bp, Artificial sequence
  sequence is synthesized
gcacggaagtcctctagaca

SEQ ID NO: 300 - DNA, 20 bp, Artificial sequence
  sequence is synthesized
actggccaccgccttcatcc

SEQ ID NO: 301 - DNA, 20 bp, Artificial sequence
  sequence is synthesized
ttaccctgctgctactcctg

SEQ ID NO: 302 - DNA, 19 bp, Artificial sequence
  sequence is synthesized
gcattgtcctttgcctttg

SEQ ID NO: 303 - DNA, 22 bp, Artificial sequence
  sequence is synthesized
cacagcccttgctagccacagg

SEQ ID NO: 304 - DNA, 20 bp, Artificial sequence
  sequence is synthesized
ggccaagatgttacagctca

SEQ ID NO: 305 - DNA, 20 bp, Artificial sequence
  sequence is synthesized
ttgctttgaggattctcctg

SEQ ID NO: 306 - DNA, 22 bp, Artificial sequence
  sequence is synthesized
cctggtttcagaatcgccgagc

SEQ ID NO: 307 - DNA, 20 bp, Artificial sequence
  sequence is synthesized
cctttctcttcgtggagctt

SEQ ID NO: 308 - DNA, 19 bp, Artificial sequence
  sequence is synthesized
gtcagaggagggagcttgg

SEQ ID NO: 309 - DNA, 23 bp, Artificial sequence
  sequence is synthesized
tccacacactggctctgtgggtc

SEQ ID NO: 310 - DNA, 19 bp, Artificial sequence
  sequence is synthesized
catctcgagggaggaggag

SEQ ID NO: 311 - DNA, 18 bp, Artificial sequence
  sequence is synthesized
cactcggcgattacagca

SEQ ID NO: 312 - DNA, 20 bp, Artificial sequence
  sequence is synthesized
tgctgctgccagggacccta

SEQ ID NO: 313 - DNA, 25 bp, Artificial sequence
  sequence is synthesized
cttatgataactatgtcccatctgc

SEQ ID NO: 314 - DNA, 22 bp, Artificial sequence
  sequence is synthesized
ctggtgagtcgttgattaaggt

SEQ ID NO: 315 - DNA, 22 bp, Artificial sequence
  sequence is synthesized
ccctgaaaggacctatcgcgcc

SEQ ID NO: 316 - DNA, 18 bp, Artificial sequence
  sequence is synthesized
gaggaggtcctgcagtgg

SEQ ID NO: 317 - DNA, 20 bp, Artificial sequence
  sequence is synthesized
tgaagctggcaaatccatag

SEQ ID NO: 318 - DNA, 21 bp, Artificial sequence
  sequence is synthesized
cgccagtccctggacaagctt

SEQ ID NO: 319 - DNA, 20 bp, Artificial sequence
  sequence is synthesized
ccgaagtcatagccacactc

SEQ ID NO: 320 - DNA, 20 bp, Artificial sequence
  sequence is synthesized
tttctgaaccaagggagctt

SEQ ID NO: 321 - DNA, 20 bp, Artificial sequence
  sequence is synthesized
aaggcaagcctcgcgaccat

SEQ ID NO: 322 - DNA, 20 bp, Artificial sequence
  sequence is synthesized
aaaggcaaggctaactgacc

SEQ ID NO: 323 - DNA, 20 bp, Artificial sequence
  sequence is synthesized
ctttggttcttccgttgagg

SEQ ID NO: 324 - DNA, 20 bp, Artificial sequence
  sequence is synthesized
cagcagcccaggctcctcct

SEQ ID NO: 325 - DNA, 19 bp, Artificial sequence
  sequence is synthesized
tccccgaagcagcactctt

SEQ ID NO: 326 - DNA, 19 bp, Artificial sequence
  sequence is synthesized
accgcaatgagccctttct

SEQ ID NO: 327 - DNA, 26 bp, Artificial sequence
  sequence is synthesized
cagtcagagtcttccttgccccatgg

SEQ ID NO: 328 - DNA, 20 bp, Artificial sequence
  sequence is synthesized
aacccaaacagaggcagagt

SEQ ID NO: 329 - DNA, 19 bp, Artificial sequence
  sequence is synthesized
cagatggtggtttccttgg

SEQ ID NO: 330 - DNA, 25 bp, Artificial sequence
  sequence is synthesized
cagcctctttatgtcaacgttgccc

SEQ ID NO: 331 - DNA, 20 bp, Artificial sequence
  sequence is synthesized
cttgatgactgccttgcctc

SEQ ID NO: 332 - DNA, 23 bp, Artificial sequence
  sequence is synthesized
ggttacattcaaaggctgttgct

SEQ ID NO: 333 - DNA, 15 bp, Artificial sequence
  sequence is synthesized
tctttagagaagtcc

SEQ ID NO: 334 - DNA, 19 bp, Artificial sequence
  sequence is synthesized
gggagcgtggcacttacct

SEQ ID NO: 335 - DNA, 23 bp, Artificial sequence
  sequence is synthesized
tgcccagttttatcatctcacaa

SEQ ID NO: 336 - DNA, 16 bp, Artificial sequence
  sequence is synthesized
tgtcccttgcttcatt

SEQ ID NO: 337 - DNA, 19 bp, Artificial sequence
  sequence is synthesized
tgcacttggtcctgcgctt

SEQ ID NO: 338 - DNA, 24 bp, Artificial sequence
  sequence is synthesized
gggaatgcaacaactttattgaaa

SEQ ID NO: 339 - DNA, 20 bp, Artificial sequence
  sequence is synthesized
tgtctaagtttcccctttta

SEQ ID NO: 340 - DNA, 25 bp, Artificial sequence
  sequence is synthesized
attgacatgctatgggatagcaaca

SEQ ID NO: 341 - DNA, 22 bp, Artificial sequence
  sequence is synthesized
ctggagatgagagtggtcttct

SEQ ID NO: 342 - DNA, 33 bp, Artificial sequence
  sequence is synthesized
tgtgttttgcaggaggaaaatccacttgctgga

SEQ ID NO: 343 - DNA, 18 bp, Artificial sequence
  sequence is synthesized
ctggcaagcggtcttacc

SEQ ID NO: 344 - DNA, 26 bp, Artificial sequence
  sequence is synthesized
gcaggtaacccatcttttaaccatac

SEQ ID NO: 345 - DNA, 26 bp, Artificial sequence
  sequence is synthesized
aagtgaaggcatttccctcgccggaa

SEQ ID NO: 346 - DNA, 19 bp, Artificial sequence
  sequence is synthesized
agggagtctgtggcatctg

SEQ ID NO: 347 - DNA, 21 bp, Artificial sequence
  sequence is synthesized
ggagtgatatccggactggta

SEQ ID NO: 348 - DNA, 22 bp, Artificial sequence
  sequence is synthesized
aggctcaaaccagacaagcggc

SEQ ID NO: 349 - DNA, 19 bp, Artificial sequence
  sequence is synthesized
aggactggatggtgtaccg

SEQ ID NO: 350 - DNA, 20 bp, Artificial sequence
  sequence is synthesized
ttcagaaccacctcagttgc

SEQ ID NO: 351 - DNA, 26 bp, Artificial sequence
  sequence is synthesized
ccacaaggtatttcaagccaacaacg

SEQ ID NO: 352 - DNA, 22 bp, Artificial sequence
  sequence is synthesized
tcagatcacattacgggagttt

SEQ ID NO: 353 - DNA, 20 bp, Artificial sequence
  sequence is synthesized
cagcttgcagatgaccttgt

SEQ ID NO: 354 - DNA, 23 bp, Artificial sequence
  sequence is synthesized
tcaatgccattatcgcaggcaaa

SEQ ID NO: 355 - DNA, 22 bp, Artificial sequence
  sequence is synthesized
acaatgtccaaacccactcatg

SEQ ID NO: 356 - DNA, 23 bp, Artificial sequence
  sequence is synthesized
gatgtgacaacagcgaggtgtaa

SEQ ID NO: 357 - DNA, 21 bp, Artificial sequence
  sequence is synthesized
tgcatgacggagccgagccat

SEQ ID NO: 358 - DNA, 25 bp, Artificial sequence
  sequence is synthesized
agaagcaaaatactgacagtcagag

SEQ ID NO: 359 - DNA, 20 bp, Artificial sequence
  sequence is synthesized
gagcaatgatcactccgatg

SEQ ID NO: 360 - DNA, 25 bp, Artificial sequence
  sequence is synthesized
ctgcaataagtcctttcttccatgg

SEQ ID NO: 361 - DNA, 18 bp, Artificial sequence
  sequence is synthesized
ctggaggacagggaccac

SEQ ID NO: 362 - DNA, 20 bp, Artificial sequence
  sequence is synthesized
gggaaacccttctctccttt

SEQ ID NO: 363 - DNA, 20 bp, Artificial sequence
  sequence is synthesized
ccaggagggcctgacaaccc

SEQ ID NO: 364 - DNA, 20 bp, Artificial sequence
  sequence is synthesized
gctaccctgagaaaggtgga

SEQ ID NO: 365 - DNA, 21 bp, Artificial sequence
  sequence is synthesized
gggaatccttgtaatcctggt

SEQ ID NO: 366 - DNA, 20 bp, Artificial sequence
  sequence is synthesized
cactggcccaggctgaccac

SEQ ID NO: 367 - DNA, 20 bp, Artificial sequence
  sequence is synthesized
aggaatcccaggagttgatg

SEQ ID NO: 368 - DNA, 20 bp, Artificial sequence
  sequence is synthesized
cctgggatataagggcactg

SEQ ID NO: 369 - DNA, 22 bp, Artificial sequence
  sequence is synthesized
cccaaaggagaaccaggcctcc

SEQ ID NO: 370 - DNA, 20 bp, Artificial sequence
  sequence is synthesized
ctcagcgagagacaggtcaa

SEQ ID NO: 371 - DNA, 22 bp, Artificial sequence
  sequence is synthesized
tttattgctttgagggttctcc

SEQ ID NO: 372 - DNA, 22 bp, Artificial sequence
  sequence is synthesized
tctcctccatttagcgcgtcga

SEQ ID NO: 373 - DNA, 21 bp, Artificial sequence
  sequence is synthesized
aggcctgttttgtgaccaaga

SEQ ID NO: 374 - DNA, 19 bp, Artificial sequence
  sequence is synthesized
gagcacgttgccccattct

SEQ ID NO: 375 - DNA, 15 bp, Artificial sequence
  sequence is synthesized
actgcacccaccact

SEQ ID NO: 376 - DNA, 20 bp, Artificial sequence
  sequence is synthesized
cggaaacggctctacatctt

SEQ ID NO: 377 - DNA, 20 bp, Artificial sequence
  sequence is synthesized
agttcctcggcatcattagg

SEQ ID NO: 378 - DNA, 20 bp, Artificial sequence
  sequence is synthesized
ccagatcccaccgtgggctt

SEQ ID NO: 379 - DNA, 19 bp, Artificial sequence
  sequence is synthesized
accagccaagacccagaaa

SEQ ID NO: 380 - DNA, 21 bp, Artificial sequence
  sequence is synthesized
gcaagtccatagttgttctgc

SEQ ID NO: 381 - DNA, 20 bp, Artificial sequence
  sequence is synthesized
cactgcagggcctcgtccag

SEQ ID NO: 382 - DNA, 21 bp, Artificial sequence
  sequence is synthesized
gaagatctcagtgcagaggct

SEQ ID NO: 383 - DNA, 21 bp, Artificial sequence
  sequence is synthesized
tgaagatcacagcttctttgg

SEQ ID NO: 384 - DNA, 26 bp, Artificial sequence
  sequence is synthesized
cgcgagctatagaagaatcaccagca

SEQ ID NO: 385 - DNA, 20 bp, Artificial sequence
  sequence is synthesized
tacaccagtggcaagtgctc

SEQ ID NO: 386 - DNA, 19 bp, Artificial sequence
  sequence is synthesized
cacacttggcggttctttc

SEQ ID NO: 387 - DNA, 23 bp, Artificial sequence
  sequence is synthesized
cccagcagtcgtctttgtcaccc

SEQ ID NO: 388 - DNA, 19 bp, Artificial sequence
  sequence is synthesized
gacggtggaaacaaggaaa

SEQ ID NO: 389 - DNA, 20 bp, Artificial sequence
  sequence is synthesized
tctctgctgaagactgggaa

SEQ ID NO: 390 - DNA, 27 bp, Artificial sequence
  sequence is synthesized
tccatgcgtgctcatttctcttaatca

SEQ ID NO: 391 - DNA, 17 bp, Artificial sequence
  sequence is synthesized
ggccaacaagcgcatca

SEQ ID NO: 392 - DNA, 20 bp, Artificial sequence
  sequence is synthesized
aaggtgtccgtctccacgat

SEQ ID NO: 393 - DNA, 13 bp, Artificial sequence
  sequence is synthesized
ccttcgcaaagct

SEQ ID NO: 394 - DNA, 19 bp, Artificial sequence
  sequence is synthesized
gctggtcctctgcctccaa

SEQ ID NO: 395 - DNA, 25 bp, Artificial sequence
  sequence is synthesized
tccctcacatgctgtgtaaaattag

SEQ ID NO: 396 - DNA, 18 bp, Artificial sequence
  sequence is synthesized
cccaggtaactgttcagt

SEQ ID NO: 397 - DNA, 21 bp, Artificial sequence
  sequence is synthesized
tctcaacactccaaactgtgc

SEQ ID NO: 398 - DNA, 23 bp, Artificial sequence
  sequence is synthesized
ccttcagattgtagcccggctga

SEQ ID NO: 399 - DNA, 20 bp, Artificial sequence
  sequence is synthesized
tttgatgtcaccggagttgt

SEQ ID NO: 400 - DNA, 18 bp, Artificial sequence
  sequence is synthesized
gcgaaagccctcaatttc

SEQ ID NO: 401 - DNA, 20 bp, Artificial sequence
  sequence is synthesized
tccacggctcaaccactgcc

SEQ ID NO: 402 - DNA, 20 bp, Artificial sequence
  sequence is synthesized
ggagtagagggaaggagcag

SEQ ID NO: 403 - DNA, 20 bp, Artificial sequence
  sequence is synthesized
ctgggttgtgggaaataaca

SEQ ID NO: 404 - DNA, 21 bp, Artificial sequence
  sequence is synthesized
ccgcgtgtcacacccatcatt

SEQ ID NO: 405 - DNA, 20 bp, Artificial sequence
  sequence is synthesized
gccattcctgttccagattc

SEQ ID NO: 406 - DNA, 20 bp, Artificial sequence
  sequence is synthesized
tcagtgggtttccatgtctc

SEQ ID NO: 407 - DNA, 20 bp, Artificial sequence
  sequence is synthesized
tcggctcctccgtttcccag

SEQ ID NO: 408 - DNA, 23 bp, Artificial sequence
  sequence is synthesized
caccatagctaatcttgggacat

SEQ ID NO: 409 - DNA, 20 bp, Artificial sequence
  sequence is synthesized
tgatggtcctgatcgagaaa

SEQ ID NO: 410 - DNA, 22 bp, Artificial sequence
  sequence is synthesized
ccacaacctgcatgaagcgacc

SEQ ID NO: 411 - DNA, 20 bp, Artificial sequence
  sequence is synthesized
cgggaacatactgccatgaa

SEQ ID NO: 412 - DNA, 21 bp, Artificial sequence
  sequence is synthesized
gcaagtgccaccgtttctaca

SEQ ID NO: 413 - DNA, 17 bp, Artificial sequence
  sequence is synthesized
atgactgtgagagcaac

SEQ ID NO: 414 - DNA, 18 bp, Artificial sequence
  sequence is synthesized
cacctgcctggaccagat

SEQ ID NO: 415 - DNA, 20 bp, Artificial sequence
  sequence is synthesized
gtctgtgttgacctcgcagt

SEQ ID NO: 416 - DNA, 20 bp, Artificial sequence
  sequence is synthesized
tctgcatgcccggctacgag

SEQ ID NO: 417 - DNA, 20 bp, Artificial sequence
  sequence is synthesized
tctgaagtgggtgacattcc

SEQ ID NO: 418 - DNA, 20 bp, Artificial sequence
  sequence is synthesized
ctgtgctgttcctcatccag

SEQ ID NO: 419 - DNA, 20 bp, Artificial sequence
  sequence is synthesized
ctcccactgcccgtccacct

SEQ ID NO: 420 - DNA, 21 bp, Artificial sequence
  sequence is synthesized
atccaggttctagcacagacg

SEQ ID NO: 421 - DNA, 20 bp, Artificial sequence
  sequence is synthesized
tgaagcaatccctgcaaata

SEQ ID NO: 422 - DNA, 20 bp, Artificial sequence
  sequence is synthesized
tcctcggttccgaagtggcc

SEQ ID NO: 423 - DNA, 20 bp, Artificial sequence
  sequence is synthesized
gaatccaagcggagagagtc

SEQ ID NO: 424 - DNA, 20 bp, Artificial sequence
  sequence is synthesized
acatcagtgaatgccagtcc

SEQ ID NO: 425 - DNA, 24 bp, Artificial sequence
  sequence is synthesized
tgcagtaaccaacattgatcgccc

SEQ ID NO: 426 - DNA, 22 bp, Artificial sequence
  sequence is synthesized
gatcagcttctcctcaggattc

SEQ ID NO: 427 - DNA, 20 bp, Artificial sequence
  sequence is synthesized
tgtctgggtcccactcatag

SEQ ID NO: 428 - DNA, 22 bp, Artificial sequence
  sequence is synthesized
cccgacagctacacggaatgca

SEQ ID NO: 429 - DNA, 19 bp, Artificial sequence
  sequence is synthesized
gagccaaggagggtgagac

SEQ ID NO: 430 - DNA, 19 bp, Artificial sequence
  sequence is synthesized
ccacagcagtcacagcatt

SEQ ID NO: 431 - DNA, 20 bp, Artificial sequence
  sequence is synthesized
acgacagctgcggcatctcc

SEQ ID NO: 432 - DNA, 21 bp, Artificial sequence
  sequence is synthesized
aggagatctgctctcgtcttg

SEQ ID NO: 433 - DNA, 18 bp, Artificial sequence
  sequence is synthesized
agccatctgacggcaaag

SEQ ID NO: 434 - DNA, 27 bp, Artificial sequence
  sequence is synthesized
ctcatctttcatagcttcgtgttcctt

SEQ ID NO: 435 - DNA, 20 bp, Artificial sequence
  sequence is synthesized
agagactccgagcatgaagg

SEQ ID NO: 436 - DNA, 19 bp, Artificial sequence
  sequence is synthesized
gggcagtggcaagttacag

SEQ ID NO: 437 - DNA, 26 bp, Artificial sequence
  sequence is synthesized
ccacaaggtctgcacttcttgttgtg

SEQ ID NO: 438 - DNA, 22 bp, Artificial sequence
  sequence is synthesized
ttctccatctagcggaacaaca

SEQ ID NO: 439 - DNA, 21 bp, Artificial sequence
  sequence is synthesized
ggtctcatcccatcacaggaa

SEQ ID NO: 440 - DNA, 18 bp, Artificial sequence
  sequence is synthesized
tgacatctgtggtgggat

SEQ ID NO: 441 - DNA, 20 bp, Artificial sequence
  sequence is synthesized
tacttggagagcactggcat

SEQ ID NO: 442 - DNA, 20 bp, Artificial sequence
  sequence is synthesized
ctgtgcagttatgtgggctt

SEQ ID NO: 443 - DNA, 23 bp, Artificial sequence
  sequence is synthesized
tgtgaagcttgccaccagtcctg

SEQ ID NO: 444 - DNA, 20 bp, Artificial sequence
  sequence is synthesized
gcaagcaggagtacgatgag

SEQ ID NO: 445 - DNA, 20 bp, Artificial sequence
  sequence is synthesized
taacagtccgcctagaagca

SEQ ID NO: 446 - DNA, 20 bp, Artificial sequence
  sequence is synthesized
cctccatcgtgcaccgcaag

SEQ ID NO: 447 - DNA, 20 bp, Artificial sequence
  sequence is synthesized
ctcccactcagaacctcctt

SEQ ID NO: 448 - DNA, 20 bp, Artificial sequence
  sequence is synthesized
agcagcaacaggacactgag

SEQ ID NO: 449 - DNA, 18 bp, Artificial sequence
  sequence is synthesized
cccaaagcccagcctggc

SEQ ID NO: 450 - DNA, 20 bp, Artificial sequence
  sequence is synthesized
ctacaagggacaggtgctga

SEQ ID NO: 451 - DNA, 20 bp, Artificial sequence
  sequence is synthesized
gagagaatttgctccaagcc

SEQ ID NO: 452 - DNA, 20 bp, Artificial sequence
  sequence is synthesized
cctgcgcctcagtgcatggt

SEQ ID NO: 453 - DNA, 20 bp, Artificial sequence
  sequence is synthesized
atgctgtgggataacaccaa

SEQ ID NO: 454 - DNA, 20 bp, Artificial sequence
  sequence is synthesized
gtgggttcctggaggtaaga

SEQ ID NO: 455 - DNA, 22 bp, Artificial sequence
  sequence is synthesized
cgagactccactgcctgggaca

SEQ ID NO: 456 - DNA, 22 bp, Artificial sequence
  sequence is synthesized
aaagtcatgttgcaaatggaag

SEQ ID NO: 457 - DNA, 21 bp, Artificial sequence
  sequence is synthesized
aatggaacctccttcttcctc

SEQ ID NO: 458 - DNA, 23 bp, Artificial sequence
  sequence is synthesized
tcttcgcccttcttctttcctgc

SEQ ID NO: 459 - DNA, 20 bp, Artificial sequence
  sequence is synthesized
ctcaggtggagtgtgctgac

SEQ ID NO: 460 - DNA, 20 bp, Artificial sequence
  sequence is synthesized
ttgccatctcctgtatggtc

SEQ ID NO: 461 - DNA, 22 bp, Artificial sequence
  sequence is synthesized
ctgaatcggccctgtcttgctg

SEQ ID NO: 462 - DNA, 20 bp, Artificial sequence
  sequence is synthesized
ctactgggctgtgaagtgga

SEQ ID NO: 463 - DNA, 20 bp, Artificial sequence
  sequence is synthesized
cacactcatccactgggttc

SEQ ID NO: 464 - DNA, 21 bp, Artificial sequence
  sequence is synthesized
cagctggaccaaccacaccca

SEQ ID NO: 465 - DNA, 20 bp, Artificial sequence
  sequence is synthesized
gcattatcctgcccagctat

SEQ ID NO: 466 - DNA, 20 bp, Artificial sequence
  sequence is synthesized
gatcgtcccttccctatcac

SEQ ID NO: 467 - DNA, 26 bp, Artificial sequence
  sequence is synthesized
tccctcgaacacgatctgatactcca

SEQ ID NO: 468 - DNA, 20 bp, Artificial sequence
  sequence is synthesized
cggacgtgaagttcaacaga

SEQ ID NO: 469 - DNA, 20 bp, Artificial sequence
  sequence is synthesized
acgcgcatacttctccatct

SEQ ID NO: 470 - DNA, 23 bp, Artificial sequence
  sequence is synthesized
cgcagctcatcaagtggttcagc

SEQ ID NO: 471 - DNA, 21 bp, Artificial sequence
  sequence is synthesized
cctggaagtaccagccactac

SEQ ID NO: 472 - DNA, 22 bp, Artificial sequence
  sequence is synthesized
gggtagctgtaaagttgaccgt

SEQ ID NO: 473 - DNA, 24 bp, Artificial sequence
  sequence is synthesized
accacaccagccatctcagagacc

SEQ ID NO: 474 - DNA, 23 bp, Artificial sequence
  sequence is synthesized
caggtctctacatctgcatgaac

SEQ ID NO: 475 - DNA, 20 bp, Artificial sequence
  sequence is synthesized
aatacgcagtccttgccttt

SEQ ID NO: 476 - DNA, 24 bp, Artificial sequence
  sequence is synthesized
aagctaattgccaagagcaacggc

SEQ ID NO: 477 - DNA, 18 bp, Artificial sequence
  sequence is synthesized
ctgcctgctgttgcactt

SEQ ID NO: 478 - DNA, 24 bp, Artificial sequence
  sequence is synthesized
ttaggtgaggactgaacagttacc

SEQ ID NO: 479 - DNA, 22 bp, Artificial sequence
  sequence is synthesized
ctggttctctgcctccaagccc

SEQ ID NO: 480 - DNA, 21 bp, Artificial sequence
  sequence is synthesized
acatccagagcttgagtgtga

SEQ ID NO: 481 - DNA, 19 bp, Artificial sequence
  sequence is synthesized
gcccttgagagtggctatg

SEQ ID NO: 482 - DNA, 20 bp, Artificial sequence
  sequence is synthesized
cccactgcgcccagacagaa

SEQ ID NO: 483 - DNA, 20 bp, Artificial sequence
  sequence is synthesized
gcccacgtcaaggagtattt

SEQ ID NO: 484 - DNA, 20 bp, Artificial sequence
  sequence is synthesized
tcgagtgacaaacacgactg

SEQ ID NO: 485 - DNA, 23 bp, Artificial sequence
  sequence is synthesized
caccagcagcaagtgctccaatc

SEQ ID NO: 486 - DNA, 21 bp, Artificial sequence
  sequence is synthesized
cagaccctcacactcagatca

SEQ ID NO: 487 - DNA, 21 bp, Artificial sequence
  sequence is synthesized
agtacctggagttgagggtga

SEQ ID NO: 488 - DNA, 19 bp, Artificial sequence
  sequence is synthesized
gtctcgggaggacagaagg

SEQ ID NO: 489 - DNA, 26 bp, Artificial sequence
  sequence is synthesized
cacccactttgaccaacttcaggatg

SEQ ID NO: 490 - DNA, 21 bp, Artificial sequence
  sequence is synthesized
ccatgaggtccttcagttgag

SEQ ID NO: 491 - DNA, 20 bp, Artificial sequence
  sequence is synthesized
tcctgcgtttcctctacaca

SEQ ID NO: 492 - DNA, 21 bp, Artificial sequence
  sequence is synthesized
cctcgtggtgttccagagcca

SEQ ID NO: 493 - DNA, 20 bp, Artificial sequence
  sequence is synthesized
cacgaaggatgctgataacg

SEQ ID NO: 494 - DNA, 20 bp, Artificial sequence
  sequence is synthesized
accaccaacctcctgttagc

SEQ ID NO: 495 - DNA, 26 bp, Artificial sequence
  sequence is synthesized
caactgtatgtgcaaatgcgctctca

SEQ ID NO: 496 - DNA, 20 bp, Artificial sequence
  sequence is synthesized
cacaaaggattcggacaatg

SEQ ID NO: 497 - DNA, 20 bp, Artificial sequence
  sequence is synthesized
aagttggaaggaccacatgc

SEQ ID NO: 498 - DNA, 23 bp, Artificial sequence
  sequence is synthesized
caaaccaccagcctcctgagagc

SEQ ID NO: 499 - DNA, 20 bp, Artificial sequence
  sequence is synthesized
cttcagcgtggaagatgcta

SEQ ID NO: 500 - DNA, 20 bp, Artificial sequence
  sequence is synthesized
tggcaggagacatagagtcg

SEQ ID NO: 501 - DNA, 20 bp, Artificial sequence
  sequence is synthesized
cgacagctgccacggaggac

SEQ ID NO: 502 - DNA, 19 bp, Artificial sequence
  sequence is synthesized
ccatgccgtctgctaacat

SEQ ID NO: 503 - DNA, 20 bp, Artificial sequence
  sequence is synthesized
gatatttccggagcccatta

SEQ ID NO: 504 - DNA, 25 bp, Artificial sequence
  sequence is synthesized
cagatcttcgttcttgaagctccgg

SEQ ID NO: 505 - DNA, 19 bp, Artificial sequence
  sequence is synthesized
acgtcagaaggtcgcttca

SEQ ID NO: 506 - DNA, 20 bp, Artificial sequence
  sequence is synthesized
acatgaggagtgaggtgtgc

SEQ ID NO: 507 - DNA, 23 bp, Artificial sequence
  sequence is synthesized
tgttcgagagagcaccacctgca

SEQ ID NO: 508 - DNA, 19 bp, Artificial sequence
  sequence is synthesized
tgtagagcgagtgttgcca

SEQ ID NO: 509 - DNA, 21 bp, Artificial sequence
  sequence is synthesized
ccagaacccacttcttcagag

SEQ ID NO: 510 - DNA, 27 bp, Artificial sequence
  sequence is synthesized
tgtatatactcacactgatcggttcca

SEQ ID NO: 511 - DNA, 20 bp, Artificial sequence
  sequence is synthesized
atgcctgggaagtatcctca

SEQ ID NO: 512 - DNA, 20 bp, Artificial sequence
  sequence is synthesized
ggcttctcactgtgtaaccg

SEQ ID NO: 513 - DNA, 27 bp, Artificial sequence
  sequence is synthesized
tggcaccttctctcctaagctcttgtc

SEQ ID NO: 514 - DNA, 20 bp, Artificial sequence
  sequence is synthesized
acatagcctgtgagccttcc

SEQ ID NO: 515 - DNA, 20 bp, Artificial sequence
  sequence is synthesized
cttgacagggttcccatcat

SEQ ID NO: 516 - DNA, 23 bp, Artificial sequence
  sequence is synthesized
cgtggccatctctgcagaagaca

SEQ ID NO: 517 - DNA, 20 bp, Artificial sequence
  sequence is synthesized
gtccaacaagacgtccagag

SEQ ID NO: 518 - DNA, 20 bp, Artificial sequence
  sequence is synthesized
cggtgctagaacctggattt

SEQ ID NO: 519 - DNA, 26 bp, Artificial sequence
  sequence is synthesized
tcaacaacaagtccctttgtgaagcc

SEQ ID NO: 520 - DNA, 21 bp, Artificial sequence
  sequence is synthesized
ttggacaagatgcaagttctg

SEQ ID NO: 521 - DNA, 21 bp, Artificial sequence
  sequence is synthesized
tctcccatttgtaccagcttc

SEQ ID NO: 522 - DNA, 21 bp, Artificial sequence
  sequence is synthesized
tcagccaggaatcacggtcca

SEQ ID NO: 523 - DNA, 20 bp, Artificial sequence
  sequence is synthesized
cactgcatggacaagatcaa

SEQ ID NO: 524 - DNA, 20 bp, Artificial sequence
  sequence is synthesized
tcatccacatcatactggca

SEQ ID NO: 525 - DNA, 20 bp, Artificial sequence
  sequence is synthesized
cccaaaggcttcaacgggca

SEQ ID NO: 526 - DNA, 20 bp, Artificial sequence
  sequence is synthesized
cacgaaggatgctgataacg

SEQ ID NO: 527 - DNA, 20 bp, Artificial sequence
  sequence is synthesized
accaccaacctcctgttagc

SEQ ID NO: 528 - DNA, 26 bp, Artificial sequence
  sequence is synthesized
caactgtatgtgcaaatgcgctctca

SEQ ID NO: 529 - DNA, 20 bp, Artificial sequence
  sequence is synthesized
ttgcaatgctgggtatgaag

SEQ ID NO: 530 - DNA, 20 bp, Artificial sequence
  sequence is synthesized
agccttgtagaagcctggtc

SEQ ID NO: 531 - DNA, 27 bp, Artificial sequence
  sequence is synthesized
aacgaggtttcatatgccaagcttgtc

SEQ ID NO: 532 - DNA, 26 bp, Artificial sequence
  sequence is synthesized
cagaattcataatgaataacacagga

SEQ ID NO: 533 - DNA, 17 bp, Artificial sequence
  sequence is synthesized
cagccagccagatttgg

SEQ ID NO: 534 - DNA, 25 bp, Artificial sequence
  sequence is synthesized
gaatggaggttgggaagatggcttc

SEQ ID NO: 535 - DNA, 20 bp, Artificial sequence
  sequence is synthesized
ttgccacgtactatggtgct

SEQ ID NO: 536 - DNA, 20 bp, Artificial sequence
  sequence is synthesized
ccataacaagccagagttgg

SEQ ID NO: 537 - DNA, 25 bp, Artificial sequence
  sequence is synthesized
tcatcatgtcctggagggctcttct

SEQ ID NO: 538 - DNA, 20 bp, Artificial sequence
  sequence is synthesized
tgggaagtctgctgtctcaa

SEQ ID NO: 539 - DNA, 20 bp, Artificial sequence
  sequence is synthesized
aatagctacgatggctgcaa

SEQ ID NO: 540 - DNA, 25 bp, Artificial sequence
  sequence is synthesized
cacagccacagaatgtaccaatggg

SEQ ID NO: 541 - DNA, 18 bp, Artificial sequence
  sequence is synthesized
ccctgcgtacattgatgc

SEQ ID NO: 542 - DNA, 21 bp, Artificial sequence
  sequence is synthesized
gctctcatccttgtcagaggt

SEQ ID NO: 543 - DNA, 20 bp, Artificial sequence
  sequence is synthesized
acagctccgtgcacacgcct

SEQ ID NO: 544 - DNA, 20 bp, Artificial sequence
  sequence is synthesized
gaggcatgcagtatctggag

SEQ ID NO: 545 - DNA, 19 bp, Artificial sequence
  sequence is synthesized
ctcggtcaccagcacattt

SEQ ID NO: 546 - DNA, 20 bp, Artificial sequence
  sequence is synthesized
ctcggaagtgcatccaccgg

SEQ ID NO: 547 - DNA, 20 bp, Artificial sequence
  sequence is synthesized
gtacgtcagcctggagagaa

SEQ ID NO: 548 - DNA, 22 bp, Artificial sequence
  sequence is synthesized
attggtaacattgccattgaac

SEQ ID NO: 549 - DNA, 25 bp, Artificial sequence
  sequence is synthesized
aaagtggcgctggatcaacaactct

SEQ ID NO: 550 - DNA, 21 bp, Artificial sequence
  sequence is synthesized
gaatgaattcaaccaaatcgc

SEQ ID NO: 551 - DNA, 20 bp, Artificial sequence
  sequence is synthesized
caggagagcacttgggagtt

SEQ ID NO: 552 - DNA, 24 bp, Artificial sequence
  sequence is synthesized
tcccaccacacagagagaggatgc

SEQ ID NO: 553 - DNA, 20 bp, Artificial sequence
  sequence is synthesized
ttgtccacccaactatgtcc

SEQ ID NO: 554 - DNA, 19 bp, Artificial sequence
  sequence is synthesized
cgtgatatcctggcatgtg

SEQ ID NO: 555 - DNA, 21 bp, Artificial sequence
  sequence is synthesized
tgcgctcgcacttcgtttctg

SEQ ID NO: 556 - DNA, 20 bp, Artificial sequence
  sequence is synthesized
agccttacctcaccacttgc

SEQ ID NO: 557 - DNA, 20 bp, Artificial sequence
  sequence is synthesized
ataggcagtccggtagatgg

SEQ ID NO: 558 - DNA, 20 bp, Artificial sequence
  sequence is synthesized
cggacacagagcctgcagca

SEQ ID NO: 559 - DNA, 20 bp, Artificial sequence
  sequence is synthesized
attcccatgagtgcttggat

SEQ ID NO: 560 - DNA, 21 bp, Artificial sequence
  sequence is synthesized
cacagtgctctcctgttgtgt

SEQ ID NO: 561 - DNA, 20 bp, Artificial sequence
  sequence is synthesized
ctgtctgcactgccagcgga

SEQ ID NO: 562 - DNA, 21 bp, Artificial sequence
  sequence is synthesized
gcagatcacttctaccacacg

SEQ ID NO: 563 - DNA, 20 bp, Artificial sequence
  sequence is synthesized
ctggccactgtccttattca

SEQ ID NO: 564 - DNA, 24 bp, Artificial sequence
  sequence is synthesized
tgatataacaccatccctccgcca

SEQ ID NO: 565 - DNA, 20 bp, Artificial sequence
  sequence is synthesized
ggcaataaaccgaggacttc

SEQ ID NO: 566 - DNA, 20 bp, Artificial sequence
  sequence is synthesized
tcaagtgctgctctgtgatg

SEQ ID NO: 567 - DNA, 22 bp, Artificial sequence
  sequence is synthesized
cgtgctacggaccctgctgaaa

SEQ ID NO: 568 - DNA, 20 bp, Artificial sequence
  sequence is synthesized
gagacaaggtggcagcctat

SEQ ID NO: 569 - DNA, 20 bp, Artificial sequence
  sequence is synthesized
tgttattgcccgtaatctgg

SEQ ID NO: 570 - DNA, 20 bp, Artificial sequence
  sequence is synthesized
cgggaagctgcggtacaccc

SEQ ID NO: 571 - DNA, 20 bp, Artificial sequence
  sequence is synthesized
gctggaacatggaattaccc

SEQ ID NO: 572 - DNA, 19 bp, Artificial sequence
  sequence is synthesized
gtactgcgggtggaacatt

SEQ ID NO: 573 - DNA, 20 bp, Artificial sequence
  sequence is synthesized
accagcaaccctgccagcaa

SEQ ID NO: 574 - DNA, 20 bp, Artificial sequence
  sequence is synthesized
gtccatgccactaagcatgt

SEQ ID NO: 575 - DNA, 20 bp, Artificial sequence
  sequence is synthesized
acagcttcctcgatgcttct

SEQ ID NO: 576 - DNA, 20 bp, Artificial sequence
  sequence is synthesized
ccctgcccattcggaggaag